(19) United States
(12) Patent Application Publication
(10) Pub. No.: US 2026/0139307 A1
(43) Pub. Date: May 21, 2026

(54) METHODS AND COMPOSITIONS FOR MOLECULAR INTERACTION MAPPING USING TRANSPOSASE

(71) Applicant: New York Genome Center, Inc., New York, NY (US)

(72) Inventors: Ivan Raimondi, New York, NY (US); Silas Maniatis, Brooklyn, NY (US); Peter Smibert, New York, NY (US)

(21) Appl. No.: 18/707,383

(22) PCT Filed: Nov. 5, 2022
(86) PCT No.: PCT/US2022/079354

§ 371 Date: May 3, 2024

Related U.S. Application Data
(60) Provisional application No. 63/276,533, filed on Nov. 5, 2021.

(51) Int. Cl. C12Q 1/6869; C07K 16/44; C12N 9/12; C12N 15/10; C12Q 1/6806; C12Q 1/686; G01N 1/30

(52) U.S. Cl. CPC C12Q 1/6869; C07K 16/44; C12N 9/1241; C12N 15/1096; C12Q 1/6806; C12Q 1/686; G01N 1/30; C07K 2317/569; C07K 2319/20; C07K 2319/80; C12Y 207/07

(57) ABSTRACT

Compositions, methods, and kits for performing multiplexed, spatially resolved, or single-cell chromatin analysis are provided.

### STATEMENT OF GOVERNMENT SUPPORT

This invention was made with government support under HG011014, NS116350, NS118570, and NS118183 awarded by the National Institutes of Health. The government has certain rights in the invention.

### BACKGROUND OF THE INVENTION

Interactions between proteins and DNA determine the 3-dimensional conformation of genomic DNA within the nucleus, thereby controlling the accessibility of genomic DNA for interactions with other factors, and ultimately the transcriptional activity of genes. Such DNA-protein interactions can include DNA coiling around histones to form nucleosomes and chromatin, binding of transcription factors to promoters, etc. By understanding the composition and arrangement of DNA-protein assemblies across the genome, it is possible to deduce the structure and activity of gene regulation networks. Technologies such as ChIPseq, ATACseq, CUT & Tag, and others can provide such information from bulk tissue samples, single cells, or single nuclei.

In ATAC seq, a transposase (typically Tn5) is used to randomly insert DNA adapters into genomic DNA. The inserted adapters harbor sequences used in downstream library prep, such that genomic DNA sequences flanked by inserted adapters can be sequenced, and the site of adapter insertion can thus be inferred. As Tn5 is unable to insert adapters into nucleosomal DNA, only regions of “open” or accessible, non-nucleosomal DNA are sequenced. In this way, the accessibility of DNA can be mapped. In single cells, ATACseq can be combined with other data modalities, yielding simultaneous measures of chromatin accessibility, RNA abundance, and proteins (ASAPseq, DOGMAseq) from each cell.

In CUT & Tag, a transposase: protein-A fusion protein (pA-Tn5) is loaded with mosaic end DNA adapters, and immobilized by binding of the protein-A domain to antibodies specific to an epitope of interest. After extensive washing to remove transposase molecules not tethered via the antibody, the transposase enzyme is activated by addition of Magnesium or other divalent cation, and inserts its adapters in nearby DNA. The goal of the method is to detect only the interaction mediated by the antibody, and not those mediated by the non-specific affinity of the transposon for DNA. To limit non-specific tagmentation at sites not associated with target epitopes, the conditions used in both single cell and spatial CUT & Tag involve non-physiologically high salt concentrations, which has the effect of causing non-nucleosomal DNA to assume a less accessible state, and preventing the transposase from binding genomic DNA. Such conditions can lead to loss of physiological DNA-protein interactions, including those involved in transcription factor binding. In nucleosomes, DNA is wrapped around histones, thereby reducing the impact of such effects for CUT & Tag against histones. High salt conditions can also distort tissue morphology.

Multiplexing of targets in a single CUT & Tag experiment is constrained by the use of pATn5 fusion transposase to immobilize the transposase at the target proteins via binding to primary and secondary antibodies. Given the non-specificity of proteinA in recognizing IgG, substantial data loss occurs through swapping of pA-Tn5 between target protein bound antibodies. Some success in overcoming such limitations inherent to proteinA mediated immobilization of transposomes has been achieved with a technique termed ‘MulTItag’ However, MulTItag has substantial drawbacks. These draw backs include complex reagent preparation steps, in which transposomes are tethered to DNA oligonucleotide conjugated antibodies via ligation of the antibody's oligonucleotide to the DNA adapter already loaded to the transposome. Further, this process must occur within 24 hours prior to reagent use, and must be conducted anew each time the experiment is run within 24 hours prior to use. Critically, each antibody used must be sequentially applied to the sample, dramatically limiting throughput. Yet, MulTItag still does not overcome the need for high salt concentrations to prevent non-specific tagmentation.

To understand how gene regulation networks in each cell of an intact tissue interact and produce coordinated activities, information regarding the spatial location of each DNA-protein interaction observation must also be captured. Recently, methods for spatially resolved ATACseq (measures chromatin accessibility) and CUT & Tag (identifies sites of protein binding to DNA or epigenetic marks) via deterministic DNA barcoding have been demonstrated. However, these techniques rely on attaching multiple complex microfluidic devices to tissue sections and multiple rounds of reagent pumping through these devices. Many, if not most, labs do not have the capability to fabricate such devices, and do not have equipment for precision pumping of reagents through the devices. Moreover, these methods are prone to failure due to microfluidic device fabrication errors, tissue disruption during attachment and removal of the devices, and the combinatorial barcoding chemistry they employ to encode a spatial coordinate. Further, the data generated from these methods is sparse, highly variable, and prone to data loss from large tissue regions due to the complexity of the microfluidic devices and the spatial-barcoding chemistry.

Recently, several methods for spatially resolved transcriptome profiling (SRT) have been developed. The most mature and widely used methods for SRT involve hybridization of mRNA onto DNA oligonucleotide probes that harbor spatial barcode and unique molecular identifier (UMI) sequences. Captured mRNA is then reverse transcribed (RT), with the capture probe functioning as a primer to initiate the RT reaction. The result is a cDNA library in which each cDNA molecule incorporates a spatial barcode, UMI, and mRNA derived sequence. As the spatial barcode sequence can be tied to a spatial coordinate, and the UMI encodes unique capture events, such methods are spatially resolved and quantitative. Examples of such methods are “Spatial Transcriptomics”, 10× Genomics Visium, seq-SCOPE, and STEREOseq, PIXELseq. One could conceive of using these methods to capture genomic DNA in situ. However, these methods are generally low sensitivity, reliably quantifying only relatively well-expressed mRNAs. With only two copies of any genomic DNA region present per cell in diploid organisms, these methods are not able to capture enough material from genomic DNA to generate accurate maps of DNA-protein interactions across the whole genome. Further, commercially available methods such as 10× Genomics Visium rely on poly(A) based capture, thereby precluding capture of most native DNA sequences.

What is needed are techniques to map chromatin accessibility, or sites of DNA-protein interactions for multiple proteins simultaneously with single cell, single nuclear, or spatial resolution.

### SUMMARY OF THE INVENTION

Provided herein, in a first aspect, is a fusion protein comprising a transposase and a ligand that binds a target epitope. In certain embodiments, the ligand that binds a target epitope is an antibody or fragment thereof. In certain embodiments, the antibody or fragment thereof is a single domain antibody. In certain embodiments, the single domain antibody is a nanobody. In other embodiments, the ligand that binds a target epitope is a G4 binding protein. Also provided are nucleic acids encoding the fusion proteins described herein.

In certain embodiments, the fusion protein is loaded with mosaic-end DNA sequence (MEDS) adapters that comprises one or more of a) a barcode sequence that identifies the target epitope of the ligand; b) a unique molecular identifier (UMI); c) a capture compatible sequence: d) a PCR handle; and e) a sequencing adapter.

In another aspect, a composition is provided that includes a plurality of sets of the complexes described herein, each set of complexes comprising a different ligand that binds a different target epitope. In some embodiments, the different target epitope is on the same target. In other embodiments, the different target epitope is on a different target. In certain embodiments, the composition includes, 10, 50, 100 or more complexes.

In another aspect, a complex or composition is provided that includes a transposase fusion protein as described herein, further comprising a double stranded DNA oligonucleotide having a sequence that is specific to the DNA sequence to which the transposase preferentially binds, wherein the T residues in the oligonucleotide are replaced with U residues.

In another aspect, a method for analyzing molecular interactions is provided. The method includes a) incubating i) a fusion protein comprising a transposase that preferentially binds to a DNA sequence, a ligand, and a mosaic-end DNA adapter; and ii) a double stranded DNA oligonucleotide having a sequence that is specific to the DNA sequence to which the transposase preferentially binds, wherein the T residues in the oligonucleotide are replaced with U residues, wherein the double stranded DNA oligonucleotide binds the transposase, thereby preventing the transposase-ligand complex from binding DNA, and preventing tagmentation from occurring:
b) incubating a sample comprising genomic DNA that comprises chromatin with a primary antibody directed to a target epitope in the chromatin, and said antibody binds said epitope if it is present in the sample;
c) incubating the complex of A with the complex of B, wherein the ligand of the fusion protein binds the primary antibody;
d) degrading or displacing the double stranded DNA oligonucleotide; and
e) activating tagmentation, thereby generating genomic DNA which has been tagmented.

In certain embodiments, the method includes performing in vitro transcription comprising contacting and incubating the tagmented DNA of E with poly A polymerase, thereby generating polyadenylated RNAs that comprise the sequence of the tagmentation fragment; performing reverse transcription to generate DNA; and sequencing DNA.

In certain embodiments, the DNA oligo is degraded by incubating the complex of C with a USER enzyme cocktail to cleave the U residues in the DNA oligonucleotide, thereby removing the blocking double stranded DNA oligonucleotide. In other embodiments, the DNA oligo is displaced by addition of 50 to 150 nM NaCl solution. In certain embodiments, the fusion protein comprises a nanobody-transposase fusion. In certain embodiments, the method includes capturing the tagmented sequences using a capture sequence; performing PCR; and/or performing sequencing.

In another aspect, a multiplexed in vitro method for analyzing molecular interactions is provided. The method includes a) incubating a sample comprising genomic DNA that comprises chromatin with a plurality of primary antibodies, each primary antibody directed to a different target epitope in the chromatin, wherein each antibody binds to the target epitope if it is present in the sample; b) incubating the complex of a) with a composition comprising plurality of fusion proteins, each fusion protein comprising a different nanobody and a transposase that preferentially binds to a DNA sequence, and mosaic-end DNA (MEDS) adapters, wherein each different nanobody binds a different primary antibody; and c) activating tagmentation, thereby generating genomic DNA which has been tagmented. In certain embodiments, the MEDS comprise one or more of: a) a barcode sequence that identifies the target epitope; b) a unique molecular identifier (UMI); c) capture compatible sequence; d) PCR handle. In certain embodiments, the method includes capturing the tagmented sequences using a capture sequence; performing PCR; and/or performing sequencing.

In another aspect, an in vitro method of spatially resolved whole genome sequencing is provided. The method includes a) sectioning a tissue sample onto a substrate comprising substrate oligonucleotides comprising a capture sequence; b) fixing the tissue and performing imaging to determine morphology and/or orientation of the tissue; c) permeabilizing the tissue; d) subjecting the tissue to tagmentation using a transposase loaded with MEDS that comprise T7 RNA polymerase promoter, a capture compatible sequence, and a sequence encoding a poly(A) tail; e) performing in vitro transcription to result in IVT-derived RNA; f) capturing the IVT-derived RNA; and g) generating cDNA from the IVT-derived RNA using fluorescently labeled dNTPs to generate a fluorescent signal wherever cDNA has been captured.

In another aspect, a spatially resolved method for analyzing molecular interactions is provided comprising a) sectioning a tissue sample onto a substrate comprising substrate oligonucleotides comprising a capture sequence; b) fixing the tissue and performing imaging to determine morphology and/or orientation of the tissue; c) permeabilizing the tissue; d) subjecting the tissue to tagmentation using a transposase loaded with MEDS that comprise T7 RNA polymerase promoter, optionally a target barcode, a capture compatible sequence, a sequence encoding a poly(A) tail, and a PCR handle, which is optionally a sequence adapter; e) performing in vitro transcription to result in IVT-derived RNA; f) capturing the IVT-derived RNA; and g) generating cDNA from the IVT-derived RNA using fluorescently labeled dNTPs to generate a fluorescent signal wherever cDNA has been captured. In certain embodiments, the method includes i) partitioning the nuclei into beads; ii) barcoding tagmented DNA; iii) generating sequencing library; and/or iv) performing single cell sequencing.

In yet another aspect, a spatially resolved method for analyzing molecular interactions is provided. The method includes a) incubating i) a fusion protein comprising a transposase that preferentially binds to a DNA sequence, a ligand, and mosaic-end DNA adapters that comprise T7 RNA polymerase promoter, optionally a target barcode, a capture compatible sequence, a sequence encoding a poly(A) tail, and a PCR handle, which is optionally a sequence adapter; and ii) a double stranded DNA oligonucleotide having a sequence that is specific to the DNA sequence to which the transposase preferentially binds, wherein the T residues in the oligonucleotide are replaced with U residues, wherein the double stranded DNA oligonucleotide binds the transposase, thereby preventing the transposase-ligand complex from binding DNA, and preventing tagmentation from occurring; b) sectioning a tissue sample onto a substrate comprising substrate oligonucleotides comprising a capture sequence; c) fixing the tissue and performing imaging to determine morphology and/or orientation of the tissue; d) permeabilizing the tissue: e) incubating the tissue with a primary antibody directed to a target epitope in the chromatin, wherein said antibody binds said epitope if it is present in the sample; f) incubating the complex of a) with the tissue sample, wherein the ligand of the fusion protein binds the primary antibody; g) degrading or displacing the double stranded DNA oligonucleotide; and e) activating tagmentation, thereby generating genomic DNA which has been tagmented. In certain embodiments, the method includes performing in vitro transcription to result in IVT-derived RNA; capturing the IVT-derived RNA; and generating cDNA from the IVT-derived RNA using fluorescently labeled dNTPs to generate a fluorescent signal wherever cDNA has been captured. In certain embodiments, the method includes i) partitioning the nuclei into beads; ii) barcoding tagmented DNA; iii) generating sequencing library; and/or iv) performing single cell sequencing.

In another aspect, a spatially resolved method for analyzing molecular interactions is provided. The method includes a) sectioning a tissue sample onto a substrate comprising substrate oligonucleotides comprising a capture sequence; b) fixing the tissue and performing imaging to determine morphology and/or orientation of the tissue; c) permeabilizing the tissue; d) incubating the tissue with a plurality of primary antibodies, each primary antibody directed to a different target epitope in the chromatin, wherein each antibody binds to the target epitope if it is present in the sample; e) incubating the tissue with a composition comprising plurality of fusion proteins, each fusion protein comprising a different nanobody and a transposase that preferentially binds to a DNA sequence, and mosaic-end DNA (MEDS) adapters that comprise T7 RNA polymerase promoter, optionally a target barcode, a capture compatible sequence, a sequence encoding a poly(A) tail, and a PCR handle, which is optionally a sequence adapter, wherein each different nanobody binds a different primary antibody; and f) activating tagmentation, thereby generating genomic DNA which has been tagmented. In certain embodiments, the method includes performing in vitro transcription to result in IVT-derived RNA; capturing the IVT-derived RNA; and generating cDNA from the IVT-derived RNA using fluorescently labeled dNTPs to generate a fluorescent signal wherever cDNA has been captured. In certain embodiments, the method includes i) partitioning the nuclei into beads; ii) barcoding tagmented DNA; iii) generating sequencing library; and/or iv) performing single cell sequencing.

Other aspects and advantages of these compositions and methods are described further in the following detailed description of the preferred embodiments thereof.

### BRIEF DESCRIPTION OF THE DRAWINGS

FIG. 1 provides a schematic of the prior art high salt Cleavage Under Target and Tagmentation (CUT & Tag) procedure.

FIG. 2 provides a schematic of an embodiment of the invention of a low salt CUT & Tag procedure as described herein.

FIG. 3 demonstrates proof of concept of the low salt CUT & Tag strategy in vitro. Blocked Tn5 is not able to tagment lambda genomic DNA once it is activated by adding Mg2+ (lane 3). The Genomic DNA is intact running as a discrete band comparable to non-activated Tn5 (lane 1). Once the blocker oligo is removed by USER enzymes treatment, Tn5 can digest genomic DNA (lane 4) with the same yield of unblocked Tn5.

FIG. 4 demonstrates blocked Tn5 does not bind open chromatin in K562 cells. An ATAC experiment was performed on the human cell line K562. The blocked Tn5 cannot bind open chromatin regions if blocked in low salt conditions (lane 3). When the blocker is removed, we made the Tn5 competent again (lane 1).

FIG. 5 provides a comparison between standard CUT & Tag (high salt), standard CUT & Tag (low salt), Blocker strategy, and ATAC-seq. CUT & Tag without blocker in low salt conditions (C & T 150 mM NaCl) results in the presence of contaminant nonspecific peaks in correspondence of open chromatin regions (ATAC). These contaminant peaks are not present in the standard CUT & Tag protocol with high salt concentration (C & T 300 mM NaCl). Our blocking strategy results in a signal perfectly overlapping the standard high salt protocol (IsC & T).

FIG. 6 demonstrates that, unlike the standard high salt protocol, our blocking strategy allow us to map proteins that would be displaced from the chromatin by the high salt concentrations. Very low signal was observed in corresponding CTCF binding sites using standard high salt CUT & Tag (CnT HS). Using the blocking strategy described herein, we were able to profile CTCF binding in K562 cells (IsC & T). Our results match the reference data obtained with ChIP-seq deposited in the ENCODE consortium (Encode CTCF). Motif enrichment analysis on the peaks identified by the blocked CUT & Tag confirmed profiling CTCF binding sites.

FIG. 7 demonstrates low salt CUT & Tag on transcription factors (TFs). Transcription factors known to be bound to DNA with lower affinity, such as GATA1 and TALI, were profiled. The results match the reference data obtained with ChIP-seq deposited in the ENCODE consortium. Motif enrichment analysis on the peaks identified by the blocked CUT & Tag confirmed profiling GATA1 binding sites.

FIG. 8 demonstrates our blocker strategy allows us to profile DNA binding proteins in single cell by using 10× chromium workflow. With our strategy we were able to profile CTCF binding in K562 and THP1 cells. Our results match the reference data obtained with ChIP-seq deposited in the ENCODE consortium. Motif enrichment analysis on the peaks identified by the blocked CUT & Tag confirmed profiling CTCF binding sites.

FIG. 9A-FIG. 9B demonstrate antibody-free IsCUT & Tag. (FIG. 9A) By fusing Tn5 with a peptide able to recognize G-quadruplex we were able to identify the DNA secondary structure in the genome. (FIG. 9B) Our results match the reference data obtained with ChIP-seq by using an antibody able to recognize G-quad structures followed by immunoprecipitation. Motif enrichment analysis on the peaks identified by the blocked CUT & Tag confirmed profiling GATA1 binding sites.

FIG. 10 is a diagram demonstrating a Multiplexed NTT-seq (Nanobody Tethered Tn5) scheme as described herein.

FIG. 11 shows two gel images showing the results of high salt CUT & Tag with 4 different antibodies and 4 different nanobody-Tn5 fusions to assess the specificity of our fusion proteins. CUT & Tag library is shown only when the antibody matches the nanobody-Tn5. Demonstrating no cross reactivity of our proteins.

FIG. 12A-FIG. 12J show bulk-cell NTT-seq enables simultaneous profiling of multiple chromatin marks. (FIG. 12A) Schematic representation of nanobody-Tn5 fusion proteins loaded with barcoded DNA adaptors. (FIG. 12B) Overview of the NTT-seq protocol. Nuclei are extracted from cells and stained with a mixture of IgG primary antibodies for targets of interest. Nanobody-Tn5 fusion proteins are then added and tagment the genomic DNA surrounding primary antibody binding sites. Released DNA fragments are amplified by PCR to obtain a sequencing library harboring barcode sequences specific for each nb-Tn5 protein used. (FIG. 12C) Genome browser tracks for a representative region of the human genome. NTT-seq was performed on PBMCs for H3K27me3 alone, H3K27ac alone, or for both together in a multiplexed experiment. Sequencing data were normalized as bins per million mapped reads (BPM). (FIG. 12D) Heatmap displaying coverage within 33,205 H3K27ac peaks identified using MACS2, for multiplexed (multi) and non-multiplexed (mono) NTT-seq PBMC experiments. (FIG. 12E) As for FIG. 12D, for 67,459 H3K27me3 peaks. (FIG. 12F) Fraction of reads in H3K27ac peaks for multiplexed and non-multiplexed NTT-seq PBMC datasets. (FIG. 12G) As for FIG. 12F, for H3K27me3 peaks. (FIG. 12H) Genome browser tracks for a representative region of the human genome for multiplexed and non-multiplexed NTT-seq K562 cell datasets. Sequencing data were normalized as bins per million mapped reads (BPM), as for the PBMC datasets. (FIG. 12I) Heatmap displaying coverage centered on H3K27ac peaks for multiplexed and non-multiplexed NTT-seq experiments using K562 cells, for RNAPII, H3K27ac, and H3K27me3 modalities. (FIG. 12J) As for FIG. 12I, for H3K27me3 peaks.

FIG. 13A-FIG. 13F show NTT-seq provides accurate single-cell multimodal chromatin profiles. (FIG. 13A) Schematic overview of the single-cell NTT-seq protocol. Cells are tagmented and processed in bulk (steps 1-3), and are encapsulated in droplets to attach cell-specific barcode sequenced to transposed DNA fragments (steps 4-5). (FIG. 13B) UMAP representations of cells profiled using multiplexed single-cell NTT-seq. Individual UMAP representations built using each assay are shown (left side), along with a visualization constructed incorporating information from all three chromatin modalities (WNN UMAP, right side). Cells are colored by their predicted cell type. (FIG. 13C) Multimodal genome browser view of a representative genomic locus, for K562 cells. Fragment counts for each assay are shown, scaled to the maximal value for each assay within the locus. Top three tracks show H3K27ac, H3K27me3, and RNAPII profiled simultaneously in a single-cell experiment. Lower three tracks show H3K27ac, H3K27me3, and RNAPII profiled individually in bulk-cell NTT-seq experiments using K562 cells. (FIG. 13D) Scatterplots showing normalized fragment counts for H3K27me3, H3K27ac, and RNAPII peaks defined by ENCODE (Nature. 2012 Sep. 6; 489(7414):57-74), for bulk and single-cell multiplexed NTT-seq experiments, for K562 cells. Peaks are colored according to their chromatin modality (red: H3K27me3 peak, yellow: H3K27ac peak, blue: RNAPII peak). Coefficient of determination (R2) between experiments are shown above each scatterplot. (FIG. 13E) Ternary plot showing the relative frequency of H3K27me3, H3K27ac, and RNAPII fragment counts within H3K27me3, H3K27ac, and RNAPII peak regions defined by ENCODE ChIP-seq datasets. (FIG. 13F) Fraction a cell's nearest neighbors belonging to the same predicted cell type, for neighbor graphs defined using a single chromatin modality or a weighted combination of modalities.

FIG. 14A-FIG. 14K show application of multiplexed single-cell NTT-seq to human tissues. (FIG. 14A) UMAP representation of PBMCs profiled using NTT-seq with protein expression. UMAPs for each assay are shown (left side), along with a multimodal UMAP constructed using all modalities (right side). Cells are shaded and labeled by cell types. (FIG. 14B) Patterns of cell-surface-protein expression in PBMCs profiled using NTT-seq. (FIG. 14C) Pearson correlation between NTT-seq and scCUT & Tag-pro (CT-pro) signal in PBMCs within H3K27me3 and H3K27ac peaks. (FIG. 14D) Scatterplot showing the number of counts per H3K27me3 and H3K27ac peak for each assay, for PBMCs profiled by NTT-seq. Peaks are colored according to their assay (red: H3K27me3; yellow: H3K27ac). Coefficient of determination (R2) is shown above. Axes: total fragment counts per million. (FIG. 14E) Genome browser view of the PAX5 and CD33 loci for B cells and CD14+ monocytes. Normalized protein expression values are shown alongside coverage tracks for each cell type for CD19 and CD33 protein. H3K27me3 and H3K27ac histone modification profiles are overlaid, with the signal for each scaled to the maximal signal within the genomic region shown. (FIG. 14F) Fraction of cells with <25% of neighbors belonging to the same cell type, for neighbor graphs defined using individual chromatin modalities, cell-surface protein expression, or a combination of chromatin modalities. (FIG. 14G) UMAP of BMMCs profiled using NTT-seq. Separate UMAPs for H3K27me3 and H3K27ac are shown (left side), and a UMAP using both H3K27me3 and H3K27ac is shown (right). Cells are shaded and labeled by their cell type. HSPC: hematopoietic stem and progenitor cells; GMP/CLP: granulocyte monocyte progenitor/common lymphoid progenitor; CD14 Mono: CD14+ monocyte; pDC; plasmacytoid dendritic cell; NK: natural killer cell. (FIG. 14H) Distribution of total fragment counts per cell for H3K27ac and H3K27me3. (FIG. 14I) Pseudotime trajectory for B cell development. Cells are colored by their pseudotime value and labeled by their annotated cell type. (FIG. 14J) Heatmap showing H3K27me3 and H3K27ac signal for 10 kb genomic bins correlated with B cell pseudotime progression. Heatmaps show the same genomic regions for both assays, with identical ordering of genomic regions. (FIG. 14K) Expression of genes close to activated (gain H3K27ac, upper plot) or repressed (gain H3K27me3, lower plot) genomic regions in a separate scRNA-seq BMMC dataset, for cells in the B cell developmental trajectory.

FIG. 15A-FIG. 15D show design and evaluation of nb-Tn5. (FIG. 15A) Nanobody-Tn5 fusion protein plasmid map schematic showing position of Tn5 and secondary nanobody sequences. (FIG. 15B) Agarose DNA gel showing size-separation of PCR-amplified DNA sequencing library products for different combinations of nb-Tn5 and primary IgG antibody. Rabbit Ab: rabbit primary IgG antibody; Mouse Ab: mouse primary IgG antibody; IgG1 Ab: mouse IgG subtype 1 primary antibody; IgG2a Ab: mouse IgG subtype 2a primary antibody; rTn5: anti-rabbit IgG secondary nanobody-Tn5 fusion; mTn5: anti-mouse IgG secondary nanobody-Tn5 fusion: GIT: anti-mouse IgG1 secondary nanobody-Tn5 fusion; G2aT: anti-mouse IgG2a secondary nanobody-Tn5 fusion. Gels shows expected library amplification product (bands between 200 and 1,000 bp) in lanes where the nb-Tn5 fusion matches the primary IgG antibody (rabbit Ab+rTn5; mouse Ab+mTn5; IgG1 Ab+GIT; IgG2a Ab+G2aT). Replicates were not performed. (FIG. 15C) Scatterplots showing normalized fragment counts for H3K27me3 and H3K27ac peaks defined by ENCODE for bulk multiplexed and non-multiplexed NTT-seq experiments in human PBMCs. Peaks are colored according to their chromatin modality (red: H3K27me3 peak, yellow: H3K27ac peak). Coefficient of determination (R2) between experiments are shown above each scatterplot. (FIG. 15D) Scatterplots showing normalized fragment counts for H3K27me3, H3K27ac, and RNAPII peaks defined by ENCODE for bulk multiplexed and non-multiplexed NTT-seq experiments in K562 cells.

FIG. 16A-FIG. 16D show data sensitivity comparison across multimodal chromatin profiling methods. (FIG. 16A) Total reads and fragment counts per cell for multiCUT & Tag (Gopalan S et al. Mol Cell. 2021 Nov. 18; 81(22):4736-46.e5) and scNTT-seq. Read and fragment counts on y-axis are on a log 10 scale. multiCUT & Tag profiled only two marks, H3K27ac and H3K27me3, and so do not have RNAPII counts. Box-plot lower and upper hinges represent first and third quartiles. Upper/lower whiskers extend to the largest/smallest value no further than 1.5× the interquartile range. Data beyond the whiskers are plotted as single points. (FIG. 16B) Fraction of fragments falling in ENCODE peak regions for H3K27me3 and H3K27ac marks, for multiCUT & Tag (left box plots) and scNTT-seq (right box plots). Box plots constructed as for panel FIG. 16A. (FIG. 16C) Scatterplot showing the normalized insertion counts in H3K27me3 and H3K27ac ENCODE peak regions for the multiCUT & Tag mESC single-cell dataset. (FIG. 16D) Multimodal genome browser view of a representative genomic locus, for K562 cells. Top three tracks show H3K27ac, H3K27me3, and RNAPII profiled simultaneously in a single-cell experiment. Lower three tracks show H3K27ac, H3K27me3, and RNAPII profiled individually in bulk-cell NTT-seq experiments using K562 cells.

FIG. 17A-FIG. 17G show sensitivity and reproducibility of scNTT-seq. (FIG. 17A) Total read and fragment counts per cell and fraction of fragments in peaks (FRiP) for scCUT & Tag and scNTT-seq PBMC datasets. Box plot lower and upper hinges represent first and third quartiles. Upper/lower whiskers extend to the largest/smallest value no further than 1.5× the interquartile range. Data beyond the whiskers are plotted as single points. (FIG. 17B) Comparison of total unique antibody-derived tag (ADT) counts sequenced per cell for CUT & Tag-pro (Zhang et al. Nat Biotechnol. 2022 August; 40(8):1220-1230) and scNTT-seq. (FIG. 17C) Spearman correlation between H3K27me3 counts (top) or H3K27ac counts (bottom) for cells profiled using multiplexed single-cell NTT-seq, or FACS-sorted bulk ChIP-seq profiled by ENCODE. (FIG. 17D) Two-dimensional UMAP projection and clustering for a second PBMC scNTT-seq replicate profiling H3K27me3 and H3K27ac. UMAP representation was constructed using both modalities, using the weighted nearest neighbors (WNN) method. (FIG. 17E) Scatterplots showing the number of fragment counts per H3K27me3 and H3K27ac ENCODE peak region for each assay profiled in the second PBMC scNTT-seq replicate dataset. (FIG. 17F) Total read and fragment count and FRIP distributions for H3K27me3 and H3K27ac assays profiled in the second PBMC scNTT-seq replicate dataset. (FIG. 17G) Pearson correlation between H3K27me3 and H3K27ac marks across PBMC scNTT-seq replicate datasets.

FIG. 18A-FIG. 18B show accuracy of scNTT-seq applied to human BMMCs. (FIG. 18A) Scatterplot showing the number of counts per H3K27me3 and H3K27ac peak for each assay, for BMMC cells profiled using single-cell multiplexed NTT-seq. Peaks are shaded according to their assay (dark gray: H3K27me3 peaks; light gray: H3K27ac peaks). (FIG. 18B) Fraction of fragments in ENCODE peaks per cell, for H3K27ac and HK27me3 marks. Box-plot lower and upper hinges represent first and third quartiles. Upper/lower whiskers extend to the largest/smallest value no further than 1.5× the interquartile range. Data beyond the whiskers are plotted as single points.

FIG. 19A-FIG. 19B show spatially resolved amplification, capture, and cDNA generation from mouse spinal cord. (FIG. 19A) Hematoxylin and eosin staining of fresh frozen mouse lumbar spinal cord tissue sections. Tissue was sectioned onto glass slides bearing poly(A) compatible capture DNA oligonucleotide probes. (FIG. 19B) Fluorescent cDNA prints from endogenous mRNA and RNA resulting from in vitro transcription (IVT) based amplification of tagmented genomic DNA. Due to the incorporation of a fluorescently labeled dCTP during reverse transcription, resulting cDNA is fluorescent. Following staining, imaging, and permeabilization, samples were tagmented with Tn5 loaded with adapters containing a T7 RNA polymerase promoter and polyadenylation sequence. In well 1, fluorescent cDNA print was generated as described in Stahl et al. (Science. 2016 Jul. 1; 353(6294):78-82). As such, the cDNA print is solely a reflection of mRNA present in the sample. In well 2, no reverse transcriptase or T7 RNA polymerase were added, resulting in no cDNA print. In wells 3 & 4, RNA from IVT amplified tagmentation products was captured and reverse transcribed as in well 1. The brighter signal in wells 3 & 4 vs 1 indicates that tagmentation products were successfully amplified, captured, and reverse transcribed in wells 3 & 4.

FIG. 20 provides results from an in situ CUT & TAG experiment demonstrating that bulk reference data (top) and spatial CUT & TAG data (bottom) are consistent.

### DETAILED DESCRIPTION

The compositions and methods described herein provide improved reagents and methods for performing multiplexed, spatially resolved, or single-cell chromatin analysis. Provided herein are compositions and methods that utilize a tagmentation step to elucidate the composition and arrangement of DNA-protein assemblies across the genome.

Described below are components that comprise, or are utilized, with one or more of the compositions or methods of the disclosure. The components used in these compositions and methods are further described below. In the descriptions of the compositions and methods discussed herein, the various components can be defined by use of technical and scientific terms having the same meaning as commonly understood by one of ordinary skill in the art to which this invention belongs and by reference to published texts. Such texts provide one skilled in the art with a general guide to many of the terms used in the present application. The definitions contained in this specification are provided for clarity in describing the components and compositions herein and are not intended to limit the claimed invention.

### I. Components of the Compositions and Methods

In certain embodiments, the compositions and methods utilize tagmentation reagents and reactions that are known in the art. Some of these reagents and/or methodologies have been modified or adapted as described herein.

### A. Fusion Proteins

In certain embodiments, the compositions and methods described herein utilize a fusion protein that includes a transposase and a ligand that binds to a target epitope on genomic DNA of a subject organism. The target epitope may be any partner biological molecule found in chromatin, including, without limitation, histones, transcription factors, transcribing RNA polymerase, chromatin interacting RNAs such as XIST, MALAT and NEAT, and DNA structures.

### Ligand

The methods and compositions described herein utilize a ligand. As used herein, the term ligand (sometimes referred to herein as binding moiety) refers to any molecule that specifically binds to another molecule, which is sometimes referred to herein as the partner molecule or target. In one embodiment, the binding moiety is an antibody. As used herein, an “antibody” is a monoclonal antibody, a synthetic antibody, a recombinant antibody, a chimeric antibody, a humanized antibody, a human antibody, a CDR-grafted antibody, a multi-specific binding construct that can bind two or more targets, a dual specific antibody, a bi-specific antibody or a multi-specific antibody, or an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one VL, one VH antigen-binding domain, and one or two constant “effector” domains optionally connected by linker domains, a univalent antibody lacking a hinge region, a single domain antibody, a dual variable domain immunoglobulin (DVD-Ig) binding protein or a nanobody. Also included in this definition are antibody mimetics such as affibodies, i.e., a class of engineered affinity proteins, generally small (˜6.5 kDa) single domain proteins that can be isolated for high affinity and specificity to any given protein target. In certain embodiments, the ligand is a single domain antibody. In certain embodiments, the ligand is an antibody to protein A, such as that used with CUT & Tag. Kaya-Okur et al. Nat Protoc. 2020 October; 15(10):3264-3283, which is incorporated herein by reference.

In some embodiments, the binding moiety is a G4 binding protein, or a fragment thereof. The guanine quadruplex (G4) structure in DNA is a secondary structure motif that plays important roles in DNA replication, transcriptional regulation, and maintenance of genomic stability. G4 binding proteins include, without limitation, SLIRP, LARK, GNL1, STM1P, CIRBP, SERBP1, eIF4G, WRN, Nucleolin, Mre11, DHX36, hnRNP A1, CNBP, BRCA1, breast cancer type 1 susceptibility protein; hnRNP, heterogeneous nuclear ribonucleoprotein; POTI, protection of telomeres 1; RPA, replication protein A; TEBP, Telomere End Binding Protein; TLS/FUS, translocated in liposarcoma/fused in sarcoma; Topo I, Topoisomerase I; TRF2, telomere repeat binding factor 2; UP1, unwinding protein 1; PARP-1, Poly [ADP-ribose] polymerase 1; CNBP, cellular nucleic-acid-binding protein; IGF-2, Insulin-like growth factor 2; MAZ, myc-associated zinc-finger; FMR2, fragile X mental retardation 2; RHAU, the RNA helicase associated with AU-rich element; SRSF, serin/arginine-rich splicing factor; BLM, Bloom syndrome protein; Dna2, DNA replication helicase/nuclease 2; G4R1, G4 Resolvase 1; FANCJ, Fanconi anemia complementation group J; Sgs1, small growth suppressor 1; and WRN, Werner syndrome ATP-dependent helicase. In one embodiment, the G4 protein is G4P as described by Zheng et al, Detection of genomic G-quadruplexes in living cells using a small artificial protein, Nucleic Acids Research. 2020 Nov. 18; 48(20): 11706-11720, which is incorporated herein by reference.

In another embodiment, the target epitope is bound by a primary antibody, and the ligand of the fusion protein recognizes a primary antibody that recognizes the target epitope, thus indirectly binding the target epitope. Thus, in certain embodiments, the ligand of the fusion protein is specific to the primary antibody's species and isotype. For example, the ligand may be anti-IgA, IgD, IgE, IgG, or IgM. In addition, the ligand may be raised against a primary antibody of any species including human, mouse, rat, rabbit, etc. The ligand and the primary antibody are independently selected from any type of antibody/ligand, as described herein and known in the art. For example, in one embodiment, the primary antibody is a monoclonal antibody, and the ligand is a nanobody. In another embodiment, the primary antibody is a scFv, and the ligand is a nanobody. As a non-limiting example, the primary antibody may be an anti-IgG1, IgG2A, IgG2B, IgG2C or IgG3 mouse antibody, or universal mouse antibody.

### Nanobody-Tn5 Fusions

In another embodiment, nanobody-Tn fusions are provided. Nanobodies are single domain antibodies derived from llama, alpaca, shark heavy-chain only antibodies, or from other animal models engineered to produce camelidae-like VHHs, that have unique properties such as nanoscale size, robust structure, stable and soluble behaviors in aqueous solution, high affinity and specificity for only one cognate target. Nanobodies achieve comparable binding affinities and specificities to classical antibodies, despite comprising only a single 15 kDa variable domain. The camelid VHH domain that forms the Nb is homologous to the Ab VH domain and contains three highly variable loops H1, H2, and H3. See, e.g., Muyldermans S., Nanobodies: natural single-domain antibodies. Annu Rev Biochem. 2013; 82:775-97 and Mitchell, Laura S, and Lucy J Colwell. Proteins vol. 86,7 (2018): 697-706, which are incorporated herein by reference. Various fusion proteins encompassing nanobody ligands are exemplified herein. These examples are not intended to limit the invention. These fusion proteins are useful with modalities such as e.g., CUT & Tag, to help overcome the limitations associated with the use of pA-Tn5, as well as being useful with the procedures described herein, such as NTT-seq.

### Target Molecule

The ligand (whether nanobody or other ligand as described herein) is capable of recognizing and binding, and binds, a partner, or target, biological molecule. Such partner molecules include, without limitation, peptides, proteins, antibodies or antibody fragments, affibodies, a ribonucleic acid sequence or deoxyribonucleic acid sequence, aptamers, lipids, polysaccharides, lectins, a chimeric molecule formed of multiples of the same or different moieties. In one embodiment, the partner molecule is a protein. In certain embodiments, the ligand is not an antibody to proteinA.

In certain embodiments, the target molecule is a protein found on, or associated with, chromatin found in the biological specimen. Chromatin is composed of a cell's DNA and associated proteins. Histone proteins and DNA are found in approximately equal mass in eukaryotic chromatin, and nonhistone proteins are also in great abundance. The basic unit of organization of chromatin is the nucleosome, a structure of DNA and histone proteins that repeats itself throughout an organism's genetic material. Histones are highly conserved basic proteins, whose positively charged character helps them to bind the negatively charged phosphate backbone of DNA.

Exemplary target molecules include histones, including H1, H2A, H2B, H3, H4, and H5. See, Annunziato, A. (2008) DNA Packaging: Nucleosomes and Chromatin. Nature Education 1(1):26, which is incorporated herein by reference. Post-translationally modified histones may also be targeted, such as phosphorylation on serine or threonine residues, methylation on lysine or arginine, acetylation and deacetylation of lysines, ubiquitylation of lysines and sumoylation of lysines. In other embodiments, the target molecule is RNA polymerase. In other embodiments, the target molecule is a transcription factor (TF), or a suspected transcription factor. A list of 1639 known and likely human transcription factors have been described in the art, and cataloged by Lambert S A, et al. (2018) The Human Transcription Factors. Cell. 172(4):650-665. doi: 10.1016/j.cell.2018.01.029. A list of the 1639 human TFs is included as Table 1 below. Other exemplary human targets are listed below in Table 2 below.

TABLE 1
Human Transcription Factors

| Gene | ID | DBD | Gene | ID | DBD |
|---|---|---|---|---|---|
| AC00877 | ENSG00000267179 | C2H2 ZF | ARID3B | ENSG00000179361 | ARID/BRIGHT |
| 0.3 | | | | | |
| AC02350 | ENSG00000267281 | bZIP | ARID3C | ENSG00000205143 | ARID/BRIGHT |
| 9.3 | | | | | |
| AC09283 | ENSG00000233757 | C2H2 ZF | ARID5A | ENSG00000196843 | ARID/BRIGHT |
| 5.1 | | | | | |
| AC13869 | ENSG00000264668 | C2H2 ZF | ARID5B | ENSG00000150347 | ARID/BRIGHT |
| 6.1 | | | | | |
| ADNP | ENSG00000101126 | Homeodomain | ARNT | ENSG00000143437 | bHLH |
| ADNP2 | ENSG00000101544 | Homeodomain | ARNT2 | ENSG00000172379 | bHLH |
| AEBP1 | ENSG00000106624 | Unknown | ARNTL | ENSG00000133794 | bHLH |
| AEBP2 | ENSG00000139154 | C2H2 ZF | ARNTL2 | ENSG00000029153 | bHLH |
| AHCTF1 | ENSG00000153207 | AT hook | ARX | ENSG00000004848 | Homeodomain |
| AHDC1 | ENSG00000126705 | AT hook | ASCL1 | ENSG00000139352 | bHLH |
| AHR | ENSG00000106546 | bHLH | ASCL2 | ENSG00000183734 | bHLH |
| AHRR | ENSG00000063438 | bHLH | ASCL3 | ENSG00000176009 | bHLH |
| AIRE | ENSG00000160224 | SAND | ASCL4 | ENSG00000187855 | bHLH |
| AKAP8 | ENSG00000105127 | C2H2 ZF | ASCL5 | ENSG00000232237 | bHLH |
| AKAP8L | ENSG00000011243 | C2H2 ZF | ASH1L | ENSG00000116539 | AT hook |
| AKNA | ENSG00000106948 | AT hook | ATF1 | ENSG00000123268 | bZIP |
| ALX1 | ENSG00000180318 | Homeodomain | ATF2 | ENSG00000115966 | bZIP |
| ALX3 | ENSG00000156150 | Homeodomain | ATF3 | ENSG00000162772 | bZIP |
| ALX4 | ENSG00000052850 | Homeodomain | ATF4 | ENSG00000128272 | bZIP |
| ANHX | ENSG00000227059 | Homeodomain | ATF5 | ENSG00000169136 | bZIP |
| ANKZF1 | ENSG00000163516 | C2H2 ZF | ATF6 | ENSG00000118217 | bZIP |
| AR | ENSG00000169083 | Nuclear receptor | ATF6B | ENSG00000213676 | bZIP |
| ARGFX | ENSG00000186103 | Homeodomain | ATF7 | ENSG00000170653 | bZIP |
| ARHGAP | ENSG00000160007 | Unknown | ATMIN | ENSG00000166454 | C2H2 ZF |
| 35 | | | ATOH1 | ENSG00000172238 | bHLH |
| ARID2 | ENSG00000189079 | ARID/BRIGHT; | ATOH7 | ENSG00000179774 | bHLH |
| | | RFX | ATOH8 | ENSG00000168874 | bHLH |
| ARID3A | ENSG00000116017 | ARID/BRIGHT | BACH1 | ENSG00000156273 | bZIP |
| BACH2 | ENSG00000112182 | bZIP | CEBPB | ENSG00000172216 | bZIP |
| BARHL1 | ENSG00000125492 | Homeodomain | CEBPD | ENSG00000221869 | bZIP |
| BARHL2 | ENSG00000143032 | Homeodomain | CEBPE | ENSG00000092067 | bZIP |
| BARX1 | ENSG00000131668 | Homeodomain | CEBPG | ENSG00000153879 | bZIP |
| BARX2 | ENSG00000043039 | Homeodomain | CEBPZ | ENSG00000115816 | Unknown |
| BATF | ENSG00000156127 | bZIP | CENPA | ENSG00000115163 | Unknown |
| BATF2 | ENSG00000168062 | bZIP | CENPB | ENSG00000125817 | CENPB |
| BATF3 | ENSG00000123685 | bZIP | CENPBD1 | ENSG00000177946 | CENPB |
| BAZ2A | ENSG00000076108 | MBD; AT hook | CENPS | ENSG00000175279 | Unknown |
| BAZ2B | ENSG00000123636 | MBD | CENPT | ENSG00000102901 | Unknown |
| BBX | ENSG00000114439 | HMG/Sox | CENPX | ENSG00000169689 | Unknown |
| BCL11A | ENSG00000119866 | C2H2 ZF | CGGBP1 | ENSG00000163320 | Unknown |
| BCL11B | ENSG00000127152 | C2H2 ZF | CHAMP1 | ENSG00000198824 | C2H2 ZF |
| BCL6 | ENSG00000113916 | C2H2 ZF | CHCHD3 | ENSG00000106554 | Unknown |
| BCL6B | ENSG00000161940 | C2H2 ZF | CIC | ENSG00000079432 | HMG/Sox |
| BHLHA15 | ENSG00000180535 | bHLH | CLOCK | ENSG00000134852 | bHLH |
| BHLHA9 | ENSG00000205899 | bHLH | CPEB1 | ENSG00000214575 | Unknown |
| BHLHE22 | ENSG00000180828 | bHLH | CPXCR1 | ENSG00000147183 | C2H2 ZF |
| BHLHE23 | ENSG00000125533 | bHLH | CREB1 | ENSG00000118260 | bZIP |
| BHLHE40 | ENSG00000134107 | bHLH | CREB3 | ENSG00000107175 | bZIP |
| BHLHE41 | ENSG00000123095 | bHLH | CREB3L1 | ENSG00000157613 | bZIP |
| BNC1 | ENSG00000169594 | C2H2 ZF | CREB3L2 | ENSG00000182158 | bZIP |
| BNC2 | ENSG00000173068 | C2H2 ZF | CREB3L3 | ENSG00000060566 | bZIP |
| BORCS8- | ENSG00000064489 | MADS box | CREB3L4 | ENSG00000143578 | bZIP |
| MEF2B | | | | | |
| BPTF | ENSG00000171634 | Unknown | CREB5 | ENSG00000146592 | bZIP |
| BRF2 | ENSG00000104221 | Unknown | CREBL2 | ENSG00000111269 | bZIP |
| BSX | ENSG00000188909 | Homeodomain | CREBZF | ENSG00000137504 | bZIP |
| C11orf95 | ENSG00000188070 | BED ZF | CREM | ENSG00000095794 | bZIP |
| CAMTA1 | ENSG00000171735 | CG-1 | CRX | ENSG00000105392 | Homeodomain |
| CAMTA2 | ENSG00000108509 | CG-1 | CSRNP1 | ENSG00000144655 | Unknown |
| CARF | ENSG00000138380 | Unknown | CSRNP2 | ENSG00000110925 | Unknown |
| CASZ1 | ENSG00000130940 | C2H2 ZF | CSRNP3 | ENSG00000178662 | Unknown |
| CBX2 | ENSG00000173894 | AT hook | CTCF | ENSG00000102974 | C2H2 ZF |
| CC2D1A | ENSG00000132024 | Unknown | CTCFL | ENSG00000124092 | C2H2 ZF |
| CCDC169- | ENSG00000250709 | bHLH | CUX1 | ENSG00000257923 | CUT; |
| SOHLH2 | | | | | Homeodomain |
| CCDC17 | ENSG00000159588 | C2H2 ZF | CUX2 | ENSG00000111249 | CUT; |
| | | | | | Homeodomain |
| CDC5L | ENSG00000096401 | Myb/SANT | CXXC1 | ENSG00000154832 | CxxC |
| CDX1 | ENSG00000113722 | Homeodomain | CXXC4 | ENSG00000168772 | CxxC |
| CDX2 | ENSG00000165556 | Homeodomain | CXXC5 | ENSG00000171604 | CxxC |
| CDX4 | ENSG00000131264 | Homeodomain | DACH1 | ENSG00000276644 | Unknown |
| CEBPA | ENSG00000245848 | bZIP | DACH2 | ENSG00000126733 | Unknown |
| DBP | ENSG00000105516 | bZIP | EBF1 | ENSG00000164330 | EBF1 |
| DBX1 | ENSG00000109851 | Homeodomain | EBF2 | ENSG00000221818 | EBF1 |
| DBX2 | ENSG00000185610 | Homeodomain | EBF3 | ENSG00000108001 | EBF1 |
| DDIT3 | ENSG00000175197 | bZIP | EBF4 | ENSG00000088881 | EBF1 |
| DEAF1 | ENSG00000177030 | SAND | EEA1 | ENSG00000102189 | C2H2 ZF |
| DLX1 | ENSG00000144355 | Homeodomain | EGR1 | ENSG00000120738 | C2H2 ZF |
| DLX2 | ENSG00000115844 | Homeodomain | EGR2 | ENSG00000122877 | C2H2 ZF |
| DLX3 | ENSG00000064195 | Homeodomain | EGR3 | ENSG00000179388 | C2H2 ZF |
| DLX4 | ENSG00000108813 | Homeodomain | EGR4 | ENSG00000135625 | C2H2 ZF |
| DLX5 | ENSG00000105880 | Homeodomain | EHF | ENSG00000135373 | Ets |
| DLX6 | ENSG00000006377 | Homeodomain | ELF1 | ENSG00000120690 | Ets |
| DMBX1 | ENSG00000197587 | Homeodomain | ELF2 | ENSG00000109381 | Ets |
| DMRT1 | ENSG00000137090 | DM | ELF3 | ENSG00000163435 | Ets; AT hook |
| DMRT2 | ENSG00000173253 | DM | ELF4 | ENSG00000102034 | Ets |
| DMRT3 | ENSG00000064218 | DM | ELF5 | ENSG00000135374 | Ets |
| DMRTA1 | ENSG00000176399 | DM | ELK1 | ENSG00000126767 | Ets |
| DMRTA2 | ENSG00000142700 | DM | ELK3 | ENSG00000111145 | Ets |
| DMRTB1 | ENSG00000143006 | DM | ELK4 | ENSG00000158711 | Ets |
| DMRTC2 | ENSG00000142025 | DM | EMX1 | ENSG00000135638 | Homeodomain |
| DMTF1 | ENSG00000135164 | Myb/SANT | EMX2 | ENSG00000170370 | Homeodomain |
| DNMT1 | ENSG00000130816 | CxxC | EN1 | ENSG00000163064 | Homeodomain |
| DNTTIP1 | ENSG00000101457 | AT hook | EN2 | ENSG00000164778 | Homeodomain |
| DOT1L | ENSG00000104885 | AT hook | EOMES | ENSG00000163508 | T-box |
| DPF1 | ENSG00000011332 | C2H2 ZF | EPAS1 | ENSG00000116016 | bHLH |
| DPF3 | ENSG00000205683 | C2H2 ZF | ERF | ENSG00000105722 | Ets |
| DPRX | ENSG00000204595 | Homeodomain | ERG | ENSG00000157554 | Ets |
| DR1 | ENSG00000117505 | Unknown | ESR1 | ENSG00000091831 | Nuclear receptor |
| DRAP1 | ENSG00000175550 | Unknown | ESR2 | ENSG00000140009 | Nuclear receptor |
| DRGX | ENSG00000165606 | Homeodomain | ESRRA | ENSG00000173153 | Nuclear receptor |
| DUX1 | DUX1_HUMAN | Homeodomain | ESRRB | ENSG00000119715 | Nuclear receptor |
| DUX3 | DUX3_HUMAN | Homeodomain | ESRRG | ENSG00000196482 | Nuclear receptor |
| DUX4 | ENSG00000260596 | Homeodomain | ESX1 | ENSG00000123576 | Homeodomain |
| DUXA | ENSG00000258873 | Homeodomain | ETS1 | ENSG00000134954 | Ets |
| DZIP1 | ENSG00000134874 | C2H2 ZF | ETS2 | ENSG00000157557 | Ets |
| E2F1 | ENSG00000101412 | E2F | ETV1 | ENSG00000006468 | Ets |
| E2F2 | ENSG00000007968 | E2F | ETV2 | ENSG00000105672 | Ets |
| E2F3 | ENSG00000112242 | E2F | ETV3 | ENSG00000117036 | Ets |
| E2F4 | ENSG00000205250 | E2F | ETV3L | ENSG00000253831 | Ets |
| E2F5 | ENSG00000133740 | E2F | ETV4 | ENSG00000175832 | Ets |
| E2F6 | ENSG00000169016 | E2F | ETV5 | ENSG00000244405 | Ets |
| E2F7 | ENSG00000165891 | E2F | ETV6 | ENSG00000139083 | Ets |
| E2F8 | ENSG00000129173 | E2F | ETV7 | ENSG00000010030 | Ets |
| E4F1 | ENSG00000167967 | C2H2 ZF | EVX1 | ENSG00000106038 | Homeodomain |
| EVX2 | ENSG00000174279 | Homeodomain | FOXJ2 | ENSG00000065970 | Forkhead |
| FAM170A | ENSG00000164334 | C2H2 ZF | FOXJ3 | ENSG00000198815 | Forkhead |
| FAM200B | ENSG00000237765 | BED ZF | FOXK1 | ENSG00000164916 | Forkhead |
| FBXL19 | ENSG00000099364 | CxxC | FOXK2 | ENSG00000141568 | Forkhead |
| FERD3L | ENSG00000146618 | bHLH | FOXL1 | ENSG00000176678 | Forkhead |
| FEV | ENSG00000163497 | Ets | FOXL2 | ENSG00000183770 | Forkhead |
| FEZF1 | ENSG00000128610 | C2H2 ZF | FOXM1 | ENSG00000111206 | Forkhead |
| FEZF2 | ENSG00000153266 | C2H2 ZF | FOXN1 | ENSG00000109101 | Forkhead |
| FIGLA | ENSG00000183733 | bHLH | FOXN2 | ENSG00000170802 | Forkhead |
| FIZ1 | ENSG00000179943 | C2H2 ZF | FOXN3 | ENSG00000053254 | Forkhead |
| FLI1 | ENSG00000151702 | Ets | FOXN4 | ENSG00000139445 | Forkhead |
| FLYWCH1 | ENSG00000059122 | FLYWCH | FOXO1 | ENSG00000150907 | Forkhead |
| FOS | ENSG00000170345 | bZIP | FOXO3 | ENSG00000118689 | Forkhead |
| FOSB | ENSG00000125740 | bZIP | FOXO4 | ENSG00000184481 | Forkhead |
| FOSL1 | ENSG00000175592 | bZIP | FOXO6 | ENSG00000204060 | Forkhead |
| FOSL2 | ENSG00000075426 | bZIP | FOXP1 | ENSG00000114861 | Forkhead |
| FOXA1 | ENSG00000129514 | Forkhead | FOXP2 | ENSG00000128573 | Forkhead |
| FOXA2 | ENSG00000125798 | Forkhead | FOXP3 | ENSG00000049768 | Forkhead |
| FOXA3 | ENSG00000170608 | Forkhead | FOXP4 | ENSG00000137166 | Forkhead |
| FOXB1 | ENSG00000171956 | Forkhead | FOXQ1 | ENSG00000164379 | Forkhead |
| FOXB2 | ENSG00000204612 | Forkhead | FOXR1 | ENSG00000176302 | Forkhead |
| FOXC1 | ENSG00000054598 | Forkhead | FOXR2 | ENSG00000189299 | Forkhead |
| FOXC2 | ENSG00000176692 | Forkhead | FOXS1 | ENSG00000179772 | Forkhead |
| FOXD1 | ENSG00000251493 | Forkhead | GABPA | ENSG00000154727 | Ets |
| FOXD2 | ENSG00000186564 | Forkhead | GATA1 | ENSG00000102145 | GATA |
| FOXD3 | ENSG00000187140 | Forkhead | GATA2 | ENSG00000179348 | GATA |
| FOXD4 | ENSG00000170122 | Forkhead | GATA3 | ENSG00000107485 | GATA |
| FOXD4L1 | ENSG00000184492 | Forkhead | GATA4 | ENSG00000136574 | GATA |
| FOXD4L3 | ENSG00000187559 | Forkhead | GATA5 | ENSG00000130700 | GATA |
| FOXD4L4 | ENSG00000184659 | Forkhead | GATA6 | ENSG00000141448 | GATA |
| FOXD4L5 | ENSG00000204779 | Forkhead | GATAD2A | ENSG00000167491 | GATA |
| FOXD4L6 | ENSG00000273514 | Forkhead | GATAD2B | ENSG00000143614 | GATA |
| FOXE1 | ENSG00000178919 | Forkhead | GBX1 | ENSG00000164900 | Homeodomain |
| FOXE3 | ENSG00000186790 | Forkhead | GBX2 | ENSG00000168505 | Homeodomain |
| FOXF1 | ENSG00000103241 | Forkhead | GCM1 | ENSG00000137270 | GCM |
| FOXF2 | ENSG00000137273 | Forkhead | GCM2 | ENSG00000124827 | GCM |
| FOXG1 | ENSG00000176165 | Forkhead | GFI1 | ENSG00000162676 | C2H2 ZF |
| FOXH1 | ENSG00000160973 | Forkhead | GFI1B | ENSG00000165702 | C2H2 ZF |
| FOXI1 | ENSG00000168269 | Forkhead | GLI1 | ENSG00000111087 | C2H2 ZF |
| FOXI2 | ENSG00000186766 | Forkhead | GLI2 | ENSG00000074047 | C2H2 ZF |
| FOXI3 | ENSG00000214336 | Forkhead | GLI3 | ENSG00000106571 | C2H2 ZF |
| FOXJ1 | ENSG00000129654 | Forkhead | GLI4 | ENSG00000250571 | C2H2 ZF |
| GLIS1 | ENSG00000174332 | C2H2 ZF | HIC2 | ENSG00000169635 | C2H2 ZF |
| GLIS2 | ENSG00000126603 | C2H2 ZF | HIF1A | ENSG00000100644 | bHLH |
| GLIS3 | ENSG00000107249 | C2H2 ZF | HIF3A | ENSG00000124440 | bHLH |
| GLMP | ENSG00000198715 | Unknown | HINFP | ENSG00000172273 | C2H2 ZF |
| GLYR1 | ENSG00000140632 | AT hook | HIVEP1 | ENSG00000095951 | C2H2 ZF |
| GMEB1 | ENSG00000162419 | SAND | HIVEP2 | ENSG00000010818 | C2H2 ZF |
| GMEB2 | ENSG00000101216 | SAND | HIVEP3 | ENSG00000127124 | C2H2 ZF |
| GPBP1 | ENSG00000062194 | Unknown | HKR1 | ENSG00000181666 | C2H2 ZF |
| GPBP1L1 | ENSG00000159592 | Unknown | HLF | ENSG00000108924 | bZIP |
| GRHL1 | ENSG00000134317 | Grainy head | HLX | ENSG00000136630 | Homeodomain |
| GRHL2 | ENSG00000083307 | Grainy head | HMBOX1 | ENSG00000147421 | Homeodomain |
| GRHL3 | ENSG00000158055 | Grainy head | HMG20A | ENSG00000140382 | HMG/Sox |
| GSC | ENSG00000133937 | Homeodomain | HMG20B | ENSG00000064961 | HMG/Sox |
| GSC2 | ENSG00000063515 | Homeodomain | HMGA1 | ENSG00000137309 | AT hook |
| GSX1 | ENSG00000169840 | Homeodomain | HMGA2 | ENSG00000149948 | AT hook |
| GSX2 | ENSG00000180613 | Homeodomain | HMGN3 | ENSG00000118418 | HMG/Sox |
| GTF2B | ENSG00000137947 | Unknown | HMX1 | ENSG00000215612 | Homeodomain |
| GTF2I | ENSG00000263001 | GTF2I-like | HMX2 | ENSG00000188816 | Homeodomain |
| GTF2IRD1 | ENSG00000006704 | GTF2I-like | HMX3 | ENSG00000188620 | Homeodomain |
| GTF2IRD2 | ENSG00000196275 | GTF2I-like | HNF1A | ENSG00000135100 | Homeodomain |
| GTF2IRD2B | ENSG00000174428 | GTF2I-like | HNF1B | ENSG00000275410 | Homeodomain |
| GTF3A | ENSG00000122034 | C2H2 ZF | HNF4A | ENSG00000101076 | Nuclear receptor |
| GZF1 | ENSG00000125812 | C2H2 ZF | HNF4G | ENSG00000164749 | Nuclear receptor |
| HAND1 | ENSG00000113196 | bHLH | HOMEZ | ENSG00000215271 | Homeodomain |
| HAND2 | ENSG00000164107 | bHLH | HOXA1 | ENSG00000105991 | Homeodomain |
| HBP1 | ENSG00000105856 | HMG/Sox | HOXA10 | ENSG00000253293 | Homeodomain |
| HDX | ENSG00000165259 | Homeodomain | HOXA11 | ENSG00000005073 | Homeodomain |
| HELT | ENSG00000187821 | bHLH | HOXA13 | ENSG00000106031 | Homeodomain |
| HES1 | ENSG00000114315 | bHLH | HOXA2 | ENSG00000105996 | Homeodomain |
| HES2 | ENSG00000069812 | bHLH | HOXA3 | ENSG00000105997 | Homeodomain |
| HES3 | ENSG00000173673 | bHLH | HOXA4 | ENSG00000197576 | Homeodomain |
| HES4 | ENSG00000188290 | bHLH | HOXA5 | ENSG00000106004 | Homeodomain |
| HES5 | ENSG00000197921 | bHLH | HOXA6 | ENSG00000106006 | Homeodomain |
| HES6 | ENSG00000144485 | bHLH | HOXA7 | ENSG00000122592 | Homeodomain |
| HES7 | ENSG00000179111 | bHLH | HOXA9 | ENSG00000078399 | Homeodomain |
| HESX1 | ENSG00000163666 | Homeodomain | HOXB1 | ENSG00000120094 | Homeodomain |
| HEY1 | ENSG00000164683 | bHLH | HOXB13 | ENSG00000159184 | Homeodomain |
| HEY2 | ENSG00000135547 | bHLH | HOXB2 | ENSG00000173917 | Homeodomain |
| HEYL | ENSG00000163909 | bHLH | HOXB3 | ENSG00000120093 | Homeodomain |
| HHEX | ENSG00000152804 | Homeodomain | HOXB4 | ENSG00000182742 | Homeodomain |
| HIC1 | ENSG00000177374 | C2H2 ZF | HOXB5 | ENSG00000120075 | Homeodomain |
| | | | HOXB6 | ENSG00000108511 | Homeodomain |
| | | | HOXB7 | ENSG00000260027 | Homeodomain |
| HOXB8 | ENSG00000120068 | Homeodomain | IRF9 | ENSG00000213928 | IRF |
| HOXB9 | ENSG00000170689 | Homeodomain | IRX1 | ENSG00000170549 | Homeodomain |
| HOXC10 | ENSG00000180818 | Homeodomain | IRX2 | ENSG00000170561 | Homeodomain |
| HOXC11 | ENSG00000123388 | Homeodomain | IRX3 | ENSG00000177508 | Homeodomain |
| HOXC12 | ENSG00000123407 | Homeodomain | IRX4 | ENSG00000113430 | Homeodomain |
| HOXC13 | ENSG00000123364 | Homeodomain | IRX5 | ENSG00000176842 | Homeodomain |
| HOXC4 | ENSG00000198353 | Homeodomain | IRX6 | ENSG00000159387 | Homeodomain |
| HOXC5 | ENSG00000172789 | Homeodomain | ISL1 | ENSG00000016082 | Homeodomain |
| HOXC6 | ENSG00000197757 | Homeodomain | ISL2 | ENSG00000159556 | Homeodomain |
| HOXC8 | ENSG00000037965 | Homeodomain | ISX | ENSG00000175329 | Homeodomain |
| HOXC9 | ENSG00000180806 | Homeodomain | JAZF1 | ENSG00000153814 | C2H2 ZF |
| HOXD1 | ENSG00000128645 | Homeodomain | JDP2 | ENSG00000140044 | bZIP |
| HOXD10 | ENSG00000128710 | Homeodomain | JRK | ENSG00000234616 | CENPB |
| HOXD11 | ENSG00000128713 | Homeodomain | JRKL | ENSG00000183340 | CENPB |
| HOXD12 | ENSG00000170178 | Homeodomain | JUN | ENSG00000177606 | bZIP |
| HOXD13 | ENSG00000128714 | Homeodomain | JUNB | ENSG00000171223 | bZIP |
| HOXD3 | ENSG00000128652 | Homeodomain | JUND | ENSG00000130522 | bZIP |
| HOXD4 | ENSG00000170166 | Homeodomain | KAT7 | ENSG00000136504 | C2H2 ZF |
| HOXD8 | ENSG00000175879 | Homeodomain | KCMF1 | ENSG00000176407 | C2H2 ZF |
| HOXD9 | ENSG00000128709 | Homeodomain | KCNIP3 | ENSG00000115041 | Unknown |
| HSF1 | ENSG00000185122 | HSF | KDM2A | ENSG00000173120 | CxxC |
| HSF2 | ENSG00000025156 | HSF | KDM2B | ENSG00000089094 | CxxC |
| HSF4 | ENSG00000102878 | HSF | KDM5B | ENSG00000117139 | ARID/BRIGHT |
| HSF5 | ENSG00000176160 | HSF | KIN | ENSG00000151657 | C2H2 ZF |
| HSFX1 | ENSG00000171116 | HSF | KLF1 | ENSG00000105610 | C2H2 ZF |
| HSFX2 | ENSG00000268738 | HSF | KLF10 | ENSG00000155090 | C2H2 ZF |
| HSFY1 | ENSG00000172468 | HSF | KLF11 | ENSG00000172059 | C2H2 ZF |
| HSFY2 | ENSG00000169953 | HSF | KLF12 | ENSG00000118922 | C2H2 ZF |
| IKZF1 | ENSG00000185811 | C2H2 ZF | KLF13 | ENSG00000169926 | C2H2 ZF |
| IKZF2 | ENSG00000030419 | C2H2 ZF | KLF14 | ENSG00000266265 | C2H2 ZF |
| IKZF3 | ENSG00000161405 | C2H2 ZF | KLF15 | ENSG00000163884 | C2H2 ZF |
| IKZF4 | ENSG00000123411 | C2H2 ZF | KLF16 | ENSG00000129911 | C2H2 ZF |
| IKZF5 | ENSG00000095574 | C2H2 ZF | KLF17 | ENSG00000171872 | C2H2 ZF |
| INSM1 | ENSG00000173404 | C2H2 ZF | KLF2 | ENSG00000127528 | C2H2 ZF |
| INSM2 | ENSG00000168348 | C2H2 ZF | KLF3 | ENSG00000109787 | C2H2 ZF |
| IRF1 | ENSG00000125347 | IRF | KLF4 | ENSG00000136826 | C2H2 ZF |
| IRF2 | ENSG00000168310 | IRF | KLF5 | ENSG00000102554 | C2H2 ZF |
| IRF3 | ENSG00000126456 | IRF | KLF6 | ENSG00000067082 | C2H2 ZF |
| IRF4 | ENSG00000137265 | IRF | KLF7 | ENSG00000118263 | C2H2 ZF |
| IRF5 | ENSG00000128604 | IRF | KLF8 | ENSG00000102349 | C2H2 ZF |
| IRF6 | ENSG00000117595 | IRF | KLF9 | ENSG00000119138 | C2H2 ZF |
| IRF7 | ENSG00000185507 | IRF | KMT2A | ENSG00000118058 | CxxC; AT hook |
| IRF8 | ENSG00000140968 | IRF | KMT2B | ENSG00000272333 | CxxC; AT hook |
| L3MBTL1 | ENSG00000185513 | C2H2 ZF | MEF2B | ENSG00000213999 | MADS box |
| L3MBTL3 | ENSG00000198945 | C2H2 ZF | MEF2C | ENSG00000081189 | MADS box |
| L3MBTL4 | ENSG00000154655 | C2H2 ZF | MEF2D | ENSG00000116604 | MADS box |
| LBX1 | ENSG00000138136 | Homeodomain | MEIS1 | ENSG00000143995 | Homeodomain |
| LBX2 | ENSG00000179528 | Homeodomain | MEIS2 | ENSG00000134138 | Homeodomain |
| LCOR | ENSG00000196233 | Pipsqueak | MEIS3 | ENSG00000105419 | Homeodomain |
| LCORL | ENSG00000178177 | Pipsqueak | MEOX1 | ENSG00000005102 | Homeodomain |
| LEF1 | ENSG00000138795 | HMG/Sox | MEOX2 | ENSG00000106511 | Homeodomain |
| LEUTX | ENSG00000213921 | Homeodomain | MESP1 | ENSG00000166823 | bHLH |
| LHX1 | ENSG00000273706 | Homeodomain | MESP2 | ENSG00000188095 | bHLH |
| LHX2 | ENSG00000106689 | Homeodomain | MGA | ENSG00000174197 | T-box |
| LHX3 | ENSG00000107187 | Homeodomain | MITF | ENSG00000187098 | bHLH |
| LHX4 | ENSG00000121454 | Homeodomain | MIXL1 | ENSG00000185155 | Homeodomain |
| LHX5 | ENSG00000089116 | Homeodomain | MKX | ENSG00000150051 | Homeodomain |
| LHX6 | ENSG00000106852 | Homeodomain | MLX | ENSG00000108788 | bHLH |
| LHX8 | ENSG00000162624 | Homeodomain | MLXIP | ENSG00000175727 | bHLH |
| LHX9 | ENSG00000143355 | Homeodomain | MLXIPL | ENSG00000009950 | bHLH |
| LIN28A | ENSG00000131914 | CSD | MNT | ENSG00000070444 | bHLH |
| LIN28B | ENSG00000187772 | CSD | MNX1 | ENSG00000130675 | Homeodomain |
| LIN54 | ENSG00000189308 | TCR/CxC | MSANTD1 | ENSG00000188981 | MADF |
| LMX1A | ENSG00000162761 | Homeodomain | MSANTD3 | ENSG00000066697 | MADF |
| LMX1B | ENSG00000136944 | Homeodomain | MSANTD4 | ENSG00000170903 | Myb/SANT |
| LTF | ENSG00000012223 | Unknown | MSC | ENSG00000178860 | bHLH |
| LYL1 | ENSG00000104903 | bHLH | MSGN1 | ENSG00000151379 | bHLH |
| MAF | ENSG00000178573 | bZIP | MSX1 | ENSG00000163132 | Homeodomain |
| MAFA | ENSG00000182759 | bZIP | MSX2 | ENSG00000120149 | Homeodomain |
| MAFB | ENSG00000204103 | bZIP | MTERF1 | ENSG00000127989 | mTERF |
| MAFF | ENSG00000185022 | bZIP | MTERF2 | ENSG00000120832 | mTERF |
| MAFG | ENSG00000197063 | bZIP | MTERF3 | ENSG00000156469 | mTERF |
| MAFK | ENSG00000198517 | bZIP | MTERF4 | ENSG00000122085 | mTERF |
| MAX | ENSG00000125952 | bHLH | MTF1 | ENSG00000188786 | C2H2 ZF |
| MAZ | ENSG00000103495 | C2H2 ZF | MTF2 | ENSG00000143033 | Unknown |
| MBD1 | ENSG00000141644 | MBD; CxxC ZF | MXD1 | ENSG00000059728 | bHLH |
| MBD2 | ENSG00000134046 | MBD | MXD3 | ENSG00000213347 | bHLH |
| MBD3 | ENSG00000071655 | MBD | MXD4 | ENSG00000123933 | bHLH |
| MBD4 | ENSG00000129071 | MBD | MXI1 | ENSG00000119950 | bHLH |
| MBD6 | ENSG00000166987 | MBD | MYB | ENSG00000118513 | Myb/SANT |
| MBNL2 | ENSG00000139793 | CCCH ZF | MYBL1 | ENSG00000185697 | Myb/SANT |
| MECOM | ENSG00000085276 | C2H2 ZF | MYBL2 | ENSG00000101057 | Myb/SANT |
| MECP2 | ENSG00000169057 | MBD; AT hook | MYC | ENSG00000136997 | bHLH |
| MEF2A | ENSG00000068305 | MADS box | MYCL | ENSG00000116990 | bHLH |
| MYCN | ENSG00000134323 | bHLH | NFIB | ENSG00000147862 | SMAD |
| MYF5 | ENSG00000111049 | bHLH | NFIC | ENSG00000141905 | SMAD |
| MYF6 | ENSG00000111046 | bHLH | NFIL3 | ENSG00000165030 | bZIP |
| MYNN | ENSG00000085274 | C2H2 ZF | NFIX | ENSG00000008441 | SMAD |
| MYOD1 | ENSG00000129152 | bHLH | NFKB1 | ENSG00000109320 | Rel |
| MYOG | ENSG00000122180 | bHLH | NFKB2 | ENSG00000077150 | Rel |
| MYPOP | ENSG00000176182 | Myb/SANT | NFX1 | ENSG00000086102 | NFX |
| MYRF | ENSG00000124920 | Ndt80/PhoG | NFXL1 | ENSG00000170448 | NFX |
| MYRFL | ENSG00000166268 | Ndt80/PhoG | NFYA | ENSG00000001167 | CBF/NF-Y |
| MYSM1 | ENSG00000162601 | Myb/SANT | NFYB | ENSG00000120837 | Unknown |
| MYT1 | ENSG00000196132 | C2H2 ZF | NFYC | ENSG00000066136 | Unknown |
| MYTIL | ENSG00000186487 | C2H2 ZF | NHLH | ENSG00000171786 | bHLH |
| MZF1 | ENSG00000099326 | C2H2 ZF | NHLH2 | ENSG00000177551 | bHLH |
| NACC2 | ENSG00000148411 | Unknown | NKRF | ENSG00000186416 | Unknown |
| NAIF1 | ENSG00000171169 | MADF | NKX1-1 | ENSG00000235608 | Homeodomain |
| NANOG | ENSG00000111704 | Homeodomain | NKX1-2 | ENSG00000229544 | Homeodomain |
| NANOGNB | ENSG00000205857 | Homeodomain | NKX2-1 | ENSG00000136352 | Homeodomain |
| NANOGP8 | ENSG00000255192 | Homeodomain | NKX2-2 | ENSG00000125820 | Homeodomain |
| NCOA1 | ENSG00000084676 | bHLH | NKX2-3 | ENSG00000119919 | Homeodomain |
| NCOA2 | ENSG00000140396 | bHLH | NKX2-4 | ENSG00000125816 | Homeodomain |
| NCOA3 | ENSG00000124151 | bHLH | NKX2-5 | ENSG00000183072 | Homeodomain |
| NEUROD1 | ENSG00000162992 | bHLH | NKX2-6 | ENSG00000180053 | Homeodomain |
| NEUROD2 | ENSG00000171532 | bHLH | NKX2-8 | ENSG00000136327 | Homeodomain |
| NEUROD4 | ENSG00000123307 | bHLH | NKX3-1 | ENSG00000167034 | Homeodomain |
| NEUROD6 | ENSG00000164600 | bHLH | NKX3-2 | ENSG00000109705 | Homeodomain |
| NEUROG1 | ENSG00000181965 | bHLH | NKX6-1 | ENSG00000163623 | Homeodomain |
| NEUROG2 | ENSG00000178403 | bHLH | NKX6-2 | ENSG00000148826 | Homeodomain |
| NEUROG3 | ENSG00000122859 | bHLH | NKX6-3 | ENSG00000165066 | Homeodomain |
| NFAT5 | ENSG00000102908 | Rel | NME2 | ENSG00000243678 | Unknown |
| NFATC1 | ENSG00000131196 | Rel | NOBOX | ENSG00000106410 | Homeodomain |
| NFATC2 | ENSG00000101096 | Rel | NOTO | ENSG00000214513 | Homeodomain |
| NFATC3 | ENSG00000072736 | Rel | NPAS1 | ENSG00000130751 | bHLH |
| NFATC4 | ENSG00000100968 | Rel | NPAS2 | ENSG00000170485 | bHLH |
| NFE2 | ENSG00000123405 | bZIP | NPAS3 | ENSG00000151322 | bHLH |
| NFE2L1 | ENSG00000082641 | bZIP | NPAS4 | ENSG00000174576 | bHLH |
| NFE2L2 | ENSG00000116044 | bZIP | NR0B1 | ENSG00000169297 | Unknown |
| NFE2L3 | ENSG00000050344 | bZIP | NR1D1 | ENSG00000126368 | Nuclear receptor |
| NFE4 | ENSG00000230257 | Unknown | NR1D2 | ENSG00000174738 | Nuclear receptor |
| NFIA | ENSG00000162599 | SMAD | NR1H2 | ENSG00000131408 | Nuclear receptor |
| | | | NR1H3 | ENSG00000025434 | Nuclear receptor |
| | | | NR1H4 | ENSG00000012504 | Nuclear receptor |
| | | | NR1I2 | ENSG00000144852 | Nuclear receptor |
| | | | NR1I3 | ENSG00000143257 | Nuclear receptor |
| NR2C1 | ENSG00000120798 | Nuclear receptor | PAX7 | ENSG00000009709 | Homeodomain; |
| | | | | | Paired box |
| NR2C2 | ENSG00000177463 | Nuclear receptor | PAX8 | ENSG00000125618 | Paired box |
| NR2E1 | ENSG00000112333 | Nuclear receptor | PAX9 | ENSG00000198807 | Paired box |
| NR2E3 | ENSG00000278570 | Nuclear receptor | PBX1 | ENSG00000185630 | Homeodomain |
| NR2F1 | ENSG00000175745 | Nuclear receptor | PBX2 | ENSG00000204304 | Homeodomain |
| NR2F2 | ENSG00000185551 | Nuclear receptor | PBX3 | ENSG00000167081 | Homeodomain |
| NR2F6 | ENSG00000160113 | Nuclear receptor | PBX4 | ENSG00000105717 | Homeodomain |
| NR3C1 | ENSG00000113580 | Nuclear receptor | PCGF2 | ENSG00000277258 | Unknown |
| NR3C2 | ENSG00000151623 | Nuclear receptor | PCGF6 | ENSG00000156374 | Unknown |
| NR4A1 | ENSG00000123358 | Nuclear receptor | PDX1 | ENSG00000139515 | Homeodomain |
| NR4A2 | ENSG00000153234 | Nuclear receptor | PEG | ENSG00000198300 | C2H2 ZF |
| NR4A3 | ENSG00000119508 | Nuclear receptor | PGR | ENSG00000082175 | Nuclear receptor |
| NR5A1 | ENSG00000136931 | Nuclear receptor | PHF1 | ENSG00000112511 | Unknown |
| NR5A2 | ENSG00000116833 | Nuclear receptor | PHF19 | ENSG00000119403 | Unknown |
| NR6A1 | ENSG00000148200 | Nuclear receptor | PHF20 | ENSG00000025293 | AT hook |
| NRF1 | ENSG00000106459 | Unknown | PHF21A | ENSG00000135365 | AT hook |
| NRL | ENSG00000129535 | bZIP | PHOX2A | ENSG00000165462 | Homeodomain |
| OLIG1 | ENSG00000184221 | bHLH | PHOX2B | ENSG00000109132 | Homeodomain |
| OLIG2 | ENSG00000205927 | bHLH | PIN1 | ENSG00000127445 | MBD |
| OLIG3 | ENSG00000177468 | bHLH | PITX1 | ENSG00000069011 | Homeodomain |
| ONECUT1 | ENSG00000169856 | CUT; | PITX2 | ENSG00000164093 | Homeodomain |
| | | Homeodomain | | | |
| ONECUT2 | ENSG00000119547 | CUT; | PITX3 | ENSG00000107859 | Homeodomain |
| | | Homeodomain | | | |
| ONECUT3 | ENSG00000205922 | CUT; | PKNOX1 | ENSG00000160199 | Homeodomain |
| | | Homeodomain | | | |
| OSR1 | ENSG00000143867 | C2H2 ZF | PKNOX2 | ENSG00000165495 | Homeodomain |
| OSR2 | ENSG00000164920 | C2H2 ZF | PLAG1 | ENSG00000181690 | C2H2 ZF |
| OTP | ENSG00000171540 | Homeodomain | PLAGL1 | ENSG00000118495 | C2H2 ZF |
| OTX1 | ENSG00000115507 | Homeodomain | PLAGL2 | ENSG00000126003 | C2H2 ZF |
| OTX2 | ENSG00000165588 | Homeodomain | PLSCR1 | ENSG00000188313 | Unknown |
| OVOL1 | ENSG00000172818 | C2H2 ZF | POGK | ENSG00000143157 | Brinker |
| OVOL2 | ENSG00000125850 | C2H2 ZF | POU1F1 | ENSG00000064835 | Homeodomain; |
| | | | | | POU |
| OVOL3 | ENSG00000105261 | C2H2 ZF | POU2AF1 | ENSG00000110777 | Unknown |
| PA2G4 | ENSG00000170515 | Unknown | POU2F1 | ENSG00000143190 | Homeodomain; |
| | | | | | POU |
| PATZ1 | ENSG00000100105 | C2H2 ZF; AT | POU2F2 | ENSG00000028277 | Homeodomain; |
| | | hook | | | POU |
| PAX1 | ENSG00000125813 | Paired box | POU2F3 | ENSG00000137709 | Homeodomain; |
| | | | | | POU |
| PAX2 | ENSG00000075891 | Homeodomain; | POU3F1 | ENSG00000185668 | Homeodomain; |
| | | Paired box | | | POU |
| PAX3 | ENSG00000135903 | Homeodomain; | POU3F2 | ENSG00000184486 | Homeodomain; |
| | | Paired box | | | POU |
| PAX4 | ENSG00000106331 | Homeodomain; | POU3F3 | ENSG00000198914 | Homeodomain; |
| | | Paired box | | | POU |
| PAX5 | ENSG00000196092 | Paired box | POU3F4 | ENSG00000196767 | Homeodomain; |
| | | | | | POU |
| PAX6 | ENSG00000007372 | Homeodomain; | POU4F1 | ENSG00000152192 | Homeodomain; |
| | | Paired box | | | POU |
| POU4F2 | ENSG00000151615 | Homeodomain; | RAX2 | ENSG00000173976 | Homeodomain |
| | | POU | | | |
| POU4F3 | ENSG00000091010 | Homeodomain; | RBAK | ENSG00000146587 | C2H2 ZF |
| | | POU | | | |
| POU5F1 | ENSG00000204531 | Homeodomain; | RBCK1 | ENSG00000125826 | Unknown |
| | | POU | | | |
| POU5F1B | ENSG00000212993 | Homeodomain; | RBPJ | ENSG00000168214 | CSL |
| | | POU | | | |
| POU5F2 | ENSG00000248483 | Homeodomain; | RBPJL | ENSG00000124232 | CSL |
| | | POU | | | |
| POU6F1 | ENSG00000184271 | Homeodomain; | RBSN | ENSG00000131381 | C2H2 ZF |
| | | POU | | | |
| POU6F2 | ENSG00000106536 | Homeodomain; | REL | ENSG00000162924 | Rel |
| | | POU | | | |
| PPARA | ENSG00000186951 | Nuclear receptor | RELA | ENSG00000173039 | Rel |
| PPARD | ENSG00000112033 | Nuclear receptor | RELB | ENSG00000104856 | Rel |
| PPARG | ENSG00000132170 | Nuclear receptor | REPIN1 | ENSG00000214022 | C2H2 ZF |
| PRDM1 | ENSG00000057657 | C2H2 ZF | REST | ENSG00000084093 | C2H2 ZF |
| PRDM10 | ENSG00000170325 | C2H2 ZF | REXO4 | ENSG00000148300 | Unknown |
| PRDM12 | ENSG00000130711 | C2H2 ZF | RFX1 | ENSG00000132005 | RFX |
| PRDM13 | ENSG00000112238 | C2H2 ZF | RFX2 | ENSG00000087903 | RFX |
| PRDM14 | ENSG00000147596 | C2H2 ZF | RFX3 | ENSG00000080298 | RFX |
| PRDM15 | ENSG00000141956 | C2H2 ZF | RFX4 | ENSG00000111783 | RFX |
| PRDM16 | ENSG00000142611 | C2H2 ZF | RFX5 | ENSG00000143390 | RFX |
| PRDM2 | ENSG00000116731 | C2H2 ZF | RFX6 | ENSG00000185002 | RFX |
| PRDM4 | ENSG00000110851 | C2H2 ZF | RFX7 | ENSG00000181827 | RFX |
| PRDM5 | ENSG00000138738 | C2H2 ZF | RFX8 | ENSG00000196460 | RFX |
| PRDM6 | ENSG00000061455 | C2H2 ZF | RHOXF1 | ENSG00000101883 | Homeodomain |
| PRDM8 | ENSG00000152784 | C2H2 ZF | RHOXF2 | ENSG00000131721 | Homeodomain |
| PRDM9 | ENSG00000164256 | C2H2 ZF | RHOXF2B | ENSG00000203989 | Homeodomain |
| PREB | ENSG00000138073 | Unknown | RLF | ENSG00000117000 | C2H2 ZF |
| PRMT3 | ENSG00000185238 | C2H2 ZF | RORA | ENSG00000069667 | Nuclear receptor |
| PROP1 | ENSG00000175325 | Homeodomain | RORB | ENSG00000198963 | Nuclear receptor |
| PROX1 | ENSG00000117707 | Prospero | RORC | ENSG00000143365 | Nuclear receptor |
| PROX2 | ENSG00000119608 | Prospero | RREB1 | ENSG00000124782 | C2H2 ZF |
| PRR12 | ENSG00000126464 | AT hook | RUNX1 | ENSG00000159216 | Runt |
| PRRX1 | ENSG00000116132 | Homeodomain | RUNX2 | ENSG00000124813 | Runt |
| PRRX2 | ENSG00000167157 | Homeodomain | RUNX3 | ENSG00000020633 | Runt |
| PTF1A | ENSG00000168267 | bHLH | RXRA | ENSG00000186350 | Nuclear receptor |
| PURA | ENSG00000185129 | Unknown | RXRB | ENSG00000204231 | Nuclear receptor |
| PURB | ENSG00000146676 | Unknown | RXRG | ENSG00000143171 | Nuclear receptor |
| PURG | ENSG00000172733 | Unknown | SAFB | ENSG00000160633 | Unknown |
| RAG1 | ENSG00000166349 | Unknown | SAFB2 | ENSG00000130254 | Unknown |
| RARA | ENSG00000131759 | Nuclear receptor | SALL1 | ENSG00000103449 | C2H2 ZF |
| RARB | ENSG00000077092 | Nuclear receptor | SALL2 | ENSG00000165821 | C2H2 ZF |
| RARG | ENSG00000172819 | Nuclear receptor | SALL3 | ENSG00000256463 | C2H2 ZF |
| RAX | ENSG00000134438 | Homeodomain | SALL4 | ENSG00000101115 | C2H2 ZF |
| | | | SATB1 | ENSG00000182568 | CUT; Homeodomain |
| SATB2 | ENSG00000119042 | CUT; | SOX10 | ENSG00000100146 | HMG/Sox |
| | | Homeodomain | | | |
| SCMH1 | ENSG00000010803 | Unknown | SOX11 | ENSG00000176887 | HMG/Sox |
| SCML4 | ENSG00000146285 | AT hook | SOX12 | ENSG00000177732 | HMG/Sox |
| SCRT1 | ENSG00000261678 | C2H2 ZF | SOX13 | ENSG00000143842 | HMG/Sox |
| SCRT2 | ENSG00000215397 | C2H2 ZF | SOX14 | ENSG00000168875 | HMG/Sox |
| SCX | ENSG00000260428 | bHLH | SOX15 | ENSG00000129194 | HMG/Sox |
| SEBOX | ENSG00000274529 | Homeodomain | SOX17 | ENSG00000164736 | HMG/Sox |
| SETBP1 | ENSG00000152217 | AT hook | SOX18 | ENSG00000203883 | HMG/Sox |
| SETDB1 | ENSG00000143379 | MBD | SOX2 | ENSG00000181449 | HMG/Sox |
| SETDB2 | ENSG00000136169 | MBD | SOX21 | ENSG00000125285 | HMG/Sox |
| SGSM2 | ENSG00000141258 | BED ZF | SOX3 | ENSG00000134595 | HMG/Sox |
| SHOX | ENSG00000185960 | Homeodomain | SOX30 | ENSG00000039600 | HMG/Sox |
| SHOX2 | ENSG00000168779 | Homeodomain | SOX4 | ENSG00000124766 | HMG/Sox |
| SIM1 | ENSG00000112246 | bHLH | SOX5 | ENSG00000134532 | HMG/Sox |
| SIM2 | ENSG00000159263 | bHLH | SOX6 | ENSG00000110693 | HMG/Sox |
| SIX1 | ENSG00000126778 | Homeodomain | SOX7 | ENSG00000171056 | HMG/Sox |
| SIX2 | ENSG00000170577 | Homeodomain | SOX8 | ENSG00000005513 | HMG/Sox |
| SIX3 | ENSG00000138083 | Homeodomain | SOX9 | ENSG00000125398 | HMG/Sox |
| SIX4 | ENSG00000100625 | Homeodomain | SP1 | ENSG00000185591 | C2H2 ZF |
| SIX5 | ENSG00000177045 | Homeodomain | SP100 | ENSG00000067066 | SAND |
| SIX6 | ENSG00000184302 | Homeodomain | SP110 | ENSG00000135899 | SAND |
| SKI | ENSG00000157933 | Unknown | SP140 | ENSG00000079263 | SAND |
| SKIL | ENSG00000136603 | Unknown | SP140L | ENSG00000185404 | SAND |
| SKOR1 | ENSG00000188779 | Unknown | SP2 | ENSG00000167182 | C2H2 ZF |
| SKOR2 | ENSG00000215474 | SAND | SP3 | ENSG00000172845 | C2H2 ZF |
| SLC2A4RG | ENSG00000125520 | C2H2 ZF | SP4 | ENSG00000105866 | C2H2 ZF |
| SMAD1 | ENSG00000170365 | SMAD | SP5 | ENSG00000204335 | C2H2 ZF |
| SMAD3 | ENSG00000166949 | SMAD | SP6 | ENSG00000189120 | C2H2 ZF |
| SMAD4 | ENSG00000141646 | SMAD | SP7 | ENSG00000170374 | C2H2 ZF |
| SMAD5 | ENSG00000113658 | SMAD | SP8 | ENSG00000164651 | C2H2 ZF |
| SMAD9 | ENSG00000120693 | SMAD | SP9 | ENSG00000217236 | C2H2 ZF |
| SMYD3 | ENSG00000185420 | Unknown | SPDEF | ENSG00000124664 | Ets |
| SNAI1 | ENSG00000124216 | C2H2 ZF | SPEN | ENSG00000065526 | Unknown |
| SNAI2 | ENSG00000019549 | C2H2 ZF | SPI1 | ENSG00000066336 | Ets |
| SNAI3 | ENSG00000185669 | C2H2 ZF | SPIB | ENSG00000269404 | Ets |
| SNAPC2 | ENSG00000104976 | Unknown | SPIC | ENSG00000166211 | Ets |
| SNAPC4 | ENSG00000165684 | Myb/SANT | SPZ1 | ENSG00000164299 | Unknown |
| SNAPC5 | ENSG00000174446 | Unknown | SRCAP | ENSG00000080603 | AT hook |
| SOHLH1 | ENSG00000165643 | bHLH | SREBF1 | ENSG00000072310 | bHLH |
| SOHLH2 | ENSG00000120669 | bHLH | SREBF2 | ENSG00000198911 | bHLH |
| SON | ENSG00000159140 | Unknown | SRF | ENSG00000112658 | MADS box |
| SOX1 | ENSG00000182968 | HMG/Sox | SRY | ENSG00000184895 | HMG/Sox |
| | | | ST18 | ENSG00000147488 | C2H2 ZF |
| STAT1 | ENSG00000115415 | STAT | TEF | ENSG00000167074 | bZIP |
| STAT2 | ENSG00000170581 | STAT | TERB1 | ENSG00000249961 | Myb/SANT |
| STAT3 | ENSG00000168610 | STAT | TERF1 | ENSG00000147601 | Myb/SANT |
| STAT4 | ENSG00000138378 | STAT | TERF2 | ENSG00000132604 | Myb/SANT |
| STAT5A | ENSG00000126561 | STAT | TET1 | ENSG00000138336 | CxxC |
| STAT5B | ENSG00000173757 | STAT | TET2 | ENSG00000168769 | Unknown |
| STAT6 | ENSG00000166888 | STAT | TET3 | ENSG00000187605 | CxxC |
| T | ENSG00000164458 | T-box | TFAP2A | ENSG00000137203 | AP-2 |
| TAL1 | ENSG00000162367 | bHLH | TFAP2B | ENSG00000008196 | AP-2 |
| TAL2 | ENSG00000186051 | bHLH | TFAP2C | ENSG00000087510 | AP-2 |
| TBP | ENSG00000112592 | TBP | TFAP2D | ENSG00000008197 | AP-2 |
| TBPL1 | ENSG00000028839 | TBP | TFAP2E | ENSG00000116819 | AP-2 |
| TBPL2 | ENSG00000182521 | TBP | TFAP4 | ENSG00000090447 | bHLH |
| TBR1 | ENSG00000136535 | T-box | TFCP2 | ENSG00000135457 | Grainyhead |
| TBX1 | ENSG00000184058 | T-box | TFCP2L1 | ENSG00000115112 | Grainyhead |
| TBX10 | ENSG00000167800 | T-box | TFDP1 | ENSG00000198176 | E2F |
| TBX15 | ENSG00000092607 | T-box | TFDP2 | ENSG00000114126 | E2F |
| TBX18 | ENSG00000112837 | T-box | TFDP3 | ENSG00000183434 | E2F |
| TBX19 | ENSG00000143178 | T-box | TFE3 | ENSG00000068323 | bHLH |
| TBX2 | ENSG00000121068 | T-box | TFEB | ENSG00000112561 | bHLH |
| TBX20 | ENSG00000164532 | T-box | TFEC | ENSG00000105967 | bHLH |
| TBX21 | ENSG00000073861 | T-box | TGIF1 | ENSG00000177426 | Homeodomain |
| TBX22 | ENSG00000122145 | T-box | TGIF2 | ENSG00000118707 | Homeodomain |
| TBX3 | ENSG00000135111 | T-box | TGIF2LX | ENSG00000153779 | Homeodomain |
| TBX4 | ENSG00000121075 | T-box | TGIF2LY | ENSG00000176679 | Homeodomain |
| TBX5 | ENSG00000089225 | T-box | THAP1 | ENSG00000131931 | THAP finger |
| TBX6 | ENSG00000149922 | T-box | THAP10 | ENSG00000129028 | THAP finger |
| TCF12 | ENSG00000140262 | bHLH | THAP11 | ENSG00000168286 | THAP finger |
| TCF15 | ENSG00000125878 | bHLH | THAP12 | ENSG00000137492 | THAP finger |
| TCF20 | ENSG00000100207 | Unknown | THAP2 | ENSG00000173451 | THAP finger |
| TCF21 | ENSG00000118526 | bHLH | THAP3 | ENSG00000041988 | THAP finger |
| TCF23 | ENSG00000163792 | bHLH | THAP4 | ENSG00000176946 | THAP finger |
| TCF24 | ENSG00000261787 | bHLH | THAP5 | ENSG00000177683 | THAP finger |
| TCF3 | ENSG00000071564 | bHLH | THAP6 | ENSG00000174796 | THAP finger |
| TCF4 | ENSG00000196628 | bHLH | THAP7 | ENSG00000184436 | THAP finger |
| TCF7 | ENSG00000081059 | HMG/Sox | THAP8 | ENSG00000161277 | THAP finger |
| TCF7L1 | ENSG00000152284 | HMG/Sox | THAP9 | ENSG00000168152 | THAP finger |
| TCF7L2 | ENSG00000148737 | HMG/Sox | THRA | ENSG00000126351 | Nuclear receptor |
| TCFL5 | ENSG00000101190 | bHLH | THRB | ENSG00000151090 | Nuclear receptor |
| TEAD1 | ENSG00000187079 | TEA | THYN1 | ENSG00000151500 | Unknown |
| TEAD2 | ENSG00000074219 | TEA | TIGD1 | ENSG00000221944 | CENPB |
| TEAD3 | ENSG00000007866 | TEA | TIGD2 | ENSG00000180346 | CENPB |
| TEAD4 | ENSG00000197905 | TEA | TIGD3 | ENSG00000173825 | CENPB |
| TIGD4 | ENSG00000169989 | CENPB | YY1 | ENSG00000100811 | C2H2 ZF |
| TIGD5 | ENSG00000179886 | CENPB | YY2 | ENSG00000230797 | C2H2 ZF |
| TIGD6 | ENSG00000164296 | CENPB | ZBED1 | ENSG00000214717 | BED ZF |
| TIGD7 | ENSG00000140993 | CENPB | ZBED2 | ENSG00000177494 | BED ZF |
| TLX1 | ENSG00000107807 | Homeodomain | ZBED3 | ENSG00000132846 | BED ZF |
| TLX2 | ENSG00000115297 | Homeodomain | ZBED4 | ENSG00000100426 | BED ZF |
| TLX3 | ENSG00000164438 | Homeodomain | ZBED5 | ENSG00000236287 | BED ZF |
| TMF1 | ENSG00000144747 | Unknown | ZBED6 | ENSG00000257315 | BED ZF |
| TOPORS | ENSG00000197579 | Unknown | ZBED9 | ENSG00000232040 | BED ZF |
| TP53 | ENSG00000141510 | p53 | ZBTB1 | ENSG00000126804 | C2H2 ZF |
| TP63 | ENSG00000073282 | p53 | ZBTB10 | ENSG00000205189 | C2H2 ZF |
| TP73 | ENSG00000078900 | p53 | ZBTB11 | ENSG00000066422 | C2H2 ZF |
| TPRX1 | ENSG00000178928 | Homeodomain | ZBTB12 | ENSG00000204366 | C2H2 ZF |
| TRAFD1 | ENSG00000135148 | C2H2 ZF | ZBTB14 | ENSG00000198081 | C2H2 ZF |
| TRERF1 | ENSG00000124496 | C2H2 ZF; | ZBTB16 | ENSG00000109906 | C2H2 ZF |
| | | Myb/SANT | | | |
| TRPS1 | ENSG00000104447 | GATA | ZBTB17 | ENSG00000116809 | C2H2 ZF |
| TSC22D1 | ENSG00000102804 | Unknown | ZBTB18 | ENSG00000179456 | C2H2 ZF |
| TSHZ1 | ENSG00000179981 | C2H2 ZF | ZBTB2 | ENSG00000181472 | C2H2 ZF |
| TSHZ2 | ENSG00000182463 | C2H2 ZF | ZBTB20 | ENSG00000181722 | C2H2 ZF |
| TSHZ3 | ENSG00000121297 | C2H2 ZF | ZBTB21 | ENSG00000173276 | C2H2 ZF |
| TTF1 | ENSG00000125482 | Myb/SANT | ZBTB22 | ENSG00000236104 | C2H2 ZF |
| TWIST1 | ENSG00000122691 | bHLH | ZBTB24 | ENSG00000112365 | C2H2 ZF; AT |
| | | | | | hook |
| TWIST2 | ENSG00000233608 | bHLH | ZBTB25 | ENSG00000089775 | C2H2 ZF |
| UBP1 | ENSG00000153560 | Grainy head | ZBTB26 | ENSG00000171448 | C2H2 ZF |
| UNCX | ENSG00000164853 | Homeodomain | ZBTB3 | ENSG00000185670 | C2H2 ZF |
| USF1 | ENSG00000158773 | bHLH | ZBTB32 | ENSG00000011590 | C2H2 ZF |
| USF2 | ENSG00000105698 | bHLH | ZBTB33 | ENSG00000177485 | C2H2 ZF |
| USF3 | ENSG00000176542 | bHLH | ZBTB34 | ENSG00000177125 | C2H2 ZF |
| VAX1 | ENSG00000148704 | Homeodomain | ZBTB37 | ENSG00000185278 | C2H2 ZF |
| VAX2 | ENSG00000116035 | Homeodomain | ZBTB38 | ENSG00000177311 | C2H2 ZF |
| VDR | ENSG00000111424 | Nuclear receptor | ZBTB39 | ENSG00000166860 | C2H2 ZF |
| VENTX | ENSG00000151650 | Homeodomain | ZBTB4 | ENSG00000174282 | C2H2 ZF |
| VEZF1 | ENSG00000136451 | C2H2 ZF | ZBTB40 | ENSG00000184677 | C2H2 ZF |
| VSX1 | ENSG00000100987 | Homeodomain | ZBTB41 | ENSG00000177888 | C2H2 ZF |
| VSX2 | ENSG00000119614 | Homeodomain | ZBTB42 | ENSG00000179627 | C2H2 ZF |
| WIZ | ENSG00000011451 | C2H2 ZF | ZBTB43 | ENSG00000169155 | C2H2 ZF |
| WT1 | ENSG00000184937 | C2H2 ZF | ZBTB44 | ENSG00000196323 | C2H2 ZF |
| XBP1 | ENSG00000100219 | bZIP | ZBTB45 | ENSG00000119574 | C2H2 ZF |
| XPA | ENSG00000136936 | Unknown | ZBTB46 | ENSG00000130584 | C2H2 ZF |
| YBX1 | ENSG00000065978 | CSD | ZBTB47 | ENSG00000114853 | C2H2 ZF |
| YBX2 | ENSG00000006047 | CSD | ZBTB48 | ENSG00000204859 | C2H2 ZF |
| YBX3 | ENSG00000060138 | CSD | ZBTB49 | ENSG00000168826 | C2H2 ZF |
| ZBTB5 | ENSG00000168795 | C2H2 ZF | ZHX3 | ENSG00000174306 | Homeodomain |
| ZBTB6 | ENSG00000186130 | C2H2 ZF | ZIC1 | ENSG00000152977 | C2H2 ZF |
| ZBTB7A | ENSG00000178951 | C2H2 ZF | ZIC2 | ENSG00000043355 | C2H2 ZF |
| ZBTB7B | ENSG00000160685 | C2H2 ZF | ZIC3 | ENSG00000156925 | C2H2 ZF |
| ZBTB7C | ENSG00000184828 | C2H2 ZF | ZIC4 | ENSG00000174963 | C2H2 ZF |
| ZBTB8A | ENSG00000160062 | C2H2 ZF | ZIC5 | ENSG00000139800 | C2H2 ZF |
| ZBTB8B | ENSG00000273274 | C2H2 ZF | ZIK1 | ENSG00000171649 | C2H2 ZF |
| ZBTB9 | ENSG00000213588 | C2H2 ZF | ZIM2 | ENSG00000269699 | C2H2 ZF |
| ZC3H8 | ENSG00000144161 | CCCH ZF | ZIM3 | ENSG00000141946 | C2H2 ZF |
| ZEB1 | ENSG00000148516 | C2H2 ZF; | ZKSCAN1 | ENSG00000106261 | C2H2 ZF |
| | | Homeodomain | | | |
| ZEB2 | ENSG00000169554 | C2H2 ZF; | ZKSCAN2 | ENSG00000155592 | C2H2 ZF |
| | | Homeodomain | | | |
| ZFAT | ENSG00000066827 | C2H2 ZF | ZKSCAN3 | ENSG00000189298 | C2H2 ZF |
| ZFHX2 | ENSG00000136367 | Homeodomain | ZKSCAN4 | ENSG00000187626 | C2H2 ZF |
| ZFHX3 | ENSG00000140836 | C2H2 ZF; | ZKSCAN5 | ENSG00000196652 | C2H2 ZF |
| | | Homeodomain | | | |
| ZFHX4 | ENSG00000091656 | C2H2 ZF; | ZKSCAN7 | ENSG00000196345 | C2H2 ZF |
| | | Homeodomain | | | |
| ZFP1 | ENSG00000184517 | C2H2 ZF | ZKSCAN8 | ENSG00000198315 | C2H2 ZF |
| ZFP14 | ENSG00000142065 | C2H2 ZF | ZMAT1 | ENSG00000166432 | C2H2 ZF |
| ZFP2 | ENSG00000198939 | C2H2 ZF | ZMAT4 | ENSG00000165061 | C2H2 ZF |
| ZFP28 | ENSG00000196867 | C2H2 ZF | ZNF10 | ENSG00000256223 | C2H2 ZF |
| ZFP3 | ENSG00000180787 | C2H2 ZF | ZNF100 | ENSG00000197020 | C2H2 ZF |
| ZFP30 | ENSG00000120784 | C2H2 ZF | ZNF101 | ENSG00000181896 | C2H2 ZF |
| ZFP37 | ENSG00000136866 | C2H2 ZF | ZNF107 | ENSG00000196247 | C2H2 ZF |
| ZFP41 | ENSG00000181638 | C2H2 ZF | ZNF112 | ENSG00000062370 | C2H2 ZF |
| ZFP42 | ENSG00000179059 | C2H2 ZF | ZNF114 | ENSG00000178150 | C2H2 ZF |
| ZFP57 | ENSG00000204644 | C2H2 ZF | ZNF117 | ENSG00000152926 | C2H2 ZF |
| ZFP62 | ENSG00000196670 | C2H2 ZF | ZNF12 | ENSG00000164631 | C2H2 ZF |
| ZFP64 | ENSG00000020256 | C2H2 ZF | ZNF121 | ENSG00000197961 | C2H2 ZF |
| ZFP69 | ENSG00000187815 | C2H2 ZF | ZNF124 | ENSG00000196418 | C2H2 ZF |
| ZFP69B | ENSG00000187801 | C2H2 ZF | ZNF131 | ENSG00000172262 | C2H2 ZF |
| ZFP82 | ENSG00000181007 | C2H2 ZF | ZNF132 | ENSG00000131849 | C2H2 ZF |
| ZFP90 | ENSG00000184939 | C2H2 ZF | ZNF133 | ENSG00000125846 | C2H2 ZF |
| ZFP91 | ENSG00000186660 | C2H2 ZF | ZNF134 | ENSG00000213762 | C2H2 ZF |
| ZFP92 | ENSG00000189420 | C2H2 ZF | ZNF135 | ENSG00000176293 | C2H2 ZF |
| ZFPM1 | ENSG00000179588 | C2H2 ZF | ZNF136 | ENSG00000196646 | C2H2 ZF |
| ZFPM2 | ENSG00000169946 | C2H2 ZF | ZNF138 | ENSG00000197008 | C2H2 ZF |
| ZFX | ENSG00000005889 | C2H2 ZF | ZNF14 | ENSG00000105708 | C2H2 ZF |
| ZFY | ENSG00000067646 | C2H2 ZF | ZNF140 | ENSG00000196387 | C2H2 ZF |
| ZGLP1 | ENSG00000220201 | GATA | ZNF141 | ENSG00000131127 | C2H2 ZF |
| ZGPAT | ENSG00000197114 | CCCH ZF | ZNF142 | ENSG00000115568 | C2H2 ZF |
| ZHX1 | ENSG00000165156 | Homeodomain | ZNF143 | ENSG00000166478 | C2H2 ZF |
| ZHX2 | ENSG00000178764 | Homeodomain | | | |
| ZNF146 | ENSG00000167635 | C2H2 ZF | ZNF227 | ENSG00000131115 | C2H2 ZF |
| ZNF148 | ENSG00000163848 | C2H2 ZF | ZNF229 | ENSG00000278318 | C2H2 ZF |
| ZNF154 | ENSG00000179909 | C2H2 ZF | ZNF23 | ENSG00000167377 | C2H2 ZF |
| ZNF155 | ENSG00000204920 | C2H2 ZF | ZNF230 | ENSG00000159882 | C2H2 ZF |
| ZNF157 | ENSG00000147117 | C2H2 ZF | ZNF232 | ENSG00000167840 | C2H2 ZF |
| ZNF16 | ENSG00000170631 | C2H2 ZF | ZNF233 | ENSG00000159915 | C2H2 ZF |
| ZNF160 | ENSG00000170949 | C2H2 ZF | ZNF234 | ENSG00000263002 | C2H2 ZF |
| ZNF165 | ENSG00000197279 | C2H2 ZF | ZNF235 | ENSG00000159917 | C2H2 ZF |
| ZNF169 | ENSG00000175787 | C2H2 ZF | ZNF236 | ENSG00000130856 | C2H2 ZF |
| ZNF17 | ENSG00000186272 | C2H2 ZF | ZNF239 | ENSG00000196793 | C2H2 ZF |
| ZNF174 | ENSG00000103343 | C2H2 ZF | ZNF24 | ENSG00000172466 | C2H2 ZF |
| ZNF175 | ENSG00000105497 | C2H2 ZF | ZNF248 | ENSG00000198105 | C2H2 ZF |
| ZNF177 | ENSG00000188629 | C2H2 ZF | ZNF25 | ENSG00000175395 | C2H2 ZF |
| ZNF18 | ENSG00000154957 | C2H2 ZF | ZNF250 | ENSG00000196150 | C2H2 ZF |
| ZNF180 | ENSG00000167384 | C2H2 ZF | ZNF251 | ENSG00000198169 | C2H2 ZF |
| ZNF181 | ENSG00000197841 | C2H2 ZF | ZNF253 | ENSG00000256771 | C2H2 ZF |
| ZNF182 | ENSG00000147118 | C2H2 ZF | ZNF254 | ENSG00000213096 | C2H2 ZF |
| ZNF184 | ENSG00000096654 | C2H2 ZF | ZNF256 | ENSG00000152454 | C2H2 ZF |
| ZNF189 | ENSG00000136870 | C2H2 ZF | ZNF257 | ENSG00000197134 | C2H2 ZF |
| ZNF19 | ENSG00000157429 | C2H2 ZF | ZNF26 | ENSG00000198393 | C2H2 ZF |
| ZNF195 | ENSG00000005801 | C2H2 ZF | ZNF260 | ENSG00000254004 | C2H2 ZF |
| ZNF197 | ENSG00000186448 | C2H2 ZF | ZNF263 | ENSG00000006194 | C2H2 ZF |
| ZNF2 | ENSG00000275111 | C2H2 ZF | ZNF264 | ENSG00000083844 | C2H2 ZF |
| ZNF20 | ENSG00000132010 | C2H2 ZF | ZNF266 | ENSG00000174652 | C2H2 ZF |
| ZNF200 | ENSG00000010539 | C2H2 ZF | ZNF267 | ENSG00000185947 | C2H2 ZF |
| ZNF202 | ENSG00000166261 | C2H2 ZF | ZNF268 | ENSG00000090612 | C2H2 ZF |
| ZNF205 | ENSG00000122386 | C2H2 ZF | ZNF273 | ENSG00000198039 | C2H2 ZF |
| ZNF207 | ENSG00000010244 | C2H2 ZF | ZNF274 | ENSG00000171606 | C2H2 ZF |
| ZNF208 | ENSG00000160321 | C2H2 ZF | ZNF275 | ENSG00000063587 | C2H2 ZF |
| ZNF211 | ENSG00000121417 | C2H2 ZF | ZNF276 | ENSG00000158805 | C2H2 ZF |
| ZNF212 | ENSG00000170260 | C2H2 ZF | ZNF277 | ENSG00000198839 | C2H2 ZF; BED |
| | | | | | ZF |
| ZNF213 | ENSG00000085644 | C2H2 ZF | ZNF28 | ENSG00000198538 | C2H2 ZF |
| ZNF214 | ENSG00000149050 | C2H2 ZF | ZNF280A | ENSG00000169548 | C2H2 ZF |
| ZNF215 | ENSG00000149054 | C2H2 ZF | ZNF280B | ENSG00000275004 | C2H2 ZF |
| ZNF217 | ENSG00000171940 | C2H2 ZF | ZNF280C | ENSG00000056277 | C2H2 ZF |
| ZNF219 | ENSG00000165804 | C2H2 ZF | ZNF280D | ENSG00000137871 | C2H2 ZF |
| ZNF22 | ENSG00000165512 | C2H2 ZF | ZNF281 | ENSG00000162702 | C2H2 ZF |
| ZNF221 | ENSG00000159905 | C2H2 ZF | ZNF282 | ENSG00000170265 | C2H2 ZF |
| ZNF222 | ENSG00000159885 | C2H2 ZF | ZNF283 | ENSG00000167637 | C2H2 ZF |
| ZNF223 | ENSG00000178386 | C2H2 ZF | ZNF284 | ENSG00000186026 | C2H2 ZF |
| ZNF224 | ENSG00000267680 | C2H2 ZF | ZNF285 | ENSG00000267508 | C2H2 ZF |
| ZNF225 | ENSG00000256294 | C2H2 ZF | ZNF286A | ENSG00000187607 | C2H2 ZF |
| ZNF226 | ENSG00000167380 | C2H2 ZF | | | |
| ZNF286B | ENSG00000249459 | C2H2 ZF | ZNF367 | ENSG00000165244 | C2H2 ZF |
| ZNF287 | ENSG00000141040 | C2H2 ZF | ZNF37A | ENSG00000075407 | C2H2 ZF |
| ZNF292 | ENSG00000188994 | C2H2 ZF | ZNF382 | ENSG00000161298 | C2H2 ZF |
| ZNF296 | ENSG00000170684 | C2H2 ZF | ZNF383 | ENSG00000188283 | C2H2 ZF |
| ZNF3 | ENSG00000166526 | C2H2 ZF | ZNF384 | ENSG00000126746 | C2H2 ZF |
| ZNF30 | ENSG00000168661 | C2H2 ZF | ZNF385A | ENSG00000161642 | C2H2 ZF |
| ZNF300 | ENSG00000145908 | C2H2 ZF | ZNF385B | ENSG00000144331 | C2H2 ZF |
| ZNF302 | ENSG00000089335 | C2H2 ZF | ZNF385C | ENSG00000187595 | C2H2 ZF |
| ZNF304 | ENSG00000131845 | C2H2 ZF | ZNF385D | ENSG00000151789 | C2H2 ZF |
| ZNF311 | ENSG00000197935 | C2H2 ZF | ZNF391 | ENSG00000124613 | C2H2 ZF |
| ZNF316 | ENSG00000205903 | C2H2 ZF | ZNF394 | ENSG00000160908 | C2H2 ZF |
| ZNF317 | ENSG00000130803 | C2H2 ZF | ZNF395 | ENSG00000186918 | C2H2 ZF |
| ZNF318 | ENSG00000171467 | C2H2 ZF | ZNF396 | ENSG00000186496 | C2H2 ZF |
| ZNF319 | ENSG00000166188 | C2H2 ZF | ZNF397 | ENSG00000186812 | C2H2 ZF |
| ZNF32 | ENSG00000169740 | C2H2 ZF | ZNF398 | ENSG00000197024 | C2H2 ZF |
| ZNF320 | ENSG00000182986 | C2H2 ZF | ZNF404 | ENSG00000176222 | C2H2 ZF |
| ZNF322 | ENSG00000181315 | C2H2 ZF | ZNF407 | ENSG00000215421 | C2H2 ZF |
| ZNF324 | ENSG00000083812 | C2H2 ZF | ZNF408 | ENSG00000175213 | C2H2 ZF |
| ZNF324B | ENSG00000249471 | C2H2 ZF | ZNF41 | ENSG00000147124 | C2H2 ZF |
| ZNF326 | ENSG00000162664 | C2H2 ZF | ZNF410 | ENSG00000119725 | C2H2 ZF |
| ZNF329 | ENSG00000181894 | C2H2 ZF | ZNF414 | ENSG00000133250 | C2H2 ZF |
| ZNF331 | ENSG00000130844 | C2H2 ZF | ZNF415 | ENSG00000170954 | C2H2 ZF |
| ZNF333 | ENSG00000160961 | C2H2 ZF | ZNF416 | ENSG00000083817 | C2H2 ZF |
| ZNF334 | ENSG00000198185 | C2H2 ZF | ZNF417 | ENSG00000173480 | C2H2 ZF |
| ZNF335 | ENSG00000198026 | C2H2 ZF | ZNF418 | ENSG00000196724 | C2H2 ZF |
| ZNF337 | ENSG00000130684 | C2H2 ZF | ZNF419 | ENSG00000105136 | C2H2 ZF |
| ZNF33A | ENSG00000189180 | C2H2 ZF | ZNF420 | ENSG00000197050 | C2H2 ZF |
| ZNF33B | ENSG00000196693 | C2H2 ZF | ZNF423 | ENSG00000102935 | C2H2 ZF |
| ZNF34 | ENSG00000196378 | C2H2 ZF | ZNF425 | ENSG00000204947 | C2H2 ZF |
| ZNF341 | ENSG00000131061 | C2H2 ZF | ZNF426 | ENSG00000130818 | C2H2 ZF |
| ZNF343 | ENSG00000088876 | C2H2 ZF | ZNF428 | ENSG00000131116 | C2H2 ZF |
| ZNF345 | ENSG00000251247 | C2H2 ZF | ZNF429 | ENSG00000197013 | C2H2 ZF |
| ZNF346 | ENSG00000113761 | C2H2 ZF | ZNF43 | ENSG00000198521 | C2H2 ZF |
| ZNF347 | ENSG00000197937 | C2H2 ZF | ZNF430 | ENSG00000118620 | C2H2 ZF |
| ZNF35 | ENSG00000169981 | C2H2 ZF | ZNF431 | ENSG00000196705 | C2H2 ZF |
| ZNF350 | ENSG00000256683 | C2H2 ZF | ZNF432 | ENSG00000256087 | C2H2 ZF |
| ZNF354A | ENSG00000169131 | C2H2 ZF | ZNF433 | ENSG00000197647 | C2H2 ZF |
| ZNF354B | ENSG00000178338 | C2H2 ZF | ZNF436 | ENSG00000125945 | C2H2 ZF |
| ZNF354C | ENSG00000177932 | C2H2 ZF | ZNF438 | ENSG00000183621 | C2H2 ZF |
| ZNF358 | ENSG00000198816 | C2H2 ZF | ZNF439 | ENSG00000171291 | C2H2 ZF |
| ZNF362 | ENSG00000160094 | C2H2 ZF | ZNF44 | ENSG00000197857 | C2H2 ZF |
| ZNF365 | ENSG00000138311 | C2H2 ZF | ZNF440 | ENSG00000171295 | C2H2 ZF |
| ZNF366 | ENSG00000178175 | C2H2 ZF | ZNF441 | ENSG00000197044 | C2H2 ZF |
| ZNF442 | ENSG00000198342 | C2H2 ZF | ZNF512 | ENSG00000243943 | C2H2 ZF; BED |
| | | | | | ZF |
| ZNF443 | ENSG00000180855 | C2H2 ZF | ZNF512B | ENSG00000196700 | C2H2 ZF |
| ZNF444 | ENSG00000167685 | C2H2 ZF | ZNF513 | ENSG00000163795 | C2H2 ZF |
| ZNF445 | ENSG00000185219 | C2H2 ZF | ZNF514 | ENSG00000144026 | C2H2 ZF |
| ZNF446 | ENSG00000083838 | C2H2 ZF | ZNF516 | ENSG00000101493 | C2H2 ZF |
| ZNF449 | ENSG00000173275 | C2H2 ZF | ZNF517 | ENSG00000197363 | C2H2 ZF |
| ZNF45 | ENSG00000124459 | C2H2 ZF | ZNF518A | ENSG00000177853 | C2H2 ZF |
| ZNF451 | ENSG00000112200 | C2H2 ZF | ZNF518B | ENSG00000178163 | C2H2 ZF |
| ZNF454 | ENSG00000178187 | C2H2 ZF | ZNF519 | ENSG00000175322 | C2H2 ZF |
| ZNF460 | ENSG00000197714 | C2H2 ZF | ZNF521 | ENSG00000198795 | C2H2 ZF |
| ZNF461 | ENSG00000197808 | C2H2 ZF | ZNF524 | ENSG00000171443 | C2H2 ZF; AT |
| | | | | | hook |
| ZNF462 | ENSG00000148143 | C2H2 ZF | ZNF525 | ENSG00000203326 | C2H2 ZF |
| ZNF467 | ENSG00000181444 | C2H2 ZF | ZNF526 | ENSG00000167625 | C2H2 ZF |
| ZNF468 | ENSG00000204604 | C2H2 ZF | ZNF527 | ENSG00000189164 | C2H2 ZF |
| ZNF469 | ENSG00000225614 | C2H2 ZF | ZNF528 | ENSG00000167555 | C2H2 ZF |
| ZNF470 | ENSG00000197016 | C2H2 ZF | ZNF529 | ENSG00000186020 | C2H2 ZF |
| ZNF471 | ENSG00000196263 | C2H2 ZF | ZNF530 | ENSG00000183647 | C2H2 ZF |
| ZNF473 | ENSG00000142528 | C2H2 ZF | ZNF532 | ENSG00000074657 | C2H2 ZF |
| ZNF474 | ENSG00000164185 | C2H2 ZF | ZNF534 | ENSG00000198633 | C2H2 ZF |
| ZNF479 | ENSG00000185177 | C2H2 ZF | ZNF536 | ENSG00000198597 | C2H2 ZF |
| ZNF48 | ENSG00000180035 | C2H2 ZF | ZNF540 | ENSG00000171817 | C2H2 ZF |
| ZNF480 | ENSG00000198464 | C2H2 ZF | ZNF541 | ENSG00000118156 | C2H2 ZF; |
| | | | | | Myb/SANT |
| ZNF483 | ENSG00000173258 | C2H2 ZF | ZNF543 | ENSG00000178229 | C2H2 ZF |
| ZNF484 | ENSG00000127081 | C2H2 ZF | ZNF544 | ENSG00000198131 | C2H2 ZF |
| ZNF485 | ENSG00000198298 | C2H2 ZF | ZNF546 | ENSG00000187187 | C2H2 ZF |
| ZNF486 | ENSG00000256229 | C2H2 ZF | ZNF547 | ENSG00000152433 | C2H2 ZF |
| ZNF487 | ENSG00000243660 | C2H2 ZF | ZNF548 | ENSG00000188785 | C2H2 ZF |
| ZNF488 | ENSG00000265763 | C2H2 ZF | ZNF549 | ENSG00000121406 | C2H2 ZF |
| ZNF490 | ENSG00000188033 | C2H2 ZF | ZNF550 | ENSG00000251369 | C2H2 ZF |
| ZNF491 | ENSG00000177599 | C2H2 ZF | ZNF551 | ENSG00000204519 | C2H2 ZF |
| ZNF492 | ENSG00000229676 | C2H2 ZF | ZNF552 | ENSG00000178935 | C2H2 ZF |
| ZNF493 | ENSG00000196268 | C2H2 ZF | ZNF554 | ENSG00000172006 | C2H2 ZF |
| ZNF496 | ENSG00000162714 | C2H2 ZF | ZNF555 | ENSG00000186300 | C2H2 ZF |
| ZNF497 | ENSG00000174586 | C2H2 ZF | ZNF556 | ENSG00000172000 | C2H2 ZF |
| ZNF500 | ENSG00000103199 | C2H2 ZF | ZNF557 | ENSG00000130544 | C2H2 ZF |
| ZNF501 | ENSG00000186446 | C2H2 ZF | ZNF558 | ENSG00000167785 | C2H2 ZF |
| ZNF502 | ENSG00000196653 | C2H2 ZF | ZNF559 | ENSG00000188321 | C2H2 ZF |
| ZNF503 | ENSG00000165655 | C2H2 ZF | ZNF560 | ENSG00000198028 | C2H2 ZF |
| ZNF506 | ENSG00000081665 | C2H2 ZF | ZNF561 | ENSG00000171469 | C2H2 ZF |
| ZNF507 | ENSG00000168813 | C2H2 ZF | ZNF562 | ENSG00000171466 | C2H2 ZF |
| ZNF510 | ENSG00000081386 | C2H2 ZF | ZNF563 | ENSG00000188868 | C2H2 ZF |
| ZNF511 | ENSG00000198546 | C2H2 ZF | | | |
| ZNF564 | ENSG00000249709 | C2H2 ZF | ZNF613 | ENSG00000176024 | C2H2 ZF |
| ZNF565 | ENSG00000196357 | C2H2 ZF | ZNF614 | ENSG00000142556 | C2H2 ZF |
| ZNF566 | ENSG00000186017 | C2H2 ZF | ZNF615 | ENSG00000197619 | C2H2 ZF |
| ZNF567 | ENSG00000189042 | C2H2 ZF | ZNF616 | ENSG00000204611 | C2H2 ZF |
| ZNF568 | ENSG00000198453 | C2H2 ZF | ZNF618 | ENSG00000157657 | C2H2 ZF |
| ZNF569 | ENSG00000196437 | C2H2 ZF | ZNF619 | ENSG00000177873 | C2H2 ZF |
| ZNF57 | ENSG00000171970 | C2H2 ZF | ZNF620 | ENSG00000177842 | C2H2 ZF |
| ZNF570 | ENSG00000171827 | C2H2 ZF | ZNF621 | ENSG00000172888 | C2H2 ZF |
| ZNF571 | ENSG00000180479 | C2H2 ZF | ZNF623 | ENSG00000183309 | C2H2 ZF |
| ZNF572 | ENSG00000180938 | C2H2 ZF | ZNF624 | ENSG00000197566 | C2H2 ZF |
| ZNF573 | ENSG00000189144 | C2H2 ZF | ZNF625 | ENSG00000257591 | C2H2 ZF |
| ZNF574 | ENSG00000105732 | C2H2 ZF | ZNF626 | ENSG00000188171 | C2H2 ZF |
| ZNF575 | ENSG00000176472 | C2H2 ZF | ZNF627 | ENSG00000198551 | C2H2 ZF |
| ZNF576 | ENSG00000124444 | C2H2 ZF | ZNF628 | ENSG00000197483 | C2H2 ZF |
| ZNF577 | ENSG00000161551 | C2H2 ZF | ZNF629 | ENSG00000102870 | C2H2 ZF |
| ZNF578 | ENSG00000258405 | C2H2 ZF | ZNF630 | ENSG00000221994 | C2H2 ZF |
| ZNF579 | ENSG00000218891 | C2H2 ZF | ZNF639 | ENSG00000121864 | C2H2 ZF |
| ZNF580 | ENSG00000213015 | C2H2 ZF | ZNF641 | ENSG00000167528 | C2H2 ZF |
| ZNF581 | ENSG00000171425 | C2H2 ZF | ZNF644 | ENSG00000122482 | C2H2 ZF |
| ZNF582 | ENSG00000018869 | C2H2 ZF | ZNF645 | ENSG00000175809 | C2H2 ZF |
| ZNF583 | ENSG00000198440 | C2H2 ZF | ZNF646 | ENSG00000167395 | C2H2 ZF |
| ZNF584 | ENSG00000171574 | C2H2 ZF | ZNF648 | ENSG00000179930 | C2H2 ZF |
| ZNF585A | ENSG00000196967 | C2H2 ZF | ZNF649 | ENSG00000198093 | C2H2 ZF |
| ZNF585B | ENSG00000245680 | C2H2 ZF | ZNF652 | ENSG00000198740 | C2H2 ZF |
| ZNF586 | ENSG00000083828 | C2H2 ZF | ZNF653 | ENSG00000161914 | C2H2 ZF; AT |
| | | | | | hook |
| ZNF587 | ENSG00000198466 | C2H2 ZF | ZNF654 | ENSG00000175105 | C2H2 ZF |
| ZNF587B | ENSG00000269343 | C2H2 ZF | ZNF655 | ENSG00000197343 | C2H2 ZF |
| ZNF589 | ENSG00000164048 | C2H2 ZF | ZNF658 | ENSG00000274349 | C2H2 ZF |
| ZNF592 | ENSG00000166716 | C2H2 ZF | ZNF66 | ENSG00000160229 | C2H2 ZF |
| ZNF594 | ENSG00000180626 | C2H2 ZF | ZNF660 | ENSG00000144792 | C2H2 ZF |
| ZNF595 | ENSG00000272602 | C2H2 ZF | ZNF662 | ENSG00000182983 | C2H2 ZF |
| ZNF596 | ENSG00000172748 | C2H2 ZF | ZNF664 | ENSG00000179195 | C2H2 ZF |
| ZNF597 | ENSG00000167981 | C2H2 ZF | ZNF665 | ENSG00000197497 | C2H2 ZF |
| ZNF598 | ENSG00000167962 | C2H2 ZF | ZNF667 | ENSG00000198046 | C2H2 ZF |
| ZNF599 | ENSG00000153896 | C2H2 ZF | ZNF668 | ENSG00000167394 | C2H2 ZF |
| ZNF600 | ENSG00000189190 | C2H2 ZF | ZNF669 | ENSG00000188295 | C2H2 ZF |
| ZNF605 | ENSG00000196458 | C2H2 ZF | ZNF670 | ENSG00000277462 | C2H2 ZF |
| ZNF606 | ENSG00000166704 | C2H2 ZF | ZNF671 | ENSG00000083814 | C2H2 ZF |
| ZNF607 | ENSG00000198182 | C2H2 ZF | ZNF672 | ENSG00000171161 | C2H2 ZF |
| ZNF608 | ENSG00000168916 | C2H2 ZF | ZNF674 | ENSG00000251192 | C2H2 ZF |
| ZNF609 | ENSG00000180357 | C2H2 ZF | ZNF675 | ENSG00000197372 | C2H2 ZF |
| ZNF610 | ENSG00000167554 | C2H2 ZF | ZNF676 | ENSG00000196109 | C2H2 ZF |
| ZNF611 | ENSG00000213020 | C2H2 ZF | | | |
| ZNF677 | ENSG00000197928 | C2H2 ZF | ZNF726 | ENSG00000213967 | C2H2 ZF |
| ZNF678 | ENSG00000181450 | C2H2 ZF | ZNF727 | ENSG00000214652 | C2H2 ZF |
| ZNF679 | ENSG00000197123 | C2H2 ZF | ZNF728 | ENSG00000269067 | C2H2 ZF |
| ZNF680 | ENSG00000173041 | C2H2 ZF | ZNF729 | ENSG00000196350 | C2H2 ZF |
| ZNF681 | ENSG00000196172 | C2H2 ZF | ZNF730 | ENSG00000183850 | C2H2 ZF |
| ZNF682 | ENSG00000197124 | C2H2 ZF | ZNF732 | ENSG00000186777 | C2H2 ZF |
| ZNF683 | ENSG00000176083 | C2H2 ZF | ZNF735 | ENSG00000223614 | C2H2 ZF |
| ZNF684 | ENSG00000117010 | C2H2 ZF | ZNF736 | ENSG00000234444 | C2H2 ZF |
| ZNF687 | ENSG00000143373 | C2H2 ZF | ZNF737 | ENSG00000237440 | C2H2 ZF |
| ZNF688 | ENSG00000229809 | C2H2 ZF | ZNF74 | ENSG00000185252 | C2H2 ZF |
| ZNF689 | ENSG00000156853 | C2H2 ZF | ZNF740 | ENSG00000139651 | C2H2 ZF |
| ZNF69 | ENSG00000198429 | C2H2 ZF | ZNF746 | ENSG00000181220 | C2H2 ZF |
| ZNF691 | ENSG00000164011 | C2H2 ZF | ZNF747 | ENSG00000169955 | C2H2 ZF |
| ZNF692 | ENSG00000171163 | C2H2 ZF | ZNF749 | ENSG00000186230 | C2H2 ZF |
| ZNF695 | ENSG00000197472 | C2H2 ZF | ZNF750 | ENSG00000141579 | C2H2 ZF |
| ZNF696 | ENSG00000185730 | C2H2 ZF | ZNF75A | ENSG00000162086 | C2H2 ZF |
| ZNF697 | ENSG00000143067 | C2H2 ZF | ZNF75D | ENSG00000186376 | C2H2 ZF |
| ZNF699 | ENSG00000196110 | C2H2 ZF | ZNF76 | ENSG00000065029 | C2H2 ZF |
| ZNF7 | ENSG00000147789 | C2H2 ZF | ZNF761 | ENSG00000160336 | C2H2 ZF |
| ZNF70 | ENSG00000187792 | C2H2 ZF | ZNF763 | ENSG00000197054 | C2H2 ZF |
| ZNF700 | ENSG00000196757 | C2H2 ZF | ZNF764 | ENSG00000169951 | C2H2 ZF |
| ZNF701 | ENSG00000167562 | C2H2 ZF | ZNF765 | ENSG00000196417 | C2H2 ZF |
| ZNF703 | ENSG00000183779 | C2H2 ZF | ZNF766 | ENSG00000196214 | C2H2 ZF |
| ZNF704 | ENSG00000164684 | C2H2 ZF | ZNF768 | ENSG00000169957 | C2H2 ZF |
| ZNF705A | ENSG00000196946 | C2H2 ZF | ZNF77 | ENSG00000175691 | C2H2 ZF |
| ZNF705B | ENSG00000215356 | C2H2 ZF | ZNF770 | ENSG00000198146 | C2H2 ZF |
| ZNF705D | ENSG00000215343 | C2H2 ZF | ZNF771 | ENSG00000179965 | C2H2 ZF |
| ZNF705E | ENSG00000214534 | C2H2 ZF | ZNF772 | ENSG00000197128 | C2H2 ZF |
| ZNF705G | ENSG00000215372 | C2H2 ZF | ZNF773 | ENSG00000152439 | C2H2 ZF |
| ZNF706 | ENSG00000120963 | C2H2 ZF | ZNF774 | ENSG00000196391 | C2H2 ZF |
| ZNF707 | ENSG00000181135 | C2H2 ZF | ZNF775 | ENSG00000196456 | C2H2 ZF |
| ZNF708 | ENSG00000182141 | C2H2 ZF | ZNF776 | ENSG00000152443 | C2H2 ZF |
| ZNF709 | ENSG00000242852 | C2H2 ZF | ZNF777 | ENSG00000196453 | C2H2 ZF |
| ZNF71 | ENSG00000197951 | C2H2 ZF | ZNF778 | ENSG00000170100 | C2H2 ZF |
| ZNF710 | ENSG00000140548 | C2H2 ZF | ZNF780A | ENSG00000197782 | C2H2 ZF |
| ZNF711 | ENSG00000147180 | C2H2 ZF | ZNF780B | ENSG00000128000 | C2H2 ZF |
| ZNF713 | ENSG00000178665 | C2H2 ZF | ZNF781 | ENSG00000196381 | C2H2 ZF |
| ZNF714 | ENSG00000160352 | C2H2 ZF | ZNF782 | ENSG00000196597 | C2H2 ZF |
| ZNF716 | ENSG00000182111 | C2H2 ZF | ZNF783 | ENSG00000204946 | C2H2 ZF |
| ZNF717 | ENSG00000227124 | C2H2 ZF | ZNF784 | ENSG00000179922 | C2H2 ZF |
| ZNF718 | ENSG00000250312 | C2H2 ZF | ZNF785 | ENSG00000197162 | C2H2 ZF |
| ZNF721 | ENSG00000182903 | C2H2 ZF | ZNF786 | ENSG00000197362 | C2H2 ZF |
| ZNF724 | ENSG00000196081 | C2H2 ZF | ZNF787 | ENSG00000142409 | C2H2 ZF |
| ZNF788 | ENSG00000214189 | C2H2 ZF | ZNF880 | ENSG00000221923 | C2H2 ZF |
| ZNF789 | ENSG00000198556 | C2H2 ZF | ZNF883 | ENSG00000228623 | C2H2 ZF |
| ZNF79 | ENSG00000196152 | C2H2 ZF | ZNF888 | ENSG00000213793 | C2H2 ZF |
| ZNF790 | ENSG00000197863 | C2H2 ZF | ZNF891 | ENSG00000214029 | C2H2 ZF |
| ZNF791 | ENSG00000173875 | C2H2 ZF | ZNF90 | ENSG00000213988 | C2H2 ZF |
| ZNF792 | ENSG00000180884 | C2H2 ZF | ZNF91 | ENSG00000167232 | C2H2 ZF |
| ZNF793 | ENSG00000188227 | C2H2 ZF | ZNF92 | ENSG00000146757 | C2H2 ZF |
| ZNF799 | ENSG00000196466 | C2H2 ZF | ZNF93 | ENSG00000184635 | C2H2 ZF |
| ZNF8 | ENSG00000278129 | C2H2 ZF | ZNF98 | ENSG00000197360 | C2H2 ZF |
| ZNF80 | ENSG00000174255 | C2H2 ZF | ZNF99 | ENSG00000213973 | C2H2 ZF |
| ZNF800 | ENSG00000048405 | C2H2 ZF | ZSCAN1 | ENSG00000152467 | C2H2 ZF |
| ZNF804A | ENSG00000170396 | C2H2 ZF | ZSCAN10 | ENSG00000130182 | C2H2 ZF |
| ZNF804B | ENSG00000182348 | C2H2 ZF | ZSCAN12 | ENSG00000158691 | C2H2 ZF |
| ZNF805 | ENSG00000204524 | C2H2 ZF | ZSCAN16 | ENSG00000196812 | C2H2 ZF |
| ZNF808 | ENSG00000198482 | C2H2 ZF | ZSCAN18 | ENSG00000121413 | C2H2 ZF |
| ZNF81 | ENSG00000197779 | C2H2 ZF | ZSCAN2 | ENSG00000176371 | C2H2 ZF |
| ZNF813 | ENSG00000198346 | C2H2 ZF | ZSCAN20 | ENSG00000121903 | C2H2 ZF |
| ZNF814 | ENSG00000204514 | C2H2 ZF | ZSCAN21 | ENSG00000166529 | C2H2 ZF |
| ZNF816 | ENSG00000180257 | C2H2 ZF | ZSCAN22 | ENSG00000182318 | C2H2 ZF |
| ZNF821 | ENSG00000102984 | C2H2 ZF | ZSCAN23 | ENSG00000187987 | C2H2 ZF |
| ZNF823 | ENSG00000197933 | C2H2 ZF | ZSCAN25 | ENSG00000197037 | C2H2 ZF |
| ZNF827 | ENSG00000151612 | C2H2 ZF | ZSCAN26 | ENSG00000197062 | C2H2 ZF |
| ZNF829 | ENSG00000185869 | C2H2 ZF | ZSCAN29 | ENSG00000140265 | C2H2 ZF |
| ZNF83 | ENSG00000167766 | C2H2 ZF | ZSCAN30 | ENSG00000186814 | C2H2 ZF |
| ZNF830 | ENSG00000198783 | C2H2 ZF | ZSCAN31 | ENSG00000235109 | C2H2 ZF |
| ZNF831 | ENSG00000124203 | C2H2 ZF | ZSCAN32 | ENSG00000140987 | C2H2 ZF |
| ZNF835 | ENSG00000127903 | C2H2 ZF | ZSCAN4 | ENSG00000180532 | C2H2 ZF |
| ZNF836 | ENSG00000196267 | C2H2 ZF | ZSCAN5A | ENSG00000131848 | C2H2 ZF |
| ZNF837 | ENSG00000152475 | C2H2 ZF | ZSCAN5B | ENSG00000197213 | C2H2 ZF |
| ZNF84 | ENSG00000198040 | C2H2 ZF | ZSCAN5C | ENSG00000204532 | C2H2 ZF |
| ZNF841 | ENSG00000197608 | C2H2 ZF | ZSCAN9 | ENSG00000137185 | C2H2 ZF |
| ZNF843 | ENSG00000176723 | C2H2 ZF | ZUFSP | ENSG00000153975 | C2H2 ZF |
| ZNF844 | ENSG00000223547 | C2H2 ZF | ZXDA | ENSG00000198205 | C2H2 ZF |
| ZNF845 | ENSG00000213799 | C2H2 ZF | ZXDB | ENSG00000198455 | C2H2 ZF |
| ZNF846 | ENSG00000196605 | C2H2 ZF | ZXDC | ENSG00000070476 | C2H2 ZF |
| ZNF85 | ENSG00000105750 | C2H2 ZF | ZZZ3 | ENSG00000036549 | Myb/SANT |
| ZNF850 | ENSG00000267041 | C2H2 ZF | | | |
| ZNF852 | ENSG00000178917 | C2H2 ZF | | | |
| ZNF853 | ENSG00000236609 | C2H2 ZF | | | |
| ZNF860 | ENSG00000197385 | C2H2 ZF | | | |
| ZNF865 | ENSG00000261221 | C2H2 ZF | | | |
| ZNF878 | ENSG00000257446 | C2H2 ZF | | | |
| ZNF879 | ENSG00000234284 | C2H2 ZF | | | |

TABLE 2
Exemplary Human Targets

| | | | | | |
|---|---|---|---|---|---|
| MYT1 | GATAD2B | ZNF100 | ZNF85 | SBNO2 | PROP1 |
| GTF2H2 | IRF7 | TRIM27 | ZNF311 | HES5 | ZNF676 |
| SSBP1 | SMARCB1 | TBX22 | POMZP3 | ZFP57 | NRM |
| MED16 | TCF19 | SALL3 | TAF9 | RAD17 | POLR1H |
| KLF13 | CDK7 | RXRB | NAPEPLD | EHMT2 | TAF4 |
| RING1 | NAP1L4 | TUBB | CLIC1 | MAPK15 | ZNF251 |
| GTF2H4 | BRD2 | GTF2H2C_2 | GLIS2 | ALOX5 | ABCF1 |
| ZNF707 | CSNK2B | ZNF623 | PBX2 | IFI27 | TSPY4 |
| PCGF2 | ZBTB12 | MLLT6 | ATF6B | TADA2A | EPOP |
| TSPY10 | TSPY1 | HNF1B | POU5F1 | LHX1 | TSPY2 |
| HSFY2 | HSFY1 | TGIF2LY | TSPY3 | PHF1 | TSPY8 |
| ZBTB22 | SRY | ZFY | ZNF445 | UTY | ZNF852 |
| ZKSCAN7 | ZNF660 | ZNF197 | ZNF35 | ZNF502 | ZNF501 |
| FOXR1 | MPHOSPH8 | SOHLH2 | PDX1 | MED22 | KPNA3 |
| CDX2 | GTF2F2 | ZNF436 | E2F2 | PHF11 | CHAF1B |
| MLXIP | LEUTX | APP | ELF1 | ZNF546 | ZNF780B |
| ZNF780A | MED15 | DACH1 | KLF5 | TMX4 | NKX2-2 |
| ZNF280A | POU4F1 | INSM1 | ITSN1 | SMOX | NXT1 |
| ZNF280B | NKX2-4 | IPO5 | HBP1 | ZNF343 | FOXO6 |
| LTC4S | MRNIP | XBP1 | EDNRB | LMO7 | ZNF70 |
| ZBTB4 | CDK8 | POLR3E | SOX21 | POLR3F | MCM3AP |
| SCRT2 | ACTN4 | FOXO1 | NCOA6 | ISX | BACH1 |
| RPRD1A | RIT2 | NUP58 | SIRT2 | MAFF | MED18 |
| ASXL1 | ETS2 | SHOX | MBD2 | SUN2 | NOBOX |
| SMAD2 | AGPAT5 | SOX1 | ZBED4 | VCX | GATA4 |
| ZBTB7C | ERG | SOX7 | ZFP1 | GATA3 | ZNF337 |
| HSF1 | NPC1 | SEPHS1 | ZBTB21 | TFDP1 | ZNF24 |
| RUNX1 | KLF6 | TOX2 | TAF3 | PCID2 | TXLNG |
| CEBPB | SIM2 | HMX1 | ZIC2 | TWIST2 | OBI1 |
| SEH1L | AGPAT3 | ZNF396 | GSX1 | GTF2H1 | WDR13 |
| CYBB | PRDM15 | ADNP2 | SCML2 | PKNOX1 | IL15RA |
| ZBTB14 | ZNF521 | NOC4L | MED14 | RAX | TXNL4A |
| NUP50 | MINDY3 | ZNF516 | MTMR8 | ZNF397 | SMAD9 |
| CREM | GABPA | ZNF334 | UNCX | INTS1 | FOXL3 |
| YY2 | PPARA | CTDNEP1 | FOXR2 | MEOX2 | GATA1 |
| FOXK1 | MKX | NRL | HMGN1 | SUN1 | RFXAP |
| POLR1D | POLRMT | EIF5A | SUN5 | KLF8 | BMI1 |
| PLCB1 | PTF1A | IRF9 | REC8 | MED4 | APBB1 |
| ZSCAN30 | RAP1GAP2 | ZNF215 | SNAI1 | HDX | EP300 |
| ARID3A | SKOR2 | TGIF1 | NUP88 | ONECUT3 | TAF4B |
| GZF1 | JMJD1C | ETV1 | PATZ1 | PHF8 | TDRD3 |
| ZNF519 | P2RX1 | VAPA | DNAJC1 | ZNF485 | CITED1 |
| ZNF214 | POLR2E | TSPYL2 | ALOX5AP | DACH2 | FOXA1 |
| AIRE | HIC2 | SMAD4 | TCF4 | TGIF2LX | NFIB |
| CC2D1A | P2RX5 | KLHDC2 | ZNF33B | OSBPL3 | RFX1 |
| ZFP3 | CREB3L3 | SAMD1 | L3MBTL2 | FOXJ2 | ZNF287 |
| RAE1 | ZBTB7A | NFATC4 | IRF4 | TFE3 | DMRTA1 |
| ZNF594 | TSHZ2 | ATP2A3 | ZNF143 | NFIC | EOMES |
| SOX18 | PSIP1 | PCBP3 | NUP62CL | KDM1B | SOX8 |
| E4F1 | XPO4 | RANBP3 | AHR | SIX1 | RAC2 |
| APTX | DBX1 | ZNF322 | FOXQ1 | ZNF705A | ZNF37A |
| TWIST1 | ATOH7 | TNKS | MX1 | SALL4 | MX2 |
| MAFK | BHLHE23 | SP4 | SIX6 | SIX4 | SAFB |
| POU3F4 | KAT2B | CAMTA2 | HIVEP1 | POLR3A | ZKSCAN3 |
| CCAR1 | JARID2 | WT1 | NUP42 | ETV6 | RREB1 |
| TFAP2C | MNAT1 | MTMR6 | NFX1 | RNF6 | ZKSCAN4 |
| NKAPL | ZNF232 | SMARCA2 | NFATC2 | CREB5 | ZNF57 |
| OLIG2 | OLIG1 | ZNF557 | INSR | RARB | ZNF22 |
| MED31 | ANXA11 | FOXF2 | ZSCAN23 | SOX4 | GLYR1 |
| ONECUT2 | ZNF705B | MORC2 | ZNF662 | BCLAF3 | GPER1 |
| PRDM16 | IKZF1 | NR1H3 | ZNF77 | EGR3 | ZNF500 |
| EBF2 | GATA5 | PRKCZ | ZIC5 | L3MBTL4 | RBL1 |
| GATA6 | KLF12 | E2F3 | RPRD1B | FOS | CPTP |
| TFAM | SKI | PHF20 | ZNF32 | CHMP7 | TBX20 |
| GTF2E2 | TSHZ1 | JDP2 | RBPJL | PAX5 | NAP1L2 |
| POM121L12 | TFDP3 | HES3 | RHOXF2 | TEAD4 | GPX4 |
| ZNF558 | CREBBP | C9orf72 | UHRF1 | BCL2 | NUP160 |
| FANCM | LEMD2 | GNAQ | FOXC1 | MYOF | XPO7 |
| CDYL | PSEN1 | FOXB2 | ZNF620 | HHEX | ZIC3 |
| ZNF713 | POLR1E | SUN3 | ZNF438 | CETN2 | RANGAP1 |
| ZNF823 | ZNF440 | HSFX1 | RHOXF1 | CSRNP1 | ZNF441 |
| ZNF136 | ZNF117 | TNKS2 | MYBL2 | ZNF491 | ARNTL2 |
| NFATC1 | ZBTB5 | FOXD4L3 | ZNF735 | TEF | NPIPA1 |
| POLR3H | TERT | ENO1 | ADRA1A | ZKSCAN2 | ZNF236 |
| NKX3-1 | PRICKLE1 | ZNF770 | ITPR3 | PARP11 | SUZ12 |
| UBP1 | PHF13 | ZNF619 | ZNF627 | MEIS2 | DISP3 |
| ZNF333 | NR4A3 | ZNF395 | OTX2 | CENPV | ZNF25 |
| IRX4 | ZNF518A | HMGN5 | CDCA5 | ZNF727 | NFIL3 |
| TMEM38B | BNIP3L | BARX1 | NANOGNB | BICD2 | MCM8 |
| SUPT3H | TMEM38A | MXD4 | SCGB1A1 | VSX2 | RELA |
| AKAP6 | ZNF595 | PLAGL2 | RANBP1 | RFXANK | NR2C2 |
| DNMT3B | MED10 | H3-5 | TAF11L4 | RERE | MSX1 |
| HIRA | ZNF14 | ZBTB33 | E2F8 | NAP1L3 | HMGA1 |
| ZNF367 | ZNF33A | ZNF101 | RNF8 | CDC45 | NELL1 |
| HOXC9 | RUNX2 | ZNF253 | POM121C | NANOG | YY1 |
| OTULINL | NUP155 | ASXL3 | ZBTB46 | RAD51 | RRP12 |
| MVP | CKS2 | CCNT1 | MLXIPL | DDX11 | ZNF66 |
| ZBTB43 | ELK1 | CDC5L | LMX1B | UXT | ZNF486 |
| ZNF682 | ZNF626 | MTA1 | BAHD1 | ZBTB40 | PBX4 |
| PRDM2 | ZNF93 | TCEA1 | RNF4 | VSX1 | BNIP2 |
| TAF11L11 | TGIF2 | TLX1 | ZBTB49 | IPO8 | TMEM201 |
| BCL11B | MTOR | PBRM1 | SHMT2 | TAF11L3 | NEUROD2 |
| TP73 | ZKSCAN1 | GCOM1 | ISL1 | FOXS1 | BCL2L1 |
| ZNF257 | ZNF729 | ZNF492 | OVOL2 | CDC6 | PPARGC1A |
| IKZF3 | SMARCC1 | TAF11L8 | TAF11L9 | ITPRIP | PRKAA1 |
| ZNF208 | TAF11L7 | TAF11L2 | SREBF2 | HOXC10 | ZNF90 |
| ZNF430 | MLIP | ZNF91 | ZNF429 | HOXC11 | HOXC8 |
| WBP2NL | SMAD6 | PRIM2 | TMEM18 | ZNF675 | NUP210 |
| ZNF681 | ZNF99 | SPDEF | MCM5 | THAP1 | POU3F2 |
| MCM4 | MSGN1 | SMC2 | MEF2B | ZNF431 | SFMBT1 |
| SVEP1 | ZNF98 | HMX3 | ZNF708 | ZNF518B | ZNF732 |
| E2F1 | RCOR1 | ZNF721 | TCF7L2 | NUP93 | ST18 |
| ZNF341 | ZNF726 | ZBTB34 | CHMP4B | LHX2 | L3MBTL1 |
| SCML1 | ZNF730 | ZNF506 | NKX3-2 | ZNF728 | HOXC4 |
| CEBPG | FAAP24 | LHX3 | TAF11L5 | TSHZ3 | SKOR1 |
| FAM169A | NR6A1 | ZNF141 | PRDM1 | COQ7 | PCNA |
| FOXB1 | CCND1 | MED21 | ZNF723 | ZNF302 | DPY19L2 |
| STAG2 | ETV2 | GCHFR | FOXA2 | MSL1 | CEBPA |
| SOX11 | PEX2 | POLR1F | POLR2F | RARA | SPIN1 |
| ZNF618 | SOX10 | OSR1 | TOP1 | ZFHX4 | PRNP |
| HMGN2 | MYCN | SOX17 | ZNF484 | BACH2 | POLR2M |
| SALL1 | ETV4 | CTNNB1 | RHOXF2B | SMARCD1 | HSFX4 |
| ZNF275 | FMR1 | ZNF718 | ZNF74 | IKZF5 | HACD3 |
| KDM4D | CABIN1 | SUV39H1 | ZNF157 | CRAMP1 | ATRAID |
| BRD3 | CPNE1 | GABRB1 | KLF4 | TOR1A | PRRX2 |
| MBD1 | NUTF2 | FOXE1 | MED27 | ATAD2B | ZNF536 |
| ZNF790 | THAP11 | CLOCK | NACC2 | RXRA | NKRF |
| ZNF280C | PBX3 | TOX3 | ZNF565 | NKX1-2 | SPIB |
| ZNF705G | ZNF704 | QSOX2 | HNF4G | ZBTB8B | MED17 |
| ZNF292 | CCNH | PAX7 | MED24 | TMEM33 | MAFB |
| ZNF737 | ZBED3 | MSH2 | POLA2 | CASZ1 | ZNF850 |
| TERB1 | EBP | SCMH1 | ZBTB45 | ZNF155 | NFAT5 |
| TRIM28 | TSPYL4 | HEY1 | TFAP2B | ZNF283 | YAP1 |
| IRX3 | NIPBL | ZNF404 | ZNF114 | ZNF716 | SOX3 |
| NFYC | URI1 | EPHA3 | MEOX1 | ATF1 | FOSL2 |
| ZNF576 | ZNF230 | ZNF45 | MED1 | CHMP2A | ZNF222 |
| MAF | PRDM14 | AJUBA | CENPA | FOXI2 | THRAP3 |
| DNTTIP1 | RARG | NKX6-2 | RORB | ZNF286A | CSE1L |
| NAV3 | HOXB9 | ZNF624 | MTDH | KAT2A | HOMEZ |
| SCML4 | RTN4 | SPZ1 | SPIC | ALX1 | ZNF345 |
| ZNF223 | ZFHX2 | ZNF284 | TCFL5 | DPY19L3 | TOR1B |
| HCFC1 | FOXC2 | BNIP3 | CTBP2 | FEZF2 | RLF |
| PTGES | TTF1 | MZF1 | ANKRD17 | CENPB | GRK5 |
| GSC2 | FOXO4 | ZNF497 | TBX1 | ZNF382 | RNF169 |
| ANXA4 | MED12 | ZNF749 | KLF3 | DMRTC2 | BBX |
| GNAZ | ZNF837 | ZNF615 | ZFP90 | VAX1 | TBC1D20 |
| ZNF225 | AR | PGR | NR5A1 | CASC3 | AUTS2 |
| FOXD4L4 | NUP107 | WRN | ZSCAN1 | TAF8 | ZNF234 |
| HNRNPD | RBMX | MYCL | ZNF568 | ZNF614 | ZNF584 |
| BARHL1 | ZNF432 | PAX4 | ZNF329 | MMS19 | MLH3 |
| CDT1 | FOXD4L5 | ZNF461 | PHOX2B | ELK3 | IRF8 |
| SNAI3 | NUDT9 | LINC02218 | ZNF182 | ZNF630 | ZNF79 |
| OIT3 | NKX2-3 | EMX2 | SLC52A3 | NR1D1 | ZNF132 |
| DLX5 | TOR2A | SMARCA1 | MNS1 | HESX1 | POLR2K |
| UGT2B28 | THAP7 | ARID1A | CDK9 | P2RX6 | HOXB4 |
| SYNE4 | WDHD1 | DPPA4 | DPPA2 | ATF4 | E2F5 |
| ZNF420 | ZNF324B | NFIA | ZNF616 | ZNF471 | HSF2 |
| ZNF408 | NR2E3 | ATRX | TFEC | TBX18 | SLC30A9 |
| CEBPD | NKX6-3 | VAX2 | HDAC2 | SPAG4 | GSX2 |
| ZNFX1 | ZNF227 | NCAPH2 | BCLAF1 | SP6 | KLF17 |
| ADNP | ZNF276 | TSPYL5 | SP2 | FOXJ3 | NR2F1 |
| TADA2B | ZNF324 | MEIS3 | CTCF | ZNF860 | ZFP28 |
| NR1D2 | MCM3 | DHRS2 | KLF18 | NEUROG1 | FOXD3 |
| NFKB2 | DPY19L4 | SORL1 | STPG4 | H2AZ1 | CUEDC2 |
| ZNF470 | DLX6 | ZNF586 | ZNF235 | H1-0 | ZBTB32 |
| SOX12 | ZNF274 | ZNF217 | TNMD | TTC5 | ZNF446 |
| SIX2 | SIX3 | FOXL1 | ZFHX3 | ZIM3 | MAZ |
| EGR4 | SMC3 | ZNF212 | PITX1 | NCOA5 | MED23 |
| HNF4A | APEX1 | IRX5 | GTF2A2 | BARX2 | DUXA |
| PLA2G4C | KMT5B | WDR61 | ZNF264 | POLR2C | EPAS1 |
| CDK19 | GMEB2 | RBM15B | SNAI2 | ZNF480 | CHD7 |
| ZNF219 | SUPT16H | ZBED2 | ZGPAT | KPNA1 | SNUPN |
| SLC2A4RG | SMC1A | LMNB1 | CREB3L2 | FOXF1 | SMARCE1 |
| HOXC6 | ZNF835 | MYO6 | ZNF667 | BCL11A | ZNF805 |
| NOTO | ZNF610 | CREB3 | MRPL19 | ZNF783 | FABP1 |
| ZNF572 | LYPLA1 | MLLT3 | ATOH8 | ZNF621 | NPAS1 |
| TAF5 | ZNF740 | NONO | NR1H4 | HOXB8 | FOXG1 |
| UTP18 | MTF1 | TAF11L14 | IRX1 | EBF4 | NOC3L |
| HELLS | ZNF880 | ZSCAN10 | ZNF366 | ETS1 | ZNF213 |
| CLMN | STAG1 | USF2 | HEY2 | OSBPL8 | ZNF263 |
| DMRT1 | DMRT3 | SDCBP | ZSCAN32 | TCF15 | ZBTB42 |
| NCAPH | MACROH2A1 | USP3 | ZIM2 | ZNF174 | ZNF597 |
| ZNF786 | CTCFL | GADD45A | ZC3H4 | FOXN2 | EI24 |
| ZNF460 | SOX14 | RNF20 | POLA1 | NDC1 | ARX |
| ZHX3 | TCF7 | ZNF8 | TCF7L1 | PAX1 | TFAP4 |
| PRICKLE2 | RB1CC1 | NSMF | ZIC1 | HIVEP2 | TBX2 |
| MED30 | RAD21 | SCAI | AEBP2 | ZNF875 | BSX |
| MECP2 | ZNF467 | FOSB | ZNF543 | ZNF133 | ISL2 |
| MYF6 | ARID1B | ZNF229 | ZNF528 | TAF7L | RAD21L1 |
| PKNOX2 | ZFX | ZNF575 | FAF1 | GCH1 | ZNF81 |
| SYNE2 | ZNF629 | SMAD5 | TMEM120A | FOXO3 | RNF180 |
| RGPD2 | ZNF668 | ZNF646 | TAF2 | MAD2L1 | ZNF578 |
| KCNIP3 | CRCP | POLR3B | HSF5 | TOX | RORA |
| ZNF296 | BHLHE22 | PGRMC2 | PTGDS | TAF11L6 | E2F7 |
| ARNT2 | MYC | BCHE | ALG14 | DMRTA2 | HIVEP3 |
| ZHX1 | ELF4 | POU2F3 | HOXC13 | ZSCAN22 | POLD1 |
| ESX1 | HOXB7 | PLAGL1 | NUP37 | BARHL2 | TOR4A |
| MEF2A | TRIM24 | GRHL2 | ATP1B4 | PHC3 | ZNF777 |
| AEN | POU5F1B | CBX1 | ZNF425 | MED13 | ZC3HC1 |
| NCOA2 | POU3F1 | ZNF548 | NFE2L1 | ZNF746 | SH3BGRL2 |
| NRF1 | HOXB6 | ZNHIT1 | MAPK3 | ZNF282 | HOXB1 |
| MEIS1 | HLF | MAJIN | BATF2 | ABL1 | ZNF398 |
| OTX1 | ZSCAN2 | ZNF696 | RELB | NKAP | TSPYL6 |
| ZNF599 | ZBTB6 | ZBTB26 | ZNF195 | GLI4 | TCF23 |
| NR3C2 | ZHX2 | ZFP41 | ZNF181 | PPARGC1B | CREBRF |
| TICRR | NR1H2 | MYF5 | FOXP2 | NR2F2 | HMGN3 |
| SKIL | GTF2H5 | NAP1L5 | CHD8 | DBX2 | FUS |
| EMX1 | EN2 | NCOR2 | SAP30L | TAL2 | SORT1 |
| HOXC12 | POLR2L | HSFX3 | MCM7 | ZFP37 | TAF11 |
| L3MBTL3 | PEG3 | IRX6 | ROGDI | OLIG3 | SIN3A |
| RANBP2 | KDM8 | TAF6 | TLE4 | FLI1 | TAF1 |
| TBP | HAND1 | ZNF879 | ZNF609 | RETSAT | CITED2 |
| FOXH1 | ATMIN | KDM4C | LEO1 | ZNF664 | SMARCA5 |
| TAF11L12 | PAF1 | ZNF26 | DUSP2 | MED29 | ZSCAN21 |
| SUPT5H | ZNF3 | DDX19B | TBPL1 | TCF21 | TRRAP |
| GCM1 | MED7 | ZNF354C | EN1 | ZNF10 | ZFAT |
| SOX2 | ATP11B | FOXP3 | STON1- | NR2E1 | HSFX2 |
| | | | GTF2A1L | | |
| TSPYL1 | SIM1 | NUP153 | ZNF75D | ZNF449 | MECOM |
| ZBTB24 | NUP214 | ATAD2 | POLR3C | TFAP2D | NEUROG2 |
| PLRG1 | TMEM170A | MCM2 | DUXB | CPHXL | ING2 |
| CDK6 | GUCY2F | FOXA3 | MYPOP | NXT2 | NPAS2 |
| GLIS1 | SIX5 | GSC | ASCL1 | ZNF426 | ZNF561 |
| ZNF562 | FAM156B | MSX2 | ZNF846 | CUX2 | POLR3K |
| MED12L | GTF2A1 | ZNF782 | TPRX1 | CRX | ZNF552 |
| ZNF587B | ZNF814 | EP400 | LHX6 | ZNF587 | ZNF92 |
| ZNF417 | BATF | ZNF256 | GTF2E1 | NFATC3 | HELT |
| RANBP17 | ELF5 | PAK1 | IRF2 | TAF9B | KDM6A |
| ZNF473 | NR3C1 | TMEM120B | DUX4 | PLPP7 | ZFP14 |
| ZFP82 | ZNF260 | ZNF529 | RAN | PHF19 | EMD |
| PCM1 | ZNF605 | NKX2-1 | NKX2-8 | PAX9 | TEAD2 |
| GCM2 | WDR3 | WTAP | NANOGP8 | NCAPD3 | P2RX7 |
| RAX2 | ZNF724 | ERCC1 | PKN1 | ZNF43 | KLHDC3 |
| NKX2-5 | ADRA1B | MED26 | ALX3 | POU6F2 | BRAP |
| NHLH2 | KLF2 | NUP62 | TMEM176B | TBX3 | TRA2B |
| ZNF354A | IFT74 | PARP2 | NPAP1 | SCX | ANHX |
| CALR3 | ZNF547 | SCRT1 | SRF | DNAJB14 | CDX4 |
| ACTRT1 | NEMP2 | FAM156A | SOX5 | MCM6 | DMRTC1B |
| ZBED1 | HPF1 | TCF12 | ESRRB | BAX | BHLHE41 |
| CEBPE | HNRNPC | DCTN5 | EBF1 | ZNF585A | YEATS4 |
| PLAG1 | ZNF585B | ZNF792 | ZFP42 | POU5F2 | ZBTB7B |
| FOXN3 | ZBTB25 | ATP5MF | NEUROG3 | ZNF789 | PHOX2A |
| ZNF394 | SOX30 | SLC22A18 | ZNF655 | HES1 | ZFP92 |
| KMT5C | TBR1 | ZSCAN25 | H2BW1 | ARID3C | FOXD4L1 |
| PHC1 | ZNF41 | ZNF628 | ZNF674 | TRPS1 | ZNF524 |
| ZNF784 | ZNF580 | ZNF581 | USP51 | DMRTC1 | SIGMAR1 |
| LDB1 | TBX5 | TAF7 | GLI1 | PITX3 | CREB3L1 |
| ACTB | POLR3GL | MBD3 | TOP2A | SMARCD3 | NFXL1 |
| TBXT | MBD6 | PCYT1A | ZNF699 | ZNF177 | DMTF1 |
| ZNF560 | NUP210L | MACROH2A2 | PHF21A | TCF24 | ZNF583 |
| TAF11L13 | ESR1 | BCL6 | CDK4 | SENP2 | DPY19L1 |
| FOSL1 | ZNF808 | ZKSCAN5 | PLAAT1 | ZNF611 | ZNF600 |
| ZNF28 | ZNF773 | ZNF549 | SNCA | ZNF550 | ZSCAN9 |
| ZNF416 | ZIK1 | BANF1 | ZNF134 | ZNF211 | TBX4 |
| ZC3H8 | ZNF527 | RNF168 | ZNF569 | ZNF793 | ZNF540 |
| TMPO | ZNF571 | ZNF607 | MYRFL | SLC29A2 | ZNF75A |
| ZFP2 | NPAS4 | TAF1L | CSRNP3 | NOTCH1 | ZNF239 |
| MLLT10 | ZNF205 | MXD3 | ZNF175 | H1-1 | H1-2 |
| H1-6 | H1-4 | EPC1 | POLG | ETV3L | SP5 |
| TBX6 | DLX1 | ZNF268 | GMNC | MYRF | ETV5 |
| TAF11L10 | ZNF354B | MCMDC2 | MYOD1 | GTF2B | EIF5A2 |
| JUND | ENY2 | GFI1B | FOXD4 | NUP205 | ATOH1 |
| TOX4 | SCRN1 | FOXM1 | MCM10 | AEBP1 | SALL2 |
| HAND2 | MED28 | MXI1 | MPO | DLX2 | STX1A |
| NUP35 | ELF2 | MED25 | MED6 | NPAS3 | MBTD1 |
| GATA2 | CBX4 | ZNF135 | ZNF221 | PCLAF | ZBTB11 |
| MGST3 | LRWD1 | POLR2J | VRK1 | FOXK2 | POLR2J3 |
| ZNF285 | GTF2A1L | SEC13 | SPATA46 | SSRP1 | P2RX3 |
| POLR2J2 | ZSCAN18 | ZNF419 | ZNF30 | POLR2B | ZNF304 |
| ZNF254 | POU2F1 | ZNF701 | CBX2 | ZNF418 | CDX1 |
| REST | SET | ZNF71 | ZNF570 | ZBTB20 | MLH1 |
| INSM2 | GTPBP4 | POLR1C | NFE2L3 | CBX3 | MRGPRF |
| NFIX | TBX15 | DNAJC2 | LYL1 | ATF5 | MAD2L1BP |
| ESR2 | MATR3 | ZNF705E | SULT1E1 | RXRG | THRB |
| TPR | SMARCD2 | PARP16 | ZNF414 | CCNI | GRWD1 |
| E2F4 | SPHK2 | DBP | NUP188 | ZNF80 | MCMBP |
| ATF2 | UBE2T | H3Y1 | TMEM43 | MEF2C | KASH5 |
| LHX4 | ETV3 | ZNF510 | SATB2 | ZNF778 | ZNF644 |
| MAX | MRPS23 | IGF2R | MESP1 | MESP2 | TFEB |
| FOXD4L6 | PAX2 | NHLH1 | NPM2 | IRF3 | TBX19 |
| SOHLH1 | PRKAA2 | WAC | NR5A2 | HR | SOX15 |
| ZNF526 | ERF | NAP1L1 | UBE2I | VENTX | ZNF511 |
| POU3F3 | EHF | PURB | CHAF1A | ERCC6 | TRPC7 |
| RGPD8 | TMEM97 | RUVBL2 | ZNF248 | POLR3D | SEBOX |
| MYO1C | DRGX | CCND2 | ZNF845 | ZNF765 | EIF5AL1 |
| HMX2 | ZNF813 | DPRX | EED | OSR2 | ZNF48 |
| KLF16 | ZNF771 | IRF6 | ZNF768 | ELK4 | ZNF764 |
| ZNF785 | ZNF689 | TENT4A | NFILZ | NPM3 | LMNB2 |
| NDN | ZNF787 | ZNF444 | ZSCAN5B | GLIS3 | ZNF169 |
| ZNF423 | GLE1 | ZBTB39 | LMX1A | POLR2H | SOX9 |
| ZNF648 | CDH5 | NEMP1 | STAT6 | ACTL6B | CMTM3 |
| TOR3A | ZEB1 | TFAP2A | MYORG | ZNF483 | RNF2 |
| KAT7 | NKX2-6 | MSC | CTBP1 | MAFA | EBF3 |
| ZBTB3 | HDAC7 | POLR2G | TAF6L | KLF7 | DLX4 |
| DLX3 | NXF1 | ZNF493 | SMARCAD1 | ZNF189 | VDR |
| TLX3 | ZNF358 | ZNF658 | FOXI1 | ELF3 | POLR1B |
| PRRX1 | RTF1 | MED19 | GBX2 | PURA | ETV7 |
| BOK | RAG2 | SENP1 | TYRO3 | MED9 | ZNF639 |
| DTL | POLR3G | GTF2H3 | BATF3 | SOX13 | ACTL6A |
| ZNF564 | ZNF490 | ZNF791 | IRF5 | ZNF678 | MRPS14 |
| KLF10 | CGAS | OSBPL6 | EGR2 | LHX9 | KLF9 |
| BHLHE40 | WDFY3 | GLI3 | HOXB13 | MYB | JUNB |
| KLF1 | ERCC3 | ZFP62 | ZNF454 | CACYBP | RFX3 |
| ZNF34 | THRA | NUPR1 | DMRT2 | POU4F2 | CETN3 |
| ZNF7 | ZNF250 | PRIMPOL | ZNF16 | IPO11 | TP53 |
| CTR9 | PURG | SMAD3 | DNMT1 | SP100 | MYOG |
| P2RX4 | ZBED5 | NELFA | ZNF705D | ZNF641 | PAX6 |
| NSD2 | DHX9 | ZNF2 | GTF2IRD1 | TFDP2 | POLE3 |
| SAMD7 | NKX1-1 | NSD1 | UPF1 | RANBP3L | GUCY2D |
| BNIP1 | SIRT1 | DNAJB12 | KLF14 | HES7 | PER1 |
| BHLHA15 | ZIC4 | SP8 | RFX4 | ESRRA | LBX1 |
| CCNT2 | CUX1 | SYNE3 | RNF13 | PROX2 | CREB1 |
| ZNF554 | ZNF555 | ZNF556 | ZBTB1 | MED13L | MYBL1 |
| HIF1A | ATF3 | PLA2G4A | ZNF596 | ZNF148 | HES2 |
| SPI1 | ZNF517 | TERB2 | OTP | ZNF331 | FOXN4 |
| PRDM5 | SFMBT2 | FEZF1 | ZNF280D | BIN1 | BCL2L10 |
| PROX1 | ZNF574 | POU2F2 | ESRRG | ZNF18 | LEF1 |
| ORC5 | APEH | RNF123 | MAD1L1 | SUMO1 | ZNF121 |
| ZNF829 | ZNF772 | RBL2 | ZNF865 | AQP1 | GHRHR |
| ORC3 | ZNF672 | HTATIP2 | ZNF17 | GMNN | HMGN4 |
| BNC1 | H1-5 | FOXL2 | PITX2 | RFX8 | NOS1AP |
| ONECUT1 | ZNF146 | ZNF112 | FOXD1 | NR4A1 | LITAF |
| ZNF514 | FIGLA | ZNF319 | MFSD10 | ZNF688 | CNEP1R1 |
| HIF3A | ARNTL | PLSCR1 | STAG3 | MEIOB | SMC4 |
| PLAC8 | NUDT1 | KPNA4 | NPM1 | ZNF695 | NUP133 |
| CENPF | IPO9 | PRIM1 | RBPJ | MGA | TCF3 |
| RFX7 | WDR82 | IRX2 | BAP1 | ERBIN | ZNF138 |
| ZNF670 | AAAS | WIZ | KLF15 | CLGN | OVOL1 |
| PWWP3A | SP7 | SP1 | BRD4 | KCNH1 | NACC1 |
| ZNF19 | CBX5 | SMAD1 | NFE2 | ZNF76 | ZNF710 |
| ZNF774 | NCAPD2 | PPARD | TEAD3 | GAPDH | TMEM109 |
| E2F6 | NKX6-1 | NR4A2 | ERCC4 | CCNC | NUP98 |
| FERD3L | RECQL5 | CHD4 | STAT4 | ITGB4 | MSH6 |
| POU4F3 | PRKCB | RRM1 | TEAD1 | H3-3B | NUDT21 |
| MNX1 | NFKB1 | NELFE | WBP2 | ZSCAN29 | EZH2 |
| ZNF281 | IST1 | IFFO1 | MNT | CALR | ZNF821 |
| PAFAH1B1 | NUCKS1 | ZNF853 | ZNF316 | ZNF12 | NLRP6 |
| EGFR | SETD7 | MGST2 | H1-3 | VEZF1 | ZNF202 |
| HOXA1 | HOXA2 | HOXA4 | LBX2 | PCGF1 | HOXA5 |
| HOXA6 | HOXA7 | HOXA9 | TLX2 | HOXA10 | NFYB |
| HOXA11 | HOXA13 | EVX1 | RFX2 | ASF1A | MCM9 |
| GTF2F1 | HMGA2 | HAX1 | GTF2I | HSF4 | NDEL1 |
| NUP54 | KDM3B | ZNF652 | EGR1 | DNASE1 | ZC3H12A |
| FBXW11 | BEND6 | FOXP4 | ZSCAN26 | KDM3A | ZNF391 |
| IRF1 | PYGO2 | GTF2IRD2B | XPOT | CLCA2 | RBAK |
| HNRNPU | TAF10 | HES6 | DTX2 | TNPO3 | RNF43 |
| GBX1 | GTF2IRD2 | OVOL3 | POLR2I | ZBTB2 | GTF2H2C |
| CDC73 | ZNF83 | TNRC18 | RFX6 | ZNF468 | ZNF479 |
| MYNN | PDCD6- | HOXA3 | ZNF679 | ZNF736 | ZNF680 |
| | AHRR | | | | |
| ZNF273 | ZNF107 | POM121 | REPIN1 | ZNF775 | NEUROD6 |
| SUPT6H | ZNF267 | INPP4A | PWWP2A | ZSCAN16 | ZKSCAN8 |
| ZNF84 | ZNF165 | RGPD3 | ZBTB41 | ZNF573 | ASCL3 |
| REL | FOXN1 | MYT1L | HPN | ZNF23 | ZNF559 |
| IPO7 | TP63 | ZSCAN5C | FOXJ1 | HIC1 | NR2F6 |
| MITF | ZNF44 | ZNF563 | ZNF442 | ZNF799 | ZNF443 |
| ZNF709 | FOXP1 | STAT1 | NUP43 | KPNB1 | FZR1 |
| TBX21 | CENPS | MTA2 | LEMD3 | ZBED6 | ZNF566 |
| ZNF69 | ZNF700 | DDX5 | CSRNP2 | ZNF763 | TFCP2 |
| ZNF433 | ZNF878 | ZNF844 | ZNF20 | POU6F1 | ZNF625 |
| ZNF606 | INTS5 | HDAC3 | FANCL | POLR1G | ERCC2 |
| AHRR | P2RX2 | ZNF131 | TAF13 | ZNF530 | ZNF577 |
| ZNF649 | ZNF613 | ZNF350 | POLE | ING5 | ZNF317 |
| CIC | PAX3 | DMPK | ZNF300 | MED20 | RGPD4 |
| SIN3B | TENT2 | JPT1 | NFYA | NUP85 | ZNF180 |
| TAL1 | FEV | FOXE3 | ERFL | ZNF415 | TM7SF2 |
| C12orf43 | PELP1 | KDM1A | ZNF266 | SMARCA4 | PRKG2 |
| ASCL2 | MED11 | ELAVL4 | ZSCAN31 | CORT | AHCTF1 |
| ACKR2 | ZBTB47 | PBX1 | POM121L2 | DST | ZSCAN5A |
| FOXD2 | ZNF567 | ZNF582 | ZNF439 | ZFP30 | LHX5 |
| NRXN1 | ZNF226 | TMC6 | ZNF841 | IKZF4 | ZNF544 |
| TMC8 | ZNF233 | ZNF534 | ZNF836 | HINFP | SYNE1 |
| ZNF320 | STAT3 | ZNF761 | ZNF383 | ZNF224 | ZNF551 |
| ZNF154 | ZNF671 | ZNF776 | ZSCAN4 | SMARCC2 | GLI2 |
| ZNF888 | ZNF816 | SP140 | ZNF347 | ZNF665 | ZNF677 |
| PGBD1 | ZNF160 | ZEB2 | GHDC | AK9 | STAT5B |
| TREX1 | LPIN1 | ZNF692 | ZSCAN12 | ZNF184 | FOXO3B |
| ARL6IP6 | STAT2 | BAHCC1 | BCAS3 | SBNO1 | DHX37 |
| CREBZF | ZBTB16 | NUCB2 | MLX | NR1I2 | GRHL3 |
| ACTR6 | ORC4 | RBBP4 | MBD5 | RGPD5 | ZNF140 |
| ERN1 | SHOX2 | TEX2 | CLCC1 | HOXC5 | SLC16A3 |
| HDAC4 | DHX30 | NR1I3 | ZNF589 | ZNF891 | KCNJ11 |
| DHCR7 | UBTF | VRK2 | DEAF1 | SATB1 | ZMPSTE24 |
| ZFP69B | NR2C1 | SIRT7 | MAFG | MTA3 | ZBTB48 |
| CREBL2 | HNF1A | UNC50 | EZH1 | PPARG | SPAST |
| LMNTD1 | ZNF691 | ZNF697 | GTF3C3 | NEUROD4 | DMBX1 |
| MEN1 | CRTC2 | CREB3L4 | KDM6B | TFCP2L1 | ALX4 |
| POU1F1 | POLQ | ARGFX | SETD5 | ZBTB18 | HSPD1 |
| DDIT3 | NFE2L2 | MDM2 | DDX1 | STAT5A | HOXB3 |
| TADA3 | SHISA5 | ZNF384 | HOXB2 | HOXB5 | ITPR1 |
| DMRTB1 | TEPSIN | BHLHA9 | TBX10 | RAB40B | IRAG2 |
| GRHL1 | PRDM4 | ASCL4 | KLF11 | RUNX3 | ZBTB38 |
| TOP2B | ATF7-NPFF | ATF7 | ZNF750 | TASOR | SP9 |
| LRRC59 | SOX6 | PRDM11 | H1-10 | H1-8 | TAF5L |
| ZNF142 | SETSIP | INTS2 | BRIP1 | RGPD1 | PRMT6 |
| ZNF683 | BCL6B | ORC1 | H3-3A | MIXL1 | HEYL |
| PUM2 | SP140L | QRICH2 | YBX1 | JUN | ASH1L |
| HDAC1 | HLX | ZBTB37 | SP110 | USF1 | TRIM37 |
| AKIRIN1 | CEPT1 | ARNT | PARP1 | HDAC5 | SREBF1 |
| RFX5 | ZNF669 | TOR1AIP2 | RIF1 | GMEB1 | CC2D1B |
| IFI16 | LRRFIP1 | TOR1AIP1 | DCTN1 | XPO1 | TTC21B |
| PTGS2 | NOC2L | HES4 | POLR2D | PAX8 | PSEN2 |
| SLC30A1 | ATF6 | SMPD4 | MPL | MED8 | RGPD6 |
| MEF2D | ORC2 | CARF | LRPPRC | SAMD11 | POLR1A |
| FOXI3 | MTF2 | MXD1 | PCBP1 | HP1BP3 | EVX2 |
| HOXD13 | HOXD12 | HOXD11 | HOXD10 | HOXD9 | HOXD8 |
| HOXD4 | HOXD1 | HOXD3 | AFF3 | DNAJB2 | SAMD13 |
| UBXN4 | AGFG1 | ASXL2 | DNMT3A | MACO1 | NEUROD1 |
| SP3 | IKZF2 | RBM15 | RORC | TFAP2E | ZFP69 |
| ZNF684 | TAF12 | NCOA1 | GOLT1A | MDM4 | SFPQ |
| RPAP2 | GFI1 | ZSCAN20 | ZBTB17 | PTGER3 | SETDB1 |
| EXO1 | LBR | LHX8 | RPRD2 | ZNF124 | ASCL5 |
| LMNA | ZNF496 | RCC1 | ZNF362 | PHC2 | S100A6 |

In other embodiments, the compositions and methods are useful for non-human cells or with non-human specimens. Other non-human animals of interest include mammals such as a mouse, rat, guinea pig, dog, cat, horse, cow, pig, or non-human primate, such as a monkey, chimpanzee, baboon, or gorilla. Other animals of interest include Drosophila melanogaster. Exemplary targets useful herein include the murine targets found in Table 3 and the Drosophila targets found in Table 4. However, the targets useful in the compositions and methods described herein are not limited to those found in these tables. Other targets in these or other organisms, or homologous or orthologous targets in other organisms may be employed.

TABLE 3
Exemplary Mouse Targets

| | | | | | |
|---|---|---|---|---|---|
| Zfy1 | Zfy2 | Sry | Vsx2 | Akap6 | Fam169a |
| Lmnb1 | Nr1i2 | Tfap2a | Gm10139 | Dync1h1 | Trim27 |
| Asxl3 | Iigp1 | Npas4 | Gcm2 | Foxd4 | Ylpm1 |
| Zscan12 | Prox2 | Zfp712 | Zfp708 | Gm28557 | Slc29a2 |
| Rslcan18 | Cbx5 | Zfp759 | Snai2 | Zscan26 | Zfp397 |
| Nkapl | Prkaa1 | Rsl1 | Zfp35 | Zfp24 | Zkscan4 |
| Srebf2 | Zfp455 | Zfp458 | Zfp457 | Zkscan8 | Zfp595 |
| Mcm4 | Zfp953 | Gm28041 | Zfp456 | E2f6 | Hivep1 |
| Dmrt1 | Zfp429 | Dmrt3 | H1f5 | Rprd1a | Zfp459 |
| Olig2 | Olig1 | Dmrt2 | Zbtb20 | Zfp874a | Ptger4 |
| Nfe2 | Rcor1 | Cdx1 | Mlh3 | Zhx2 | Zfp184 |
| Insm2 | Zfp874b | Pom121l2 | Zfp58 | Cebpd | Zfp87 |
| Ctcf | Zfp748 | Nr3c2 | Zfp729b | Gm49345 | Zfp729a |
| Dach1 | Pcm1 | Cetn2 | Wbp2nl | Fos | Wdhd1 |
| Jdp2 | Zfp738 | Batf | Banf1 | Zfp65 | Ppargc1b |
| Zfp85 | Zfp493 | Fosl1 | Foxd1 | Zfp273 | Zfp983 |
| Six2 | Gm10226 | Zfp760 | Zfp229 | Zfp820 | Zfp995 |
| Zfp942 | Zfp943 | Zfp947 | Zfp994 | Ednra | Gm7072 |
| Zfp322a | Klf5 | Rit2 | 5730507C01Rik | Klf12 | Thap11 |
| Xpo4 | Zfp944 | Pou4f2 | Rbmxl1 | Smc4 | Zfp758 |
| Epas1 | Ovol1 | Zfp946 | Smarca2 | Atrx | Kpna4 |
| Yeats4 | Polr3b | Foxp4 | Nutf2 | Tent4a | H3c7 |
| Zfp945 | H1f3 | Sox1 | H3c6 | H3c4 | H1f4 |
| H1f2 | H3c3 | Rfx4 | H3c2 | Zfp40 | Polr2d |
| H1f1 | Zfp366 | Tcf20 | Phf11a | Phf11b | Lmo7 |
| Kat5 | Nkrf | Zfp213 | Stpg4 | Jarid2 | Phf11d |
| Zfp13 | Rela | Phf11c | Zbtb16 | Mdm2 | Nkx2-1 |
| Zfp534 | Zfp275 | Nkx2-9 | Pax9 | Zfp984 | Zfp933 |
| Zfp92 | Med10 | Prkaa2 | Taf9b | Ercc3 | Exo1 |
| Zscan10 | Smad1 | Msh2 | Bche | Myc | Bin1 |
| Nupl1 | Msh6 | Rfx3 | Wdr82 | Nkap | Ring1 |
| Gm19965 | Nup107 | Gm28363 | Gm49336 | B020011L13Rik | Gm28360 |
| Gm28168 | Gm7145 | Gm29106 | Rhox3a | Rhox3a2 | Smarca5 |
| Mlip | Irx1 | Rhox3c | Irx2 | Foxn2 | Atxn1 |
| Nfatc3 | Irx4 | Mtmr6 | Nfya | Nkx6-1 | Zfp853 |
| Prdm4 | Hdac1 | Ascl4 | Cdk6 | Rtcb | Rhox3e |
| Rhox3f | Rhox3g | Tbx22 | Foxa1 | Rhox3h | Rnf2 |
| Nup153 | Ednrb | Zfp90 | Mybl1 | Nup155 | Gtf2a1l |
| Pou4f1 | Obi1 | H1f10 | Glis3 | Hic2 | Nipbl |
| Esrrb | Rxrb | Rhox10 | Rhox11 | Rhox13 | Zbtb33 |
| Hesx1 | Ccar1 | Dppa4 | Tert | Dppa2 | Onecut2 |
| Mllt6 | Gm32717 | Gm32802 | Hmgn5 | Nup85 | Gcm1 |
| Gtf2h2 | Gm9040 | Gm9044 | Gm9045 | Gm9046 | Gm9048 |
| Gm9049 | Clgn | AI987944 | Rpa1 | AW146154 | Pou3f4 |
| Atp1b4 | Zbtb18 | Itsn1 | Xpo1 | Gm6871 | Tasor |
| Kdm1b | Pcgf2 | 2610021A01 | Nrxn1 | Nanog | Zfp788 |
| | | Rik | | | |
| Gata2 | Nup50 | Gm12258 | 2810021J22 | Wdfy3 | Rad17 |
| | | | Rik | | |
| Zfp39 | Zfp169 | Barx2 | Ak6 | Foxj2 | Taf9 |
| Pax3 | Barx1 | Cdk7 | Egr1 | Satb1 | Grhl1 |
| Cgas | Mtor | Klf11 | Foxi3 | Hey2 | Ascl1 |
| Sox21 | Hdx | Gm9376 | Rfx5 | Fli1 | Zfp141 |
| Hnrnpu | Gata4 | Fabp1 | Rrm2 | Zfp977 | Zfp976 |
| Zfp975 | Vax1 | Gm17067 | Ets1 | Gm2381 | Emx2 |
| Kat2b | Thap7 | Sox2 | Runx1 | Atoh7 | Sox7 |
| Nup37 | Ndc1 | Phf8 | Gmnn | Zfp119a | Zfp959 |
| Zfp119b | Glis1 | P2rx6 | Ndn | Ppara | Alg14 |
| Dmrtb1 | Zfp715 | Pura | Nobox | Nfat5 | Klf13 |
| Sirt1 | Hdgfl3 | Mcmdc2 | Bnc1 | Tspyl1 | Dach2 |
| Tcf24 | Grk5 | Bbx | Zfp950 | Pkn1 | Pcid2 |
| Gm21060 | Zfp395 | Sim2 | Tgif2lx2 | Tgif2lx1 | Tspyl4 |
| Nap1l3 | Sox11 | Zfhx3 | 4932411N23 | Spic | Stag2 |
| | | | Rik | | |
| Kdm3a | Ahctf1 | Jmjd1c | P2rx7 | Rtf1 | Mcm2 |
| Hdac2 | Rel | Rfpl4b | Egr2 | Smpd4 | Tox |
| Gm14444 | Zfp37 | Gm14393 | Casz1 | Gm14399 | Fancm |
| Gm14443 | Med1 | Scrt2 | Gm14391 | Cenps | Helt |
| Tcf15 | Dhx9 | Gm4631 | Brd2 | Rbm15b | Ccnd1 |
| Zfp968 | Gm4724 | Zfp965 | Zfp267 | Gm11007 | Majin |
| Zfp969 | Bicd2 | Gm2007 | Zfp658 | Gabrb1 | Zfp719 |
| Gm6710 | Zfp966 | Zfp819 | Tbc1d20 | Tfdp1 | Gm2026 |
| Gm2004 | 2210418O1 | Gm11009 | Gtf2a1 | Zfp72 | Ctbp1 |
| | 0Rik | | | | |
| Zfp825 | Bcl11a | Gm14434 | Recql5 | Obox8 | Sox4 |
| Bhlha15 | Zfp967 | Men1 | Ezh2 | Zbtb11 | Med15 |
| Myt1l | Ist1 | Gm14308 | Neurod2 | Fancl | Zfp973 |
| E2f3 | Gm14305 | Gm14295 | Gm14408 | Zfp786 | Sox12 |
| Zfp398 | E4f1 | Gm14419 | Gm14401 | Zfp282 | Vrk2 |
| Tnmd | Gm14410 | Gm14409 | Klhdc2 | Gm14412 | Actrt1 |
| Zfp821 | Smc1a | Primpol | Zfp867 | Irf4 | Nkx1-1 |
| Gm14418 | Zfp970 | Uhrf1 | Myo6 | Gm14403 | Spin1 |
| Myo1c | Erbin | Gm14406 | Gm14322 | Samd1 | Zfp648 |
| Zfp212 | Gsc2 | Tnks2 | Gm14325 | Gm14327 | Zfp972 |
| Nfxl1 | Zfat | Gm14326 | Plrg1 | Zfp971 | Smarca1 |
| Zfp956 | Irf2 | Zfp777 | Rax | Taf7 | Zfp746 |
| Zfp931 | Rfx1 | Tmem18 | Zkscan17 | Cks2 | Foxq1 |
| Hmga2 | Foxf2 | Foxc1 | Nr1h4 | Zfp612 | Adra1a |
| Mrgprf | Ikzf3 | Cdk1 | Obox7 | Nudt9 | Rpap1 |
| Bnip3l | Polr1a | Pbx2 | Lemd3 | Cc2d1a | Ebf2 |
| Atoh8 | Ing2 | Ei24 | Tspyl2 | Obox2 | Hcfc1 |
| Kmt5b | Hmgn3 | Bcl2l1 | Itgb4 | Xpot | Obox1 |
| Rpf2 | Klf15 | Usp51 | Hdac3 | Zfp467 | Zfp951 |
| Sh3bgrl2 | Tyro3 | Pknox2 | Lhx8 | Sox5 | Foxr2 |
| Klf8 | Cdk19 | Otulinl | Seh1l | Arnt2 | Myof |
| Obox3 | Elf4 | Ak9 | Med24 | Ncaph2 | Zbtb24 |
| Nupr1 | Nfil3 | Obox5 | Obox6 | Safb | Brd3 |
| Lhx4 | Msx2 | Stat4 | H3f3b | Thra | Foxs1 |
| Nr1d1 | Crx | Ranbp3 | Nkx2-6 | Nkx3-1 | Rfx2 |
| Zfp386 | Taf4 | Sfmbt1 | Mycs | Taf7l | Noc3l |
| Zfp280c | Hells | Rnf180 | Nr3c1 | Rxra | Barhl2 |
| Ddx19b | Ss18l1 | Chmp7 | Nacc1 | Agpat5 | Gtf2f1 |
| Mbtps2 | Msl1 | Atf6b | Egr3 | Yy2 | Casc3 |
| Zfp644 | Tbx1 | Foxn3 | Wdr61 | Pou4f3 | Tcerg1 |
| Setdb1 | Ipo11 | Rtn4 | Prdm14 | Erg | Tmem201 |
| Mapk3 | Orc1 | Nup160 | Apbb1 | Dlx6 | Cc2d1b |
| Cdc45 | Nup210 | Ets2 | Mga | Dlx5 | Tmpo |
| Repin1 | Zfp775 | Polr3d | Hand2 | AI854703 | Hira |
| Tspyl5 | Cdc6 | Arnt | Eno1 | Fezf2 | Epha3 |
| Retsat | Pou5f2 | Lmntd1 | Rere | Zfp654 | Tbx6 |
| Hr | Csrnp3 | Rara | Mtdh | Nr2f1 | Pou1f1 |
| Bhlhe41 | Top2a | Nfix | Mecp2 | Foxo3 | Cdk4 |
| Eno1b | Lyl1 | Tmem43 | Sephs1 | Nxf2 | Zfp384 |
| Elk3 | Calr | Nr2e1 | Isl2 | Med9 | Polr1f |
| Hmgn1 | Ferd3l | Twist1 | Sp4 | Sp8 | Wbp2 |
| Tmem176b | Gata5 | Hpf1 | Meis3 | Tor1aip1 | Ttc21b |
| Bclaf3 | Nelfe | Tcf4 | Tspyl3 | Scml4 | Tfcp2l1 |
| Atp5j2 | Klf1 | Plagl2 | Zkscan14 | Zfp518a | Zkscan5 |
| Foxp3 | Mcm10 | Tor1aip2 | Gli2 | Zfp655 | Asxl1 |
| Zscan25 | Tmem170 | Ncoa2 | Nsd2 | Osr2 | Cdyl |
| Zfp41 | Lcor | Bhlha9 | Parp1 | Srebf1 | Prdm1 |
| Polr2k | Mafa | Mbd6 | Ahr | Ddit3 | Tcfl5 |
| Polr3g | Fam3b | Mixl1 | Rrp12 | Gmeb1 | Cetn3 |
| Junb | Meiob | Tbx18 | Taf12 | Klf6 | Phf13 |
| Zbtb48 | Nelfa | Zbtb12 | Spib | Grhl2 | Stat1 |
| Nxf1 | Nr2c1 | Prdm15 | Prickle1 | Taf6l | Gli1 |
| Zbtb21 | Polr2g | Zbtb3 | Med21 | Tmem120b | A630089N0 |
| | | | | | 7Rik |
| Emd | Taf10 | Gfi1b | Nr2c2 | Npas1 | Meox2 |
| Rnf6 | Pold1 | Ehmt2 | Sim1 | Tle1 | Dbx2 |
| Sin3a | Rreb1 | Bhlhe23 | Iffo1 | Tor3a | Maf |
| Rcc1 | H3f3a | Yap1 | Cdk8 | Klf10 | C130026I21 |
| | | | | | Rik |
| Prickle2 | Rprd2 | Ints5 | Ankrd2 | Rpap2 | Gapdh |
| Mef2a | Rfx6 | Gabpa | Vapa | Mrpl19 | Pgrmc2 |
| Atmin | Arntl2 | Pgr | Gfi1 | A530032D15 | Zfp202 |
| | | | | Rik | |
| Rnf4 | Gpbp1 | Msc | Mxd3 | Mta2 | App |
| Med18 | Nr1h2 | Scgb1a1 | Zfp791 | Etv1 | Sp110 |
| Mbd2 | Kdm4c | Smad4 | Foxj1 | Six6 | Six1 |
| Six4 | Ddx11 | Mnat1 | Clic1 | Nkx2-3 | Bach1 |
| Barhl1 | Faf1 | Zfp449 | Dmrta2 | Eya3 | En1 |
| Nemp2 | Ttf1 | Esx1 | Zfp263 | Foxp2 | Csnk2b |
| Zfp174 | Zfp597 | Zbtb7c | Smad2 | Elavl4 | Myrf |
| Maz | Npm2 | Skor2 | Qrich2 | Hif1a | Fbxw11 |
| Eny2 | Dnase1 | Mef2c | Otx2 | Hdac7 | Sp100 |
| Tfec | Osbpl3 | Xpo7 | Zfp949 | Ranbp17 | Lbr |
| Eed | H3c15 | Ifi27 | A630001G2 | Vdr | Med27 |
| | | | 1Rik | | |
| Mlxip | Hes2 | Ifi27l2a | H3c14 | Etv3 | Zfp607b |
| Etv3l | H3c13 | Pitx2 | Zfp626 | Txnl4a | Gtpbp4 |
| Cav2 | Zfp607a | Hes3 | Nfe2l3 | Dnmt3b | Ttc5 |
| Pax2 | Arid1b | Stau2 | Parp2 | Zfp974 | Zfp780b |
| Zfp850 | Nr2f2 | Bsx | Hif3a | Apex1 | Polr3gl |
| Nfatc1 | Senp1 | Crebbp | Gsx2 | Chd5 | Zfp423 |
| Ccnh | Foxg1 | Zfp553 | Gm43517 | Cnep1r1 | Zfp771 |
| Mtf2 | Foxi1 | Zfp641 | Kdm4d | Syne2 | Kat2a |
| Tlx1 | Lbx1 | Elf2 | 1700123L14 | Sall3 | Polr2h |
| | | | Rik | | |
| Tfap4 | Glis2 | Spi1 | Tmem109 | Zfp930 | Rasa1 |
| Irf8 | Sun5 | Foxf1 | Trps1 | Zfp868 | Zfp964 |
| Zfp869 | Zfp963 | Gm20422 | Zfp866 | Gtf3c3 | Rad21 |
| Zfp236 | Shmt2 | Foxc2 | Foxl1 | Ghdc | Med30 |
| Gsc | Med4 | Esr2 | Ccnt1 | Zfp473 | Zfp516 |
| Stat5b | Tshz1 | Sorl1 | Npm3 | Setd7 | Mgst2 |
| Pbrm1 | Ddn | Tfap2d | Atf5 | Stat6 | Morf4l1 |
| Ccnd2 | Ipo8 | Pbx4 | Zbtb25 | Nup62 | Gata3 |
| Zbtb1 | Parp11 | Stat5a | Rogdi | Mcm9 | Clmn |
| Mfsd10 | Glyr1 | Taf3 | Gm45871 | Asf1a | Taf2 |
| Kmt2d | Supt5 | Polr3c | Nemp1 | Syne3 | Polr1d |
| Med17 | Gsx1 | Pdx1 | Cdx2 | Nup62cl | Foxo1 |
| Tfam | Clip1 | Ldb1 | Npc1 | Zbtb39 | Foxd2 |
| Tfe3 | Hdgf | Creb5 | Foxe3 | Tead4 | Arid1a |
| Zfp768 | Mypop | Zfp747 | Foxa3 | 9130019O22 | E430018J23 |
| | | | | Rik | Rik |
| Tfap2b | Pitx1 | Zic1 | Rfxank | Pou2f3 | Zfp764 |
| Lef1 | Mitf | Zic4 | Rbmx | Macroh2a1 | Senp2 |
| Prim1 | Zbtb14 | Zfp689 | Cramp1l | Pitx3 | Hsf2 |
| Tal1 | Plscr1 | Stat3 | Foxm1 | Rnf123 | Isl1 |
| Zic3 | Trim66 | Sall1 | Vrk1 | Pou5f1 | Alx1 |
| Nfkb2 | Tra2b | Ccnt2 | Trp73 | Etv5 | Tox3 |
| Scml2 | Tle4 | AW822073 | Duxf3 | Gm4981 | Snai3 |
| Med29 | Tmc6 | Bcl11b | Paf1 | Gnaq | Dmtfl |
| Mcm3 | Cuedc2 | Gm20379 | Smad5 | Taf4b | Mef2d |
| Tmc8 | Dmpk | Hmgn2 | Smarcd1 | Ranbp2 | Vax2 |
| Spz1 | Foxb2 | Six5 | Apeh | Crem | Trpc7 |
| Gtf2f2 | Ascl3 | Anxa7 | Pwwp2a | Adra1b | Clock |
| Ryr2 | Scx | Myf5 | Myf6 | Scrn1 | Tsc22d1 |
| Gtf2h5 | Tgif1 | Hsf1 | Yy1 | Rorb | Oit3 |
| Hsfy2 | Nxt2 | Scrt1 | Med6 | Ebf1 | Bcl6 |
| Taf5 | Txlng | Atf1 | Mcm6 | Satb2 | Bap1 |
| Nkx6-3 | E2f1 | Sox30 | Gtf2h4 | Zfp341 | Chmp4b |
| Mlx | Dnajb12 | Tbl1x | Litaf | Zfp438 | Zeb1 |
| Tbx15 | E2f7 | Zfp558 | Hlx | Zfp131 | Aqp1 |
| Zfp683 | Dmbx1 | Csrnp2 | Ghrhr | Epc1 | Cyhr1 |
| Tfcp2 | Prdm16 | Trp63 | Mkx | Smad9 | Rfxap |
| Wdr3 | Cdt1 | Pml | Foxh1 | Insr | Gsto1 |
| Sp5 | Pou6fl | Hnf4g | Itprip | Wac | Otp |
| Zfp219 | Nfib | Zfhx4 | Stat2 | Ube2i | Pex2 |
| Zfp317 | Rbl2 | Cbfa2t3 | Ercc4 | Zfp251 | Zfp7 |
| Gmnc | Zfp629 | Sohlh2 | Zfp647 | Foxp1 | Zbtb2 |
| Zfp560 | Hey1 | Osbpl8 | Elf1 | Gata1 | Zfp358 |
| Med7 | 5430403G1 | 4930522L14 | Gm15446 | Hmx1 | Zfp932 |
| | 6Rik | Rik | | | |
| Ezh1 | Gm17655 | Gm35315 | Aen | Mxi1 | Zfp426 |
| Med25 | Esr1 | Gata6 | Nutf2-ps1 | Smc3 | Zfp266 |
| Zfp846 | Ipo7 | Fosb | Nap1l1 | Psen1 | Zfp605 |
| Hes5 | Lmna | Ercc1 | Irx3 | Cd3eap | Dnmt3a |
| Nhlh2 | Zfp143 | Irx5 | Syne1 | Irx6 | Wdr13 |
| Sirt2 | Mrps14 | Cbx2 | Cacybp | Sox8 | Hes1 |
| Zfp704 | Neurod6 | Fezf1 | Rest | Tamalin | Cbx4 |
| Nr4a1 | Myct1 | Nfkb1 | Hinfp | Tdrd3 | Smarcc2 |
| Gm9833 | Psip1 | Tubb5 | Polr2b | Nrm | Chd8 |
| Maco1 | Runx3 | Zfp410 | Zfp668 | Alox5ap | Ncoa1 |
| Ercc2 | Zfp276 | Rnf168 | Ebp | Rac2 | Gli3 |
| Pcyt1a | Tox4 | Tfdp2 | Tcf7l2 | Nr1h5 | Sall2 |
| Parg | Pole | Pou6f2 | Nudt21 | Zfp148 | H2az1 |
| Fcor | Dnajb14 | Ski | Arx | Pola1 | Atad2b |
| Gle1 | P2rx2 | Zbtb37 | Orc2 | Ercc6 | Crebzf |
| Zfp740 | Prkcz | Pum2 | Rarg | Prrxl1 | Nup93 |
| Noc4l | Tbx10 | Etv6 | Zbtb38 | Rnf13 | Esrrg |
| Taf7l2 | Zfx | Ikzf4 | Grhl3 | H1f0 | Abcf1 |
| Crebl2 | Dnmt1 | Aaas | Osr1 | E2f5 | Sp7 |
| Polr2f | Sp1 | Sox10 | Zfp62 | Actn4 | Msgn1 |
| Mllt3 | Zfp296 | Zfp808 | Gm3604 | Gm49359 | Zfp935 |
| Zfp934 | Platr25 | Gm5141 | Fus | Mycn | Ddx1 |
| Brca1 | Ep400 | Sarnp | Ctr9 | Maff | Il15ra |
| Igf2r | Polq | Pola2 | Polg | Cybb | Ajuba |
| Zbtb42 | Spast | Nup43 | Bhlhe22 | Nup133 | Prim2 |
| Tada2b | E2f2 | Pus1 | Cenpf | Taf5l | Tm7sf2 |
| Foxr1 | Mta1 | Gtf2e1 | Ranbp1 | Zfp46 | Myrfl |
| Foxl2 | Zscan29 | Zfp367 | Batf2 | Esrra | Nr2e3 |
| Polr2c | Gmeb2 | Dmrta1 | Zfp352 | Max | Cebpe |
| Zfp997 | Gm10772 | Neurod4 | Zfp998 | Neurog1 | Med12l |
| Mindy3 | Gm28047 | Sun2 | Wtap | Cenpv | Bend6 |
| Prox1 | Atf7 | Hhex | Ticrr | Nucks1 | Kdm1a |
| Relb | Irf3 | Elk4 | Calcoco1 | Supt6 | Hoxc13 |
| Hoxc12 | Hoxc11 | Hoxc10 | Hoxc5 | Hoxc9 | Hoxc8 |
| Hoxc6 | Nap1l5 | Ift74 | Hsf3 | Hoxc4 | Cbx7 |
| Emx1 | Zfhx2 | Apoe | Thap1 | Itpr1 | Atf4 |
| Zfp287 | Zfp286 | Gm12845 | Zbtb40 | Dhrs2 | Klf17 |
| Zfp958 | Med14 | Mrnip | Snca | Noto | Ar |
| Bhlhe40 | Cptp | Mrtfa | Jun | Batf3 | Atoh1 |
| Atf3 | Sox14 | Foxn1 | Nrl | Ltc4s | Ep300 |
| Plagl1 | Etv4 | Dlx1 | Spag4 | L3mbtl2 | Zfp709 |
| Dlx2 | Zfp882 | Egr4 | Meox1 | Mesp1 | Mesp2 |
| Zfp617 | Pax4 | Rangap1 | Macroh2a2 | Shisa5 | Smarcad1 |
| Tef | Sumo1 | Zfp961 | Polr3h | Dtl | Ash1l |
| Tepsin | Nat8f7 | Nat8f6 | Msx1 | Neurog3 | Crebrf |
| Hivep2 | Bnip1 | Kdm6a | Nkx2-5 | Kmt2a | Cited2 |
| Ncor2 | Stag1 | Tmem97 | Gm10282 | Ranbp3l | Prdm5 |
| Klf2 | Irf9 | Zfp319 | Zfp354c | Zfp879 | Mad2l1 |
| Phf1 | Cpne1 | Slc30a1 | Foxo4 | Olig3 | Zfp454 |
| Zfp2 | Zfp710 | Tbp | Zfp354b | Figla | Calr3 |
| Prop1 | Bclaf1 | Med12 | Zfp354a | Gadd45a | Chd1 |
| Zfp623 | Zfp707 | Mapk15 | Carf | Nfia | Myb |
| Setd5 | Zfp960 | Zfp97 | Med26 | Gtf2b | Tmem38a |
| Med8 | Bahcc1 | Elk1 | Uxt | Zkscan6 | Mpl |
| Tbpl1 | Tcf21 | Sin3b | Zfp300 | Noc2l | Nono |
| Isx | Kcnh1 | Samd11 | Mcm5 | Dst | Itpr3 |
| Taf1 | Hdac5 | Rec8 | Lemd2 | Gch1 | L3mbtl3 |
| Cited1 | Hdac8 | Dhx37 | Tada3 | Prrx1 | Gbx2 |
| Hp1bp3 | Zfp160 | Mdm4 | Tead1 | Irf5 | Runx2 |
| Dmrtc1b | Nfatc4 | Dmrtc1c1 | Dmrtc1c2 | Wrn | Zfp677 |
| Zfp54 | Dmrtc1a | Zfp51 | Zfp53 | Supt3 | Tnpo3 |
| Purg | Nap1l2 | Cdx4 | Cdh5 | Zfp52 | Zfp948 |
| Cdc5l | Hmga1 | Kash5 | Irf6 | Ggn | Aebp2 |
| Pak1 | Rhox5 | Ybx1 | Mphosph8 | Nr1h3 | Sox3 |
| Nr1d2 | Thrb | Zgpat | Rarb | Top2b | Suz12 |
| Clca2 | Napepld | Tead2 | Sirt7 | Skor1 | Mafg |
| Zhx1 | Smarca4 | Atad2 | Spdef | Golt1a | Mcmbp |
| Gm28040 | Gtf2e2 | Dnajc2 | Foxd3 | Chil3 | Shox2 |
| Taf11 | Akr7a5 | Nkx3-2 | Mllt10 | Hsd11b1 | Arntl |
| Dnajc1 | Sox13 | Cmtm3 | Tcf7 | 2610044O15 | Sp3 |
| | | | | Rik8 | |
| Sec13 | Terb1 | Ugt2b37 | Zbtb7b | Tnks | Foxj3 |
| Cept1 | Smad3 | Lrrfip1 | Phf20 | Smad6 | Ugt2b38 |
| Hivep3 | Foxo6 | Scmh1 | Bmi1 | Atxn7 | Dhx30 |
| Ran | Zfp180 | Zfp112 | Nrf1 | Zfp235 | Zfp114 |
| Ubtf | Zfp111 | Zfp109 | Mta3 | Orc5 | Pygo2 |
| Zc3hc1 | Sp9 | Zfp108 | Zfp93 | Rbm15 | Tbx19 |
| Arid5a | Zfp61 | Nfyc | Zfp94 | Zfp523 | Foxn4 |
| Pax7 | Alx3 | Zfp69 | Zmpste24 | Kmt5c | Anxa11 |
| Cphx1 | Duxbl1 | Rlf | Pparg | Mad2l1bp | Ppard |
| Hsf4 | Smarcc1 | E2f4 | Polr3a | Samd13 | Hes6 |
| Zfp628 | Hmga1b | Lrpprc | Mycl | Polr1c | Tead3 |
| Heyl | Upf1 | Hax1 | Zscan2 | Zfp84 | Ptf1a |
| Zfp790 | Zfp524 | Gm44973 | Zfp940 | Ndel1 | Zfp865 |
| Ruvbl2 | Myog | Nupl2 | Klf14 | H1f8 | Zfp420 |
| Set | Hdac4 | Tchp | Six3 | Zfp27 | Zfp383 |
| Zfp74 | Zfp784 | Zfp580 | Crcp | Zfp872 | Zfp809 |
| Zfp599 | Sox18 | Cabin1 | Zfp810 | Srf | Hacd3 |
| Eif5a2 | Irf1 | Nr2f6 | Zfp568 | Zfp14 | Nradd |
| Bax | Zfp280b | Dmrtc2 | Alox5 | Zfp82 | Zbtb17 |
| Zfp422 | Zfp566 | Zfp260 | Zfp382 | Zfp146 | Sox6 |
| Klhdc3 | Prdm2 | Zfp637 | Terb2 | Zfp239 | Mcm3ap |
| Klf7 | Sort1 | 2210016L21 | Mecom | Hnf1a | Pcbp3 |
| | | Rik | | | |
| Ube2t | Zfp990 | Zfp268 | Zfp980 | Creb1 | Zfp986 |
| Zfp987 | Zfp600 | Zfp992 | Zfp981 | Zfp989 | Rex2 |
| Slc16a3 | Zfp991 | Zfp988 | Zfp978 | Zfp982 | Zfp985 |
| Polr2i | Zfp979 | Parp16 | Mynn | Ovol3 | Pou2f1 |
| Zfp248 | Zfp9 | Samd7 | Zfp787 | Nup205 | Elf3 |
| Gbx1 | Nup210l | Zfp444 | Zscan5b | Tgif2 | Per1 |
| Taf13 | Aire | Creb3l2 | Dpy19l1 | Creb3l4 | Trim24 |
| Clcc1 | Jund | Dpy19l2 | Hes7 | Crtc2 | Zfp667 |
| Zfp583 | Gm3854 | Tbx20 | Phc3 | Syne4 | Zfp78 |
| Med28 | Zfp28 | Smarcd3 | Rnf8 | Gm28043 | Tfeb |
| Skil | Zfp609 | Akirin1 | Agpat3 | Zfp408 | Pou3f1 |
| Mkrn1 | Zim1 | Foxk2 | Gm28038 | Mtf1 | Rab40b |
| Myt1 | Pclaf | Peg3 | Ssbp1 | Zc3h12a | Inpp4a |
| 2610008E11 | Ncapd3 | Zfp639 | Thrap3 | Ankrd17 | Zfp750 |
| Rik | | | | | |
| Mlh1 | Polrmt | Prmt6 | Polr3k | Dbp | Zfp954 |
| Zfp773 | Unc50 | Sphk2 | Mgst3 | Actl6a | Zfp418 |
| Zfp772 | Nup188 | Tfap2e | Rxrg | Lmx1a | S100a6 |
| Usp3 | Sfpq | Med16 | Pbx1 | Arid3a | Kdm6b |
| Zscan20 | Phc2 | Zfp362 | Rbl1 | Rorc | Pknox1 |
| Atf2 | Polr2e | Gpx4 | Sbno2 | Klf4 | Ppargc1a |
| Bok | Pou3f2 | Brd4 | Pwwp3a | Ubp1 | Akap8 |
| Evx2 | Wiz | Hoxd13 | Hoxd12 | Trp53 | Hoxd11 |
| Hoxd10 | Hoxd9 | Ing5 | Hoxd8 | Mbd3 | Hoxd3 |
| Tcf3 | Hoxd4 | Gm28230 | Hoxd1 | Sox15 | Zfp871 |
| Zfp811 | Zfp799 | Svep1 | Zfp870 | Nos1ap | Spata46 |
| Zfp472 | Atf6 | En2 | Zfp952 | Erfl | Zfp763 |
| Nucb2 | Zfp563 | Zfp955a | Zfp955b | Zfp81 | Zfp101 |
| Gm4125 | Aff3 | Rnf169 | Onecut3 | Klf16 | Bach2 |
| Nfe2l2 | Rprd1b | Kdm5a | Prrx2 | Zkscan16 | Ptges |
| Polr2a | Grwd1 | Tor1b | Lmnb2 | Rora | Taf8 |
| Zbtb4 | Zfp574 | Npas2 | Tor1a | Zik1 | Mnx1 |
| Foxb1 | Hnf1b | Bnip2 | Creb3l3 | Med20 | Ikzf2 |
| Gm20517 | Gtf2a2 | Zbtb7a | Plag1 | Ascl5 | Polr1h |
| Zfp57 | Tlx2 | Pcgf1 | Lbx2 | Zscan4b | T |
| Nup54 | Zscan4c | Zscan4-ps1 | Coq7 | Rbpj | Rfx8 |
| Zscan4d | Dctn1 | Myef2 | Eomes | Sdcbp | Nr1i3 |
| Orc3 | Zscan4e | Zscan4f | Creb3l1 | Zscan4-ps2 | Zscan4-ps3 |
| Hoxa4 | Hoxa5 | Osbpl6 | Hoxa6 | Hoxa7 | Hoxa9 |
| Hoxa10 | Hoxa11 | Tada2a | Fzr1 | Chd7 | Nos1 |
| Kcnj11 | Hoxa1 | Hoxa2 | Nfic | Hoxa3 | Hoxa13 |
| Evx1 | Zfp292 | Polr2m | C9orf72 | Zbtb32 | Etv2 |
| Lhx1 | Zfp551 | Zfp606 | Sirt6 | Zfp281 | Nr5a2 |
| Gm10778 | Zfp433 | Gm4767 | Gm32687 | Zfp873 | BC024063 |
| AU041133 | Zfp938 | Gm4924 | Aptx | Nfyb | Phf21a |
| Med13l | Zscan18 | Tcf12 | Zfp329 | Zfp128 | Zscan22 |
| Sox17 | Zfp324 | Nfx1 | Ikzf5 | Phlpp1 | Hmx3 |
| Hmx2 | Mafb | Pou2f2 | Bcl2 | Dpy19l4 | Top1 |
| Pcbp1 | Mxd1 | Tbx3 | Gmcl1 | Klf3 | Anxa4 |
| Csrnp1 | Tbx5 | Zfp526 | Zfp280d | Zhx3 | Myorg |
| Lypla1 | Sap30l | Hand1 | Lhx5 | Mns1 | Tcea1 |
| Ctnnb1 | Eif5a | Erf | Arid3c | Pax8 | Rfx7 |
| Sigmar1 | Nkx1-2 | Myod1 | Patz1 | Pou3f3 | Lhx9 |
| Prdm13 | Ccnc | Zfp692 | Prdm11 | Zfp672 | Ctdnep1 |
| Psmc5 | Prkg2 | Zfp651 | Cic | Smarcd2 | L3mbtl1 |
| Onecut1 | Ackr2 | Rb1cc1 | Bcas3 | Zbtb41 | Ctbp2 |
| Nhlh1 | Tbx2 | Tbx4 | Ern1 | Preb | Bcl2l10 |
| Brip1 | Leo1 | Tcf23 | Hnrnpd | Tex2 | Ints2 |
| Creb3 | Med13 | Mybl2 | Tox2 | St18 | Atraid |
| 2010315B0 | Zfp445 | Zkscan7 | Alx4 | Zfp105 | Gtf2h1 |
| 3Rik | | | | | |
| Cdc73 | Pax5 | Hnf4a | Zbtb5 | Polr1e | Usf2 |
| Trim37 | Foxe1 | Nup214 | Foxi2 | Pla2g4a | Ptgs2 |
| Nr4a3 | Plpp7 | Ifi206 | Ifi213 | Ifi209 | Ifi208 |
| Ifi207 | Ifi204 | Arap1 | Zfp189 | Mndal | Rnf20 |
| Ifi211 | Smc2 | Phox2b | Brap | Tmem33 | Ifi205 |
| Slc30a9 | Neurod1 | Ncaph | Tpr | Tal2 | E2f8 |
| Rbpjl | Zfp513 | Ciao1 | Rgs7 | Cux2 | Bcl6b |
| Dbx1 | Dusp2 | Htatip2 | Rag2 | Hpn | Ebf3 |
| Hsf5 | Rnf43 | Pelp1 | Dnttip1 | Dnajb2 | Fosl2 |
| Nell1 | Kcnip3 | Med11 | Phox2a | Stx1a | Usf1 |
| Mlxipl | Zfp661 | Polr3e | Pom121 | Fev | Vezf1 |
| Nkx6-2 | Tmem120a | Mrps23 | Dtx2 | Nup35 | Polr2j |
| Lrwd1 | Cux1 | Trpc2 | Cebpg | Ncoa5 | Cebpa |
| Zkscan1 | Dctn5 | Zscan21 | Xbp1 | Zfp113 | Mcm7 |
| Zc3h8 | Hlf | Taf6 | Polr1b | Stag3 | Zfp157 |
| Zfp68 | Prkcb | A430033K0 | Foxl3 | Faap24 | Sun1 |
| | | 4Rik | | | |
| Nup98 | Sox9 | Utp18 | Ehf | Elf5 | Aebp1 |
| Nsmf | Gper1 | Mbtd1 | Uncx | Mafk | Mad1l1 |
| Nudt1 | Camta2 | Tor4a | Rrm1 | Zfp941 | Foxk1 |
| Tnrc18 | Zfp3 | Lmo2 | Dpy19l3 | Rbak | Zfp12 |
| Zfp507 | Tshz3 | Zkscan2 | Zfp663 | Zfp334 | Nlrp6 |
| Gtf2h3 | Zfp536 | Ebf4 | Kdm8 | Zfp11 | Uri1 |
| Nup88 | Zfp619 | Plac8 | Gtf2i | Irf7 | Deaf1 |
| Jpt1 | Purb | Cdk9 | Gtf2ird1 | Med31 | Cse1l |
| Tor2a | Sun3 | Ascl2 | Zbtb49 | Ikzf1 | Slc22a18 |
| Znfx1 | Nap1l4 | Auts2 | Znhit1 | Dhcr7 | Ache |
| Pax6 | P2rx1 | Polr2l | Ptgds | Cenpb | Actl6b |
| Pla2g4c | Lrrc59 | P2rx5 | Snai1 | Msx3 | Pom121l12 |
| Cebpb | Bnip3 | Adnp | Egfr | Zfp446 | Trim28 |
| Zbtb45 | Chmp2a | H1f9 | Mzf1 | Zbtb34 | Zfp735 |
| Zfp616 | Dlx3 | Dlx4 | Meis1 | Rap1gap2 | Pafah1b1 |
| Kat7 | Lmx1b | Mnt | Nfatc2 | Med19 | Pbx3 |
| Ngfr | Zfp652 | Smox | Bmyc | Prnp | Sohlh1 |
| Hic1 | Otx1 | Pcna | Hoxb13 | Hoxb9 | Hoxb8 |
| Hoxb7 | Hoxb6 | Hoxb5 | Hoxb4 | Hoxb3 | Hoxb2 |
| Hoxb1 | Nfe2l1 | Sall4 | P2rx3 | Tshz2 | Sp2 |
| Sp6 | Tbx21 | Kpnb1 | Zfp217 | Epop | Morc2a |
| Ssrp1 | Npm1 | Tlx3 | Phf19 | Mcm8 | Tfap2c |
| Tmx4 | Rae1 | Ctcfl | Plcb1 | Nacc2 | Lhx3 |
| Zfp770 | Notch1 | Meis2 | Ovol2 | Polr3f | Lhx6 |
| Ptgs1 | Med22 | Insm1 | Zbtb6 | Zbtb26 | Nkx2-4 |
| Nkx2-2 | Pax1 | Foxa2 | Nxt1 | Gzf1 | Cst3 |
| Zfp120 | Gm10770 | Gm14139 | Zfp937 | Gm21994 | Gm14124 |
| Zfp442 | Zfp345 | Bahd1 | Vsx1 | Lhx2 | Rad51 |
| Gchfr | Nr5a1 | Nr6a1 | Rad21l | Scai | Zeb2 |
| Orc4 | Mbd5 | Rif1 | Arl6ip6 | Nr4a2 | Tbr1 |

TABLE 4
Exemplary Drosophila Targets

| | | | | | |
|---|---|---|---|---|---|
| Taf5 | Taf10b | Prdm13 | sna | CG8009 | dve |
| CG11247 | Nup44A | CG14006 | His3:CG338 | Mcm10 | Rad9 |
| | | | 48 | | |
| Arp6 | CG4709 | EcR | ham | CG3430 | tio |
| Wdr82 | Taf8 | CG43902 | CG7339 | bi | His1:CG33834 |
| His1:CG33858 | CrebA | CG12674 | nclb | CG17385 | HP1c |
| Taf12 | byn | CG32006 | topi | Lpt | skd |
| unc-4 | Gas41 | opa | CG33288 | Alg14 | Rpb4 |
| Nup358 | Ranbp9 | CG31224 | Kah | Dll | H2.0 |
| E(spl)mgam | piwi | Tfb1 | Gle1 | CG2678 | Sox15 |
| ma-HLH | | | | | |
| His1:CG33852 | CG44247 | chn | Pcl | PCNA | MESR4 |
| Alh | Fs(2)Ket | wor | l(3)neo38 | Orc2 | Cdk4 |
| CG11906 | pum | MED25 | mod(mdg4) | MED18 | CG13773 |
| TfIIA-L | Torsin | gt | dati | CG43347 | E2f1 |
| hbn | amos | CG15436 | SV | e(y)1 | barr |
| Tip60 | Su(Tpl) | Nulp1 | bru1 | lab | Nup214 |
| gcl | Six4 | edl | Nxt1 | dmrt11E | rn |
| CG8478 | DNApol- | Mcm6 | Met | 1-Dec | Med |
| | alpha50 | | | | |
| ssp | Lk6 | CG18262 | CG42726 | foxo | CG31917 |
| kuk | CG11695 | Clamp | Orc5 | B-H2 | His3:CG338 |
| | | | | | 51 |
| CG15160 | rgr | btn | Fer2 | dan | His1:CG338 |
| | | | | | 10 |
| MED23 | klu | osa | repo | CG18599 | Top2 |
| SuUR | TfIIEbeta | LamC | Hr96 | Ssrp | Blimp-1 |
| hay | Clk | CG3065 | FoxP | odd | CG12942 |
| Ibf1 | CG6813 | Rpb5 | Msp300 | CCDC53 | Hr3 |
| cad | ocm | svp | dar1 | CG42741 | koi |
| CG15269 | CG10631 | kay | croc | ac | Rbf2 |
| Sfmbt | sa | Sry-delta | Taf7 | CG17801 | CG3407 |
| ear | dpa | net | tin | CG8712 | l(2)gd1 |
| fd19B | Strump | CG31388 | CG31441 | CG2662 | CycH |
| CG15011 | sqz | RpII215 | ADD1 | pita | caup |
| CG17612 | CG5098 | RAF2 | Tor | Su(var)3-3 | Ada2a |
| crp | CG12391 | Hr78 | CG9609 | NfI | Lime |
| Lam | Rae1 | GV1 | Hr38 | pan | Taf11 |
| CG12267 | bon | CG10462 | OdsH | Poxm | CG17568 |
| Pof | RunxB | Su(var)3-9 | btz | CG7786 | sisA |
| Dbp80 | CrebB | CG31612 | MED14 | bigmax | mia |
| Tfb5 | mei-218 | CG7744 | CG1234 | su(sable) | CG8319 |
| Mi-2 | CG9723 | His1:CG338 | CG6220 | Abd-B | Mondo |
| | | 01 | | | |
| peb | Xbp1 | klar | gwl | Caf1-55 | tsh |
| phol | CG16779 | msk | Rad17 | CG11085 | onecut |
| EloC | Neu2 | HLH4C | Spt3 | CG12605 | Nlp |
| so | Zif | Ice1 | CG1647 | wda | row |
| Octbeta2R | M1BP | fd96Ca | cic | CG8089 | Nup153 |
| CG4424 | usp | CG4707 | Trf | CG12769 | Hr39 |
| ouib | CkIIalpha-i1 | CG42304 | twi | scro | Saf6 |
| Ets97D | Taf13 | Orc4 | Mabi | Ctf4 | ich |
| REPTOR-BP | Ptx1 | zfh 1 | tx | zfh2 | Taf12L |
| MED8 | Ets21C | ZIPIC | CG2199 | Sp1 | Sirt1 |
| CG18600 | eve | Oxp | srp | inv | vtd |
| oc | baf | woc | Pdp1 | CG5245 | LBR |
| CG4328 | ERR | Stat92E | His3:CG338 | lmd | His1:CG338 |
| | | | 09 | | 49 |
| SREBP | His3:CG338 | Ulp1 | CG30431 | Uxt | sage |
| | 45 | | | | |
| CG8944 | His3:CG338 | CG9876 | His1:CG338 | Rx | JHDM2 |
| | 21 | | 07 | | |
| CG3032 | CG3756 | His1:CG338 | fd3F | Asx | E(spl)m8- |
| | | 61 | | | HLH |
| His3:CG3382 | MED27 | ph-d | CG6066 | MED19 | Arpc1 |
| 7 | | | | | |
| vnd | CG11456 | CG12782 | l(1)sc | HP1Lcsd | rhi |
| CG13609 | Erk7 | His3:CG338 | l(3)mbt | His3:CG338 | MED4 |
| | | 57 | | 54 | |
| Cdk9 | GATAe | knrl | NSD | bip2 | HP6 |
| CG17802 | wek | CG12316 | Nup58 | cg | Rpb7 |
| Charon | His1:CG338 | RpII18 | hang | esc | Nup107 |
| | 25 | | | | |
| tHMG1 | TFAM | Kr-h1 | CG34031 | MED22 | D19A |
| His1:CG33843 | msl-3 | Fas3 | CG7655 | toc | ey |
| Su(z)2 | MED7 | Sox21a | PCNA2 | dmrt99B | nht |
| Ndc1 | CG9018 | CG3328 | mil | CG33557 | Awh |
| Trf5 | achi | ix | gcm | Nup35 | CG9932 |
| Nup205 | vg | l(2)37Cg | CG10274 | bocks | ash1 |
| Patj | bin | pdm3 | bun | Set | MED6 |
| grh | Hey | Sox14 | mtRNApol | HmgZ | maf-S |
| His3:CG33860 | bcd | Cap-D2 | His3:CG338 | CG17359 | bs |
| | | | 30 | | |
| CG4318 | gce | Vsx1 | His3:CG338 | scrt | Hmx |
| | | | 12 | | |
| Hand | lms | RanBP3 | Crg-1 | Myb | Bap60 |
| gl | ebi | Spps | CG32532 | trx | His3:CG338 |
| | | | | | 33 |
| DNApol- | tplus3b | bap | gem | CG7386 | Smr |
| alpha180 | | | | | |
| E(spl)m7- | atms | CG6689 | shn | MED11 | Atf-2 |
| HLH | | | | | |
| CG10543 | His3:CG338 | MESR3 | Cdc45 | l(3)73Ah | Cap-H2 |
| | 36 | | | | |
| Trf4 | CG42390 | CG2889 | trem | acj6 | MED30 |
| Fer3 | ind | His1:CG338 | vri | His1:CG338 | Elba2 |
| | | 19 | | 64 | |
| CG33213 | Sox21b | fd102C | Cdk8 | CG10321 | zld |
| CG4744 | Ets98B | SWIP | kud | TfIIEalpha | CG12236 |
| hkb | CG1529 | sim | Mad | CG15478 | RpII140 |
| Hers | Rel | Hr51 | His1:CG338 | fd96Cb | CG4360 |
| | | | 37 | | |
| CG8159 | gcm2 | pho | ewg | uri | Psc |
| tll | dsx | Chrac-14 | TfIIA-S-2 | Lmx1a | fkh |
| Nipped-B | toy | MED20 | CG4854 | CG12081 | CG4730 |
| CG34224 | mof | CG13287 | CG7963 | CTCF | tap |
| stc | sr | TER94 | CG3491 | His3:CG338 | His3:CG338 |
| | | | | 66 | 18 |
| Tbp | brk | Nup188 | CG3281 | tplus3a | Nup98-96 |
| Lis-1 | Sin | Max | MEP-1 | Ankle2 | Dif |
| en | Atf3 | B-H1 | HmgD | CG11294 | HLH54F |
| cnc | Optix | CG6204 | Chd1 | Odj | BigH1 |
| moon | lbl | BEAF-32 | CG32772 | ken | glu |
| RpIIIC160 | fd59A | Kr | CG4374 | grn | HLH3B |
| GATAd | His1:CG316 | CG15696 | CG6791 | CG10654 | esg |
| | 17 | | | | |
| Nup54 | His1:CG338 | SC | Cap-D3 | dwg | RpIIIC53 |
| | 13 | | | | |
| sno | Rtf1 | Ubx | CG1421 | crc | Ets96B |
| Sbf | Sox100B | Nph | CG11696 | cas | Drgx |
| Rpb10 | Dbx | sd | omd | Taf10 | His1:CG338 |
| | | | | | 40 |
| neb | Su(z)12 | CG32971 | MTA1-like | thoc5 | ci |
| kni | eyg | sens-2 | vib | RpI135 | His3:CG338 |
| | | | | | 24 |
| mrn | CG13137 | CG9727 | HGTX | pb | His1:CG338 |
| | | | | | 22 |
| Spt5 | HIPP1 | Xpd | Pk34A | CG12071 | cato |
| D19B | htk | Eip74EF | Mcm7 | CG12609 | Sry-beta |
| CG4496 | crol | ph-p | tbrd-1 | DNApol- | RpII33 |
| | | | | alpha60 | |
| CG14710 | Rcc1 | His3:CG338 | Mcm3 | TfAP-2 | Hcf |
| | | 06 | | | |
| rib | h | ord | Dlip3 | danr | CG33051 |
| mld | grau | Cdc6 | Ref1 | dysf | CG6659 |
| ara | yuri | CG7101 | exd | ase | Ntf-2 |
| bsh | CG43689 | tj | Mcm5 | H15 | Sec13 |
| CG8111 | CG4880 | MTF-1 | Spt6 | Cdk7 | CG10669 |
| nom | CG2129 | Gp210 | al | ftz-f1 | dpy |
| CG10959 | Pdi | CG30020 | slbo | CG31365 | His1:CG338 |
| | | | | | 16 |
| Nup43 | MED16 | otp | N | wge | CG14431 |
| Mnt | Usf | dpn | Mnn1 | emb | spag4 |
| SA-2 | CG34367 | His1:CG338 | aop | chb | Hira |
| | | 55 | | | |
| CG15725 | bab2 | CG12219 | run | mirr | Myc |
| NK7.1 | erm | polybromo | Nf-YC | SMC1 | TfIIA-S |
| Hnf4 | CG1663 | Pc | msl-1 | insv | Wbp2 |
| Dd | E5 | CWO | CycC | SMC2 | trh |
| CG17829 | ato | Ote | Scr | dl | HP1b |
| org-1 | Doc3 | His3:CG316 | kn | drm | gsb-n |
| | | 13 | | | |
| MRG15 | Su(var)3-7 | Taf1 | Etl1 | CoRest | RpI1 |
| apt | Dsp1 | nej | corto | e(y)2b | msl-2 |
| sbr | bif | Nup75 | CG17806 | MAN1 | Eip78C |
| MED28 | RpIII128 | eg | escl | CG7691 | CG17803 |
| E(spl)mbeta- | Orc1 | mRpL12 | Sidpn | CG1024 | Su(H) |
| HLH | | | | | |
| Rpb11 | slou | sens | Sin3A | HP1D3csd | ap |
| tup | yrt | REPTOR | rogdi | CG11617 | tHMG2 |
| zen2 | MED1 | Skadu | CG32767 | Bsg25A | Nup62 |
| pros | CG10431 | Gcn5 | Uggt | Ran | Rcp |
| Dp | CG10147 | Nf-YA | His3:CG338 | Doc1 | Ntf-2r |
| | | | 39 | | |
| Atf6 | fs(1)Ya | kmg | Smox | nub | MED31 |
| tgo | polo | IntS2 | NFAT | CG18476 | Nap1 |
| ATbp | Taf6 | Rab11 | CG33785 | CG14712 | CG14711 |
| hb | CG17328 | CG14667 | ab | Ctr9 | zen |
| p53 | sima | mbo | pad | salm | E(spl)mdelta- |
| | | | | | HLH |
| Dlic | Ibf2 | cbt | CG9899 | lz | D |
| Vsx2 | rec | CG18764 | Ddx1 | zf30C | Gdn1 |
| Jra | Lim1 | Glut4EF | Rfx | ova | CG1602 |
| bab1 | CG10348 | Cf2 | xmas | cyc | Meics |
| Orc3 | Hr4 | Ets65A | mle | su(Hw) | CG30389 |
| Taf2 | pdm2 | Plzf | Sce | FoxK | Nup37 |
| lbe | CG10887 | CG9650 | nerfin-2 | her | E(spl)m3- |
| | | | | | HLH |
| fru | MED9 | 14-3-3zeta | Cp190 | Camta | E2f2 |
| MED10 | Mad1 | CG2712 | Opbp | hyx | CG1792 |
| prg | Hr83 | Xrp1 | da | wash | Gsc |
| CG5199 | Su(var)205 | FoxL1 | dac | Ovo | CG9215 |
| Dr | CG18011 | Non2 | schlank | Ranbp16 | MED17 |
| ranshi | Bap55 | RunxA | Ada2b | cry | SoxN |
| abo | bowl | RanGAP | Phs | Cse1 | CG11398 |
| calypso | CG1233 | sob | Sgf11 | pnt | Mat1 |
| CG4820 | hng3 | CG17724 | CG6654 | CG3515 | RpI12 |
| toe | sba | RpL29 | abd-A | vvl | hth |
| Nup93-2 | nau | mid | ro | az2 | Taf4 |
| JMJD5 | MED24 | CG5380 | e(y)2 | fs(1)h | fuss |
| His1:CG33804 | Cdc5 | Atu | His1:CG33831 | RpII15 | ems |
| Mrtf | Samuel | Nup50 | Lim3 | Adf1 | nudE |
| Nup160 | DNApol- | gsb | wdn | Nup154 | Dad |
| | alpha73 | | | | |
| comr | CG8388 | MBD-R2 | crm | slp1 | MED26 |
| pnr | prd | Dsor1 | Axud1 | Ada3 | His3:CG338 |
| | | | | | 42 |
| PHDP | Hsf | Nf-YB | Utx | dsf | sw |
| Rpb12 | upSET | His1:CG338 | p23 | mad2 | Ndf |
| | | 28 | | | |
| Elys | His3:CG338 | Antp | Pph13 | exex | Eip75B |
| | 03 | | | | |
| Trl | CG2202 | caz | jim | E(spl)m5- | CG31875 |
| | | | | HLH | |
| Klf15 | pzg | Dfd | Irbp18 | CG2116 | CG8301 |
| Chd3 | ftz | dre4 | HHEX | TfIIFbeta | C15 |
| Ssl1 | e(y)3 | Mitf | Pur-alpha | Impbeta11 | br |
| Trf2 | HP1e | ttk | FAM21 | dmrt93B | slp2 |
| lola | can | Scm | salr | Poxn | dimm |
| CG11902 | Asciz | His3:CG338 | CHES-1-like | CG13204 | TflIFalpha |
| | | 63 | | | |
| CG14655 | nsl1 | CG4282 | CG12299 | Rpb8 | SMC3 |
| His3:CG33815 | CG41106 | Snoo | Iswi | CG4936 | vis |
| kto | CG2120 | Oli | MED21 | btd | Nup93-1 |
| luna | His1:CG338 | ct | nerfin-1 | IntS1 | CG15073 |
| | 46 | | | | |
| mamo | sug | retn | Doc2 | Tfb4 | Axs |
| Parp16 | unpg | CG3708 | CG6808 | CG13123 | CG7987 |
| Mef2 | Nup133 | Fer1 | Mcm2 | Aladin | jumu |
| Mtor | bbx | CG44002 | Sox102F | Deaf1 | MED15 |
| ss | | | | | |

In other embodiments, the target is a G4 binding protein, or a fragment thereof. G4 binding proteins include, without limitation, SLIRP, LARK, GNL1, STM1P, CIRBP, SERBP1, eIF4G, WRN, Nucleolin, Mre11, DHX36, hnRNP A1, CNBP, BRCA1, breast cancer type 1 susceptibility protein; hnRNP, heterogeneous nuclear ribonucleoprotein; POTI, protection of telomeres 1; RPA, replication protein A; TEBP, Telomere End Binding Protein; TLS/FUS, translocated in liposarcoma/fused in sarcoma; Topo I, Topoisomerase I; TRF2, telomere repeat binding factor 2; UP1, unwinding protein 1; PARP-1, Poly [ADP-ribose] polymerase 1; CNBP, cellular nucleic-acid-binding protein; IGF-2, Insulin-like growth factor 2; MAZ, myc-associated zinc-finger; FMR2, fragile X mental retardation 2; RHAU, the RNA helicase associated with AU-rich element; SRSF, serin/arginine-rich splicing factor; BLM, Bloom syndrome protein; Dna2, DNA replication helicase/nuclease 2; G4R1, G4 Resolvase 1; FANCJ, Fanconi anemia complementation group J; Sgs1, small growth suppressor 1; and WRN, Werner syndrome ATP-dependent helicase.

### Transposase

The fusion protein further includes a transposase for use in tagmentation. A “transposase” is an enzyme that binds to the end of a transposon and catalyzes its movement to another part of the genome by a cut and paste mechanism or a replicative transposition mechanism. In one embodiment, such enzyme is a member of the RNase superfamily of proteins which includes retroviral integrases. Examples of transposases include Tn3, Tn5, and hyperactive mutants thereof. Tn5 can be found in Shewanella and Escherichia bacteria. An example of a hyperactive mutant Tn5 comprises a mutation of E54K and/or L372P. In certain embodiments of this method, the transposase is TnY or Tn5.

An exemplary coding sequence for Tn5 transposase is shown in SEQ ID NO: 1:

atgattaccagtgcactgcatcgtgcggcggattgggcgaaaagcgtgtt
ttctagtgctgcgctgggtgatccgcgtcgtaccgcgcgtctggtgaatg
ttgcggcgcaactggccaaatatagcggcaaaagcattaccattagcagc
gaaggcagcaaagccatgcaggaaggcgcgtatcgttttattcgtaatcc
gaacgtgagcgcggaagcgattcgtaaagcgggtgccatgcagaccgtga
aactggcccaggaatttccggaactgctggcaattgaagataccacctct
ctgagctatcgtcatcaggtggcggaagaactgggcaaactgggtagcat
tcaggataaaagccgtggttggtgggtgcatagcgtgctgctgctggaag
cgaccacctttcgtaccgtgggcctgctgcatcaagaatggtggatgcgt
ccggatgatccggcggatgcggatgaaaaagaaagcggcaaatggctggc
cgctgctgcaacttcgcgtctgagaatgggcagcatgatgagcaacgtga
ttgcggtgtgcgatcgtgaagcggatattcatgcgtatctgcaagataaa
ctggcccataacgaacgttttgtggtgcgtagcaaacatccgcgtaaaga
tgtggaaagcggcctgtatctgtatgatcacctgaaaaaccagccggaac
tgggcggctatcagattagcattccgcagaaaggcgtggtggataaacgt
ggcaaacgtaaaaaccgtccggcgcgtaaagcgagcctgagcctgcgtag
cggccgtattaccctgaaacagggcaacattaccctgaacgcggtgctgg
ccgaagaaattaatccgccgaaaggcgaaaccccgctgaaatggctgctg
ctgaccagcgagccggtggaaagtctggcccaagcgctgcgtgtgattga
tatttatacccatcgttggcgcattgaagaatttcacaaagcgtggaaaa
cgggtgcgggtgcggaacgtcagcgtatggaagaaccggataacctggaa
cgtatggtgagcattctgagctttgtggcggtgcgtctgctgcaactgcg
tgaatcttttactccgccgcaagcactgcgtgcgcagggcctgctgaaag
aagcggaacacgttgaaagccagagcgcggaaaccgtgctgaccccggat
gaatgccaactgctgggctatctggataaaggcaaacgcaaacgcaaaga
aaaagcgggcagcctgcaatgggcgtatatggcgattgcgcgtctgggcg
gctttatggatagcaaacgtaccggcattgcgagctggggtgcgctgtgg
gaaggttgggaagcgctgcaaagcaaactggatggctttctggccgcgaa
agacctgatggcgcagggcattaaaatc

The amino acid sequence for Tn5 transposase is shown in SEQ ID NO: 2:

MITSALHRAADWAKSVFSSAALGDPRRTARLVNVAAQLAKYSGKSITISS
EGSKAMQEGAYRFIRNPNVSAEAIRKAGAMQTVKLAQEFPELLAIEDTTS
LSYRHQVAEELGKLGSIQDKSRGWWVHSVLLLEATTFRTVGLLHQEWWMR
PDDPADADEKESGKWLAAAATSRLRMGSMMSNVIAVCDREADIHAYLQDK
LAHNERFVVRSKHPRKDVESGLYLYDHLKNQPELGGYQISIPQKGVVDKR
GKRKNRPARKASLSLRSGRITLKQGNITLNAVLAEEINPPKGETPLKWLL
LTSEPVESLAQALRVIDIYTHRWRIEEFHKAWKTGAGAERQRMEEPDNLE
RMVSILSFVAVRLLQLRESFTPPQALRAQGLLKEAEHVESQSAETVLTPD
ECQLLGYLDKGKRKRKEKAGSLQWAYMAIARLGGFMDSKRTGIASWGALW
EGWEALQSKLDGFLAAKDLMAQGIKI

In certain embodiments, the transposase is TnY. TnY is a hyperactive mutant of the transposase from Vibrio parahemolyticus (ViPar) with P50K and M53Q mutations. The inside and outside ends (IE and OE, respectively) of the ViPar transposon utilize the same sequence as the IE and OE of the Tn5 transposon (see, WO 2021/011433, which is incorporated herein by reference).

An exemplary coding sequence for TnY transposase is shown in SEQ ID NO: 3:

atgacccact ccgatgcgaa actgtgggct caggagcaat tcggtcaggc ccaactgaaagatccgcgcc cacccagcg
cctgatttct ctggcgacca gcattgctaa ccagccgggtgttagcgttg cgaaactgcc gttttctaaa gccgatcagg
agggcgcgta ccgtttcattcgtaacgata acatcgacgc gaaagacatc gctgaagcag gctttcagtc
caccgtatcccgcgctaacg aacacaaaga gctgctggcg ctggaagaca ctacgaccct gtctttcccgcatcgttcca
tcaaagaaga actgggccat acgaaccagg gtgatcgcac ccgcgccctgcacgttcact ctaccctgct gttcgcgccg
cagaaccaga ctatcgtggg tctgatcgag cagcagcgtt ggtctcgtga tattactaaa cgcggtcaga aacatcagca
cgctacccgt ccttataaag aaaaagaatc ctataaatgg gagcaggctt cccgtcgtgt tgtggagcgc ctgggtgata
aaatgctgga tgtcatttct gtttgcgacc gcgaggcaga tctgtttgaa tacctgacct acaaacgtca acaccagcag
cgtttcgttg ttcgtagcat gcagtctcgc tgtctggaag aacacgctca gaaactgtat gactacgcac aggcgctgcc
atctgtaaaa acgaaggcac tgaccatccc tcaaaaaggt ggccgtaaag cacgtgacgt taaactggac gttaaatacg
gccaggttac tctgaaagcg ccggccaaca aaaaggagca cgcaggcatt ccggtttact acgtgggctg cctggaacag
ggtacttcca aagataaact ggcgtggcac ctgctgacct ctgaacctat taacaacgtc gaggatgcca tgcgtatcat
cggctactac gaacgtcgtt ggctgatcga ggattttcac aaagtatgga aatccgaagg tactgacgta gaatccctgc
gtctgcagag caaagacaac ctggaacgtc tgtccgttat ctacgcgttt gttgctaccc gcctgctggc actgcgtttt
atcaaggaag ttgatgaact gaccaaagaa agctgtgaaa aagttctggg ccagaaagcg tggaaactgc tgtggctgaa
gctggaatct aaaaccctgc cgaaagaggt accggacatg ggttgggctt ataaaaacct ggctaaactg ggtggctgga
aggacactaa gcgtaccggt cgcgcttcta tcaaagttct gtgggagggt tggttcaaac tgcagaccat cctggagggc
tatgaactgg cgatgtccct ggaccac

The amino acid sequence for TnY transposase is shown in SEQ ID NO: 4:

MTHSDAKLWAQEQFGQAQLKDPRRTQRLISLATSIANQPGVSVAKLPFSK
ADQEGAYRFIRNDNIDAKDIAEAGFQSTVSRANEHKELLALEDTTTLSFP
HRSIKEELGHTNQGDRTRALHVHSTLLFAPQNQTIVGLIEQQRWSRDITK
RGQKHQHATRPYKEKESYKWEQASRRVVERLGDKMLDVISVCDREADLFE
YLTYKRQHQQRFVVRSMQSRCLEEHAQKLYDYAQALPSVKTKALTIPQKG
GRKARDVKLDVKYGQVTLKAPANKKEHAGIPVYYVGCLEQGTSKDKLAWH
LLTSEPINNVEDAMRIIGYYERRWLIEDFHKVWKSEGTDVESLRLQSKDN
LERLSVIYAFVATRLLALRFIKEVDELTKESCEKVLGQKAWKLLWLKLES
KTLPKEVPDMGWAYKNLAKLGGWKDTKRTGRASIKVLWEGWFKLQTILEG
YELAMSLDH

Other useful transposases include those having sequences set forth in the table below:

| | | |
|---|---|---|
| P. luminescens | MFSTSAEQWANDTFQHAELGDKRRTNRLVKV | SEQ ID NO: 5 |
| | ACSLANHIGQSLVQSLDSPADVEAAYRLTRNS | |
| | AI | |
| sarSeaEAK | MDPEQWAQCQFGHANLNDPRRTQRLVSLATS | SEQ ID NO: 6 |
| | ITQQPGVAVSKLPLSPAEM EGAYRFIRNE | |
| | NIQ | |
| V. campbelli | MTHSDAKLWAQEQFGQAQLKDPRRTQRLISL | SEQ ID NO: 7 |
| | ATSIANQPGVSVAKLPFSPADMEGAYRFI | |
| | RNENIN | |
| V. parahemolyticus | MTHSDAKLWAQEQFGQAQLKDPRRTQRLIS | SEQ ID NO: 8 |
| | LATSIANQPGVSVAKLPFSPADMEGAYRFI | |
| | RNDNID | |
| Tn5 HA | MITSALHRAADWAKSVFSSAALGDPRRTAR | SEQ ID NO: 9 |
| | LVNVAAQLAKYSGKSITISSEGSKAMQEGA | |
| | YRFIRNPNVS | |
| C. glomeribacter | MFRREAGDWAHQTFGECNLGDERRTKRLVEV | SEQ ID NO: 10 |
| | GKRLANQIGCSLPKCCEGDKAALLGSYRLLRN | |
| | DAVN | |
| L. longbeachae | MDLAIEDAAAWSEAIFGSVDLGDKRLTRRL | SEQ ID NO: 11 |
| | TQIGKQLSSM PGGSLPESCEGQDALIEGSY | |
| | RFLRNKRVT | |
| L. pneumophila | MDLAIEDAAAWSEAIFGSVALGDKRLTRRL | SEQ ID NO: 12 |
| | IQIGKQLSSIPGGSLSESCEGQDALIEGSY | |
| | RFLRNKRVT | |

In certain embodiments, the fusion protein also includes a protein “tag” useful for purification, detection, solubilization, localization, and/or protease protection. Various protein tags are known in the art. In some embodiments, an affinity tag is included which allows affinity purification of the fusion protein. For example, in one embodiment, the fusion protein harbors a chitin binding domain (CBD) sequence, enabling affinity purification using chitin resin, followed by elution of the purified fusion protein in reducing conditions. In certain embodiments, the protein tag is a chitin binding domain, FLAG, 6×-His, GST, CBP, HA, or c-myc. Other protein tags are known in the art.

### Nucleic Acids

Provided herein are nucleic acid molecules, expression cassettes, vectors, and host cells comprising the same, that encode the fusion proteins described herein. The nucleic acid encoding the fusion protein may be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein for production of the same. The nucleic acid encoding the fusion protein can also be cloned into an expression vector, for administration to a plant cell, animal cell, preferably a mammalian cell or a human cell, fungal cell, bacterial cell, or protozoan cell.

To obtain expression, a sequence encoding a fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

Methods for introducing polypeptides and nucleic acids into a target cell (host cell) are known in the art, and any known method can be used to introduce a nuclease or a nucleic acid into a cell. Non-limiting examples of suitable methods include electroporation, viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Exemplary constructs encoding fusion proteins described herein are provided in SEQ ID NOs: 13 to 16. These examples are meant to represent, but not limit, the fusion proteins described herein.

| | | Nanobody | Transposase | Mxe Gyr A | |
|---|---|---|---|---|---|
| Construct | SEQ ID NO | coding seq | coding seq | Intein | CBD |
| pTXB1- | 13 | 1-375 | 445-1872 | 1877-2466 | 2497-2652 |
| alnbMMIgG | | | | | |
| 1-Tn5 | | | | | |
| pTXB1- | 14 | 1-390 | 460-1887 | 1888-2481 | 2512-2667 |
| alnbMMIgG | | | | | |
| 2a-Tn5 | | | | | |
| pTXB1- | 15 | 1-394 | 463-1890 | 1891-2484 | 2515-2670 |
| alnbMmKap | | | | | |
| pa-Tn5 | | | | | |
| pTXB1- | 16 | 1-363 | 433-1860 | 1861-2454 | 2485-2640 |
| alnbOc-Tn5 | | | | | |

### Transposome Complex

The compositions and methods described herein utilize a transposome complex which includes a transposase-ligand fusion protein (or transposase alone) and a transposon. The transposome complex can vary depending upon the application for which the compositions are being used.

As used herein, the term “transposon” is used interchangeably with mosaic-end DNA sequence (MEDS) adapter, referring to a nucleic acid molecule that is capable of being incorporated into a nucleic acid by a transposase enzyme. The MEDS adapter includes two transposon ends (also termed “arms” and “mosaic end” or “ME”, for example, a double-stranded mosaic end). In one embodiment, the two transposon ends are linked by a sequence that is sufficiently long to form a loop in the presence of a transposase. The formation of a complex between the Tn5 transposase and the 19-bp MEs is necessary for the transposition to occur, and the intervening DNA must be long enough to bring 2 of these sequences close together to form an active transposase homodimer. Transposons can be double-, single-stranded, or mixed, containing single- and double-stranded region(s), depending on the transposase used to insert the transposon. For Tn5 transposases, the transposon ends are double-stranded, but the linking sequence need not be double-stranded. In a transposition event, these transposons are inserted into double-stranded DNA. The term “transposon end” refers to the sequence region that interacts with transposase. In a transposition event, single-stranded transposons are inserted into single-stranded DNA by a transposase enzyme. See, for example, US2015/0337298A1, which is incorporated herein by reference.

In one embodiment, the transposome complex comprises a transposase assembled with a transposon comprising two mosaic end (ME) double-stranded (MEDS) adapters, for recognition by a transposase. Such mosaic end sequences are known in the art, for example, for use with the Tn5 transposase. The top strand of an exemplary ME sequence for use with Tn5 transposase is: 5′-AGATGTGTATAAGAGACAG-3′ (SEQ ID NO: 17). In one embodiment, the ME sequence is contained on the 5′ end of the adapter, the 3′ end, or both. In one embodiment the ME sequence is contained on the 3′ end of the adapter. See, e.g., Picelli et al., Genome Research, Jul. 30, 2014, 24:2033-40, which is incorporated herein by reference. Other sequences which may be used in place of a ME include inverted 19-bp end sequences (ESs), including outside end (OE) and inside end (IE) sequences of the transposon. An example of an OE sequence is: 5′-CTGACTCTTATACACAAGT-3′ (SEQ ID NO: 18). An example of an IE sequence is: 5′ CTGTCTCTTGATCAGATCT-3′ (SEQ ID NO: 19). See, e.g., Reznikoff, Molecular Microbiology, 47(5):1199-1206 (February 2003), which is incorporated herein by reference.

In addition to the sequences required for completing tagmentation, the MEDS adapters may include one or more additional sequences for further sample processing. The additional sequence(s) will depend on the application for which the transposome complex will be used. Examples of MEDS composition components (in addition to ME) are provided in Table 6 below. This table provides representative embodiments for each assay methodology, as known in the art, and further described herein. However, the MEDS components can be modified by the person of skill in the art, based on the requirements of the assay being performed.

TABLE 6

| | | | | Substrate Oligo |
|---|---|---|---|---|
| | TnBlocker | nb-Tn5 | MEDS Components | Components |
| low salt C&T | x | o | ME, target barcode, | N/A |
| | | | UMI (o), seq | |
| | | | adapter/PCR handle | |
| NTT-seq | o | x | ME, target barcode, | N/A |
| (multiplexed | | | UMI (o), seq | |
| C&T) | | | adapter/PCR handle | |
| single cell | x | o | ME, seq adapter/PCR | seq adapter/PCR handle, |
| low salt C&T | | | handle/capture | bead/cell capture barcode, |
| | | | compatible sequence, | capture sequence |
| | | | target barcode (o) | |
| single cell | o | x | ME, seq adapter/PCR | seq adapter/PCR handle, |
| NTT-seq | | | handle/capture | bead/cell capture barcode, |
| | | | compatible sequence, | capture sequence |
| | | | target barcode (x), | |
| spatial WGS | | | ME, T7 promoter, seq | seq adapter/PCR handle, |
| | | | adapter/PCR handle, | spatial feature capture |
| | | | capture compatible | barcode, capture UMI(o), |
| | | | sequence | capture sequence |
| spatial | o | | ME, T7 promoter, seq | seq adapter/PCR handle, |
| ATAC | | | adapter/PCR handle, | spatial feature capture |
| | | | capture compatible | barcode, capture UMI(o), |
| | | | sequence | capture sequence |
| spatial C&T | o | o | ME, T7 promoter, seq | seq adapter/PCR handle, |
| | | | adapter/PCR handle, | spatial feature capture |
| | | | target barcode (o), | barcode, capture UMI(o), |
| | | | capture compatible | capture sequence |
| | | | sequence | |
| spatial NTT- | o | x | ME, T7 promoter, seq | seq adapter/PCR handle, |
| seq | | | adapter/PCR handle, | spatial feature capture |
| | | | target barcode (x), | barcode, capture UMI(o), |
| | | | capture compatible | capture sequence |
| | | | sequence | |

x= required;
o=optional

The additional MEDS components are further described briefly herein. These components are, in most cases, known in the art, and may be readily designed by the person of skill based on the teachings of the specification, and the art. Examples of such nucleic acid molecules and uses thereof, as may be used with compositions and methods of the present disclosure, are provided in U.S. Patent Pub. Nos. 2020/0248176A1, 2014/0378345, and 2015/0376609, each of which is incorporated herein by reference in its entirety.

In certain embodiments, the MEDS adapter includes a PCR handle or priming region to enable PCR amplification subsequent to tagmentation. Optionally, the PCR handle is compatible with a capture sequence that is attached to a bead, glass slide, or other solid support. In some embodiments, the MEDS adapter includes a sequencing priming region such as, for example, a P5 sequence or P7 sequence for Illumina sequencing. For example, a P5 priming region may be annealed to a first MEDS and a P7 priming region may be annealed to a second MEDS. In some embodiments, the primer can comprise an R1 primer sequence for Illumina sequencing. R1 primer: SEQ ID NO: 20: 5′ TCGTCGGCAGCGTCAGATGTGTATAAGAGACAG. In some cases, the primer can comprise an R2 primer sequence for Illumina sequencing: R2 primer: SEQ ID NO: 21: 5′ GTCTCGTGGGCTCGGAGATGTGTATAAGAGACAG. Other priming regions for use with other systems are known and may be used.

The MEDS adapter may comprise a specific priming sequence, such as an mRNA specific priming sequence (e.g., poly-T sequence for priming reverse transcription of RNA), a targeted priming sequence, and/or a random priming sequence. In certain embodiments, the MEDS adapter includes the promoter for the T7 RNA polymerase to allow for in vitro transcription (IVT) during sample processing.

In certain embodiments, the MEDS adapter further includes a barcode sequence that identifies the target epitope of the ligand incorporated into the transposome complex, referred to herein as the “target barcode”. The target barcode sequence is useful, inter alia, for identification of a binding moiety, as further described herein. This sequence is a unique sequence which allows identification of the specific fusion protein or ligand (e.g., nanobody) being tested or employed. The target barcode can be designed to any length available using synthesis technology, and the length of the barcode limits the number of formulations that may be tested simultaneously. For example, using a 10 bp barcode, there are a total of 1048576 possible combinations. Thus, the target barcode sequence is, in one embodiment, between 5 nt to 100 nt in length. In another embodiment, the target barcode sequence is between 10 nt to 20 nt in length. In one embodiment, the target barcode is 10 nt in length. In another embodiment, the target barcode is 5, 6, 7, 8, 9, 10, 11, 12, 13, 14, 15, 16, 17, 18, 19 or 20 nt in length.

In certain embodiments, the MEDS adapter includes a unique molecular identifier (UMI) specific to each individual MEDS adapter. The UMI are randomly generated sequences which serve to detect duplicates of original molecules generated by amplification during deep sequencing. Inclusion of these UMI in the first steps of sequencing library preparation offers several benefits. UMI create a distinct identity for each input molecule: this makes it possible to estimate the efficiency with which input molecules are sampled, identify sampling bias, and most importantly, identify and correct for the effects of PCR amplification bias. The UMI can be designed to any length available using synthesis technology. The UMI is, in one embodiment, between 5 nt to 100 nt in length. In another embodiment, the UMI is between 10 nt to 20 nt in length. In one embodiment, the UMI is 10 nt in length. In another embodiment, the UMI is 5, 6, 7, 8, 9, 10, 11, 12, 13, 14, 15, 16, 17, 18, 19 or 20 nt in length. Design of UMI is known in the art, for example, Clement et al., AmpUMI: design and analysis of unique molecular identifiers for deep amplicon sequencing, Bioinformatics, Volume 34, Issue 13, 1 Jul. 2018, Pages i202-i210, which is incorporated herein by reference. In certain embodiments, the UMI is omitted. The UMI associated with the MEDS is sometimes referred to herein as the tagmentation UMI, or tUMI, as all nucleic acids produced from a single tagmentation event will harbor the same tUMI.

In certain embodiments, the MEDS adapter includes a capture compatible sequence that allows binding of the adapter to a bead, chip, slide, or other substrate. In some embodiments, the capture sequence is a unique nucleotide sequence, not found in the genome, that is complementary to a sequence that is conjugated to a bead, chip, slide or other substrate, as further described herein. In certain embodiments, the capture compatible sequence is a polyT sequence. In certain embodiments, the capture sequence is found in the 5′ end of the MEDS adapter.

In certain embodiments, the transposase exists as a dimer, wherein said transpose dimer comprises a first transposase bound to a first MEDS (sometimes referred to as MEDS-A) comprising a first MEDS adapter sequence; and a second transposase bound to a second MEDS (sometimes referred to as MEDS-B) comprising a second MEDS adapter sequence wherein said first adapter sequence is different from said second adapter sequence.

### Substrate

In certain embodiments of the methods described herein, a physical substrate is used to enable capture of tagmented DNA (or product thereof) at some stage of sample processing. Such physical substrates are known in the art and include beads, glass or other slides, plates, chips, chambers, etc. For example, the Visium Spatial Gene Expression Slide is an example of a substrate useful with some of the methods described herein. Another nonlimiting example of a useful substrate is the Chromium Next GEM Gel beads. Such physical substrates generally have oligonucleotides attached thereto that allow capture of the tagmented DNA (or product thereof). Exemplary components of the substrate oligonucleotide useful for various methods discussed herein, are shown in Table 6, and further described herein. In some embodiments, the substrate oligonucleotide molecules are releasably attached to the bead or substrate. In some embodiments, the method further comprises releasing the plurality of substrate oligonucleotide molecules from the bead or substrate. In some embodiments, the bead is a gel bead. In some embodiments, the gel bead is a degradable gel bead.

In certain embodiments, a capture sequence may be included on the substrate oligonucleotide. The capture sequence may include a universal capture sequence and, optionally, a unique UMI, referred to as a capture UMI (cUMI) that identifies a specific capture event, i.e., the binding of a single oligo to its target molecule. When present on the MEDS, the capture sequence on the substrate oligonucleotide must be complementary to the capture compatible sequence in the MEDS. The sequence may be any unique sequence, as long as the capture sequence and the capture compatible sequence are complementary.

In some embodiments, the substrate oligonucleotide contains a barcode sequence, that is used to identify the source/location of the sample, such that all oligos on a specific bead, or in a specific spot on a slide share the same barcode. Such barcode may be termed a “cellular barcode” or “spatial barcode”. Similarly, the cellular barcode sequence is, in one embodiment, between 5 nt to 100 nt in length. In another embodiment, the cellular barcode sequence is between 10 nt to 20 nt in length. In one embodiment, the cellular barcode is 10 nt in length. In another embodiment, the cellular barcode is 5, 6, 7, 8, 9, 10, 11, 12, 13, 14, 15, 16, 17, 18, 19 or 20 nt in length.

In certain embodiments, the substrate oligonucleotide includes a PCR handle or priming region to enable PCR amplification subsequent to tagmentation. Optionally, the PCR handle is compatible with a capture sequence that is attached to a bead, glass slide, or other solid support. In some embodiments, the substrate oligonucleotide includes a sequencing priming region such as, for example, a P5 sequence (SEQ ID NO: 22-5′-AATGATACGGCGACCACCGAGATCTACAC) or P7 (SEQ ID NO: 23-5′-CAAGCAGAAGACGGCATACGAGAT) sequence for Illumina sequencing. In some embodiments, the primer can comprise an R1 primer sequence for Illumina sequencing. R1 primer: SEQ ID NO: 20. In some cases, the primer can comprise an R2 primer sequence for Illumina sequencing: R2 primer: SEQ ID NO: 21. Other priming regions for use with other systems are known and may be used. Any suitable nucleic acid sequencing method can be used to sequence the nucleic acids described herein, and/or to detect the presence, absence or amount of the various nucleic acids, constructs, targets, oligonucleotides, amplification products and barcodes described herein.

In certain embodiments, the substrate oligonucleotide includes a sequencing primer (e.g., partial read I sequencing primer), a spatial barcode, optionally a UMI, and a polyT sequence. In other embodiments, the substrate oligonucleotide includes a sequencing primer (e.g., partial read 1 sequencing primer), a cellular barcode, optionally a UMI, and a sequencing adapter sequence (e.g., an Illumina P5 sequence).

### Blocking Oligonucleotide

In certain embodiments, the methods and compositions described herein utilize a blocking oligonucleotide, sometimes referred to herein as the “Tn Blocker”. As used herein, the term oligonucleotide (sometimes referred to as “oligo”) refers to a short nucleic acid molecule, usually between about 5 nucleotides and about 100 nucleotides. The blocking oligonucleotide is a short nucleic acid sequence that contains a sequence that is complementary to the DNA sequence to which the transposase preferentially binds. In certain embodiments, the thymine residues are replaced with uracil residues in the oligonucleotide. Preferentially, the oligonucleotide is double stranded.

As noted above, the oligonucleotide is usually between about 5 nucleotides and about 100 nucleotides. However, other lengths are possible. For example, the oligonucleotide may range from about 5 nucleotides to about 200 nucleotides, from 5 nucleotides to 100 nucleotides, from 5 nucleotides to 50 nucleotides, from 5 nucleotides to 40 nucleotides, from 5 nucleotides to 30 nucleotides, from 5 nucleotides to 20 nucleotides, including endpoints and all integers therebetween. In another embodiment, the oligonucleotide may range from about 10 nucleotides to about 200 nucleotides, from 10 nucleotides to 150 nucleotides, from 10 nucleotides to 125 nucleotides, from 20 nucleotides to 100 nucleotides, from 25 nucleotides to 75 nucleotides, from 30 nucleotides to 60 nucleotides, including endpoints and all integers therebetween. In one embodiment, the oligonucleotide may range from 40 nucleotides to 70 nucleotides, including endpoints. In one embodiment, the oligonucleotide may range from 30 nucleotides to 80 nucleotides, including endpoints. In one embodiment, the oligonucleotide may range from 50 nucleotides to 75 nucleotides, including endpoints. In one embodiment, the oligonucleotide may range from 35 nucleotides to 85 nucleotides, including endpoints. In one embodiment, the oligonucleotide is 54 nucleotides. In another embodiment, the oligonucleotide is 50 nucleotides. In one embodiment, the oligo has 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides.

In another embodiment, the oligo has a sequence found in the table below.

| Transposase | Oligo Sequence | SEQ ID NO: |
|---|---|---|
| TNY- | CGA UCG AUA AAA ACC CGC | 24 |
| BLOCKER | CUA UAU AGC GCU AUA UAG | |
| | GCG GGU UUU UAU CGA UCG | |
| TN5- | UAU AUU UAU UUA AAC AGU | 25 |
| BLOCKER | UUU AAA CGT UUA AAA CUG | |
| | UUU AAA UAA AUA UA | |

In one embodiment, the oligo has the sequence of SEQ ID NO 24. In another embodiment, the oligo has the sequence of SEQ ID NO: 24, with 1, 2, 3, 4, 5, 6, 7, 8, 9, or 10 substitutions. In another embodiment, a Tn blocker is provided where the U residues of SEQ ID NO: 24 are replaced with Thymine residues. In one embodiment, the oligo has the sequence of SEQ ID NO 25. In another embodiment, the oligo has the sequence of SEQ ID NO: 25, with 1, 2, 3, 4, 5, 6, 7, 8, 9, or 10 substitutions. In another embodiment, a Tn blocker 25 is provided where the U residues of SEQ ID NO: 25 are replaced with Thymine residues.

Tn5 and TnY transposases preferentially bind certain DNA sequences. The consensus target site for Tn5 has been reported as A-GNTYWRANC-T, where N=all 4 bases, Y=T or C, W=A or T, and R=A or G. In certain embodiments, the blocking nucleotide comprises a sequence that shares 100% complementarity with the to the DNA sequence to which the transposase preferentially binds, e.g., A-GNTYWRANC-T. In other embodiments, the blocking nucleotide contains 1, 2, 3, 4, 5, 6, 7, 8, 9, or 10 mismatches as compared to the DNA sequence to which the transposase preferentially binds.

Methods of generating oligonucleotides are known in the art, as well as being commercially available. The commonly used phosphoramidite synthesis chemistry consists of a four-step chain elongation cycle that adds one base per cycle onto a growing oligonucleotide chain attached to a solid support matrix. See, e.g., Hughes, Randall A, and Andrew D Ellington. “Synthetic DNA Synthesis and Assembly: Putting the Synthetic in Synthetic Biology.” Cold Spring Harbor perspectives in biology vol. 9,1 a023812. 3 January 2017, doi: 10.1101/cshperspect.a023812, which is incorporated herein by reference.

### II. Compositions

Provided herein, in one aspect, are compositions which contain one or more of the components described above, optionally in addition to other features, molecules or components. In one embodiment, a composition is provided which allows for interaction mapping of molecules found in a biological sample. The selection of the components of the composition will depend upon the identity of the partner molecule sought, the methodology being employed and interactions being elucidated. The method used may dictate the selection and compositions of the various components described above which make up the composition. Thus, the following description of compositions is not exhaustive, and one of skill in the art can design many different compositions based on the teachings provided herein. The composition may also contain the constructs in a suitable buffer, diluent, carrier, or excipient. The elements of each composition will depend upon the assay format in which it will be employed. Several embodiments of compositions are described below, but are not to limit the compositions encompassed herein, which are intended to extend to compositions comprising any component(s) herein described.

In one embodiment, a composition is provided which comprises a reagent. The reagent includes fusion protein as described herein which includes a nanobody and a transposase.

In another embodiment, a composition comprising a plurality of reagents as described herein is provided. Each reagent comprises a different nanobody conjugated to a transposase, wherein each nanobody is capable of recognizing and binding a different partner biological molecule. The plurality may comprise any number of different nanobody fusion proteins as is needed to obtain the required information from the assay. In certain embodiments, the composition is contains 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nanobody fusion constructs. In certain embodiments, the composition contains at least 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more different nanobody constructs.

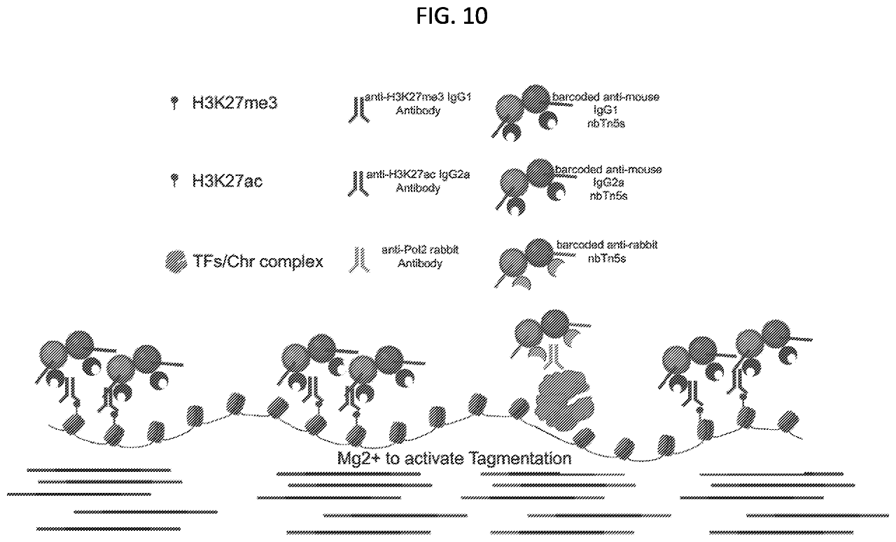

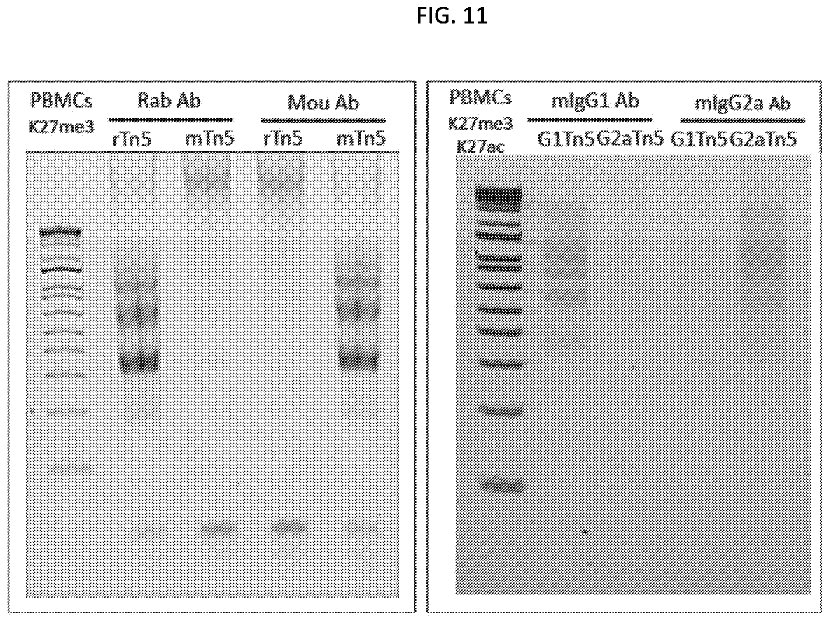

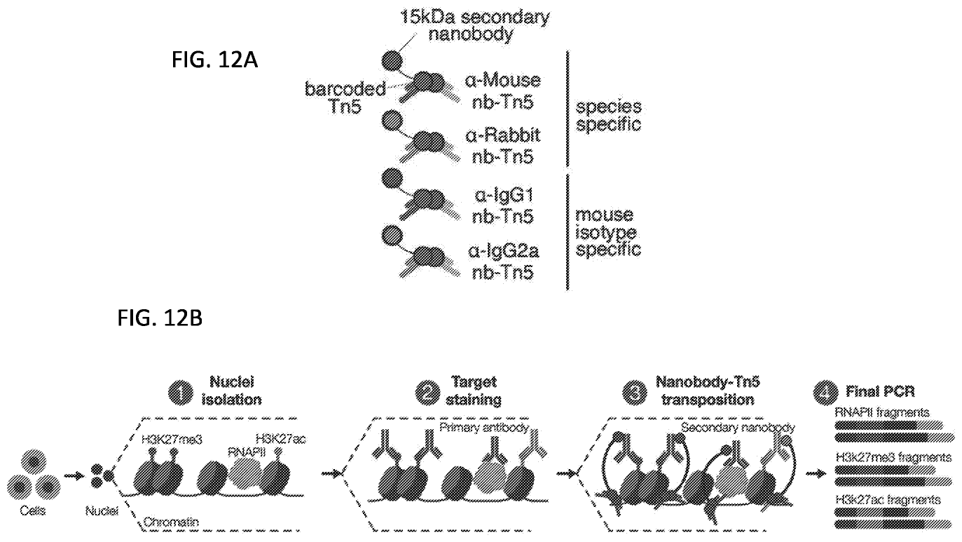

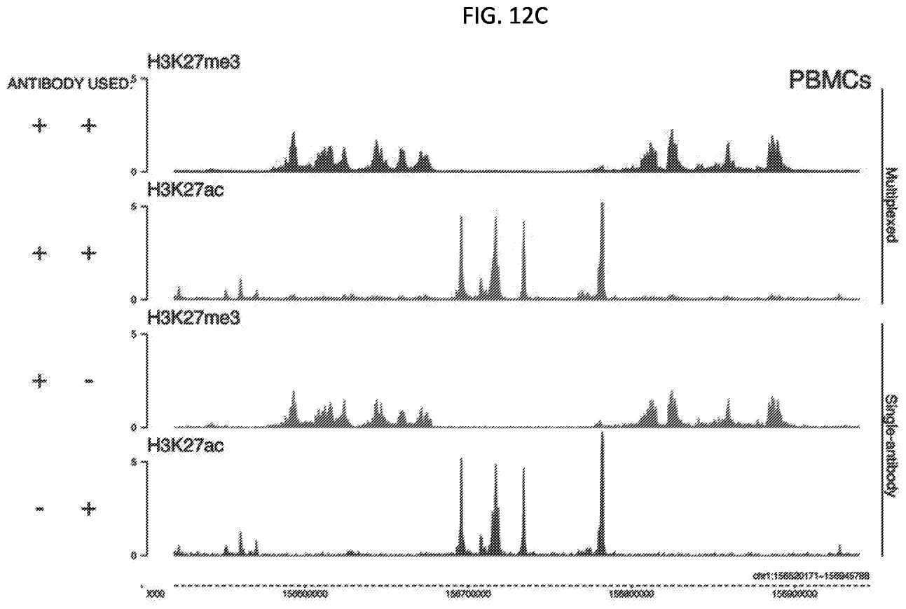

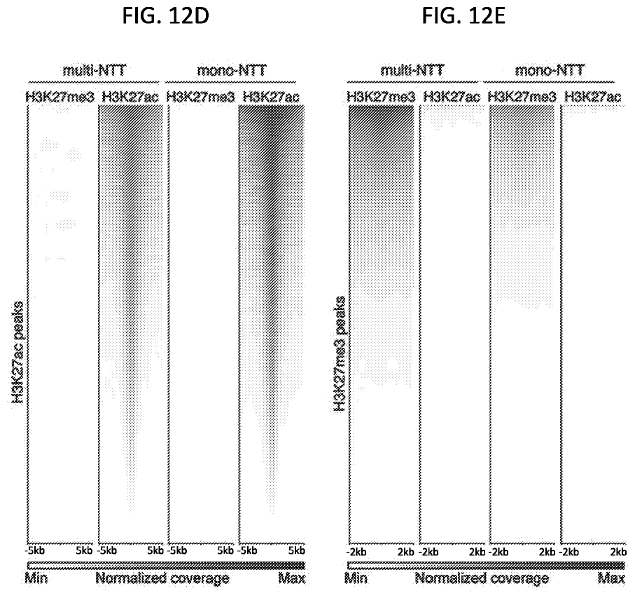

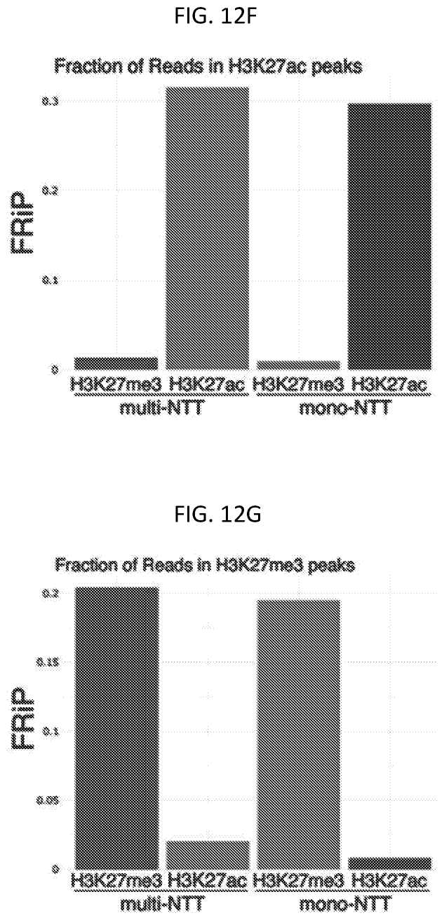

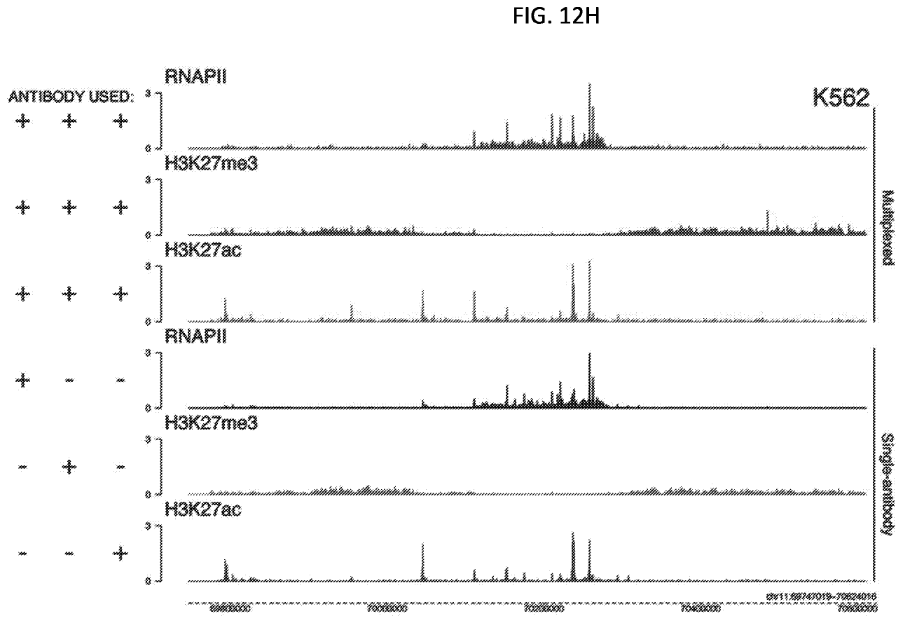

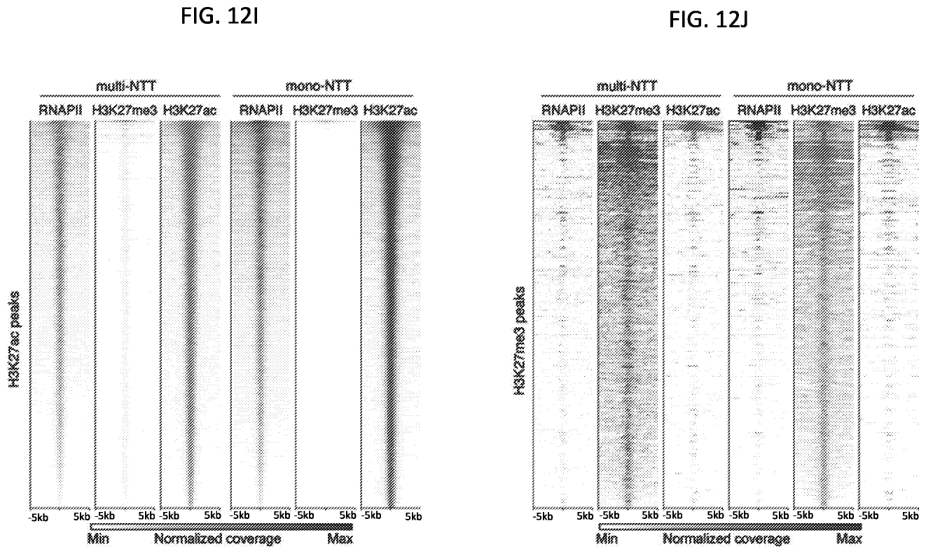

In another embodiment, a composition comprises a nanobody-transposase fusion protein as described herein that has been incubated with, and thus, “loaded” with MEDS adapters. See, FIG. 10 and FIG. 12A. In certain embodiments, the adapter-loaded nanobody-transposase fusion protein exists as a dimer. In certain embodiments, the nb-Tn fusion is loaded with MEDS-A and MEDS-B.

In another embodiment, the adapter-loaded nanobody-transposase fusion protein composition further comprises a blocking oligo that prevents tagmentation from occurring. In another embodiment, a composition is provided which includes the adapter-loaded nanobody-transposase fusion protein composition, optionally in combination with a blocking oligo, bound to chromatin by a protein-specific primary antibody, to which the nanobody binds.

In yet another embodiment, a composition is provided which includes the adapter-loaded nanobody-transposase fusion protein composition, optionally in combination with a blocking oligo, bound to chromatin by a protein-specific primary antibody, to which the nanobody binds, wherein the chromatin-bound composition is bound to a substrate, e.g., a gel bead or glass slide.

In another embodiment, a composition is provided which includes the adapter-loaded nanobody-transposase fusion protein composition, optionally in combination with a blocking oligo, bound to chromatin by the nanobody.

In yet another embodiment, a composition is provided which includes the adapter-loaded nanobody-transposase fusion protein composition, optionally in combination with a blocking oligo, bound to chromatin by the nanobody, wherein the chromatin-bound composition is bound to a substrate, e.g., a gel bead or glass slide.

Kits containing the compositions are also provided. Such kits will contain one or more of the following: fusion proteins as described herein, Tn blockers, MEDS adapters, substrates, substrate oligonucleotides, one or more preservatives, stabilizers, or buffers, and such suitable assay and amplification reagents depending upon the amplification and analysis methods and protocols with which the composition will be used. Still other components in a kit include optional reagents for cleavage of the linker, fixative, ligase, wash buffer, detectable labels, immobilization substrates, optional substrates for enzymatic labels, as well as other laboratory items.

### III. Methods

The components, compositions and kits described above can be used in diverse environments for detection of different targets, by employing any number of assays and methods for detection of targets in general. In certain aspects, the methods and compositions described herein rely on the nanobody-transposase fusion proteins described herein, which replace standard reagents, such as protein A-Tn5 fusions in methods that rely on targeted transposition events, such as CUT & Tag, ACT-seq, ChIL-seq, and TAM-ChIP. Furthermore, in other aspects, the nb-Tn fusions, as well as standard reagents, are useful in the low salt CUT & Tag strategy described herein, which utilizes the Tn blocker described herein. In addition, the reagents described herein, as well as standard reagents, are useful in the spatial resolved targeting strategy described herein. Table 6 provides a listing of multiple embodiments of methods that utilize the technologies described herein. These embodiments are not meant to be exhaustive of the uses of the compositions and methods described herein. A sample protocol for each embodiment is provided in the Examples below (as shown in Table 6). Such protocols may be adapted as needed by the person of skill in the art. Low Salt CUT & Tag (See Example 1)

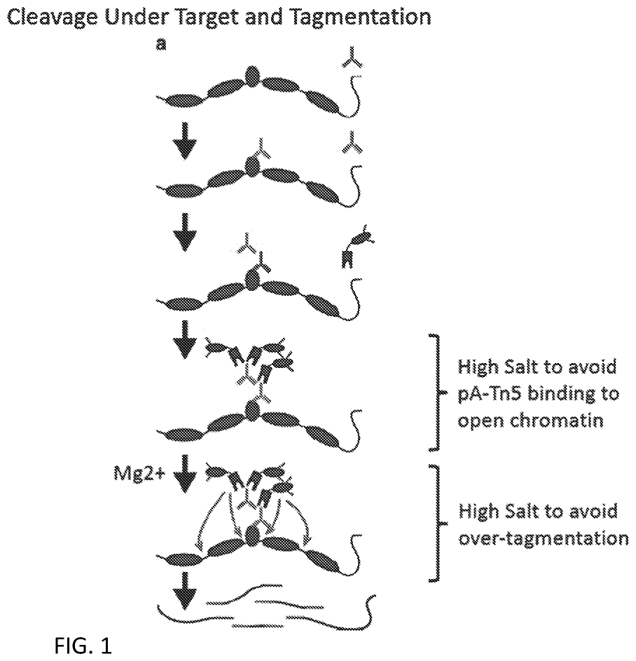

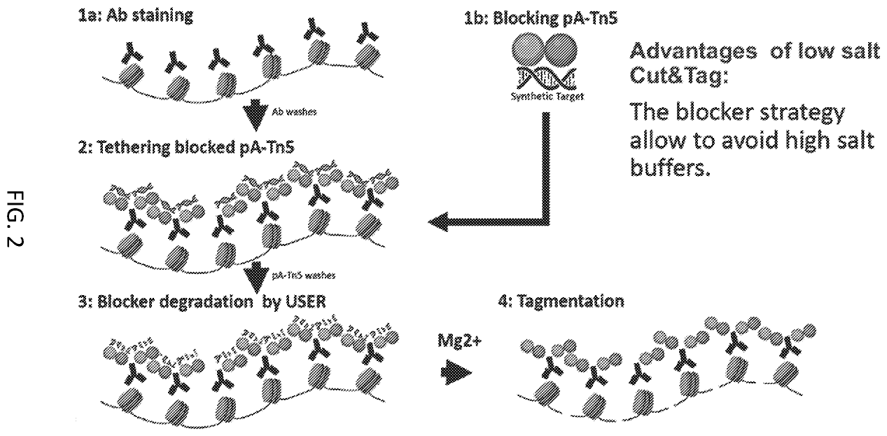

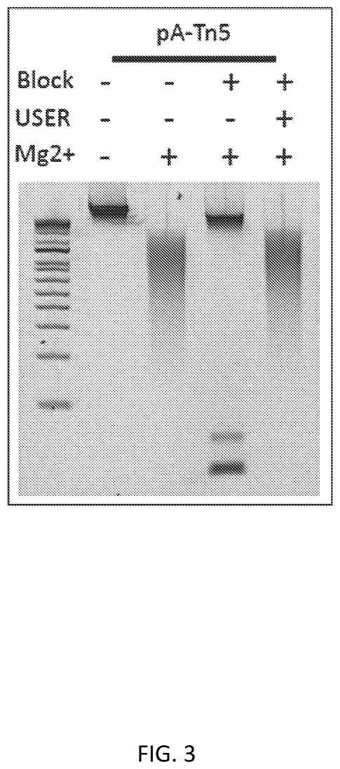

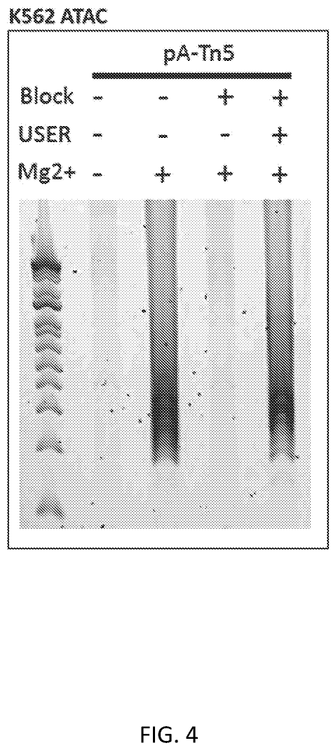

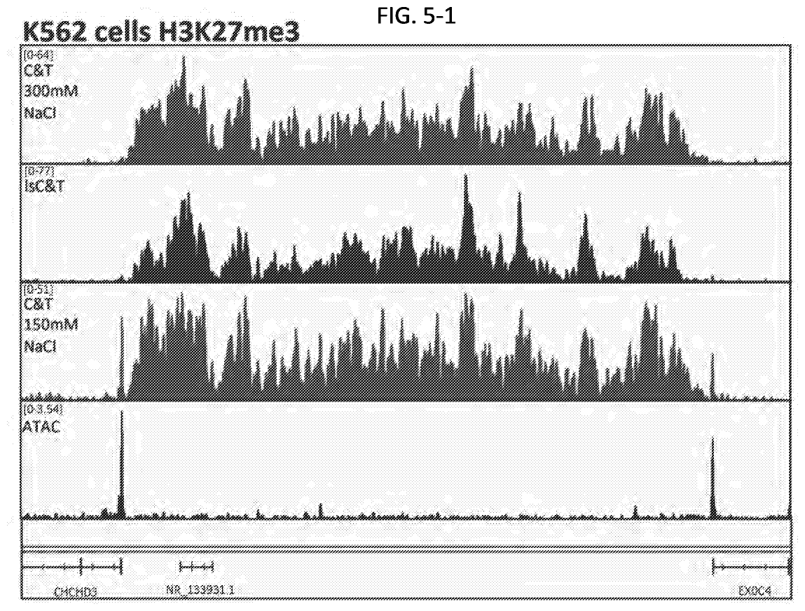

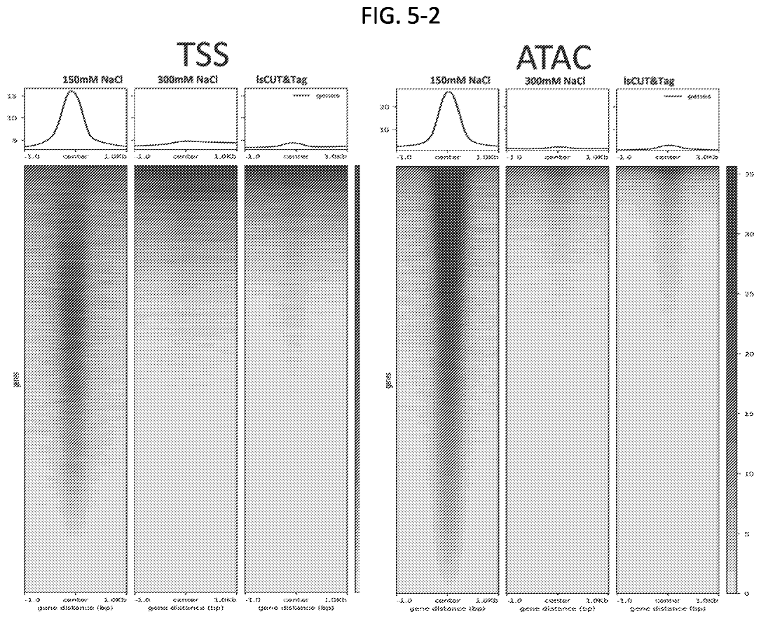

Provided herein, in one aspect, is an efficient synthetic target blocking strategy for CUT & Tag applications. This method is referred to herein, at times, as low salt CUT & Tag, (or lsCUT & Tag, IsC & T), as the high salt washes required for standard CUT & Tag protocols are not required. The low salt CUT & Tag strategy overcomes weaknesses of standard CUT & Tag (FIG. 1), which include the requirement for a second antibody step and low intact cell recovery for single cell applications. Further, while CUT & Tag generates robust data for histone PTMs, its compatibility with other chromatin interactors has not been shown. It is believed that they will be displaced during the high salt washes required for the standard procedure. Kaya-Okur et al. Nat Protoc. 2020 October; 15(10):3264-3283, which is incorporated herein by reference, provides a standard CUT & Tag which protocol, which may be amended to incorporate the low salt strategy described herein. An embodiment of the IsCUT & Tag strategy is shown in FIG. 2 and described in Example 1.

To overcome the need for non-physiologically high salt concentrations in CUT & Tag, and thereby enabling more faithful preservation of native DNA-protein interactions and reducing disruptions to tissue morphology, the IsCUT & Tag strategy employs methods and compositions for reversibly blocking the interaction of transposase with genomic DNA, i.e., a Tn blocker. As described hereinabove, the Tn blocker is an oligonucleotide duplex that is designed to be specific to the DNA binding preference of the transposon to be blocked. Importantly, in certain embodiments, the T residues in the duplex are replaced with U residues. Incubation of the transposon with the blocking reagent results in complexes that are unable to bind DNA, avoiding the unspecific interaction of the transposon with open chromatin regions of the genome. However, upon addition of a reagent that displaces the Tn blocker, the transposase is freed to perform tagmentation. In some embodiments, the reagent is e.g., a USER enzyme cocktail (a commercially available mixture of enzymes that specifically cleaves DNA containing uracils) and the blocking duplex is cleaved at every uracil residue, destroying it and freeing the transposase to perform tagmentation.

In another embodiment, the Tn blocker oligo is displaced using a wash buffer having at least about 50 mM NaCl. In certain embodiments, a wash is performed using a buffer having about 50 mM to about 150 mM NaCl (including endpoints). In this embodiment, it is not necessary to use a Tn blocker in which the T residues have been replaced with U residues.

Provided herein are methods of utilizing the Tn blockers and specific buffers for performing CUT & Tag with low salt concentrations. These blocking reagents are useful with standard CUT & Tag reagents such as pA-Tn5, as well as the novel nanobody-transposase fusion proteins described herein. For convenience, reference in this section to “pA-Tn5” will be used, but should not be read to limit the invention to use with only pA-Tn5 compositions. In one embodiment, the method includes one or more of the following steps:

Referring to FIG. 2: 1a) Optionally fixed or permeabilized cells are stained with primary, and optionally, secondary, antibody directed to the target of interest. 1b) Tn blocking oligo is incubated with pA-Tn5 loaded with MEDS adapters. The MEDS adapters comprise the required sequences necessary for the further processing steps of the sample, as may be determined by the person of skill. For example, in one embodiment, MEDS comprise a target barcode, an optional UMI, a sequence adapter, which may be the same sequence as a PCR handle, or an optional additional PCR handle. In another embodiment, the target barcode is optional.
2) Stained cells are washed using a no salt or low salt buffer to remove salt, and incubated with Tn-blocked-pA-Tn5 complexes to tether the same to the stained chromatin (FIG. 2, step 2).

Low salt wash buffers are known in the art. A buffer that includes 10 mM TAPS, 0.5 mM Spermidine, 1 or 2% BSA is used as an example, but other low salt wash buffers may be employed by the person of skill in the art. For example, as shown in Example 1, the chromatin is washed once in Dig-150 wash buffer, and 3 times in TAPS-BSA-Spermidine to desalt.

In certain embodiments, the Tn blocking oligo is incubated with pA-Tn5 for from about 5 minutes to about 24 hours, inclusive of end points. In certain embodiments, incubation is about 10 minutes, 15 minutes, 20 minutes, 25 minutes, 30 minutes, 35 minutes, 40 minutes, 45 minutes, 50 minutes, 55 minutes, 60 minutes. In certain embodiments, incubation is about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. Incubation may be performed at room temperature, 37° C., 55° C., or any other temperature deemed acceptable by the person of skill.

After the antibody-stained chromatin is contacted with the Tn-blocked-transposase complex (FIG. 2, step 2), the chromatin is washed with in a buffer lacking NaCl to remove excess (unbound) Tn-blocked-transposase complex. For example, as shown in Example 1, the chromatin is washed 6 times in TAPS-BSA-Spermidine to remove excess Tn-blocked-transposase complex.
3) The antibody-stained chromatin, which now has Tn-blocked-transposase tethered thereto is then contacted with a reagent that displaces the Tn blocker oligo. In certain embodiments, the reagent is a USER enzyme cocktail. USER (Uracil-Specific Excision Reagent) Enzyme generates a single nucleotide gap at the location of a uracil. USER Enzyme is a mixture of Uracil DNA glycosylase (UDG) and the DNA glycosylase-lyase Endonuclease VIII. UDG catalyses the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released. USER enzyme is available commercially from e.g., New England Biolabs (Cat No. M5505S).

In certain embodiments, the chromatin-Tn blocking oligo composition is incubated with USER enzyme for from about 5 minutes to about 4 hours, inclusive of end points. In certain embodiments, incubation is about 10 minutes, 15 minutes, 20 minutes, 25 minutes, 30 minutes, 35 minutes, 40) minutes, 45 minutes, 50) minutes, 55 minutes, 60) minutes. In certain embodiments, incubation is about 1 hour, 2 hours, 3 hours, or 4 hours. Incubation may be performed at room temperature, 37° C., 55° C., or any other temperature deemed acceptable by the person of skill. In certain embodiments, the incubation is performed at 37° C.

In another embodiment, the Tn blocker oligo is displaced using a wash buffer having at least about 50 mM NaCl. In certain embodiments, a wash is performed using a buffer having about 50 mM to about 150 mM NaCl (including endpoints). Multiple washes using a buffer having about 50 mM to about 150 mM NaCl may be performed. In this embodiment, it is not necessary to use a Tn blocker in which the T residues have been replaced with U residues.

After the Tn blocker oligo has been displaced or degraded, tagmentation is then activated by addition of magnesium or cobalt. The tagmentation activated by using cobalt is a key step to increase the specificity of the library. The remainder of the protocol then proceeds according to established procedures that may be adapted if needed by the person of skill in the art. For example, in certain embodiments, the DNA is extracted, and PCR amplification is performed. The library is prepared and sequencing is performed using established procedures.

### Single Cell Low Salt CUT & Tag (See Example 2)

In certain embodiments, a method of performing single cell CUT & Tag is provided. The method employs the Tn blocker and low salt system as described above, and further utilizes a substrate to which the cell, nuclei, chromatin, or DNA is bound. The substrate may be selected from those known in the art, including those described herein such as a bead, plate, chip, or chamber. In brief, in one embodiment, optionally fixed or permeabilized cells or nuclei are incubated with a primary antibody followed, optionally, by incubation with a secondary antibody to increase the number of IgG molecules at each epitope bound by the primary antibody. During secondary staining (if applicable, not necessary with nb-Tn fusion proteins), Tn blocking oligo is annealed, and incubated with pA-Tn5 loaded with MEDS adapters. The cells or nuclei are washed to remove salt and incubated with Tn-blocked-pA-Tn5 complexes. Tn5 is then activated by addition of magnesium or cobalt.

In another embodiment, nuclei are fixed. Nuclei are incubated with a primary antibody, followed, optionally, by incubation with a secondary antibody to increase the number of IgG molecules at each epitope bound by the primary antibody. During secondary staining (if applicable, not necessary with nb-Tn fusion proteins), Tn blocking oligo is annealed, and incubated with pA-Tn5 loaded with MEDS adapters. The nuclei are washed to remove salt and incubated with Tn-blocked-pT-Tn5 complexes. Tn5 is then activated by addition of magnesium or cobalt.

In one embodiment, the method includes one or more of the following steps: Referring to FIG. 2: 1a) Optionally fixed or permeabilized cells are stained with primary, and optionally, secondary, antibody directed to the target of interest. In certain embodiments, the sample is native nuclei, fixed nuclei, fixed permeabilized nuclei, permeabilized cells, or fixed permeabilized cells. 1b) Tn blocking oligo is incubated with pA-Tn5 loaded with MEDS adapters. The MEDS adapters comprise the required sequences necessary for the further processing steps of the sample, as may be determined by the person of skill. For example, in 0) one embodiment, MEDS comprise an optional target barcode, an optional UMI, a sequence adapter, which may be the same sequence as a PCR handle, or an optional additional PCR handle.
2) Stained cells are washed using a no salt or low salt buffer to remove salt and incubated with Tn-blocked-pA-Tn5 complexes to tether the same to the stained chromatin (FIG. 2, step 2).

Low salt wash buffers are known in the art. A buffer that includes 10 mM TAPS, 0.5 mM Spermidine, 1 or 2% BSA is used as an example, but other low salt wash buffers may be employed by the person of skill in the art. For example, as shown in Example 1, the chromatin is washed once in Dig-150 wash buffer, and 3 times in TAPS-BSA-Spermidine to desalt.

In certain embodiments, the Tn blocking oligo is incubated with pA-Tn5 for from about 5 minutes to about 24 hours, inclusive of end points. In certain embodiments, incubation is about 10 minutes, 15 minutes, 20 minutes, 25 minutes, 30 minutes, 35 minutes, 40 minutes, 45 minutes, 50 minutes, 55 minutes, 60 minutes. In certain embodiments, incubation is about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. Incubation may be performed at room temperature, 37° C., 55° C., or any other temperature deemed acceptable by the person of skill.

After the antibody-stained chromatin is contacted with the Tn-blocked-transposase complex (FIG. 2, step 2), the chromatin is washed with in a buffer lacking NaCl to remove excess (unbound) Tn-blocked-transposase complex. For example, as shown in Example 1, the chromatin is washed 6 times in TAPS-BSA-Spermidine to remove excess Tn-blocked-transposase complex.
3) The antibody-stained chromatin, which now has Tn-blocked-transposase tethered thereto is then contacted with a reagent that displaces the Tn blocker oligo. In certain embodiments, the reagent is a USER enzyme cocktail. USER (Uracil-Specific Excision Reagent) Enzyme generates a single nucleotide gap at the location of a uracil. USER Enzyme is a mixture of Uracil DNA glycosylase (UDG) and the DNA glycosylase-lyase Endonuclease VIII. UDG catalyses the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released. USER enzyme is available commercially from e.g., New England Biolabs (Cat No. M5505S).

In certain embodiments, the chromatin-Tn blocking oligo composition is incubated with USER enzyme for from about 5 minutes to about 4 hours, inclusive of end points. In certain embodiments, incubation is about 10 minutes, 15 minutes, 20 minutes, 25 minutes, 30) minutes, 35 minutes, 40 minutes, 45 minutes, 50 minutes, 55 minutes, 60 minutes. In certain embodiments, incubation is about 1 hour, 2 hours, 3 hours, or 4 hours. Incubation may be performed at room temperature, 37° C., 55° C., or any other temperature deemed acceptable by the person of skill. In certain embodiments, the incubation is performed at 37° C.

In another embodiment, the Tn blocker oligo is displaced using a wash buffer having at least about 50 mM NaCl. In certain embodiments, a wash is performed using a buffer having about 50 mM to about 150 mM NaCl (including endpoints). Multiple washes using a buffer having about 50 mM to about 150 mM NaCl may be performed. In this embodiment, it is not necessary to use a Tn blocker in which the T residues have been replaced with U residues.

After the Tn blocker oligo has been displaced or degraded, tagmentation is then activated by addition of magnesium or cobalt. The cells are then further processed using a commercial reagent-Chromium Next GEM Single Cell ATAC Library & Gel Bead Kit v1.1, 10× Genomics. Other suitable reagents are known in the art: Chromium Single Cell ATAC Library & Gel Bead Kit, 10× Genomics.

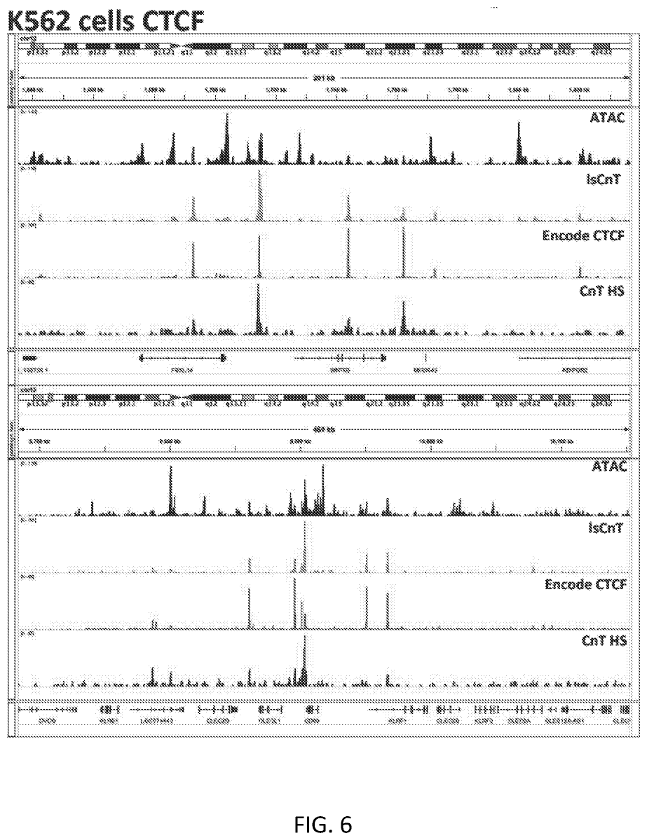

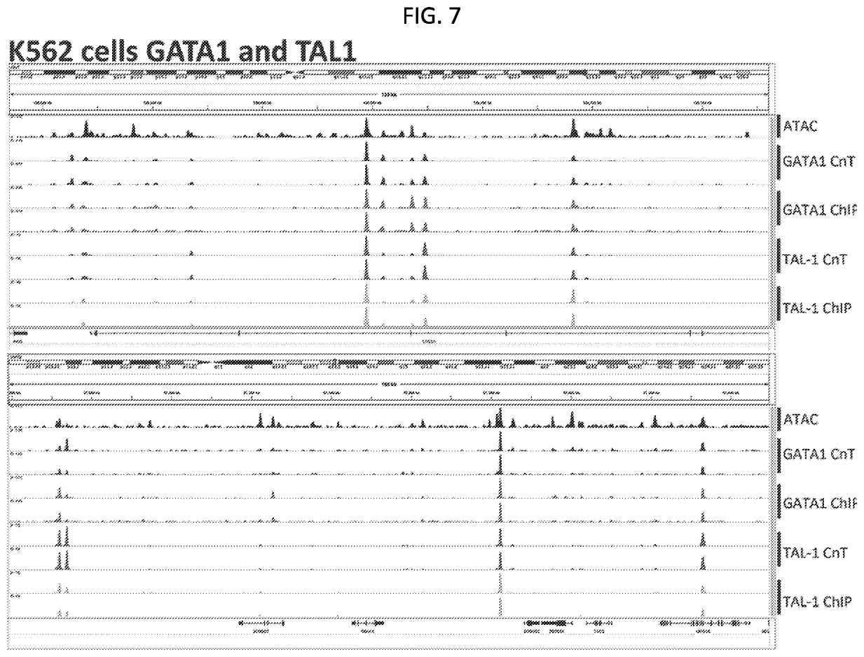

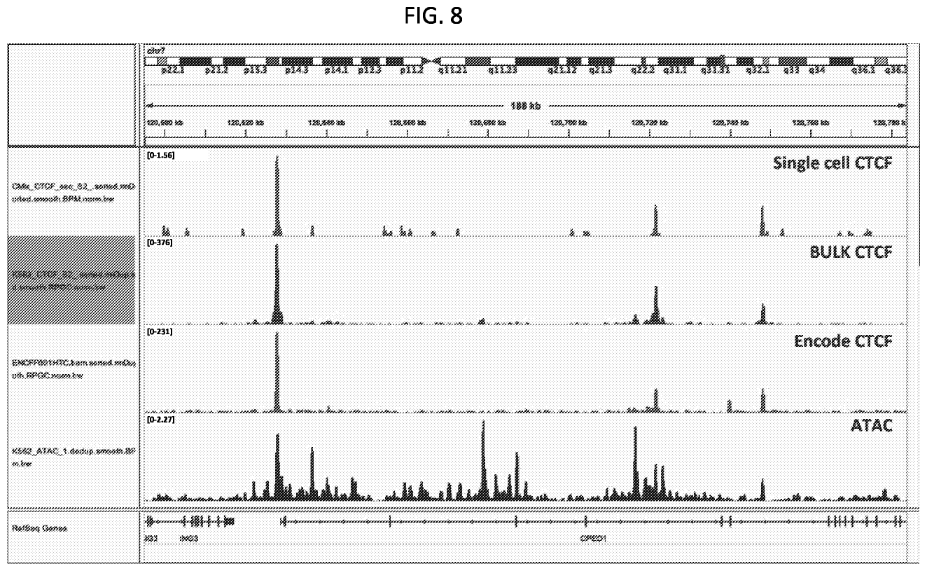

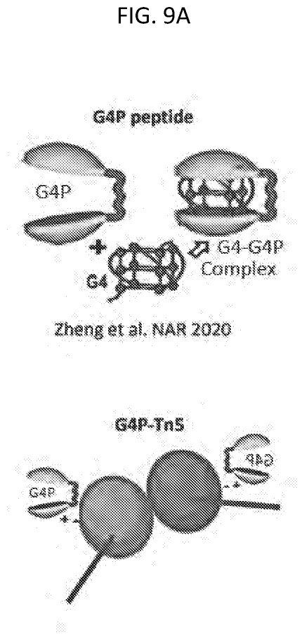

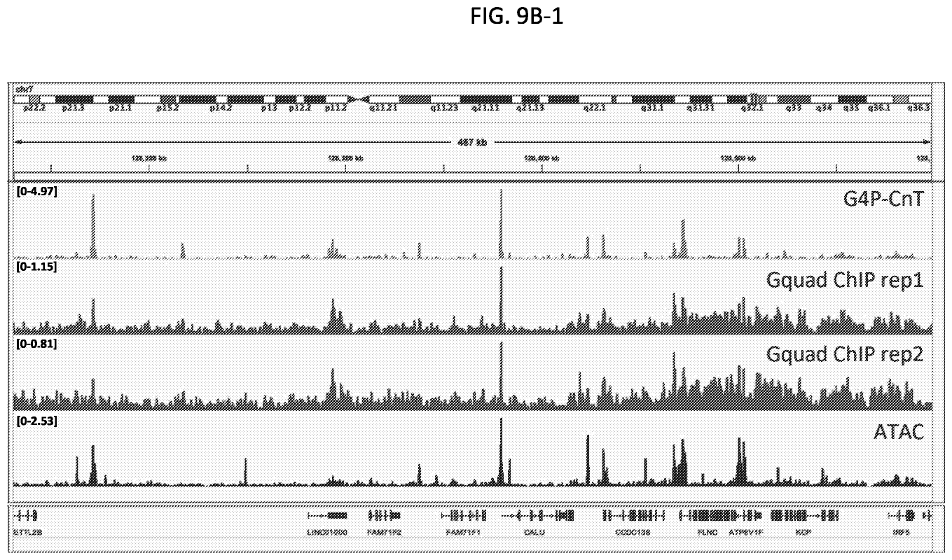

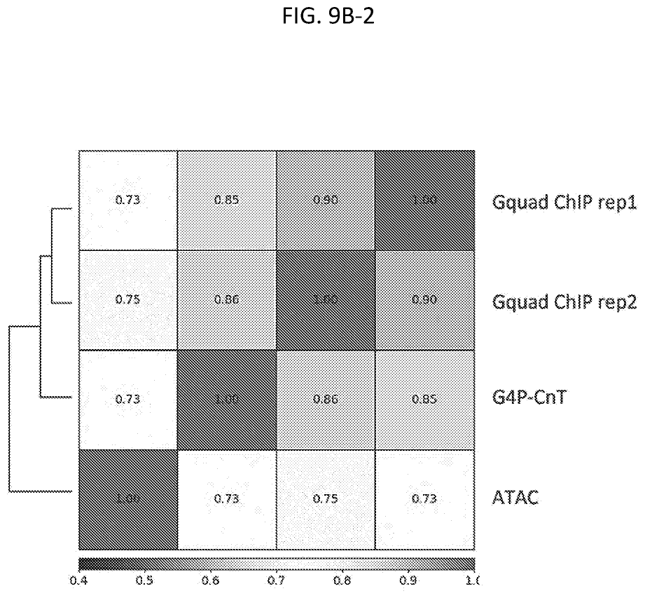

As described herein, the inventors have demonstrated that the low salt CUT & Tag strategy provides data as rigorous as the standard high salt version, but also allows for mapping of proteins that would be displaced under high salt conditions (FIG. 6) and lower affinity transcription factors (FIG. 7). In addition, the low salt CUT & Tag strategy is effective for single-cell applications, and using antibody-free CUT & Tag (using G4P as the targeting ligand).

### Nanobody-Tethered Tn5 (NTT-Seq) (See Examples 3 and 4)

To overcome limitations in sensitivity, specificity, and the number of protein targets that can be simultaneously interrogated in CUT & Tag, provided herein is a method and composition that replaces pA-Tn5 with Tn5 fused at the N terminus to a nanobody (nb-Tn5). This method is sometimes referred to as Nanobody-tethered Tn5 (NTT-seq) and is used for multiplexed single cell epigenetic profiling.

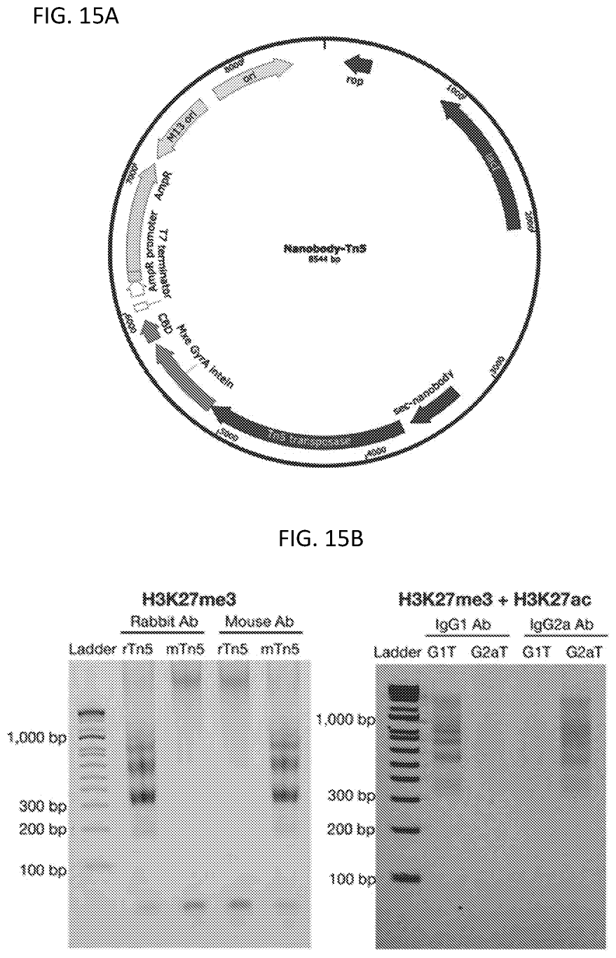

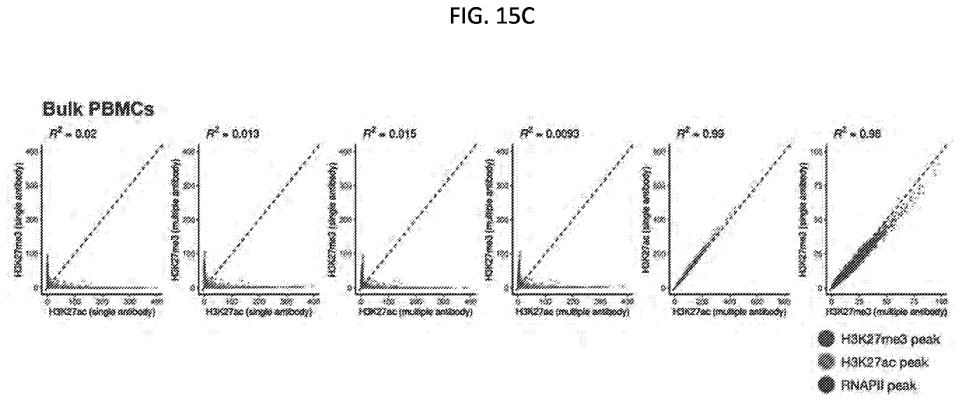

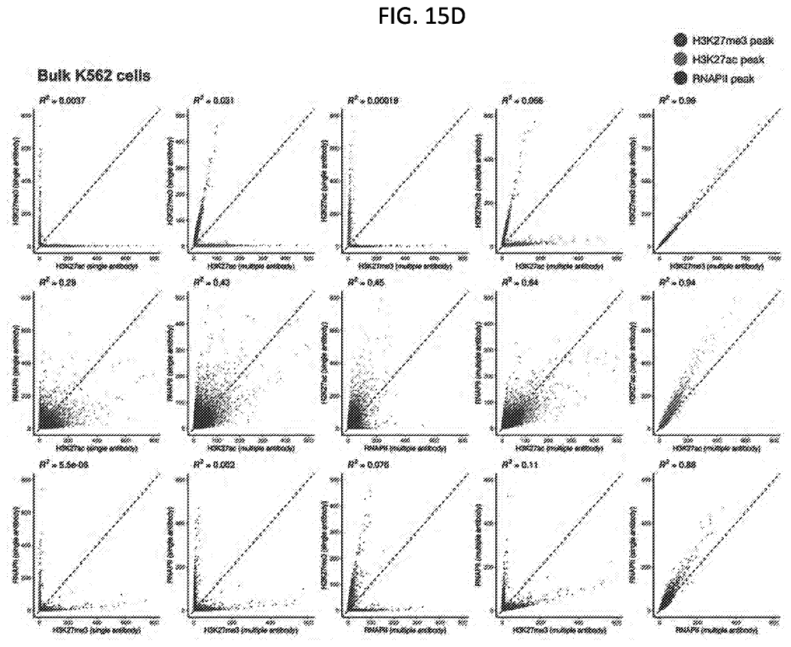

Nanobodies are very short single variable domain antibodies. Like antibodies, nanobodies bind specific epitopes with high affinity, but are only ˜12-15 kDa in size. A map of a plasmid harboring the sequences encoding nbTn5 fusions, as described herein, is provided in FIG. 15A. Plasmids encoding the nbTN5 fusions are used to transform E. Coli, which are then used to express the fusion protein. The resulting nbTn5 fusion is suitable for use in CUT & Tag experiments, as known in the art, including the low salt CUT & Tag experiments discussed and exemplified herein. Multiple nbTn5 fusions having affinity for distinct target epitopes can be loaded with mosaic end DNA sequences (MEDS) that incorporate barcode sequences corresponding to the target epitope of the nbTn5 fusion being loaded. Such target barcoded transposomes can be used together in the same CUT & Tag experiment, enabling multiplexed interrogation of DNA associated epitopes such as transcription factors bound to DNA, post-translational histone modifications, or transcribing RNA polymerase. In certain embodiments, 2, 3, 4, 5, 6 7, 8, 9, 10 or more nb-Tn fusions are utilized.

A schematic for NTT-seq is shown in FIG. 10. As can be seen, multiple targets can be interrogated in a single reaction, using antibodies and nb-Tn5 fusions that are each specific to a different target. A nanobody directed to any suitable target, as further discussed hereinabove, may be employed. Methods of performing CUT & Tag are known in the art. See, e.g., Kaya-Okur et al. Nat Protoc. 2020 October; 15(10):3264-3283, which is incorporated herein by reference. The nb-Tn fusions can be used in place of the pA-Tn fusions in the published CUT & Tag protocol. Additionally, unlike with the standard protocols, multiple nbTn5 fusions having affinity for distinct target epitopes may be pooled and used in the procedure, and stained with antibodies specific for each nanobody.

Fusion proteins comprising nanobodies and Tn5 to nanobodies instead of protein A, provide a substantial improvement of the protocol resulting in a cleaner and more specific signal for the target of interest and the possibility to multiplex different targets at the same time by using species-specific Tn5 fusions.

The fusion proteins provide significant advantages in any method that relies on a targeted transposition event. E.g., CUT & Tag, ACT-seq (Carter et al. Nat Commun. 2019 Aug. 20; 10(1):3747), ChIL-seq (Harada et al. Nat Cell Biol. 2019 February; 21(2):287-296), and TAM-ChIP (U.S. Pat. Nos. 9,938,524 and 10,689,643; EP Pat. Nos. 2783001 and 2999784). All of the aforementioned documents are incorporated herein by reference. Using Tn-blocker, the invention also enables execution of CUT & Tag at physiological salt concentrations, i.e., low salt CUT & Tag, thereby more faithfully capturing native DNA-protein interactions and minimizing disruptions of tissue morphology.

In one embodiment, the method includes preparation of nanobody-Tn fusion proteins. Fusion proteins can be generated according to standard protocols using methods known in the art. A sample protocol using a chitin binding domain for purification of the fusion protein is described by Mitchell & Lorsch. Methods Enzymol. 2015:559:111-25, which is incorporated herein by reference. Sequences encoding several nb-Tn fusion proteins are provided in SEQ ID NOs: 13-16. The method further includes loading the MEDS onto the nb-Tn fusion proteins.

The cells are stained with primary antibodies prior to being stained with a mixture of the nb-Tn fusion proteins. In certain embodiments, a primary antibody is provided for each target, with a nanobody-Tn fusion being provided for each target as well. In other embodiments, a primary antibody is provided for each target, and a single nanobody-Tn fusion is provided that is universal to all or a subset of the primary antibodies, i.e., where less nanobody-fusion proteins are provided than the number of primary antibodies. Tagmentation is then initiated. After tagmentation, PCR amplification and sequencing are performed according to established protocols.

By enabling capture on widely used substrates (droplet based single cell capture beads, commercial solid phase capture spatial arrays such as 10× Visium, or other substrates such as SCOPEseq or PIXELseq surfaces), the fusion proteins described herein provide flexibility in downstream processing and eliminate the need for complex bespoke microfluidic devices and associated workflows. Thus, in certain embodiments, methods of performing single cell NTT-seq are provided. The cells are stained with primary antibodies prior to being stained with a mixture of the nb-Tn fusion proteins. In certain embodiments, a primary antibody is provided for each target, with a nanobody-Tn fusion being provided for each target as well. In other embodiments, a primary antibody is provided for each target, and a single nanobody-Tn fusion is provided that is universal to all or a subset of the primary antibodies, i.e., where less nanobody-fusion proteins are provided than the number of primary antibodies. In certain embodiments, 2, 3, 4, 5, 6, 7, 8, 9, 10 or more nb-Tn fusions are utilized.

Cells or nuclei are incubated with a primary antibody. washed and incubated with nb-Tn5 fusion proteins loaded with mosaic-end adapters and washed under stringent conditions. Tn5 is activated by addition of Mg2+, whereupon integration of adapters effectively inactivates the nbTn5 transposome. The cells are then further processed using a commercial reagent-Chromium Next GEM Single Cell ATAC Library & Gel Bead Kit v1.1, 10× Genomics. Other suitable reagents are known in the art: Chromium Single Cell ATAC Library & Gel Bead Kit, 10× Genomics.

Optionally, the method is performed using the Tn blocker under low salt conditions, as described above, and in Examples 1 and 2.

### Spatially Resolved Methods

Recently, several methods for spatially resolved transcriptome profiling (SRT) have been developed. The most mature and widely used methods for SRT involve hybridization of mRNA onto DNA oligonucleotide probes that harbor spatial barcode and unique molecular identifier (UMI) sequences. Captured mRNA is then reverse transcribed (RT), with the capture probe functioning as a primer to initiate the RT reaction. The result is a cDNA library in which each cDNA molecule incorporates a spatial barcode, UMI, and mRNA derived sequence. As the spatial barcode sequence can be tied to a spatial coordinate, and the UMI encodes unique capture events, such methods are spatially resolved and quantitative. Examples of such methods are “Spatial Transcriptomics”, 10× Genomics Visium, seq-SCOPE, and STEREOseq, PIXELseq. One could conceive of using these methods to capture genomic DNA in situ. However, these methods are generally low sensitivity, reliably quantifying only relatively well-expressed mRNAs. With only 2 copies of any genomic DNA region present per cell in diploid organisms, these methods are not able to capture enough material from genomic DNA to generate accurate maps of DNA-protein interactions across the whole genome. Further, commercially available methods, such as 10× Genomics Visium, are designed to capture mRNA and rely on poly(A) based capture, thereby precluding capture transposed DNA.

To overcome the sparse sampling of spatially resolved methods such as 10× Genomics Visium, it is necessary to amplify DNA fragments resulting from tagmentation in ATACseq or CUT & Tag. The amplification step also provides the opportunity to append sequences to the tagmentation fragments that enable their capture. Amplification of tagmentation fragments can be achieved by in vitro transcription from a promoter sequence present in the MEDs. The MEDs can also incorporate a poly(T) sequence on the 3′ MEDs, thereby generating polyadenylated RNA that contains the sequence of the tagmentation fragment. These embodiments demonstrate the range of capabilities of the methods described herein, which enable spatial elucidation of genomic information and/or DNA-protein interactions, optionally in combination with spatial transcriptomics, in simultaneous experiments.

As discussed above, in certain embodiments, to enable identification of unique tagmentation events (as opposed to capture events), in certain embodiments, the MEDs used for tagmentation also contain UMIs (termed tagmentation UMIs, or tUMIs). Thus, all RNAs produced from a single tagmentation event will harbor the same tUMI. Following capture and reverse transcription, the end product cDNA will incorporate a CUT & Tag target barcode, a tUMI, the genomic DNA sequence captured during tagmentation, a poly(A) sequence, a capture UMI, a spatial or cellular barcode, and sequences enabling Illumina library preparation. These cDNA molecules can then be prepared for sequencing on an Illumina platform following standard library prep workflows. The resulting sequence data is then demultiplexed by CUT & Tag target barcode, tUMI, capture UMI, and spatial/cellular barcode. Demultiplexed genomic DNA sequences can then be mapped to a reference genome and peak calling used to identify sites of DNA-protein interaction (spatial CUT & Tag) or regions of open chromatin (spatial ATAC).

The methods described herein can be used for localized or spatial detection of DNA in a biological specimen. Thus one or more DNA molecules can be located with respect to its native position or location within a cell or tissue or other biological specimen. For example, one or more nucleic acids can be localized to a cell or group of adjacent cells, or type of cell, or to particular regions of areas within a tissue sample. The native location or position of individual DNA molecules can be determined using a method or composition of the present disclosure. The compositions and methods described herein may be used with existing protocols, reagents, and apparatus, where applicable, using the teachings provided herein, and known in the art.

### Spatially Resolved Whole Genome Sequencing (See Example 5)

Provided herein is a method for spatially profiling DNA of a biological specimen. In certain embodiments, the method includes contacting a biological sample with a solid support having attached thereto substrate oligonucleotides, wherein the oligonucleotides each includes a different spatial barcode sequence, optionally a UMI, and a universal capture sequence. The method further includes contacting the sample with a transposase loaded with MEDS that comprise a T7 RNA polymerase promoter and a capture compatible sequence complementary to the universal capture sequence on the substrate oligonucleotides. In certain embodiments, the MEDS capture compatible sequence is a poly(T) tail. In vitro transcription is performed using T7 RNA polymerase resulting in IVT-derived polyadenylated RNA. The substrate oligo incorporates a poly(T) capture sequence that binds to the poly(A) on the IVT-derived RNA. Captured IVT derived RNAs are then reverse transcribed in the presence of a fluorescently labeled nucleotide to yield a fluorescent signal wherever cDNA has been captured.

In some embodiments, this method is performed using the Tn blockers described herein.

In some embodiments the biological specimen is a tissue section. A tissue section can be contacted with a solid support, for example, by laying the tissue on the surface of the solid support. The tissue can be freshly excised from an organism or it may have been previously preserved for example by freezing, embedding in a material such as paraffin (e.g., formalin fixed paraffin embedded samples), formalin fixation, infiltration, dehydration (using e.g., methanol) or the like.

### Spatially Resolved ATAC (See Example 6)

In another embodiment, a method for spatially profiling chromatin accessibility-genome wide is provided. In certain embodiments, the method includes contacting a biological sample with a solid support having attached thereto oligonucleotide probes, wherein the oligonucleotide probes each includes a different spatial barcode sequence, optionally a UMI, and a universal capture sequence. The sample is then fixed prior to contacting the sample with a transposase-fusion protein loaded with MEDS. The transposase fusion protein may comprise the protein A-Tn fusion known in the art, or, in some embodiments, the fusion proteins comprise a nanobody-Tn fusion as described herein. The MEDS comprise a target barcode, optionally a target UMI, a T7 RNA polymerase promoter, a capture sequence complementary to the universal capture sequence on the oligonucleotide probes, and a sequence encoding a poly(A) tail to produce tagmented fragments suitable for amplification via in vitro transcription (IVT). In vitro transcription is performed using T7 RNA polymerase resulting in captured IVT-derived RNA. Captured IVT derived RNAs are then reverse transcribed in the presence of a fluorescently labeled nucleotide to yield a fluorescent signal wherever cDNA has been captured.

### Spatially Resolved CUT & Tag (See Example 7)

In yet another embodiment, a method for spatially resolved Cleavage Under Targets and Tagmentation (CUT & Tag) is provided. In certain embodiments, the method includes contacting a biological sample with a solid support having attached thereto oligonucleotide probes, wherein the oligonucleotide probes each includes a different spatial barcode sequence, optionally a UMI, and a universal capture sequence. The sample is then fixed prior to contacting the sample with a transposase-fusion protein that has been loaded with MEDS and optionally blocked with a Tn blocker as described herein. The transposase fusion protein may comprise a protein A-Tn fusion known in the art, or, in some embodiments, the fusion protein comprises a nanobody-Tn fusion as described herein. The MEDS comprise an optional target barcode, a T7 RNA polymerase promoter, a capture sequence complementary to the universal capture sequence on the oligonucleotide probes, and a sequence encoding a poly(A) tail. The sample is then subjected to the low salt CUT & Tag procedure as described herein. In brief, the fixed biological sample is stained with a primary and, optionally, secondary, antibody. The antibody-stained chromatin is then contacted with the Tn-blocked-transposase complex. After the antibody-stained chromatin is contacted with the Tn-blocked-transposase complex, the chromatin is washed with a buffer lacking NaCl to remove excess Tn-blocked-transposase complex. The antibody-stained chromatin, which now has Tn-blocked-transposase tethered thereto, is then contacted with a reagent that displaces the Tn blocker oligo. In certain embodiments, the reagent is a USER enzyme cocktail. Magnesium is then added, to produce tagmented fragments suitable for amplification via in vitro transcription (IVT). In vitro transcription is performed using T7 RNA polymerase resulting in captured IVT-derived RNA. Captured IVT derived RNAs are then reverse transcribed in the presence of a fluorescently labeled nucleotide to yield a fluorescent signal wherever cDNA has been captured.

### Spatially Resolved NTT-Seq (See Example 8)

In yet another embodiment, a method for spatially resolved NTT-seq is provided. In certain embodiments, the method includes contacting a biological sample with a solid support having attached thereto oligonucleotide probes, wherein the oligonucleotide probes each includes a different spatial barcode sequence, optionally a UMI, and a universal capture sequence. The sample is then fixed prior to contacting the sample with a plurality of nanobody-transposase-fusion proteins, each directed to a different target. Each fusion protein has been loaded with MEDS and optionally blocked with a Tn blocker as described herein.

The MEDS comprise an target barcode, a T7 RNA polymerase promoter, a capture sequence complementary to the universal capture sequence on the oligonucleotide probes, and a sequence encoding a poly(A) tail. The sample is then subjected to the low salt CUT & Tag procedure, as described herein. In brief, the fixed biological sample is stained with a primary antibody, and then with the plurality of (optionally blocked) nb-Tn fusion proteins. After the antibody-stained chromatin is contacted with the Tn-blocked-transposase complex, the sample is washed with a buffer lacking NaCl to remove excess Tn-blocked-transposase complex. The antibody-stained sample, which now has Tn-blocked-transposase tethered thereto, is then contacted with a reagent that displaces the Tn blocker oligo. In certain embodiments, the reagent is a USER enzyme cocktail. Magnesium is then added, to produce tagmented fragments suitable for amplification via in vitro transcription (IVT). In vitro transcription is performed using T7 RNA polymerase resulting in captured IVT-derived RNA. Captured IVT derived RNAs are then reverse transcribed in the presence of a fluorescently labeled nucleotide to yield a fluorescent signal wherever cDNA has been captured.

The methods described herein may also, in some embodiments include cell fixing, histology and imaging, cell permeabilizing, staining, template switching, transcript extension, single strand synthesis, gap filling, denaturing double strand nucleic acids, hybridization, PCR, and sequencing steps. These procedures are known in the art, and relevant protocols can be found, e.g., Corces et al., Nat Methods. 2017 October; 14(10):959-962; Kaya-Okur et al., Nat Commun. 2019 Apr. 29; 10(1):1930; Mimitou E P, et al. Nat Biotechnol. 2021 October; 39(10):1246-1258; Meers M P et al., Multifactorial chromatin regulatory landscapes at single cell resolution. BioRxiv 2021:2021.07.08.451691; Deng Y et al. Spatial-ATAC-seq: spatially resolved chromatin accessibility profiling of tissues at genome scale and cellular level. BioRxiv 2021:2021.06.06.447244; Fan R et al., Nature. 2022 September; 609(7926):375-383; Stahl P L et al. Science 2016:353:78-82. Cho C S et al. Cell 2021:184:3559-3572.e22; Chen A et al. Large field of view-spatially resolved transcriptomics at nanoscale resolution. Cold Spring Harbor Laboratory 2021:2021.01.17.427004. Fu X, et al. Continuous Polony Gels for Tissue Mapping with High Resolution and RNA Capture Efficiency. Cold Spring Harbor Laboratory 2021:2021.03.17.435795, each of which is incorporated herein by reference.

As used herein, the term “universal sequence” refers to a series of nucleotides that is common to two or more nucleic acid molecules even if the molecules also have regions of sequence that differ from each other. A universal sequence that is present in different members of a collection of molecules can allow capture of multiple different nucleic acids using a population of universal capture nucleic acids that are complementary to the universal sequence. Similarly, a universal sequence present in different members of a collection of molecules can allow the replication or amplification of multiple different nucleic acids using a population of universal primers that are complementary to the universal sequence. Thus, a universal capture nucleic acid or a universal primer includes a sequence that can hybridize specifically to a universal sequence. Target nucleic acid molecules may be modified to attach universal adapters, for example, at one or both ends of the different target sequences.

In some embodiments, a biological sample is utilized. As used herein, a “biological sample” refers to a naturally-occurring sample or deliberately designed or synthesized sample or library containing one or more biological molecules, such as DNA, RNA, proteins and the like. In one embodiment, a sample contains a population of cells or cell fragments, including without limitation cell membrane components, exosomes, and sub-cellular components. In one embodiment, the sample contains genomic DNA (gDNA) from a single cell or a population of cells. The cells may be a homogenous population of cells, such as isolated cells of a particular type, or a mixture of different cell types, such as from a biological fluid or tissue of a human or mammalian or other species subject. In other embodiments, the sample is derived from a single cell. In one embodiment, the sample contains chromatin.

Still other samples for use in the methods and with the compositions include, without limitation, blood samples, including serum, plasma, whole blood, and peripheral blood, saliva, urine, vaginal or cervical secretions, amniotic fluid, placental fluid, cerebrospinal fluid, or serous fluids, mucosal secretions (e.g., buccal, vaginal, or rectal). Still other samples include a blood-derived or biopsy-derived biological sample of tissue or a cell lysate (i.e., a mixture derived from tissue and/or cells). Such samples may further be diluted with saline, buffer, or a physiologically acceptable diluent. Alternatively, such samples are concentrated by conventional means. A sample is often obtained from, or derived from a specific source, subject, or patient. In some embodiments, a sample is often obtained from, derived from, or associated with a specific experiment, lot, run or repetition. Accordingly, in certain embodiments, each of a plurality of samples (e.g., samples derived from different sources, different subjects, or different runs, for example) can be identified and/or differentiated using a method or composition described herein.

As used herein, the term “biological specimen” is intended to mean one or more cell, tissue, organism, or portion thereof. A biological specimen can be obtained from any of a variety of organisms. Exemplary organisms include, but are not limited to, a mammal such as a rodent, mouse, rat, rabbit, guinea pig, ungulate, horse, sheep, pig, goat, cow, cat, dog, primate (i.e. human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish: a reptile: an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum. Target molecules can also be derived from a prokaryote such as a bacterium, Escherichia coli, Staphylococci or Mycoplasma pneumoniae; an archae: a virus such as Hepatitis C virus or human immunodeficiency virus: or a viroid. In one embodiment, the sample contains chromatin. Chromatin is a complex of gDNA and proteins (comprised largely of histones), in which the DNA strands wrap around the histones to efficiently pack the genomic DNA into the physical space of the cell nucleus. The compositions and methods described herein provide a means to determine the interactions between gDNA and proteins, which are located in close proximity in the chromatin complex, but not necessarily in the linear space of the DNA helix.

As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, poly butylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers.

As used herein, the term “poly T” or“poly A,” when used in reference to a nucleic acid sequence, is intended to mean a series of two or more thymine (T) or adenine (A) bases, respectively. A poly T or poly A can include at least about 2, 5, 8, 10, 12, 15, 18, 20 or more of the T or A bases, respectively. Alternatively or additionally, a poly T or poly A can include at most about, 30, 20, 18, 15, 12, 10, 8, 5 or 2 of the T or A bases, respectively.

The terms “a” or “an” refers to one or more. For example, “a fusion protein” is understood to represent one or more such fusion proteins. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps.

The words “consist”, “consisting”, and its variants, are to be interpreted exclusively, rather than inclusively, i.e., to exclude components or steps not specifically recited.

As used herein, the phrase “consisting essentially of” limits the scope of a described composition or method to the specified materials or steps and those that do not materially affect the basic and novel characteristics of the described or claimed method or composition. Wherever in this specification, a method or composition is described as “comprising” certain steps or features, it is also meant to encompass the same method or composition consisting essentially of those steps or features and consisting of those steps or features.

For simplicity and ease of understanding, throughout this specification, certain specific examples are provided to teach the construction, use and operation of the various elements of the compositions and methods described herein. Such specific examples are not intended to limit the scope of this description.

### EXAMPLES

Each and every patent, patent application, and publication, including websites cited throughout the specification, and sequence identified in the specification, is incorporated herein by reference. While the invention has been described with reference to particular embodiments, it will be appreciated that modifications can be made without departing from the spirit of the invention. Such modifications are intended to fall within the scope of the appended claims.

### Example 1: Low Salt CUT & Tag

Cell fixation and lysis. 2 million K562 cells were resuspended in 100 μl PBS, 3 μl 16% formaldehyde was added (0.1% final concentration) and incubated for 5 minutes at room temperature. Cells were swirled and inverted occasionally. Reaction was quenched by adding 40 μl 1.25 M glycine (to 0.125 M final concentration). Cells were spun for 5 minutes 800 g at 4° C. Supernatant was discarded and repeat wash with 1 ml 1× ice-cold PBS. Cells were spun for 5 minutes 800 g at 4° C., and supernatant discarded. The cell pellet was resuspended in 400 μl chilled lysis buffer, and mixed by pipetting, and incubated on ice for 7 mins. The reaction was split into two tubes and 1 ml chilled wash buffer was added to the lysed cells, and mix by pipetting. The cells were spun for 5 minutes 1000 g at 4° C.

Primary antibody binding. Cells were resuspended with 200 μl antibody binding buffer in small PCR tube. 1.5 μl antibody K27me3 was added to each tube. Reaction was incubated overnight at 4° C. or 1 h RT.

Secondary antibody binding (optional). (There should be around 1.5 M cells at this step, no cell clumping can be seen after overnight incubation). The next day cells were spun for 5 minutes 1300 g to remove the supernatant. The cells were resuspended in 150 μl Dig-150 buffer. 1.5 μl secondary antibody was added and incubated for 1 hour at room temperature. No wash was performed.

During secondary staining blocking oligo was annealed. 20 μl of blocking oligo (100 uM) was annealed in a thermocycler at 95° C. for 2 minutes, then 95° C. to 22° C.-0.01° C. per cycle.

### Blocking Oligo Sequences:

| | |
|---|---|
| TNY- | CGA UCG AUA AAA ACC CGC CUA UAU AGC |
| BLOCKER | GCU AUA UAG GCG GGU UUU UAU CGA UCG |
| | (SEQ ID NO: 24) |
| TN5- | UAU AUU UAU UUA AAC AGU UUU AAA CGT |
| BLOCKER | UUA AAA CUG UUU AAA UAA AUA UA |
| | (SEQ ID NO: 25) |

pA-Tn5 blocking. 2 μl of pA-Tn5 (pre-loaded with MEDS harboring target barcode, optional UMI, PCR handle/sequencing adapter (e.g., R1 primer, R2 primer)) was added to 100 μl TAPS-BSA-Spermidine, and mixed by pipetting. 3 μl annealed blocking oligo was added and incubated at RT for 45 min-1 h.

Sample desalting. User Enzyme does not work in presence of NaCl, moreover salt can unblock the pA-Tn5. So, it is necessary remove the excess of NaCl by washing secondary stained cells. Thus, cells were washed one time with 150 μl Dig-150 buffer to remove Abs and washed 3 times with TAPS-BSA-Spermidine.

pA-Tn5 binding. The cells were resuspended in TAPS-BSA-Spermidine/pA-Tn5 blocked and incubated for 1 h at room temperature with slow rotation. Then, cells were centrifuged 5 minutes at 1500×g, and washed six times with 100 μl of TAPS-BSA-Spermidine.

pA-Tn5 Unblocking. Cells were resuspended cells in TAPS-BSA-Spermidine and 3 μl of USER enzyme was added and incubated for at 37° C. for 1 hr.

Tagmentation. 10 μl of 100 mM Mg2+ (or 10 μl 200 mM Co2+) was added to the cells to initiate tagmentation. The cells were incubated at 37° C. for 1 hr in an incubator, and centrifuged at 1400 g for 5 minutes. Nothing was used to stop the tagmentation. Supernatant was removed and then pellet was resuspended with 30 μl Nuclei buffer. The cell concentration of is around 4800/μl.

Loading to 10×. 8 μl cells in nuclei buffer+7 μl ATAC Buffer B are loaded.

Steps 2-5 of the Chromium Next GEM Single Cell ATAC Protocol are then performed according to manufacturer specifications. see, found at support. 10×genomics.com/single-cell-atac/library-prep/doc/user-guide-chromium-single-cell-atac-reagent-kits-user-guide-v11-chemistry which is incorporated herein by reference.

### Buffers:

Isotonic Perm Buffer: (2 ml) 20 mM Tris-HCl pH 7.4 (40 μl 1 M) 150 mM NaCl (60 μl 5 M) 3 mM MgCl2 (6 μl 1 M) 0.1% NP-40 (20 μl 10%) 0.1% Tween-2 (20 μl 10%) 40 μl Proteinase inhibitor 1800 μl H2O
Wash buffer: (Dig-150) 1 mL 1 M HEPES pH 7.5 1.5 mL 5 M NaCl 16.7 μL 1.5 M spermidine, bring the final volume to 50 mL with dH2O, and add 1 Roche Complete Protease Inhibitor EDTA-Free tablet. Store the buffer at 4° C. for up to several months.
Antibody buffer: 8 μL 0.5 M EDTA 200 μl 10% BSA (final 1.0%) 40 μl proteinase inhibitor, 0.67 μl 1.5 M spermidine 2 mL Wash buffer and chill on ice. 300-wash buffer: 1 mL 1 M HEPES pH 7.5 3 mL 5 M NaCl 16.7 μL 1.5 M spermidine, bring the final volume to 50 mL with dH2O and add 1 Roche Complete Protease Inhibitor EDTA-Free tablet. Store at 4° C. for up to several months.
Tagmentation solution: 1 mL 300-wash buffer and 10 μL 1 M MgCl₂ (to 10 mM).
TAPS-BSA-Spermidine: 10 mM TAPS, 0.5 mM Spermidine, 1 or 2% BSA

### Example 2: Single Cell Low Salt CUT & Tag

Buffers are the same as in Example 1, unless specified. Cell fixation and lysis. 2 million K562 cells were resuspended in 100 μl PBS, 3 μl 16% formaldehyde was added (0.1% final concentration) and incubated for 5 minutes at room temperature. Cells were swirled and inverted occasionally. Reaction was quenched by adding 40 μl 1.25 M glycine (to 0.125 M final concentration). Cells were spun for 5 minutes 800 g at 4° C. Supernatant was discarded and repeat wash with 1 ml 1× ice-cold PBS. Cells were spun for 5 minutes 800 g at 4° C., and supernatant discarded. The cell pellet was resuspended in 400 μl chilled lysis buffer, and mixed by pipetting, and incubated on ice for 7 minutes. The reaction was split into two tubes and 1 ml chilled wash buffer was added to the lysed cells and mixed by pipetting. The cells were spun for 5 minutes 1000 g at 4° C.

Primary antibody binding. Cells were resuspended with 200 μl antibody binding buffer in small PCR tube. 1.5 μl antibody K27me3 was added to each tube. Reaction was incubated overnight at 4° C. or 1 h RT.

Secondary antibody binding (optional). (There should be around 1.5 M cells at this step, no cell clumping can be seen after overnight incubation). The next day cells were spun for 5 minutes at 1300 g to remove the supernatant. The cells were resuspended in 150 μl Dig-150 buffer. 1.5 μl secondary antibody was added and incubated for 1 hour at room temperature. No wash was performed.
During secondary staining blocking oligo was annealed. 20 μl of blocking oligo (100 uM) was annealed in a thermocycler at 95° C. for 2 minutes, then 95° C. to 22° C.-0.01° C. per cycle.

### Blocking Oligo Sequence:

| | |
|---|---|
| TNY- | CGA UCG AUA AAA ACC CGC CUA UAU AGC |
| BLOCKER | GCU AUA UAG GCG GGU UUU UAU CGA UCG |
| | (SEQ ID NO: 24) |
| TN5- | UAU AUU UAU UUA AAC AGU UUU AAA CGT |
| BLOCKER | UUA AAA CUG UUU AAA UAA AUA UA |
| | (SEQ ID NO: 25) |

Tn5-adapter complex formation. Anneal each of Mosaic end-adapter A (ME-A) and Mosaic end-adapter B (ME-B) oligonucleotides with Mosaic end-reverse oligonucleotides (SEQ ID NOs: 22, 23, and 26). To anneal, dilute oligonucleotides to 200 UM in annealing buffer (10 mM Tris pH8, 50 mM NaCl, 1 mM EDTA). Each pair of oligos, ME-A+ME-Reverse and ME-B+ME-Reverse, is mixed separately resulting in 100 uM annealed product. Place the tubes in a 90-95° C. hot block and leave for 3-5 minutes, then remove the hot block from the heat source allowing for slow cooling to room temperature (˜45 minutes). Mix 16 μL of 100 uM equimolar mixtures of preannealed ME-A and ME-B oligonucleotides with 100 μL of 5.5 uM protein A-Tn5 fusion protein. Incubate the mixture on a rotating platform for 1 hour at room temperature and then store at −20° C. for up to 1 year.

pA-Tn5 blocking. 2 μl of pA-Tn5 (pre-loaded with MEDS harboring target barcode, optional UMI, PCR handle/sequencing adapter/capture compatible sequence (e.g., R1 primer)) was added to 100 μl TAPS-BSA-Spermidine and mixed by pipetting. 3 μl annealed blocking oligo was added and incubated at RT for 45 min-1 h.

Sample desalting. User Enzyme does not work in the presence of NaCl, moreover salt can unblock the pA-Tn5. So, it is necessary to remove the excess of NaCl by washing secondary stained cells. Thus, cells were washed one time with 150 μl Dig-150 buffer to remove Abs and washed 3 times with TAPS-BSA-Spermidine.

pA-Tn5 binding. The cells were resuspended in TAPS-BSA-Spermidine/pA-Tn5 blocked and incubated for 1 h at room temperature with slow rotation. Then, cells were centrifuged 5 minutes at 1500×g, and washed six times with 100 μl of TAPS-BSA-Spermidine.

pA-Tn5 Unblocking. Cells were resuspended cells in TAPS-BSA-Spermidine and 3 μl of USER enzyme was added and incubated at 37° C. for 1 hr.

Tagmentation. 10 μl of 100 mM Mg2+ (or 10 μl 200 mM Co2+) was added to the cells to initiate tagmentation. The cells were incubated at 37° C. for 1 hr in an incubator and centrifuged at 1400 g for 5 minutes. Nothing was used to stop the tagmentation. Supernatant was removed and then pellet was resuspended with 30 μl Nuclei buffer. The cell concentration is around 4800/μl.

Loading to 10×. The Chromium Next GEM Single Cell ATAC Library & Gel Bead Kit v1.1, 10× Genomics was used. Mastermix was prepared: 8 μl nuclei suspension (in 1×PBS+1% BSA or 1×DNB+2% BSA), ATAC buffer B 7 μl, barcoding reagent B 56.5 μl, reducing agent B 1.5 μl, and barcoding enzyme 2 μl and chromium chip H loaded. 16-20 PCR cycles were used to perform the final library amplification according to Chromium Single Cell ATAC Library kit manual.

### Example 3: Materials and Methods

### Cell Culture

K562 cells were acquired from ATCC (nos. CCL-243). HEK293FT cells were acquired from Thermo Fisher (no. R70007). HEK293FT cells were maintained at 37° C., and 5% CO2 in D10 medium (DMEM with high glucose and stabilized L-glutamine (Caisson, no. DML23) supplemented with 10% fetal bovine serum (FBS: Thermo Fisher, no. 16000044)). K562 cells were maintained at 37° C., and 5% CO2 in R10 medium (RPMI with stabilized L-glutamine (Thermo Fisher, no. 11875119) supplemented with 10% FBS).

### Primary Cells Acquisition and Processing

Fresh mobilized peripheral blood mononuclear cells (PBMCs) used for scNTT-seq with cell surface protein measurement were isolated within 48 hours of blood collection utilizing a Ficoll (Thermo Fisher Scientific, #45-001-750) gradient according to manufacturer's recommendations and cryopreserved. Isolated mononuclear cells were thawed and stained according to standard procedures, beginning with resuspension in staining buffer (Biolegend, #420201) and incubation with Human TruStain FxC (10 minutes at 4° C.; Biolegend, #422302) to block Fc receptor-mediated binding. Cells were then stained with a CD34-PE-Vio770) antibody (20 minutes at 4° C.; Miltenyi Biotec, clone AC136, #130-113-180) and DAPI (Invitrogen, #D1306). The samples were then sorted for DAPI-negative, CD34-positive cells using a BD Influx cell sorter. Live CD34-positive and CD34-negative were mixed 1:10 and processed with NTT-seq. BMMCs and PBMCs profiled by scNTT-seq without cell surface protein measurement were purchased from AllCells. After thawing into DMEM with 10% FBS, the cells were spun down at 4° C. for 5 minutes at 400 g and washed twice with PBS with 2% BSA. After centrifugation, the cell pellet was resuspended in staining buffer (2% BSA and 0.01% Tween in PBS).

### Cloning of Nb-Tn5 Plasmid Constructs

Previously published sequences coding for secondary nanobodies (Pleiner et al., J Cell Biol. 2018 Mar. 5: 217 (3): 1143-1154) were synthesized as a gene fragment (IDT) flanked by restriction enzyme sites NcoI and EcoRI. To replace protein-A with a nanobody, 3×Flag-pA-Tn5-Fl (addgene #124601) and gene fragments were digested with NcoI and EcoRI 1 h at 37° C., ligated overnight at 16° C., and subsequently transformed into competent cells (NEB C2992H).

### Nanobody-Tn5 Transposase Production

The pTXB1-nbTn5 vector was transformed into BL21 (DE3)-competent Escherichia coli cells (NEB, no. C2527), and nb-Tn5 was produced via intein purification with an affinity chitin-binding tag. 400 mL of Luria broth (LB) culture was grown at 37° C. to optical density (OD600)=0.6. nb-Tn5 expression was then induced with isopropyl-β-d-thiogalactopyranoside (IPTG) 0.25 mM at 22° C. 6 hours. After induction, cells were pelleted and then frozen at −80° C. overnight. Cells were then lysed by sonication in 100 mL pf HEGX (20 mM HEPES-KOH PH 7.5, 0.8 M NaCl, 1 mM EDTA, 10% glycerol, 0.2% Triton X-100) with a protease inhibitor cocktail (Roche, no. 04693132001). The lysate was pelleted at 30,000 g for 20 minutes at 4° C. The supernatant was transferred to a new tube, and 3 μL of neutralized 8.5% polyethylenimine (Sigma-Aldrich, P3143) was added dropwise to each 100 μL of bacterial extract, gently mixed and centrifuged at 30,000 g for 30 minutes at 4° C. to precipitate DNA. The supernatant was loaded on four 2 mL chitin columns (NEB, no. S6651S). Columns were washed with 10 mL of HEGX, then 1.5 mL of HEGX containing 100 mM DTT was added to the column with incubation for 48 h at 4° C. to allow cleavage of nb-Tn5 from the intein tag. nb-Tn5 was eluted directly into two 30 kDa molecular-weight cutoff (MWCO) spin columns (Millipore, no. UFC903008) by the addition of 2 mL of HEGX. Protein was dialyzed in five dialysis steps using 15 mL of 2× dialysis buffer (100 HEPES-KOH PH 7.2, 0.2 M NaCl, 0.2 mM EDTA, 2 mM DTT, 20% glycerol) and concentrated to 1 mL by centrifugation at 5,000 g. The protein concentrate was transferred to a new tube and mixed with an equal volume of 100% glycerol. nb-Tn5 aliquots were stored at −80° C.

### Transposome Assembly

We obtained barcoded Tn5 adaptors from IDT, as described by Amini et al. (Nat Genet. 2014 December; 46(12):1343-9) with 8 bp barcode sequences designed using FreeBarcodes (Proc Natl Acad Sci USA. 2018 Jul. 3; 115(27):E6217-E6226). To produce mosaic-end, double-stranded (MEDS) oligos, we annealed each barcoded T5 tagmentation oligo with the pMENT common oligo (100 μM each) as follows, in TE buffer: 95° C. for 5 minutes then cooling at 0.2° C. per second to 4° C. (bcMEDS-A). The same process was used to anneal a single T7 tagment oligo with the pMENT common oligo (MEDS-B). bcMEDS-A and MEDS-B were mixed 1:1 and 6 μL was transferred to a new tube and mixed with 10 μL of nb-Tn5 enzyme. After 1 hour at room temperature to allow for transposome assembly.

### Antibodies

Antibodies used were H3K27ac (1:50, Active Motif, 39133), H3K27ac (1:50, Active Motif, 91193), H3K27ac (1:50, AbCam, ab4729), H3K27me3 (1:50, Active Motif, 61017), Phospho-Rpb1 CTD (Ser2/Ser5) (1:50, Cell Signaling, 13546). For NTT-seq with surface markers readout on primary cells, the TotalSeq-A conjugated Human Universal Cocktail v1.0 panel was obtained from BioLegend (399907).

### NTT-Seq

We performed NTT-seq using similar methods to those described previously by Kaya-Okur et al., Nat Commun. 2019 Apr. 29; 10(1):1930, described in detail below.

### Antibody Staining

For NTT-seq with surface markers readout on primary cells, 1 million thawed PBMCs were resuspended in 200 μL staining buffer (2% BSA and 0.01% Tween in PBS) and incubated for 15 minutes with 20 μL Fc receptor block (TruStain FcX, BioLegend) on ice. Cells were then washed three times with 1 mL staining buffer and pooled together. The panel of oligo-conjugated antibodies was added to the cells to incubate for 30 minutes on ice. After staining, cells were washed three times with 1 mL staining buffer and resuspended in 100 μL staining buffer. After the final wash, cells were resuspended 200 μL PBS ready for fixation.

### Fixation and Permeabilization

For human cell lines, nuclei were extracted and resuspended in 150 μL of PBS. Then, 16% methanol-free formaldehyde (Thermo Fisher Scientific, PI28906) was added for fixation (final concentration: 0.1%) at room temperature for 3 minutes. The cross-linking reaction was stopped by addition of 12 μL 1.25 M glycine solution. Subsequently, nuclei were washed once with 150 μL antibody buffer (20 mM HEPES pH 7.6, 150 mM NaCl, 2 mM EDTA, 0.5 mM spermidine, 1% BSA, 1× protease inhibitors).

For NTT-seq on PBMCs and BMMCs, 16% methanol-free formaldehyde (Thermo Fisher Scientific, PI28906) was added for fixation (final concentration: 0.1%) at room temperature for 5 minutes. The cross-linking reaction was stopped by addition of 12 μL 1.25 M glycine solution. Subsequently, cells were washed twice with PBS. The permeabilization was performed by adding isotonic lysis buffer (20 mM Tris-HCl pH 7.4, 150 mM NaCl, 3 mM MgCl2, 0.1% NP40, 0.1% Tween-20, 1% BSA, 1× protease inhibitors) on ice for 7 minutes. Subsequently, 1 mL of cold wash buffer (20 mM HEPES pH 7.6, 150 mM NaCl, 0.5 mM spermidine, 1× protease inhibitors) was added, and cells were centrifuged at 800 g for 5 minutes at 4° C.

### Tagmentation

Nuclei or permeabilized cells were directly suspended with 150 μL antibody buffer (20 mM HEPES pH 7.6, 150 mM NaCl, 2 mM EDTA, 0.5 mM spermidine, 1% BSA, 1× protease inhibitors) with a cocktail of primary antibodies and incubated overnight on a rotator at 4° C. The next day cells were washed twice with 150 μL wash buffer to remove the remaining antibodies. The cells were then resuspended in 150 μL high salt wash buffer (20 mM HEPES pH 7.6, 300 mM NaCl, 0.5 mM spermidine, 1× protease inhibitors) with 2.5 μL nb-Tn5 for each target of interest and incubated for 1 h on a rotator at room temperature. The cells were then washed twice with high salt wash buffer and resuspended in 50 μL tagmentation buffer (20 mM HEPES pH 7.6, 300 mM NaCl, 0.5 mM spermidine, 10 mM MgCl2, 1× protease inhibitors). The samples were incubated for 1 h at 37° C. Tagmentation steps were performed in 0.2 mL tubes to minimize cell loss.

### NTT-Seq Bulk

To stop tagmentation, 1 μL of 0.5 M EDTA, 1 μL of 10% SDS and 0.25 μL of 20 mg/mL Proteinase K was added to the sample, incubated at 55° C. for 1 hour. DNA was extracted with Chip DNA clean & Concentrator kit (Zymo Research, D5201) following manufacturer instructions. To amplify libraries, 21 μL DNA was mixed with 2 μL of a universal i5 and a uniquely barcoded i7 primer, using a different barcode for each sample. A volume of 25 μL NEBNext HiFi 2×PCR Master mix was added and mixed. The sample was placed in a Thermocycler with a heated lid using the following cycling conditions: 72° C. for 5 minutes (gap filling): 98° C. for 30 s: 14 cycles of 98° C. for 10 s and 63° C. for 30 s: final extension at 72° C. for 1 minutes and hold at 8° C. Post-PCR clean-up was performed by adding 1.1× volume of Ampure XP beads (Beckman Coulter), and libraries were incubated with beads for 15 minutes at RT, washed twice gently in 80% ethanol, and eluted in 30 μL 10 mM Tris pH 8.0.

NTT-Seq Single Cell Encapsulation. PCR. And Library Construction

After tagmentation, cells were centrifuged for 5 minutes at 1,000 g and the supernatant was discarded. Cells were resuspended with 30 μL 1× Diluted Nuclei Buffer (10× Genomics, #2000207), counted, and diluted to a concentration based on the targeted cell number. The transposed cell mix was prepared as following: 7 μL of ATAC buffer and 8 μL cells in 1× Diluted Nuclei Buffer. All remaining steps were performed according to the 10× Chromium Single Cell ATAC protocol. For NTT-seq with surface markers readout on primary cells, the library construction method was adapted from ASAP-seq (Mimitou et al., Nat Biotechnol. 2021 October; 39(10):1246-1258). Briefly, 0.5 μL of 1 μM bridge oligo A (SEQ ID NO: 27-TCGTCGGCAGCGTCAGATGTGTATAAGAGACAGNNNNNNNNNVTTTTTTTTTTTT TTTTTTTTTTTTTTTTTT/3InvdT/) was added to the barcoding mix. Linear amplification was performing using the following PCR program: (40° C. for 5 minutes, 72° C. for 5 minutes, 98° C. for 30 s: 12 cycles of 98° C. for 10 s, 59° C. for 30 s and 72° C. for 1 minutes: ending with hold at 15° C.). The remaining steps were performed according to the 10× Genomics scATAC-seq protocol (v1.1), with the following additional modifications:

Antibody-derived tags: during silane bead elution (Step 3.1s), beads were eluted in 43.5 μL of elution solution I. The extra 3 μL was used for the surface protein tags library. During SPRI cleanup (Step 3.2d), the supernatant was saved and the short DNA derived from antibody oligos was purified with 2×SPRI beads. The eluted DNA was combined with the 3 μL left aside after the silane purification to be used as input for protein tag amplification. PCR was set up to generate the protein tag library with Kapa Hifi Master Mix (P5 and RPI-x primers): 95° C. for 3 minutes: 14-16 cycles of 95° C. for 20 s, 60° C. for 30 s and 72° C. for 20 s; followed by 72° C. for 5 minutes and ending with hold at 4° C.

RPI-x primer:
(SEQ ID NO: 28)
CAAGCAGAAGACGGCATACGAGATNNNNNNNNGTGACTGGAGTTCCTTG
GCACCCGAGAATTCCA
P5 Primer:
(SEQ ID NO: 22)
AATGATACGGCGACCACCGAGATCTACAC

### Sequencing

The final libraries were sequenced on NextSeq 550 by using custom primers (table below) with the following strategy: i5: 38 bp, i7: 8 bp, read1: 60 bp, read2: 60 bp (for PBMC single-cell NTT-seq without cell surface proteins, read1: 50 bp, read2: 50 bp).

| | | SEQ |
|---|---|---|
| Oligo | | ID |
| name | Oligo sequence (Barcode) | NO |
| MEDSA_1 | TCGTCGGCAGCGTCGGATTGCTGCGATCGAGGAC | 29 |
| | GGCAGATGTGTATAAGAGACAGGGATTGCT | |
| MEDSA_2 | TCGTCGGCAGCGTCGTAATGCAGCGATCGAGGAC | 30 |
| | GGCAGATGTGTATAAGAGACAGGTAATGCA | |
| MEDSA_3 | TCGTCGGCAGCGTCGTCAAGGAGCGATCGAGGAC | 31 |
| | GGCAGATGTGTATAAGAGACAGGTCAAGGA | |
| MEDSA_4 | TCGTCGGCAGCGTCGTGAGCGTGCGATCGAGGAC | 32 |
| | GGCAGATGTGTATAAGAGACAGGTGAGCGT | |
| MEDSA_5 | TCGTCGGCAGCGTCGTGTGACCGCGATCGAGGAC | 33 |
| | GGCAGATGTGTATAAGAGACAGGTGTGACC | |
| MEDSA_6 | TCGTCGGCAGCGTCTAAGGTGGGCGATCGAGGAC | 34 |
| | GGCAGATGTGTATAAGAGACAGTAAGGTGG | |
| MEDSB | GTCTCGTGGGCTCGGAGATGTGTATAAGAGACAG | 35 |
| Custom | GCGATCGAGGACGGCAGATGTGTATAAGAGACAG | 36 |
| R1 | | |
| Custom | CTGTCTCTTATACACATCTGCCGTCCTCGATCGC | 37 |
| i5 | | |

### Bulk-Cell Data Analysis

Bulk-cell data for the cell culture and PBMC datasets were mapped to the hg38 analysis set using bwa-mem2 with default parameters. Output BAM files were sorted and indexed using samtools, and bigwig files created using the deeptools bamCoverage function with the—normalizeUsing BPM option set. Fragment files were created using the Sinto (github.com/timoast/sinto), which uses the Pysam and htslib packages. Multi-NTT-seq heatmaps were generated in DeepTools. ChIP-seq peak coordinates for H3K27me3 and H3K27ac for bulk PBMCs, and for H3K27me3, H3K27ac, and RNAPII serine-2 and serine-5 phosphate for K562 cells were downloaded from ENCODE (Nature. 2012 Sep. 6; 489(7414):57-74). We counted sequenced DNA fragments falling within each peak region for each bulk-cell PBMC or K562-cell NTT-seq dataset using custom R code and the scanTabix function in Rsamtools, and normalized counts according to the total number of mapped reads for each dataset (counts per million mapped reads normalization). The coefficient of determination (R2) between peak counts across pairs of experiments was computed using the 1 m function in R.

### Single-Cell Data Analysis

CELL culture dataset

### Read Mapping

Reads were mapped to the hg38 analysis set using bwa-mem2 with default parameters, the output sorted and indexed using samtools, and the resulting BAM file used to create a fragment file using the Sinto package (github.com/timoast/sinto). We ran the sinto fragments command with the—barcode_regex “[\hat{ }:]*” parameter set to extract cell barcodes from the read name. Output files were coordinate-sorted, bgzip-compressed and indexed using tabix, and the resulting fragment files used as input to downstream analyses.

### Quantification, Quality Control, and Dimension Reduction

Genomic regions were quantified using the AggregateTiles function in Signac with binsize=10000 and min_counts=1, using the hg38 genome. Cells with <10,000 total counts, >75H3K27ac counts, >150H3K27me3 counts, and >100 RNAPII counts were retained for further analysis. Each assay was processed by performing TF-IDF normalization on the count matrix for the assay, followed by latent semantic indexing (LSI) using the RunTFIDF and RunSVD functions in Signac with default parameters. Two-dimensional visualizations were created for each assay using UMAP, using LSI dimensions 2 to 10 for each assay. Weighted nearest neighbor (WNN) analysis was performed using the FindMultiModalNeighbors function in Seurat, with reduction.list=list(“lsi.k27ac”, “lsi.k27me”, “lsi.pol2”) and dims=list(2:10, 2:10, 2:10) to use LSI dimensions 2 to 10 for each assay. Cell clustering was performed using the resulting WNN graph using the Smart Local Moving community detection algorithm by running the FindClusters function in Seurat, with algorithm=3, graph.name=“wsnn”, and resolution=0.05. This resulted in two cell clusters, which were assigned as HEK or K562 based on their correlation with bulk-cell chromatin data for HEK and K562 cells.

### Specificity Analysis

K562-cell bulk ChIP-seq peaks for H3K27ac, H3K27me3, and RNA Pol2 Ser-2 and Ser-5 phosphate were downloaded from ENCODE (Nature. 2012 Sep. 6; 489(7414):57-74). Since the fraction of reads in peaks metric can be sensitive to the peak set used, we opted to use previously reported ENCODE peaks throughout our analysis as much as possible. Ser-2 and Ser-5 phosphate peaks were combined using the reduce function from the GenomicRanges R package. Fragment counts for K562 cells in the bulk and single-cell dataset were quantified for each peak using the scanTabix function in the Rsamtools R package, with counts normalized according to the total sequencing depth for each dataset. To assess the targeting specificity in single-cell NTT-seq, we computed the coefficient of determination (R2) between peak counts for each pair of assays, and between bulk and single-cell data for the same assay. We visualized relative peak counts for each assay for each peak by creating a ternary plot using the ggtern R package. To assess the low-dimensional neighbor structure obtained using each assay or combinations of assays, we computed the fraction of k-nearest neighbors for each cell i that belonged to the same cell type classification as cell i (k=50 for single-modality neighborhoods, variable k per-cell for multimodal neighbor graph due to the weighted nearest neighbor method).

### Multi-CUT & Tag Comparison

To create a fragment file for the published multi-CUT & Tag dataset, raw sequencing data from Gopalan et al. (Mol Cell. 2021 Nov. 18; 81(22):4736-4746.e5) were downloaded from NCBI SRA and split into separate FASTQ files according to their Tn5 barcode using a custom Python script. Reads were mapped to the hg38 genome using bwa-mem2 and fragment files created as described above for the NTT-seq datasets. Code to reproduce this analysis is available on GitHub: github.com/timoast/multi-ct. We ran the CountFragments function in Signac to count the total number of fragments per cell for each multi-CUT & Tag assay, and retained cells with >200 total counts for further analysis, as described in the original publication (Mol Cell. 2021 Nov. 18; 81(22):4736-4746.e5). For mixed-barcode fragments we counted ½ count to the total of each assay matching the pair of Tn5 barcodes. To compute the targeting specificity, we downloaded published ENCODE ChIP-seq peaks for H3K27me3 and H3K27ac for mESCs (ENCFF008XKX and ENCFF360VIS), and computed the fraction of fragments in peak regions using the scanTabix function in the Rsamtools R package, normalizing counts according to the total sequencing depth for the dataset. We also computed the R2 between H3K27me3 and H3K27ac as described above, using the ENCODE peak regions.

### PBMC Datasets

### Read Mapping

Genomic reads were mapped and processed as described above for the cell culture single-cell dataset. Antibody-derived tag (ADT) reads were processed using Alevin. We first created a salmon index for the BioLegend TotalSeq-A antibody panel, with the—features-k7 parameters. We quantified counts for each ADT barcode using the salmon alevin command with the following parameters: —naiveEqclass, —keepCBFraction 0.8, —bc-geometry 1[1-16], —umi-geometry 2[1-10], —read-geometry 2[71-85].

### Quantification, Quality Control, and Dimension Reduction

Genomic bins were quantified using the AggregateTiles function in Signac, with binsize=5000 and min_counts=1 to quantify 5 kb bins genome-wide, retaining bins with at least one count. We retained cells with <40,000 and >300H3K27me3 counts, <10,000 and >100H3K27ac counts, and <10,000 and >100 antibody-derived tag (ADT) counts. We normalized the ADT data using a centered log ratio transformation using the NormalizeData function in Seurat, with normalization.method=“CLR” and margin=2. We reduced the dimensionality of the ADT assay by first scaling and centering the protein expression values, and running PCA (ScaleData and RunPCA functions in Seurat). We computed a 2-dimensional UMAP visualization using the first 40 principal components (PCs), and clustered cells using the Louvain community detection algorithm. We identified and removed two low-quality clusters containing higher overall ADT counts, as well as higher counts for naive IgG antibodies included in the staining panel. After removing low-quality ADT clusters, we reduced the dimensionality of the H3K27me3 and H3K27ac assays using LSI (FindTopFeatures, RunTFIDF, RunSVD functions in Signac) and created 2-dimensional UMAPs using LSI dimensions 2 to 30 for each chromatin assay. To construct a low-dimensional representation using all three data modalities, we ran the weighted nearest neighbors (WNN) algorithm, using the first 40 ADT PCs, and LSI dimensions 2 to 30 for H3K27me3 and H3K27ac (FindMultiModalNeighbors function in Seurat). We clustered cells using the WNN neighbor graph using the Smart Local Moving algorithm (32) (FindClusters function in Seurat with algorithm=3 and resolution=1). Cell clusters were manually annotated as cell types using the protein expression information. To compare the low-dimensional structure obtained using individual chromatin modalities or combinations of modalities, we computed for each cell i the fraction of neighboring cells annotated as the same cell type as cell i. We repeated this computation using neighbor graphs computed using single data modalities, or weighted combinations of modalities computed using the WNN method.

### ENCODE Data Comparison

Peaks and genomic coverage bigWig files for H3K27me3 and H3K27ac ChIP-seq published by the ENCODE consortium (Nature. 2012 Sep. 6: 489 (7414): 57-74) for B cells, CD34+ CMPs, and CD14+ monocytes were downloaded from the ENCODE website (encodeproject.org). bigWig files were created for each corresponding cell type identified in the single-cell multiplexed NTT-seq PBMC dataset by writing sequenced fragments for those cells to a separate BED file, creating a bedGraph file using the bedtools genomecov command, and creating a bigWig file using the UCSC bedGraphToBigWig tool. Genomic coverage for NTT-seq datasets and ChIP-seq datasets within H3K27me3 and H3K27ac regions were computed using the deeptools multiBigwigSummary function with the—outRaw Counts option set to output the raw correlation matrix as a text file. We computed the correlation between peak region coverage in NTT-seq and ENCODE ChIP-seq datasets using the cor function in R with method=“spearman”. The fraction of fragments per cell falling in ENCODE H3K27me3 and H3K27ac ChIP-seq peak regions for PBMCs for each assay were computed as described above.

### CUT & Tag-Pro Data Comparison

Processed CUT & Tag-pro H3K27me3 and H3K27ac datasets for human PBMCs were downloaded from Zenodo (available at zenodo.org/record/5504061). We compared the number of antibody-derived tag (ADT) counts in NTT-seq and scCUT & Tag-pro datasets by extracting the total number of ADT counts per cell from the scCUT & Tag-pro and NTT-seq Seurat objects and plotting the distribution of total ADT counts per cell for each dataset. We created bigWig files for each scCUT & Tag-pro dataset by first creating a bedGraph file using the bedtools genomecov function, and then creating a bigWig file using the UCSC bedGraphToBigWig function. We computed the coverage for scCUT & Tag-pro datasets within H3K27me3 and H3K27ac PBMC ENCODE peaks using the multiBigwigSummary function in deeptools as described above for the ENCODE data comparison.

### BMMC Dataset

### Read Mapping

Raw genomic reads were mapped and processed as described above for the cell culture single-cell dataset.

### Quantification, Quality Control, and Dimension Reduction

Genomic bins were quantified using the AggregateTiles function in Signac, with binsize=5000 and min_counts=1 to quantify 5 kb bins genome-wide, retaining bins with at least one count. We retained cells with <10,000 and >100H3K27me3 counts, and <10,000 and >75H3K27ac counts for further analysis. We normalized the counts and reduced dimensionality for each assay by running the RunTFIDF, RunSVD, and RunUMAP functions in Signac and Seurat for each assay. We computed a WNN graph for H3K27me3 and H3K27ac using the FindMultiModalNeighbors function in Seurat, with reduction=list (“lsi.me3”, “lsi.ac”) and dims.list=list (2:50, 2:80) to use LSI dimensions 2 to 50 and 2 to 80 for H3K27me3 and H3K27ac, respectively. A 2-dimensional UMAP was created using the WNN graph by running the RunUMAP function in Seurat with nn.name=“weighted.nn” to use the pre-computed neighbor graph. We clustered cells using the WNN graph using the Smart Local Moving community detection algorithm (FindClusters function in Seurat with algorithm=3, resolution=3, graph.name=“wsnn”). We computed the fraction of fragments per cell falling in ENCODE PBMC H3K27me3 and H3K27ac ChIP-seq peak regions for each assay as described above.

### Cell Annotation

To annotate cell types, we performed label transfer (Mimitou et al., Nat Biotechnol. 2021 October; 39(10):1246-1258) using the H3K27ac assay and a previously published scATAC-seq dataset containing healthy human bone marrow cells (Granja et al., Nat Biotechnol. 2019 December; 37(12):1458-1465). As the original publication mapped reads to the hg19 genome, we re-processed the original reads using the 10× Genomics cellranger-atac v2 software with default parameters, aligning to the hg38 genome. Code to reproduce this analysis is available on GitHub: github.com/timoast/MPAL-hg38. To transfer cell type labels from the scATAC-seq dataset to our multimodal NTT-seq dataset, we quantified scATAC-seq peaks using the H3K27ac assay, then performed TF-IDF normalization on the resulting count matrix using the IDF value from the scATAC-seq dataset. We performed LSI on the scATAC-seq BMMC dataset using the RunTFIDF and RunSVD functions in Signac with default parameters. We next ran the FindTransferAnchors function in Seurat, with reduction=“lsiproject”, dims=2:30, and reference.reduction=“Isi” to project the query data onto the reference scATAC-seq LSI using dimensions 2 to 30, and find anchors between the reference and query dataset. We ran TransferData with weight.reduction=bmmc_ntt[[“lsi.me3”]] dims=2:50 to weight anchors using LSI dimensions 2 to 50 from the H3K27me3 assay. We used these unsupervised cell type predictions as a guide when assigning cell clusters to cell types.

### Trajectory Analysis

We subsetted the BMMC dataset to contain cells annotated as HSPC, GMP/CMP, Pre-B, B, or Plasma cells. Using the subset object, we constructed a new UMAP dimension reduction by running FindTopFeatures, RunTFIDF, and RunSVD in Signac, followed by RunUMAP in Seurat with reduction=“Isi”, for each assay. We then constructed a joint low-dimensional space using the WNN method by running the FindMultiModalNeighbors function in Seurat. We converted the Seurat object containing these cells to a SingleCellExperiment object using the as.cell_data_set function in the SeuratWrappers package (github.com/satijalab/seurat-wrappers). We next ran Monocle 3 using the pre-computed UMAP dimension reduction constructed using both chromatin modalities by running the cluster_cells, learn_graph, and order_cells functions, setting the HSPC cells as the root of the trajectory. To find genomic features in each assay whose signal depended on pseudotime state, we quantified fragment counts for each cell in each 10 kb genome bin for the H3K27me3 and H3K27ac assays. To reduce the sparsity of the measured signal, we averaged counts for each genomic region across the cell's 50 nearest neighbors, defined using the H3K27me3 neighbor graph with LSI dimensions 2 to 20, and normalized the fragment counts by the total neighbor-averaged counts per cell. For each genomic region we computed the Pearson correlation between the signal in the genomic region and the cell's position in pseudotime. To find regions that underwent coordinated activation or repression we selected regions with a Pearson correlation >0.2 or <−0.2 and a difference in Pearson correlation between the H3K27me3 and H3K27ac assays greater than 0.5 (e.g., −0.25 correlation for H3K27me3 and +0.25 for H3K27ac). To display genomic regions in a heatmap representation we ordered cells based on their pseudotime rank and ordered genomic regions based on the position in pseudotime showing maximal H3K27me3 signal. For the purpose of visualization, we smoothed the signal for each genomic region by applying a rolling sum function with cells ordered based on pseudotime, summing the signal over 100-cell windows. This was performed using the roll_sum function in the RcppRoll R package (version 0.3.0).

We used the ClosestFeature function in Signac to identify the closest gene to each genomic region correlated with pseudotime. Genomic regions where the closest gene was >50,000 bp away were removed (21 genes for H3K27me3 and 7 genes for H3K27ac). To examine the gene expression patterns of these genes, we downloaded a previously integrated and annotated scRNA-seq dataset for the human bone marrow; produced as part of the HuBMAP consortium (zenodo.org/record/5521512). We subset the scRNA-seq object to contain the same cell states that we examined in the NTT-seq data (HSC, LMPP, CLP, pro-B, pre-B, transitional B, naive B, mature B, plasma) and computed a gene module score for the active and repressed genes using the AddModuleScore function in Seurat.

To compare changes in scATAC-seq signal across the B cell developmental trajectory, we also downloaded a previously published BMMC scATAC-seq dataset, and subset the cells belonging to the B cell trajectory using the published cell type annotations provided by the original authors. We quantified the same set of genomic regions used in the scNTT-seq BMMC analysis, and created a similar B cell developmental trajectory by assigning a numeric value to each B cell type according to its relative position along the known developmental trajectory (1=HSC, 2=CMP/LMPP, 3=CLP, 4=B, 5=Plasma), and computed the Pearson correlation between each genomic region and the B cell trajectory.

### Example 4: Multifactorial Chromatin Profiling Using Nanobody-Tethered Transposition Followed by Sequencing (NTT-Seq)

Chromatin states are functionally defined by a complex combination of histone modifications, transcription factor binding, DNA accessibility, and other factors. Current methods for defining chromatin states cannot measure more than one aspect in a single experiment at single-cell resolution. Here, we describe nanobody-tethered transposition followed by sequencing (NTT-seq), an assay capable of measuring the genome-wide presence of up to three histone modifications and protein-DNA binding sites at single-cell resolution. NTT-seq utilizes recombinant Tn5 transposase fused to a set of secondary nanobodies (nb). Each nb-Tn5 fusion protein specifically binds to different immunoglobulin-G antibodies, enabling a mixture of primary antibodies binding different epitopes to be used in a single experiment. We apply bulk- and single-cell NTT-seq to generate high-resolution multimodal maps of chromatin states in cell culture and in human immune cells. We also extend NTT-seq to enable simultaneous profiling of cell-surface protein expression and multimodal chromatin states to study cells of the immune system.

We engineered and produced four different recombinant nb-Tn5 fusion proteins, specific for IgG antibodies from different species or IgG subtypes (FIG. 12A, FIG. 15A). This included anti-mouse and anti-rabbit IgG nanobodies, as well as isotype-specific nanobodies for mouse IgG1 and IgG2a. Loading nb-Tn5 fusion proteins with barcoded DNA adaptor sequences enables the identity of individual nb-Tn5 fusion proteins that generated the sequenced DNA fragment to be determined through DNA sequencing.

We tested each recombinant nb-Tn5 fusion in a bulk-cell NTT-seq experiment and obtained an NTT-seq library only when the nb-Tn5 matched the target antibody, while the incubation of nb-Tn5 with the unmatched Ab resulted in no library amplification via PCR (FIG. 15B). Motivated by this result, we performed multiplexed NTT-seq aiming to profile multiple different chromatin features in a single experiment. In our protocol, extracted nuclei are stained in a single step using primary antibodies for multiple epitopes simultaneously, the excess antibody is washed and nuclei are incubated with a mixture of adapter-barcoded nb-Tn5s, with each nb-Tn5 recognizing a specific IgG antibody. Subsequently, nb-Tn5s are activated by adding Mg2+ resulting in the tagmentation of genomic DNA in proximity of the primary antibody. The released DNA fragments harbor specific barcodes enabling the assignment of sequenced fragments to an individual nb-Tn5 and its associated primary antibody (FIG. 12B).

To test the targeting specificity of our species-specific nb-Tn5 fusion proteins, we used antibodies for H3K27me3 and H3K27ac in bulk human peripheral blood mononuclear cells (PBMCs), as these marks do not co-occur in the genome. Multiplexed NTT-seq resulted in libraries with nearly identical genomic distributions for each separate mark to matched NTT-seq performed on the same cells for each histone mark separately (FIG. 12C). The enrichment of sequenced fragments falling in H3K27me3 and H3K27ac peaks was approximately the same across the multiplexed and non-multiplexed experiments (FIG. 12D and FIG. 12E), and showed mutual exclusivity (FIG. 12F, FIG. 12G, FIG. 15C). This suggests that multiplexed NTT-seq results in highly accurate localization of chromatin marks genome-wide. Then, we tested our isotype-specific nb-Tn5 profiling of three primary antibodies in a single experiment, repeating similar experiments using K562 cells staining with mouse IgG1 antibody against H3K27me3, mouse IgG2a antibody against H3K27ac, and including an additional rabbit IgG antibody for RNA Polymerase II (RNAPII) with phosphorylated Serine 2 and Serine 5 (elongating RNAPII, enriched on actively transcribed genes). In comparison with a control experiment in which each of the three targets was profiled individually, multiplexed NTT-seq again produced comparable target enrichment specificity in peaks (FIG. 12H, FIG. 12I, FIG. 12J, FIG. 15D), demonstrating the ability to profile three targets simultaneously, as well as the ability to profile non-histone proteins.

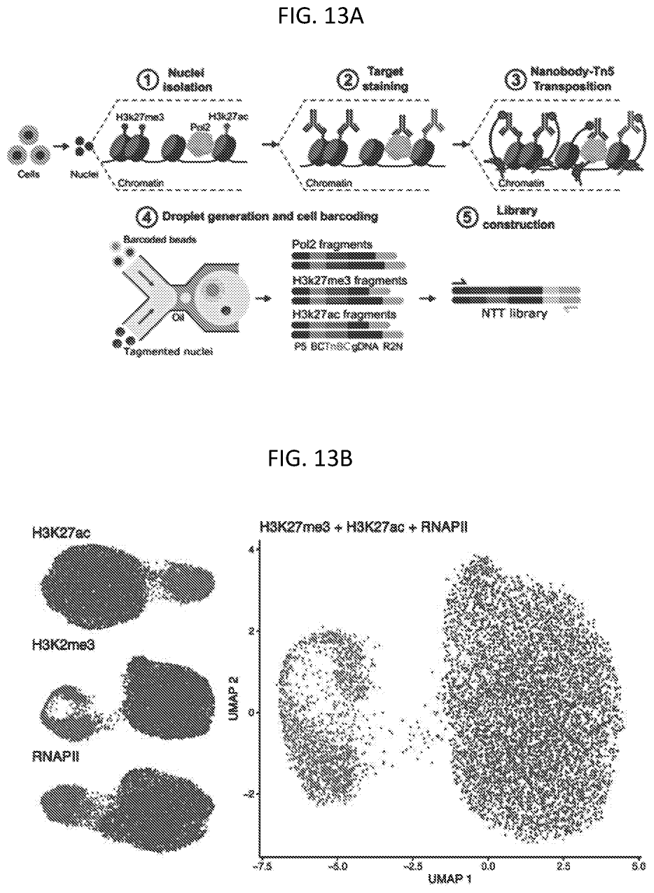

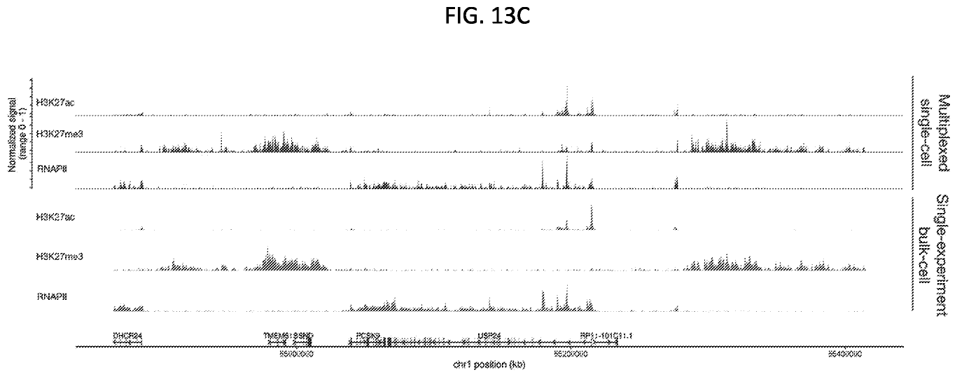

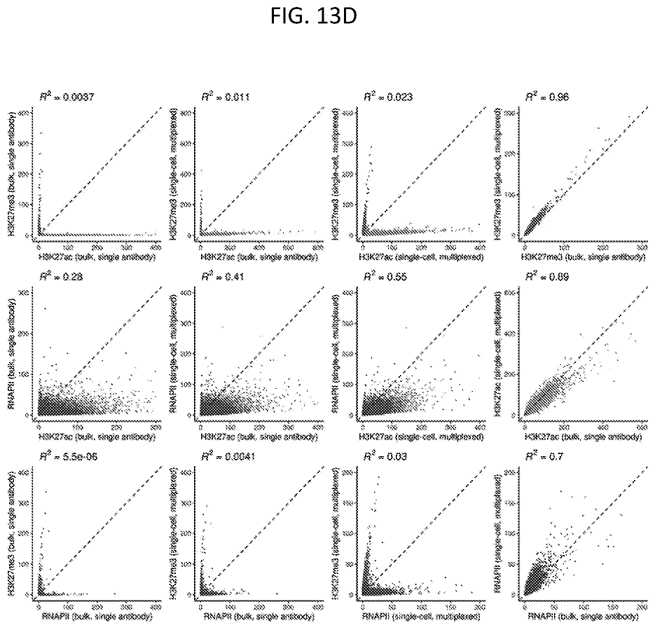

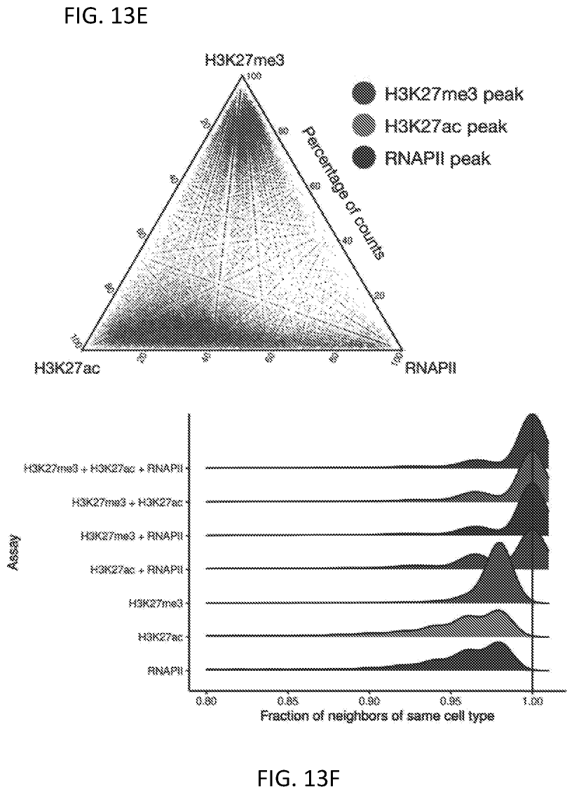

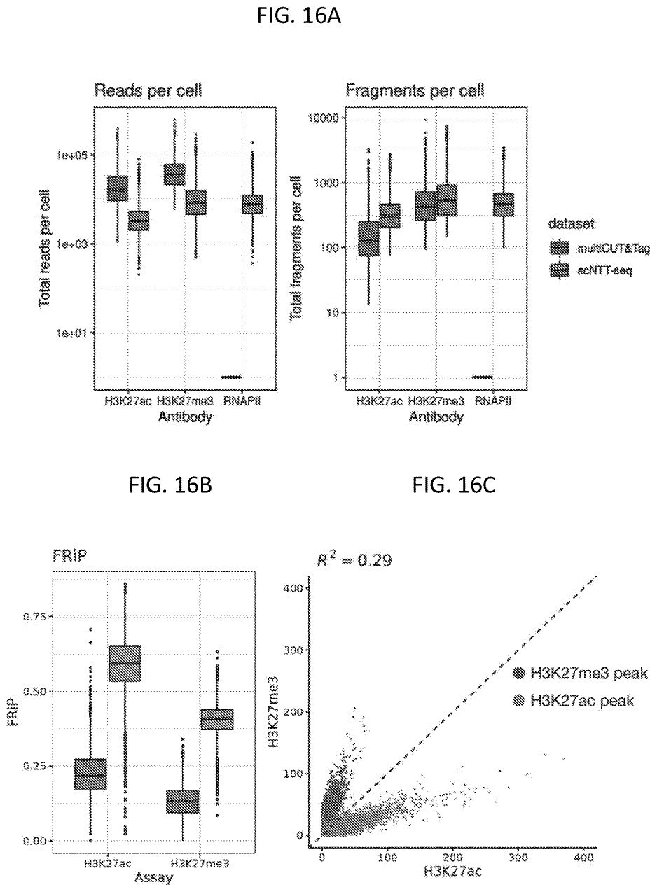

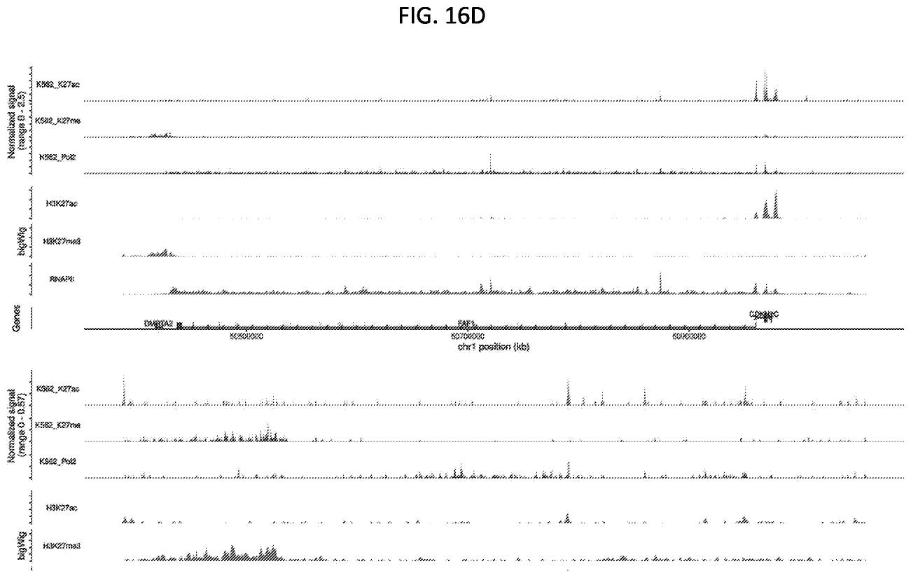

Encouraged by the results obtained in bulk cells, we next applied NTT-seq to characterize multimodal chromatin states at single-cell resolution using the 10× Genomics scATAC-seq kit (FIG. 13A). We profiled H3K27me3, H3K27ac and elongating RNAPII in a mixture of 8,617 K562 and HEK293 cells. We obtained on average 743 (s.d. 699) fragments for H3K27me3, 382 (s.d. 282) fragments for H3K27ac and 542 (s.d. 350) fragments for RNAPII per cell, outperforming the recently developed multiCUT & Tag method (Gopalan S et al., Mol Cell. 2021 Nov. 18: 81 (22): 4736-4746.e5) in terms of sensitivity and specificity (FIG. 16A, FIG. 16B, FIG. 16C).

| | | | | | Total fragments standard | | | Mean fraction of fragments in | | |
|---|---|---|---|---|---|---|---|---|---|---|
| | Total | Mean fragments per cell | | | deviation | | | ENCODE peaks | | |
| Dataset | cells | H3K27me3 | H3K27ac | RNAPII | H3K27me3 | H3K27ac | RNAPII | H3K27me3 | H3K27ac | RNAPII |
| K562 | 8617 | 743 | 382 | 542 | 699 | 282 | 350 | 0.4 | 0.59 | 0.2 |
| PBMC + | 4684 | 2854 | 412 | — | 2953 | 356 | — | 0.11 | 0.21 | — |
| protein | | | | | | | | | | |
| PBMC | 4770 | 670 | 731 | — | 1243 | 1035 | — | 0.1 | 0.28 | — |
| BMMC | 5236 | 1217 | 326 | — | 1274 | 334 | — | 0.18 | 0.26 | — |

We projected cells into a low-dimensional space using latent semantic indexing (LSI) and UMAP (14.15), and clustered cells using a weighted combination of all three data modalities (FIG. 13B). We identified two groups of cells corresponding to K562 and HEK293 cells. The genomic distribution of reads for each mark obtained in the multiplexed single-cell experiment was highly similar to data from the same cell lines where each feature was profiled individually in bulk (FIG. 13C, FIG. 16B). Examining the distribution of fragments at ATAC. H3K27me3, H3K27ac, and RNAPII peaks further showed the co-occupancy of RNAPII and H3K27ac in open chromatin regions, while the signal for H3K27me3 was mutually exclusive with the other profiled marks (FIG. 13D, FIG. 13E). Furthermore, multiplexed single-cell-derived signals were highly correlated with bulk-cell signal for each assay profiled individually (FIG. 13D). Using a combination of cellular modalities provided the strongest separation of the two cell types in low-dimension space. When constructing a neighbor graph, we observed a higher fraction of a cell's neighbors belonging to the same cell type as that cell when using multiple modalities (FIG. 13F). This highlights the value of multimodal chromatin data in measuring cellular states, and together these results show that NTT-seq is an effective method for profiling multiple chromatin modalities at single-cell resolution.

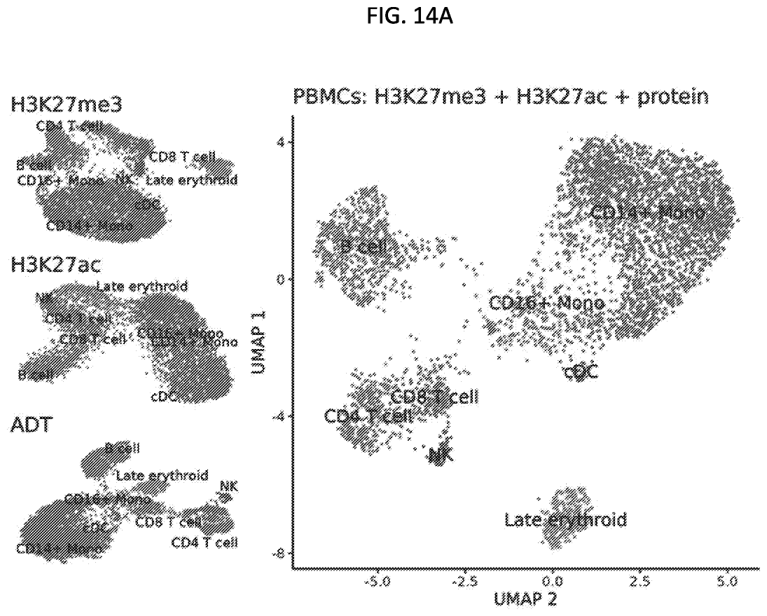

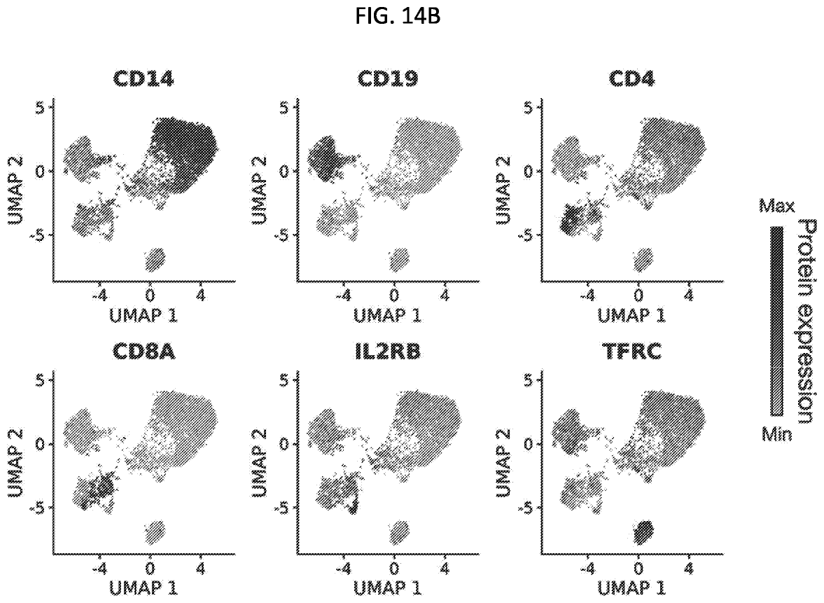

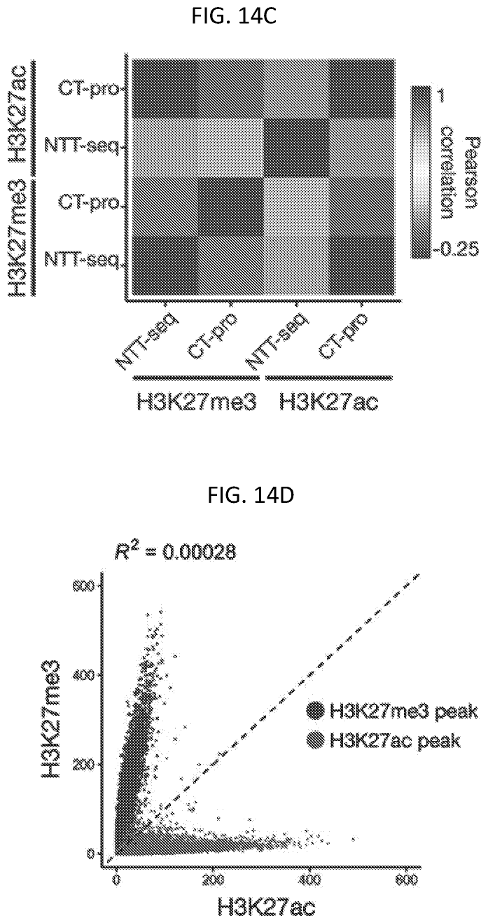

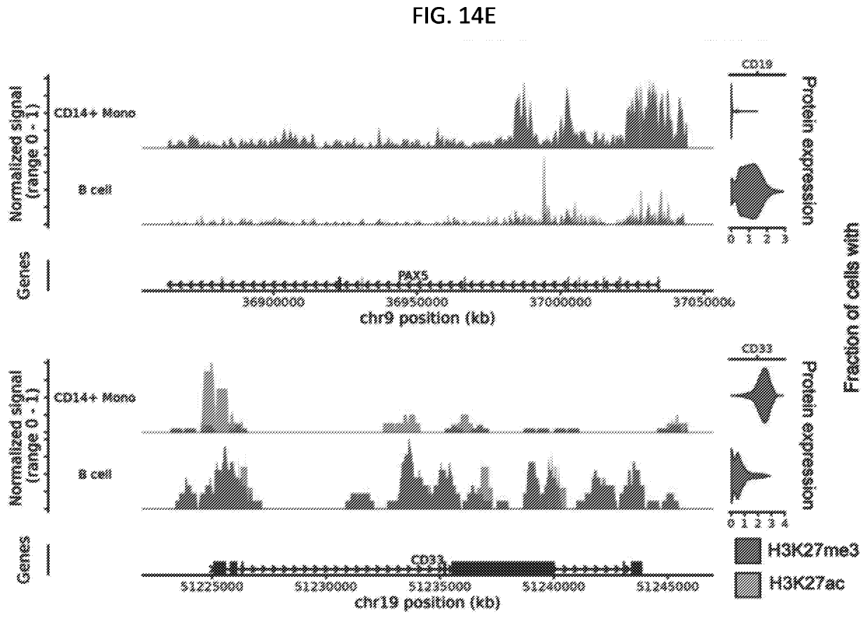

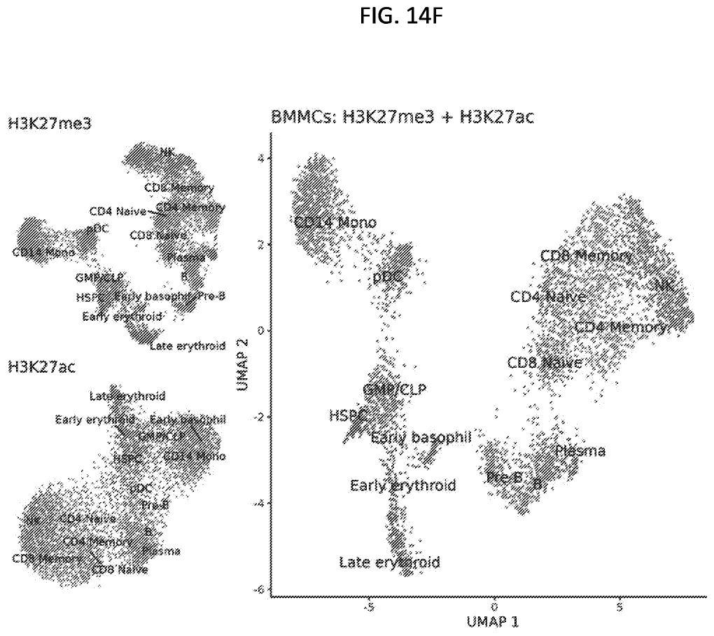

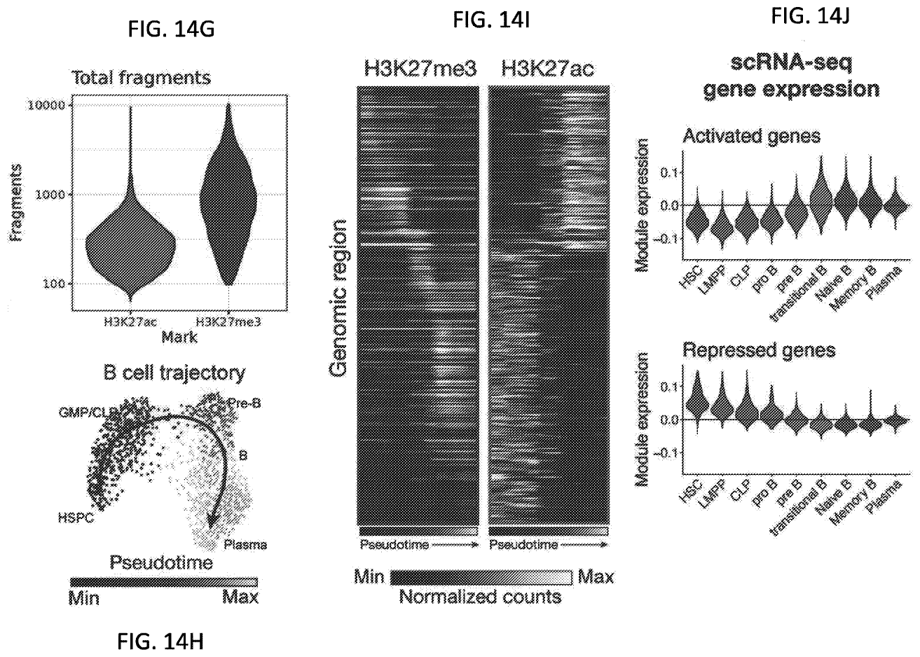

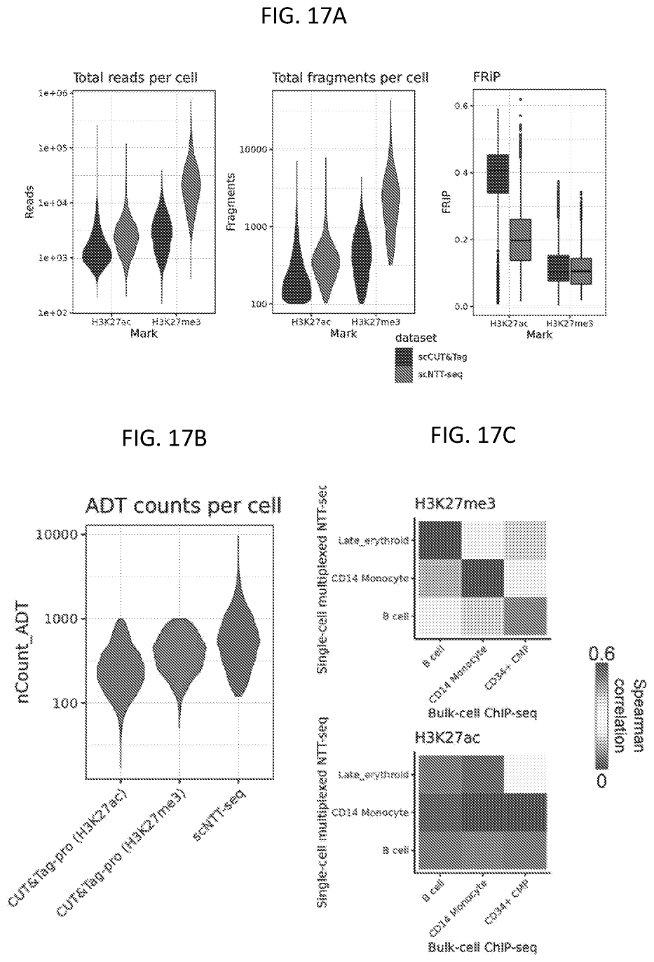

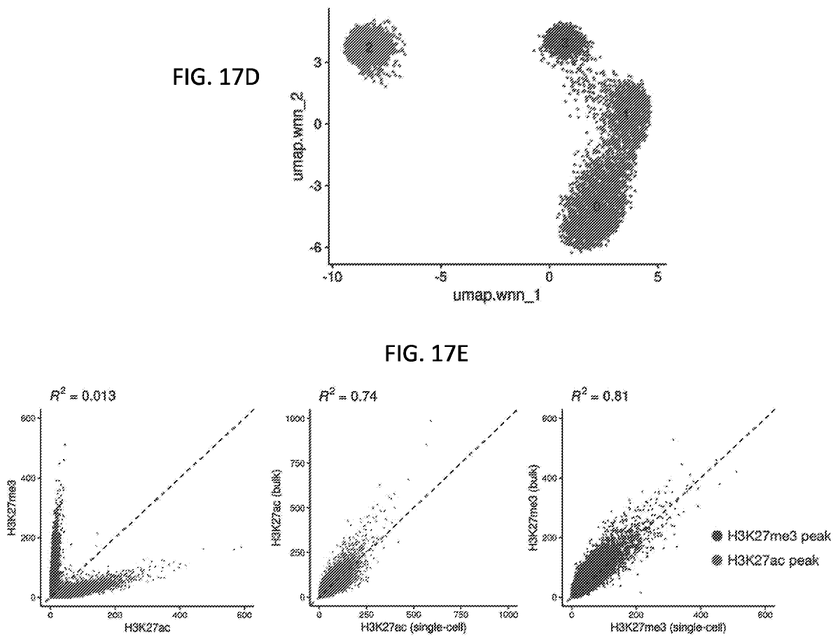

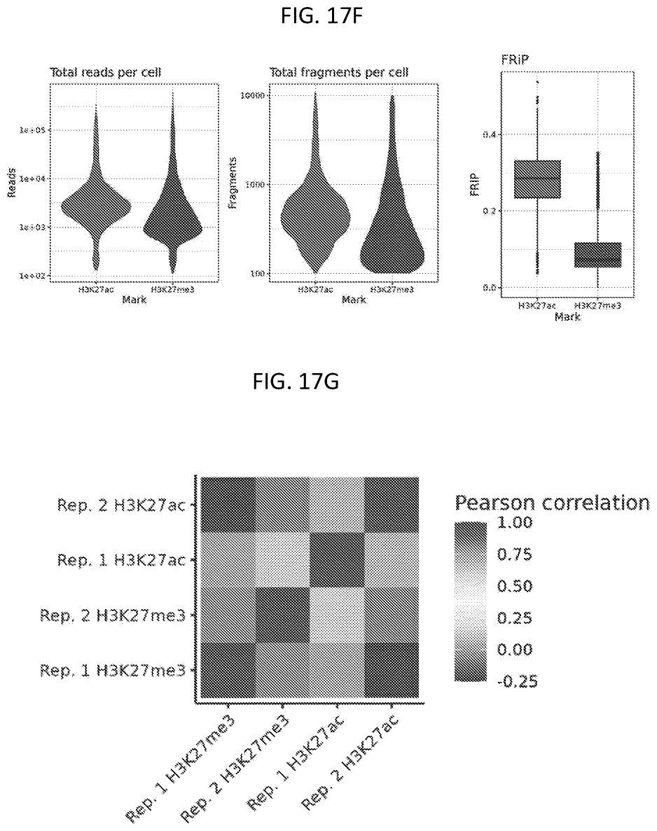

We next sought to extend the NTT-seq method to enable simultaneous measurement of cell surface protein expression alongside multimodal chromatin states at single-cell resolution. Building on the recently developed CUT & Tag-pro method, we stained a population of mobilized PBMCs with an oligonucleotide-conjugated panel of 173 antibodies targeting immune-relevant cell surface proteins. Cells were then crosslinked, permeabilized, and incubated with antibodies against H3K27me3 and H3K27ac, and our standard NTT-seq protocol followed to generate single-cell libraries. This resulted in a dataset of 4,684 cells with a mean of 2,854 H3K27me3 and 412H3K27ac fragments per cell (s.d. 2,953, 356 respectively), with similar sensitivity and specificity to PBMC scCUT & Tag (FIG. 17A). We further quantified 690 antibody-derived tag (ADT) counts per cell (s.d. 613), achieving a sensitivity similar to the recently demonstrated scCUT & Tag-pro method (FIG. 17B) (18). We clustered cells using a weighted combination of each modality and annotated cell clusters based on their patterns of protein expression (FIG. 14A). Protein expression patterns were concordant with cell clusters determined from a chromatin-based clustering, and we observed uniform expression of CD3 in T cells, mutually exclusive expression of CD4 and CD8, expression of CD14 in monocytes, CD19 in B cells, and IL2RB in NK cells (FIG. 14B). Pseudobulk H3K27me3 and H3K27ac NTT-seq profiles were highly correlated with individual single-cell CUT & Tag-pro profiles for human PBMCs for the same histone marks (FIG. 14C). Consistent with our previous results, we also observed an extremely low coefficient of determination (R2=0.00028) between H3K27me3 and H3K27ac levels within peaks (FIG. 14D), further supporting the accuracy of multiplexed NTT-seq single-cell profiles when applied to complex tissues. We observed consistency between chromatin states and protein expression patterns for each cell type, supporting accurate cell-surface protein quantification. For example, the PAX5 locus was repressed in non-B cells with low CD19 protein expression, and active in B cells with high CD19 expression (FIG. 14E). Similarly, the CD33 locus was active in monocytes with high CD33 protein expression and repressed in B cells with low CD33 expression. To evaluate the accuracy of our cell type classifications and multimodal chromatin landscapes measured by NTT-seq, we compared the results of our single-cell NTT-seq experiment with FACS-sorted ChIP-seq profiles for CD14 monocytes, CD34+ CMPs, and B cells previously published by the ENCODE consortium. Pseudobulk profiles generated from our NTT-seq cell types recapitulated the expected cell-type-specific ENCODE ChIP-seq profiles (FIG. 17C). To evaluate the reproducibility of single-cell chromatin profiles measured by scNTT-seq, we generated a second scNTT-seq dataset measuring H3K27me3 and H3K27ac in human PBMCs (FIG. 17D). This dataset achieved a similar level of sensitivity and specificity (FIG. 17E, FIG. 17F), and was highly correlated with the genome-wide chromatin profiles obtained in our first PBMC dataset (FIG. 17G), supporting the reproducibility of the assay.

While cell-surface protein expression information provides a powerful method of studying immune cells, these methods are of limited value outside of the immunology field. To test whether a low-dimensional structure similar to that obtained using protein expression could be learned using the chromatin data alone, we compared the neighbor graphs obtained using protein expression data to that obtained using individual or combined chromatin modalities. While individual chromatin marks were unable to faithfully recapitulate the low-dimensional structure observed when including protein expression data, the combination of H3K27me3 and H3K27ac modalities provided a similar low-dimensional neighbor structure (FIG. 14F). This again highlights the unique power of multimodal chromatin data in resolving cellular states, and indicates that multiplexed NTT-seq may be a powerful method capable of characterizing heterogeneous tissues without the need for cell surface protein measurements.

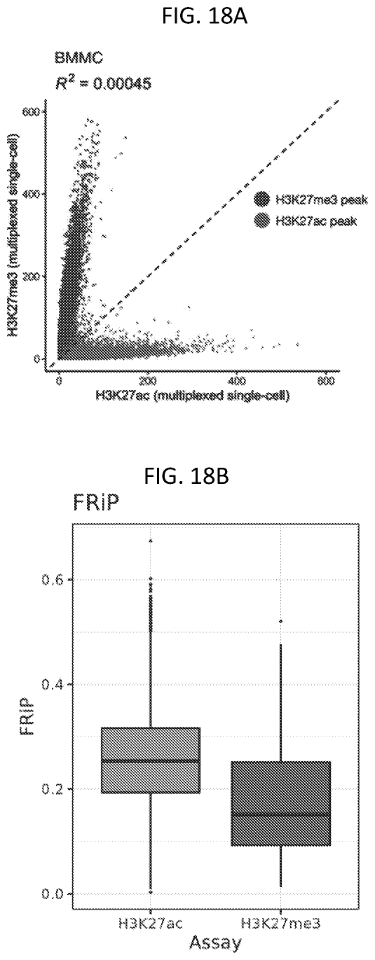

We next sought to apply NTT-seq in a complex tissue that contains differentiating cells to capture chromatin remodeling dynamics that shape cellular identity. We profiled H3K27me3 and H3K27ac in human bone marrow mononuclear cells (BMMCs) (FIG. 14G). This resulted in 5.236 cells with a mean of 1.217 and 326 fragments per cell for H3K27me3 and H3K27ac respectively (FIG. 14H). We annotated cell clusters using a combination of label transfer using an annotated BMMC scATAC-seq dataset using the H3K27ac assay, and manual annotation inspecting the presence of active and repressive histone marks at key marker genes for each cell type. We identified the expected cell types present in the immune system, including hematopoietic stem and progenitor cells (HSPCs) (FIG. 14G). Consistent with results obtained using cells in culture and PBMCs, we observed mutual exclusivity between H3K27ac and H3K27me3 across regions of the genome for BMMCs, and a mean fraction of fragments in ENCODE peaks of 0.18 and 0.26 for H3K27me3 and H3K27ac, respectively (FIG. 18A. FIG. 18B). To study how multimodal chromatin states may change during cell development, we ordered cells belonging to the B cell lineage, including HSPCs, common lymphoid progenitors (CLPs), pre-B, B, and plasma cells along a developmental pseudotime trajectory using Monocle 3 (FIG. 14I).

While the H3K27ac data were sparser than the H3K27me3 data, combining data from both modalities enabled a trajectory to be identified that revealed the expected ordering of cells in a trajectory leading from HSPCs through CLP, pre-B, B, and plasma cells. To identify regions of the genome that changed their H3K27me3 and H3K27ac state across this trajectory, we quantified fragment counts for each cell in 10 kb bins spanning the entire genome for each chromatin modality. We identified genome bins with signal correlated with pseudotime (Pearson correlation >0.2, Bonferroni-corrected p-value <1 e−08), and identified a set of 514 regions with opposing relationships between H3K27me3 and H3K27ac signal (>0.5 difference in Pearson correlation between the marks). Sorting these regions by the point at which they reached maximal H3K27me3 signal revealed an ordered sequence of sites that became repressed or activated during B cell development (FIG. 14J). The genome bin with the strongest gain in H3K27ac and loss of H3K27me3 signals across pseudotime was located at the PAX5 promoter (H3K27me3 r=−0.70. H3K27ac r=0.53). a B-cell-specific transcription factor. Of the 514 dynamic sites, we further identified 87 of these sites that displayed dynamic H3K27me3 and H3K27ac states across the B cell trajectory, but were static in their DNA accessibility profile (|r|<0.05. Bonferroni-corrected p>0.01), as quantified in an existing BMMC scATAC-seq dataset. This suggests that additional chromatin state dynamics can be identified using multimodal epigenomic data generated by scNTT-seq. Further experimental analysis will be required to fully characterize the function of these chromatin-dynamic sites in B cell development. To systematically assess the cell-type-specific expression pattern of genes located near genomic bins that were repressed or activated along the B cell pseudotime trajectory, we examined a published scRNA-seq dataset for healthy human BMMCs. We identified the closest gene to each pseudotime-correlated genome bin, and classified these as activated (positive correlation between H3K27ac and pseudotime) or repressed (positive correlation between H3K27me3 and pseudotime). Examining the expression of repressed and activated genes in the scRNA-seq dataset revealed concordant patterns of gene expression, with chromatin-activated genes becoming expressed later in B cell development, and repressed genes being expressed in HSPCs but turned off later in B cell development (p<2.2 e−16, t-test; FIG. 14K).

Together these analyses demonstrate that NTT-seq datasets provide accurate multimodal chromatin landscapes at single-cell resolution, contain sufficient information to identify major cell types and states in primary human tissues, and can be generated in conjunction with accurate cell-surface protein expression measurements. Our results demonstrate the high accuracy of multiplexed chromatin profiles obtained by NTT-seq in comparison to non-multiplexed CUT & Tag or ChIP-seq experiments. Existing multimodal chromatin technologies require complex experimental workflows and have not been demonstrated to work with complex tissue samples, or are strictly limited in the chromatin states that they can measure. NTT-seq overcomes both of these limitations, providing a streamlined experimental workflow applicable to complex tissues.

### Example 5: Spatially Resolved Capture of Chromatin Derived Material

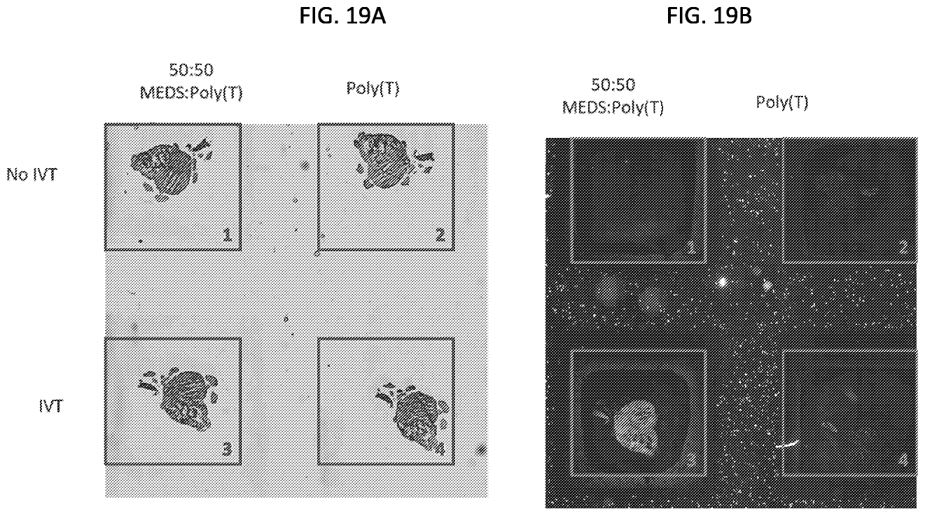

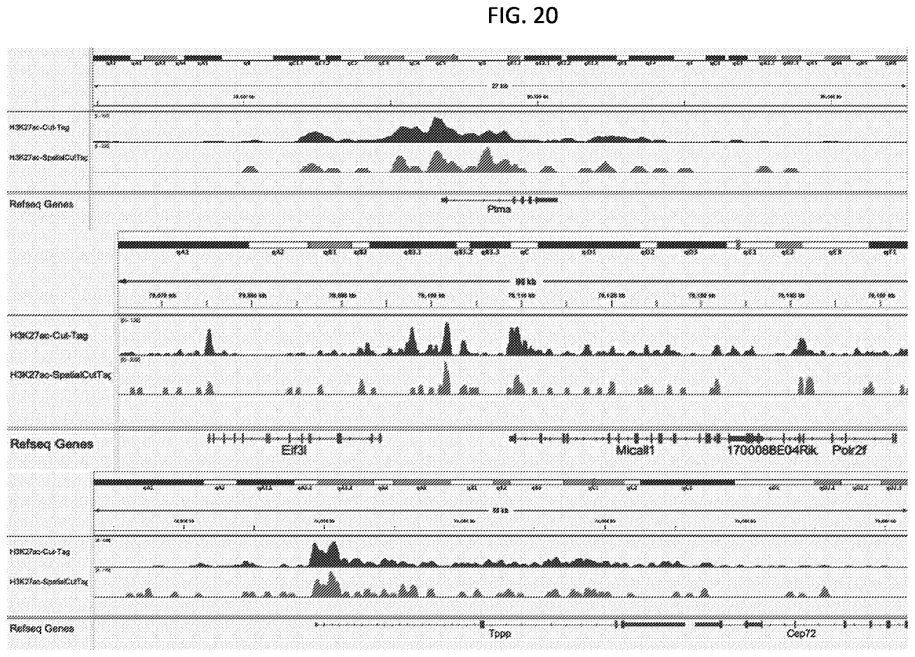

We performed a modified version of the “Tissue Optimization” workflow described in Stahl P L et al., Visualization and analysis of gene expression in tissue sections by spatial transcriptomics. Science. 2016 Jul. 1; 353(6294):78-82 (which is incorporated herein by reference), in which material from tagmentation of tissue chromatin is captured onto a glass slide and visualized by fluorescence microscopy. Briefly, fresh frozen mouse spinal cord tissue was sectioned onto a glass slide that was coated with DNA oligonucleotide capture probes. The tissue was fixed with methanol and stained with hematoxylin and eosin (H & E). The stained tissue was then imaged to capture the tissue morphology and orientation (FIG. 19A). The tissue was then gently permeabilized and subjected to tagmentation using MEDS that harbor a T7 RNA polymerase promoter, a capture sequence, and a sequence encoding a poly(A) tail. The resulting fragments are suitable for amplification via in vitro transcription (IVT) and the resulting IVT derived RNAs are compatible with slide capture. Following tagmentation, gap filling occurs via T4 DNA polymerase and T4 DNA ligase. Gap filled fragments were then subjected to IVT using T7 RNA polymerase. IVT derived RNAs hybridize with slide capture probes. Captured IVT derived RNAs were then reverse transcribed in the presence of a Cy3 labeled dCTP, yielding a fluorescent signal wherever cDNA has been captured (FIG. 19B). If the experiment is successful, the result should be a fluorescent signal matching the morphology of the tissue section as visualized via H & E imaging at the beginning of the experiment. In the above experiment, capture areas 1 & 3 harbor a 50:50 mixture of MEDS compatible capture probes and poly(T) capture probes, while capture areas 2 & 4 harbor only poly(T) capture probes. Further, T7 RNA polymerase was not added to capture areas 1 & 2, meaning that no IVT from tagmentation fragments occurred in these capture areas.

### Example 6: Spatially Resolved ATAC

Briefly, fresh frozen mouse spinal cord tissue is sectioned onto a glass slide that was coated with DNA oligonucleotide capture probes. The tissue is fixed with methanol and stained with hematoxylin and eosin (H & E). The stained tissue is then imaged to capture the tissue morphology and orientation. The tissue is then gently permeabilized and subjected to tagmentation using MEDS that harbor a T7 RNA polymerase promoter, optionally a target barcode, a capture compatible sequence, and a sequence encoding a poly(A) tail, and a sequence adapter/PCR handle.

GEMs are generated by combining barcoded Gel Beads, transposed nuclei, a Master Mix, and Partitioning Oil on a Chromium Next GEM Chip H. To achieve single nuclei resolution, the nuclei are delivered at a limiting dilution, such that the majority (˜90-99%) of generated GEMs contains no nuclei, while the remainder largely contain a single nucleus. Upon GEM generation, the Gel Bead is dissolved. Oligonucleotides containing (i) an Illumina P5 sequence, (ii) a 16 nt 10× Barcode and (iii) a Read 1 (Read IN) sequence are released and mixed with DNA fragments and Master Mix. Thermal cycling of the GEMs produces 10× barcoded single stranded DNA. After incubation, the GEMs are broken and pooled fractions are recovered. P7 and a sample index are added during library construction via PCR. The final libraries contain the P5 and P7 sequences used in Illumina bridge amplification. The Chromium Next GEM Single Cell ATAC Reagent Kits v1.1 protocol produces Illumina-ready sequencing libraries. Derived from the Chromium Next GEM Single Cell ATAC Reagent Kits v1.1 user guide.

### Example 7: Spatially Resolved IsCUT & Tag

Briefly, fresh frozen mouse spinal cord tissue is sectioned onto a glass slide that was coated with DNA oligonucleotide capture probes. The tissue is fixed with methanol and stained with hematoxylin and eosin (H & E). The stained tissue is then imaged to capture the tissue morphology and orientation. The tissue is then gently permeabilized and subjected to tagmentation using MEDS that harbor a T7 RNA polymerase promoter, optionally a target barcode, a capture sequence, and a sequence encoding a poly(A) tail, and a sequence adapter/PCR handle.

Buffers are the same as in Example 1, unless specified. Cell fixation and lysis. 2 million K562 cells were resuspended in 100 μl PBS, 3 μl 16% formaldehyde was added (0.1% final concentration) and incubated for 5 minutes at room temperature. Cells were swirled and inverted occasionally. Reaction was quenched by adding 40 μl 1.25 M glycine (to 0.125 M final concentration). Cells were spun for 5 minutes 800 g at 4° C. Supernatant was discarded and repeat wash with 1 ml 1× ice-cold PBS. Cells were spun for 5 minutes 800 g at 4° C., and supernatant discarded. The cell pellet was resuspended in 400 μl chilled lysis buffer, and mixed by pipetting, and incubated on ice for 7 minutes. The reaction was split into two tubes and 1 ml chilled wash buffer was added to the lysed cells, and mix by pipetting. The cells were spun for 5 minutes 1000 g at 4° C.

Primary antibody binding. Cells were resuspended with 200 μl antibody binding buffer in small PCR tube. 1.5 μl antibody K27me3 was added to each tube. Reaction was incubated overnight at 4° C. or 1 h RT.

Secondary antibody binding (optional). (There should be around 1.5 M cells at this step, no cell clumping can be seen after overnight incubation). The next day cells were spun for 5 mins 1300 g to remove the supernatant. The cells were resuspended in 150 μl Dig-150 buffer. 1.5 μl secondary antibody was added and incubated for 1 hour at room temperature. No wash was performed.

During secondary staining blocking oligo was annealed. 20 μl of blocking oligo (100 μM) was annealed in a thermocycler at 95° C. for 2 minutes, then 95° C. to 22° C.-0.01° C. per cycle.

### Blocking Oligo Sequence:

| | |
|---|---|
| TNY- | CGA UCG AUA AAA ACC CGC CUA UAU AGC |
| BLOCKER | GCU AUA UAG GCG GGU UUU UAU CGA UCG |
| | (SEQ ID NO: 24) |
| TN5- | UAU AUU UAU UUA AAC AGU UUU AAA CGT |
| BLOCKER | UUA AAA CUG UUU AAA UAA AUA UA |
| | (SEQ ID NO: 25) |

Tn5-adapter complex formation. Anneal each of Mosaic end-adapter A (ME-A) and Mosaic end-adapter B (ME-B) oligonucleotides with Mosaic end-reverse oligonucleotides. To anneal, dilute oligonucleotides to 200 μM in annealing buffer (10 mM Tris pH8, 50 mM NaCl, 1 mM EDTA). Each pair of oligos, ME-A+ME-Reverse and ME-B+ME-Reverse, is mixed separately resulting in 100 μM annealed product. Place the tubes in a 90-95° C. hot block and leave for 3-5 minutes, then remove the hot block from the heat source allowing for slow cooling to room temperature (˜45 minutes). Mix 16 μL of 100 μM equimolar mixtures of preannealed ME-A and ME-B oligonucleotides with 100 μL of 5.5 μM protein A-Tn5 fusion protein. Incubate the mixture on a rotating platform for 1 hour at room temperature and then store at −20° C. for up to 1 year.

pA-Tn5 blocking. 2 μl of pA-Tn5 (pre-loaded with MEDS) was added to 100 μl TAPS-BSA-Spermidine, and mixed by pipetting. 3 μl annealed blocking oligo was added and incubated at RT for 45 min-1 h.

Sample desalting. User Enzyme does not work in presence of NaCl, moreover salt can unblock the pA-Tn5. So, it is necessary remove the excess of NaCl by washing secondary stained cells. Thus, cells were washed one time with 150 μl Dig-150 buffer to remove Abs and washed 3 times with TAPS-BSA-Spermidine.

pA-Tn5 binding. The cells were resuspended in TAPS-BSA-Spermidine/pA-Tn5 blocked and incubated for 1 h at room temperature with slow rotation. Then, cells were centrifuged 5 minutes at 1500×g, and washed six times with 100 μl of TAPS-BSA-Spermidine.

pA-Tn5 Unblocking. Cells were resuspended cells in TAPS-BSA-Spermidine and 3 μl of USER enzyme was added and incubated for at 37° C. for 1 hr.

Tagmentation. 10 μl of 100 mM Mg2+ (or 10 μl 200 mM Co2+) was added to the cells to initiate tagmentation. The cells were incubated at 37° C. for 1 hr in an incubator, and centrifuged at 1400 g for 5 min. Nothing was used to stop the tagmentation. Supernatant was removed and then pellet was resuspended with 30 μl Nuclei buffer. The cell concentration of is around 4800/μl.

Loading to 10×. The Chromium Next GEM Single Cell ATAC Library & Gel Bead Kit v1.1, 10× Genomics was used. Mastermix was prepared: 8 μl nuclei suspension (in 1×PBS+1% BSA or 1×DNB+2% BSA), ATAC buffer B 7 μl, barcoding reagent B 56.5 μl, reducing agent B 1.5 μl, and barcoding enzyme 2 μl and chromium chip H loaded. 16-20 PCR cycles were used to perform the final library amplification according to Chromium Single Cell ATAC Library kit manual.

### Example 8: Spatially Resolved NTT-Seq

Briefly, fresh frozen mouse spinal cord tissue is sectioned onto a glass slide that was coated with DNA oligonucleotide capture probes. The tissue is fixed with methanol and stained with hematoxylin and eosin (H & E). The stained tissue is then imaged to capture the tissue morphology and orientation. The tissue is then gently permeabilized and subjected to tagmentation using MEDS that harbor a T7 RNA polymerase promoter, optionally a target barcode, a capture sequence, and a sequence encoding a poly(A) tail, and a sequence adapter/PCR handle.

Nb-Tn5 fusion proteins. Nanobody-Tn5 fusion proteins are produced using published protocols. For example, the plasmids exemplified herein utilize a chitin binding domain protein tag for purification of the fusion protein. A sample protocol is described by Mitchell, S. F., & Lorsch, J. R., Methods Enzymol. 2015:559:111-25, which is incorporated herein by reference. Briefly, the fusion protein comprising the nanobody, transposase and Intein/Chitin Binding Protein Tag is expressed in E. coli. The cells are harvested and lysed. The CBD domain fused to the intein sequence to is bound chitin beads on a column, washed, and cleaved. The cleaved protein is then eluted from the column. A separate preparation is performed for each nanobody-Tn fusion desired, including universal mouse, IgG1 mouse, IgG2a mouse, and IgG1 rabbit.

Nb-Tn5-adapter complex formation. Anneal each of Mosaic end-adapter A (ME-A) and Mosaic end-adapter B (ME-B) oligonucleotides with Mosaic end-reverse oligonucleotides. To anneal, dilute oligonucleotides to 200 μM in annealing buffer (10 mM Tris pH8, 50 mM NaCl, 1 mM EDTA). Each pair of oligos, ME-A+ME-Reverse and ME-B+ME-Reverse, is mixed separately resulting in 100 μM annealed product. MEDS harbor target barcode, optional UMI, PCR handle/sequencing adapter (e.g., R1 primer, R2 primer). Place the tubes in a 90-95° C. hot block and leave for 3-5 minutes, then remove the hot block from the heat source allowing for slow cooling to room temperature (˜45 minutes). Mix 16 μL of 100 μM equimolar mixtures of preannealed ME-A and ME-B oligonucleotides with 100 μL of 5.5 μM of each nb-Tn5 fusion protein. Incubate the mixture on a rotating platform for 1 hour at room temperature and then store at −20° C. for up to 1 year.

Bind antibodies. Incubate tissue with primary antibodies. As shown in FIG. 10 anti-H3K27me3 IgG1, anti-H3K27ac IgG2a, and anti-Pol2 rabbit antibodies were used.

Place on a Rotator at room temperature and incubate at least 1 hr. Wash with low salt wash buffer (from Example 1) and bind nb-Tn5 adapter complex. Mix equal amounts of each nb-Tn5 adapter complex in 300-wash buffer to a final concentration of 1:200. Incubate 50 μL per sample of the nb-Tn5 mix with tissue with gentle rocking. Place on a Rotator at room temperature for 1 hr. Wash with wash buffer.

Tagmentation. 10 μl of 100 mM Mg2+ (or 10 μl 200 mM Co2+) was added to the cells to initiate tagmentation. The cells were incubated at 37° C. for 1 hr in an incubator, and centrifuged at 1400 g for 5 min. Nothing was used to stop the tagmentation. Supernatant was removed and then pellet was resuspended with 30 μl Nuclei buffer. The cell concentration of is around 4800/μl.

Loading to 10×. The Chromium Next GEM Single Cell ATAC Library & Gel Bead Kit v1.1, 10× Genomics was used. Mastermix was prepared: 8 μl nuclei suspension (in 1×PBS+1% BSA or 1×DNB+2% BSA), ATAC buffer B 7 μl, barcoding reagent B 56.5 μl, reducing agent B 1.5 μl, and barcoding enzyme 2 μl and chromium chip H loaded. 16-20 PCR cycles were used to perform the final library amplification according to Chromium Single Cell ATAC Library kit manual.

All publications cited in this specification are incorporated herein by reference. U.S. Provisional Patent Application No. 63/276,533, filed Nov. 5, 2021, is incorporated herein by reference. While the invention has been described with reference to particular embodiments, it will be appreciated that modifications can be made without departing from the spirit of the invention. Such modifications are intended to fall within the scope of the appended embodiments.

1. A fusion protein comprising a transposase and a ligand that binds a target epitope.

2. The fusion protein of claim 1, wherein the ligand that binds the target epitope is an antibody or fragment thereof.

3. The fusion protein of claim 2, wherein the antibody or fragment thereof is a single domain antibody.

4. The fusion protein of claim 3, wherein the single domain antibody is a nanobody.

5. The fusion protein of claim 2, wherein the ligand that binds a target epitope is a G4 binding protein.

6. The fusion protein of any one of claims 1 to 5, wherein the transposase is a Tn5 or TnY transposase.

7. The fusion protein of claim any one of claims 1 to 6, further comprising a protein tag that allows for purification of the fusion protein during production.

8. The fusion protein of claim 7, wherein the protein tag is a chitin binding domain, FLAG, 6×-His, or GST.

9. A nucleic acid encoding the fusion protein of any one of claims 1 to 8.

10. A complex comprising the fusion protein of any one of claims 1 to 9 and a mosaic-end DNA sequence (MEDS) adapter that comprises one or more of:
a) a barcode sequence that identifies the target epitope of the ligand;
b) a unique molecular identifier (UMI);
c) a capture compatible sequence;
d) a PCR handle; and
e) a sequencing adapter.

11. A composition comprising a plurality of sets of the complexes of claim 10, each set of complexes comprising a different ligand that binds a different target epitope.

12. The composition of claim 11, wherein the different target epitope is on the same target.

13. The composition of claim 11, wherein the different target epitope is on a different target.

14. The composition of claim 11, comprising 10 or more complexes.

15. The composition of claim 11, comprising 50, 100, or more complexes.

16. The complex or composition of any one of claims 10 to 15, further comprising a double stranded DNA oligonucleotide having a sequence that is specific to the DNA sequence to which the transposase preferentially binds, wherein the T residues in the oligonucleotide are replaced with U residues.

17. The complex or composition of claim 16, wherein the DNA oligonucleotide is 40 to 70 nucleotides in length.

18. The complex or composition of any one of claims 10 to 15, further comprising a double stranded DNA oligonucleotide having a sequence that is specific to the DNA sequence to which the transposase preferentially binds.

19. An in vitro method for analyzing molecular interactions, the method comprising
a) incubating
i) a fusion protein comprising a transposase that preferentially binds to a DNA sequence, a ligand, and a mosaic-end DNA adapter; and
ii) a double stranded DNA oligonucleotide having a sequence that is specific to the DNA sequence to which the transposase preferentially binds, wherein the T residues in the oligonucleotide are replaced with U residues, wherein the double stranded DNA oligonucleotide binds the transposase, thereby preventing the transposase-ligand complex from binding DNA, and preventing tagmentation from occurring;
b) incubating a sample comprising genomic DNA that comprises chromatin with a primary antibody directed to a target epitope in the chromatin, and said antibody binds said epitope if it is present in the sample;
c) incubating the complex of A with the complex of B, wherein the ligand of the fusion protein binds the primary antibody;
d) degrading or displacing the double stranded DNA oligonucleotide;
e) activating tagmentation, thereby generating genomic DNA which has been tagmented.

20. The method according to claim 19, further comprising one or more of:
f) performing in vitro transcription comprising contacting and incubating the tagmented DNA of E with poly A polymerase, thereby generating polyadenylated RNAs that comprise the sequence of the tagmentation fragment;
g) performing reverse transcription to generate DNA; and
h) sequencing DNA.

21. The method according to claim 19 or 20, wherein the MEDS comprise one or more of:
a) a barcode sequence that identifies the target epitope;
b) a unique molecular identifier (UMI);
c) capture compatible sequence;
d) PCR handle; and
e) sequencing adapter.

22. The method according to any one of claims 19 to 21, wherein tagmentation is activated by addition of Cobalt or Mg²⁺.

23. The method according to any one of claims 19 to 22, wherein step d) comprises incubating the complex of C with a USER enzyme cocktail to cleave the U residues in the DNA oligonucleotide, thereby removing the blocking double stranded DNA oligonucleotide, and allowing tagmentation to occur;

24. The method according to any one of claims 19 to 22, wherein the double stranded DNA oligonucleotide is displaced by addition of 50 to 150 nM NaCl solution.

25. The method according to any one of claims 19 to 24, wherein the fusion protein comprises a nanobody and a transposase.

26. The method according to any one of claims 19 to 25, wherein the fusion protein comprises the fusion protein of any one of claims 1 to 6.

27. The method according to any one of claims 19 to 25, wherein the sample comprises a single cell, or a single cell nucleus.

28. The method of claim 27, further comprising one or more of
d) capturing the tagmented sequences using a capture sequence;
e) performing PCR; and
f) performing sequencing.

29. The method according to any one of claims 19 to 25, wherein the sample comprises a tissue section.

30. The method of claim 27, further comprising one or more of
d) capturing the tagmented sequences using a capture sequence;
e) performing PCR; and
f) performing sequencing.

31. A multiplexed in vitro method for analyzing molecular interactions, the method comprising
a) incubating a sample comprising genomic DNA that comprises chromatin with a plurality of primary antibodies, each primary antibody directed to a different target epitope in the chromatin, wherein each antibody binds to the target epitope if it is present in the sample;
b) incubating the complex of a) with a composition comprising plurality of fusion proteins, each fusion protein comprising a different nanobody and a transposase that preferentially binds to a DNA sequence, and mosaic-end DNA (MEDS) adapters, wherein each different nanobody binds a different primary antibody; and
c) activating tagmentation, thereby generating genomic DNA which has been tagmented.

32. The method according to claim 31, wherein the MEDS comprise one or more of:
a) a barcode sequence that identifies the target epitope;
b) a unique molecular identifier (UMI);
c) capture compatible sequence;
d) PCR handle; and

33. The method of claim 29, further comprising one or more of
d) capturing the tagmented sequences using a capture sequence;
e) performing PCR; and
f) performing sequencing.

34. The method according to any one of claims 31 to 33, wherein the fusion protein comprises the fusion protein of any one of claims 1 to 6.

35. The method according to any one of claims 31 to 34, wherein the sample comprises a single cell, or a single cell nucleus.

36. The method according to any one of claims 31 to 34, wherein the sample comprises a tissue section.

37. An in vitro method of spatially resolved whole genome sequencing, the method comprising
a) sectioning a tissue sample onto a substrate comprising substrate oligonucleotides comprising a capture sequence;
b) fixing the tissue and performing imaging to determine morphology and/or orientation of the tissue;
c) permeabilizing the tissue;
d) subjecting the tissue to tagmentation using a transposase loaded with MEDS that comprise T7 RNA polymerase promoter, a capture compatible sequence, and a sequence encoding a poly(A) tail;
e) performing in vitro transcription to result in IVT-derived RNA
f) capturing the IVT-derived RNA;
g) generating cDNA from the IVT-derived RNA using fluorescently labeled dNTPs to generate a fluorescent signal wherever cDNA has been captured.

38. The method according to claim 37, further comprising performing gap filling.

39. An in vitro method of spatially resolved ATAC, the method comprising
a) sectioning a tissue sample onto a substrate comprising substrate oligonucleotides comprising a capture sequence;
b) fixing the tissue and performing imaging to determine morphology and/or orientation of the tissue;
c) permeabilizing the tissue;
d) subjecting the tissue to tagmentation using a transposase loaded with MEDS that comprise T7 RNA polymerase promoter, optionally a target barcode, a capture compatible sequence, a sequence encoding a poly(A) tail, and a PCR handle, which is optionally a sequence adapter;
e) performing in vitro transcription to result in IVT-derived RNA;
f) capturing the IVT-derived RNA;
g) generating cDNA from the IVT-derived RNA using fluorescently labeled dNTPs to generate a fluorescent signal wherever cDNA has been captured.

40. The method according to claim 39, further comprising performing gap filling.

41. The method according to claim 39, further comprising
i) partitioning the nuclei into beads;
ii) barcoding tagmented DNA;
iii) generating sequencing library; and/or
iv) performing single cell sequencing.

42. A spatially resolved method for analyzing molecular interactions, the method comprising
a) incubating
i) a fusion protein comprising a transposase that preferentially binds to a DNA sequence, a ligand, and mosaic-end DNA adapters that comprise T7 RNA polymerase promoter, optionally a target barcode, a capture compatible sequence, a sequence encoding a poly(A) tail, and a PCR handle, which is optionally a sequence adapter; and
ii) a double stranded DNA oligonucleotide having a sequence that is specific to the DNA sequence to which the transposase preferentially binds, wherein the T residues in the oligonucleotide are replaced with U residues, wherein the double stranded DNA oligonucleotide binds the transposase, thereby preventing the transposase-ligand complex from binding DNA, and preventing tagmentation from occurring;
b) sectioning a tissue sample onto a substrate comprising substrate oligonucleotides comprising a capture sequence;
c) fixing the tissue and performing imaging to determine morphology and/or orientation of the tissue;
d) permeabilizing the tissue;
e) incubating the tissue with a primary antibody directed to a target epitope in the chromatin, wherein said antibody binds said epitope if it is present in the sample;
f) incubating the complex of a) with the tissue sample, wherein the ligand of the fusion protein binds the primary antibody;
g) degrading or displacing the double stranded DNA oligonucleotide; and
e) activating tagmentation, thereby generating genomic DNA which has been tagmented.

43. The method according to claim 42, further comprising
f) performing in vitro transcription to result in IVT-derived RNA;
g) capturing the IVT-derived RNA; and
h) generating cDNA from the IVT-derived RNA using fluorescently labeled dNTPs to generate a fluorescent signal wherever cDNA has been captured.

44. The method according to claim 42 or 43, further comprising performing gap filling.

45. The method according to any of claims 42 to 44, further comprising
i) partitioning the nuclei into beads;
ii) barcoding tagmented DNA;
iii) generating sequencing library; and/or
iv) performing single cell sequencing.

46. The method according to any one of claims 42 to 45, wherein tagmentation is activated by addition of Cobalt or Mg²⁺.

47. The method according to any one of claims 42 to 46, wherein step d) comprises incubating the complex of C with a USER enzyme cocktail to cleave the U residues in the DNA oligonucleotide, thereby removing the blocking double stranded DNA oligonucleotide, and allowing tagmentation to occur.

48. The method according to any one of claims 42 to 46, wherein the double stranded DNA oligonucleotide is displaced by addition of 50 to 150 nM NaCl solution.

49. A spatially resolved method for analyzing molecular interactions, the method comprising
a) sectioning a tissue sample onto a substrate comprising substrate oligonucleotides comprising a capture sequence;
b) fixing the tissue and performing imaging to determine morphology and/or orientation of the tissue;
c) permeabilizing the tissue;
d) incubating the tissue with a plurality of primary antibodies, each primary antibody directed to a different target epitope in the chromatin, wherein each antibody binds to the target epitope if it is present in the sample;
e) incubating the tissue with a composition comprising plurality of fusion proteins, each fusion protein comprising a different nanobody and a transposase that preferentially binds to a DNA sequence, and mosaic-end DNA (MEDS) adapters that comprise T7 RNA polymerase promoter, optionally a target barcode, a capture compatible sequence, a sequence encoding a poly(A) tail, and a PCR handle, which is optionally a sequence adapter, wherein each different nanobody binds a different primary antibody; and
f) activating tagmentation, thereby generating genomic DNA which has been tagmented.

50. The method according to claim 49, further comprising
g) performing in vitro transcription to result in IVT-derived RNA;
h) capturing the IVT-derived RNA; and
i) generating cDNA from the IVT-derived RNA using fluorescently labeled dNTPs to generate a fluorescent signal wherever cDNA has been captured.

51. The method according to claim 49 or 50, further comprising performing gap filling.

52. The method according to any of claims 49 to 51, further comprising
i) partitioning the nuclei into beads;
ii) barcoding tagmented DNA;
iii) generating sequencing library; and/or
iv) performing single cell sequencing.


SEQUENCE LISTING (37 sequences)

SEQ ID NO: 1 - DNA, 1428 bp, synthetic construct
  source 1..1428: mol_type = other DNA
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatc

SEQ ID NO: 2 - protein, 476 aa, synthetic construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 3 - DNA, 1376 bp, synthetic construct
  source 1..1376: mol_type = other DNA
atgacccactccgatgcgaaactgtgggctcaggagcaattcggtcaggcccaactgaaagatccgcgcccacccagcgcctgatttctctggcgaccagcattgctaaccagccgggtgttagcgttgcgaaactgccgttttctaaagccgatcaggagggcgcgtaccgtttcattcgtaacgataacatcgacgcgaaagacatcgctgaagcaggctttcagtccaccgtatcccgcgctaacgaacacaaagagctgctggcgctggaagacactacgaccctgtctttcccgcatcgttccatcaaagaagaactgggccatacgaaccagggtgatcgcacccgcgccctgcacgttcactctaccctgctgttcgcgccgcagaaccagactatcgtgggtctgatcgagcagcagcgttggtctcgtgatattactaaacgcggtcagaaacatcagcacgctacccgtccttataaagaaaaagaatcctataaatgggagcaggcttcccgtcgtgttgtggagcgcctgggtgataaaatgctggatgtcatttctgtttgcgaccgcgaggcagatctgtttgaatacctgacctacaaacgtcaacaccagcagcgtttcgttgttcgtagcatgcagtctcgctgtctggaagaacacgctcagaaactgtatgactacgcacaggcgctgccatctgtaaaaacgaaggcactgaccatccctcaaaaaggtggccgtaaagcacgtgacgttaaactggacgttaaatacggccaggttactctgaaagcgccggccaacaaaaaggagcacgcaggcattccggtttactacgtgggctgcctggaacagggtacttccaaagataaactggcgtggcacctgctgacctctgaacctattaacaacgtcgaggatgccatgcgtatcatcggctactacgaacgtcgttggctgatcgaggattttcacaaagtatggaaatccgaaggtactgacgtagaatccctgcgtctgcagagcaaagacaacctggaacgtctgtccgttatctacgcgtttgttgctacccgcctgctggcactgcgttttatcaaggaagttgatgaactgaccaaagaaagctgtgaaaaagttctgggccagaaagcgtggaaactgctgtggctgaagctggaatctaaaaccctgccgaaagaggtaccggacatgggttgggcttataaaaacctggctaaactgggtggctggaaggacactaagcgtaccggtcgcgcttctatcaaagttctgtgggagggttggttcaaactgcagaccatcctggagggctatgaactggcgatgtccctggaccac

SEQ ID NO: 4 - protein, 459 aa, synthetic construct
MTHSDAKLWAQEQFGQAQLKDPRRTQRLISLATSIANQPGVSVAKLPFSKADQEGAYRFIRNDNIDAKDIAEAGFQSTVSRANEHKELLALEDTTTLSFPHRSIKEELGHTNQGDRTRALHVHSTLLFAPQNQTIVGLIEQQRWSRDITKRGQKHQHATRPYKEKESYKWEQASRRVVERLGDKMLDVISVCDREADLFEYLTYKRQHQQRFVVRSMQSRCLEEHAQKLYDYAQALPSVKTKALTIPQKGGRKARDVKLDVKYGQVTLKAPANKKEHAGIPVYYVGCLEQGTSKDKLAWHLLTSEPINNVEDAMRIIGYYERRWLIEDFHKVWKSEGTDVESLRLQSKDNLERLSVIYAFVATRLLALRFIKEVDELTKESCEKVLGQKAWKLLWLKLESKTLPKEVPDMGWAYKNLAKLGGWKDTKRTGRASIKVLWEGWFKLQTILEGYELAMSLDH

SEQ ID NO: 5 - protein, 65 aa, Photorhabdus luminescens
MFSTSAEQWANDTFQHAELGDKRRTNRLVKVACSLANHIGQSLVQSLDSPADVEAAYRLTRNSAI

SEQ ID NO: 6 - protein, 63 aa, synthetic construct
MDPEQWAQCQFGHANLNDPRRTQRLVSLATSITQQPGVAVSKLPLSPAEMEGAYRFIRNENIQ

SEQ ID NO: 7 - protein, 66 aa, Vibrio campbellii
MTHSDAKLWAQEQFGQAQLKDPRRTQRLISLATSIANQPGVSVAKLPFSPADMEGAYRFIRNENIN

SEQ ID NO: 8 - protein, 66 aa, Vibrio parahaemolyticus
MTHSDAKLWAQEQFGQAQLKDPRRTQRLISLATSIANQPGVSVAKLPFSPADMEGAYRFIRNDNID

SEQ ID NO: 9 - protein, 70 aa, synthetic construct
MITSALHRAADWAKSVFSSAALGDPRRTARLVNVAAQLAKYSGKSITISSEGSKAMQEGAYRFIRNPNVS

SEQ ID NO: 10 - protein, 67 aa, Candidatus glomeribacter
MFRREAGDWAHQTFGECNLGDERRTKRLVEVGKRLANQIGCSLPKCCEGDKAALLGSYRLLRNDAVN

SEQ ID NO: 11 - protein, 69 aa, Legionella longbeachae
MDLAIEDAAAWSEAIFGSVDLGDKRLTRRLTQIGKQLSSMPGGSLPESCEGQDALIEGSYRFLRNKRVT

SEQ ID NO: 12 - protein, 69 aa, Legionella pneumophila
MDLAIEDAAAWSEAIFGSVALGDKRLTRRLIQIGKQLSSIPGGSLSESCEGQDALIEGSYRFLRNKRVT

SEQ ID NO: 13 - DNA, 2652 bp, synthetic construct
  source 1..2652: mol_type = other DNA
atgggtcaggtgcaattggtggagtctgggggaggcttggtgcagcctggggggtctctgaggctctcctgtgcagcctctggattcacattcagtgatacctggatgaactgggtccgccaggctccagggaaggggctctattggatctcagccatcaatccggacggtggtaacacagcctatgcagactccgtcaagggccggttcaccatctccagagacaacgccaagaacatggtgtatctgcagatggacaacctgagacctgaggacacggccatgtattactgcgcaaagggttgggttcgccttccagatcccgatctggtaaggggccaggggacccaggtcacagtctcctccggtggatgtgaattcggtggcggtggctctggcggtggtgggagtggaggtgggggatcaggaggaggcggttcccatatgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcgggaaacccgaatttgcgcccacaacctacacagtcggcgtccctggactggtgcgtttcttggaagcacaccaccgagacccggacgcccaagctatcgccgacgagctgaccgacgggcggttctactacgcgaaagtcgccagtgtcaccgacgccggcgtgcagccggtgtatagccttcgtgtcgacacggcagaccacgcgtttatcacgaacgggttcgtcagccacgctactggcctcaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaa

SEQ ID NO: 14 - DNA, 2667 bp, synthetic construct
  source 1..2667: mol_type = other DNA
atgggtcaggtgcaattggtagagtgtgggggaggcttggtgcagcctggggggtctctgagactctcctgtgtagcctctggattcaccttcagtagcgcctacatgagctgggtccgccaggctccaggaaaggggcccgagtgggtctcaactattagtacaggtggtggtatcgtaaactatgcagactccgtgaagggccgattcgccatctccagagacaacgccaagaacaccctgtatctgcaaatgaacaaactgaaacctgaggacacggccctctattactgtgcgagcaataaaggcccgcattaccatagcgactattttgattcgaatctgtatgacttctggggccaggggacccgggtcacagtctcatcatgtgaattcggtggcggtggctctggcggtggtgggagtggaggtgggggatcaggaggaggcggttcccatatgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcgggaaacccgaatttgcgcccacaacctacacagtcggcgtccctggactggtgcgtttcttggaagcacaccaccgagacccggacgcccaagctatcgccgacgagctgaccgacgggcggttctactacgcgaaagtcgccagtgtcaccgacgccggcgtgcagccggtgtatagccttcgtgtcgacacggcagaccacgcgtttatcacgaacgggttcgtcagccacgctactggcctcaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaa

SEQ ID NO: 15 - DNA, 2640 bp, synthetic construct
  source 1..2640: mol_type = other DNA
atgggtcaggtgcaattggtagagtctgggggaggcttggtgcaggctggtgactctctgcgactctcctgtgtagcctctgggcgctccttggatggggccaccatgcgctggtaccgccaggctccagggaaggagcgagaatttgttgcaggcattttttgggatgagattggtacggaatatgcagacactgcgaagggccgattcaccatctccagagacaatgccaagaacacgatatatctacaaatgaccaacctgaggtccgaagacacggccatgtattattgtaatggattagtattcgggggtgaatactggggacaggggacccaggtcacagtctcctccggtggatgtgaattcggtggcggtggctctggcggtggtgggagtggaggtgggggatcaggaggaggcggttcccatatgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcgggaaacccgaatttgcgcccacaacctacacagtcggcgtccctggactggtgcgtttcttggaagcacaccaccgagacccggacgcccaagctatcgccgacgagctgaccgacgggcggttctactacgcgaaagtcgccagtgtcaccgacgccggcgtgcagccggtgtatagccttcgtgtcgacacggcagaccacgcgtttatcacgaacgggttcgtcagccacgctactggcctcaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaa

SEQ ID NO: 16 - DNA, 2670 bp, synthetic construct
  source 1..2670: mol_type = other DNA
atgggtcaggtgcaattggtagagtctgggggaggctgggtgcagcctggggggtctctgagactctcctgtgcagcctctggattcaccttcagtgacactgccatgatgtgggtccgccaggctccagggaaggggcgagagtgggtcgcagctattgatactggtggtggatacacatactatgcagactccgtgaagggccgattcaccatctccagagacaacgccaagaacacgctgtatctgcaaatgaacagcctgaaaccggaggacacggcccgatactactgtgcgaagacctactccggtaattactatagcaactacacggttgccaactatggcaccacgggccgggggaccctggtcacagtctcctccggtggatgtgaattcggtggcggtggctctggcggtggtgggagtggaggtgggggatcaggaggaggcggttcccatatgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcgggaaacccgaatttgcgcccacaacctacacagtcggcgtccctggactggtgcgtttcttggaagcacaccaccgagacccggacgcccaagctatcgccgacgagctgaccgacgggcggttctactacgcgaaagtcgccagtgtcaccgacgccggcgtgcagccggtgtatagccttcgtgtcgacacggcagaccacgcgtttatcacgaacgggttcgtcagccacgctactggcctcaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaa

SEQ ID NO: 17 - DNA, 19 bp, synthetic construct
  source 1..19: mol_type = other DNA
agatgtgtataagagacag

SEQ ID NO: 18 - DNA, 19 bp, synthetic construct
  source 1..19: mol_type = other DNA
ctgactcttatacacaagt

SEQ ID NO: 19 - DNA, 19 bp, synthetic construct
  source 1..19: mol_type = other DNA
ctgtctcttgatcagatct

SEQ ID NO: 20 - DNA, 33 bp, synthetic construct
  source 1..33: mol_type = other DNA
tcgtcggcagcgtcagatgtgtataagagacag

SEQ ID NO: 21 - DNA, 34 bp, synthetic construct
  source 1..34: mol_type = other DNA
gtctcgtgggctcggagatgtgtataagagacag

SEQ ID NO: 22 - DNA, 29 bp, synthetic construct
  source 1..29: mol_type = other DNA
aatgatacggcgaccaccgagatctacac

SEQ ID NO: 23 - DNA, 24 bp, synthetic construct
  source 1..24: mol_type = other DNA
caagcagaagacggcatacgagat

SEQ ID NO: 24 - DNA, 54 bp, synthetic construct
  source 1..54: mol_type = other DNA
  modified_base 4: mod_base = OTHER; note = uracil
  modified_base 8: mod_base = OTHER; note = uracil
  modified_base 20: mod_base = OTHER; note = uracil
  modified_base 22: mod_base = OTHER; note = uracil
  modified_base 24: mod_base = OTHER; note = uracil
  modified_base 30: mod_base = OTHER; note = uracil
  modified_base 32: mod_base = OTHER; note = uracil
  modified_base 34: mod_base = OTHER; note = uracil
  modified_base 42: mod_base = OTHER; note = uracil
  modified_base 43: mod_base = OTHER; note = uracil
  modified_base 44: mod_base = OTHER; note = uracil
  modified_base 45: mod_base = OTHER; note = uracil
  modified_base 46: mod_base = OTHER; note = uracil
  modified_base 48: mod_base = OTHER; note = uracil
  modified_base 52: mod_base = OTHER; note = uracil
cgatcgataaaaacccgcctatatagcgctatataggcgggtttttatcgatcg

SEQ ID NO: 25 - DNA, 50 bp, synthetic construct
  source 1..50: mol_type = other DNA
  modified_base 1: mod_base = OTHER; note = uracil
  modified_base 3: mod_base = OTHER; note = uracil
  modified_base 5: mod_base = OTHER; note = uracil
  modified_base 6: mod_base = OTHER; note = uracil
  modified_base 7: mod_base = OTHER; note = uracil
  modified_base 9: mod_base = OTHER; note = uracil
  modified_base 10: mod_base = OTHER; note = uracil
  modified_base 11: mod_base = OTHER; note = uracil
  modified_base 18: mod_base = OTHER; note = uracil
  modified_base 19: mod_base = OTHER; note = uracil
  modified_base 20: mod_base = OTHER; note = uracil
  modified_base 21: mod_base = OTHER; note = uracil
  modified_base 27: mod_base = OTHER; note = uracil
  modified_base 28: mod_base = OTHER; note = uracil
  modified_base 29: mod_base = OTHER; note = uracil
  modified_base 35: mod_base = OTHER; note = uracil
  modified_base 37: mod_base = OTHER; note = uracil
  modified_base 38: mod_base = OTHER; note = uracil
  modified_base 39: mod_base = OTHER; note = uracil
  modified_base 43: mod_base = OTHER; note = uracil
  modified_base 47: mod_base = OTHER; note = uracil
  modified_base 49: mod_base = OTHER; note = uracil
tatatttatttaaacagttttaaacgtttaaaactgtttaaataaatata

SEQ ID NO: 26 - DNA, 19 bp, synthetic construct
  source 1..19: mol_type = other DNA
ctgtctcttatacacatct

SEQ ID NO: 27 - DNA, 73 bp, synthetic construct
  source 1..73: mol_type = other DNA
tcgtcggcagcgtcagatgtgtataagagacagnnnnnnnnnvtttttttttttttttttttttttttttttt

SEQ ID NO: 28 - DNA, 65 bp, synthetic construct
  source 1..65: mol_type = other DNA
caagcagaagacggcatacgagatnnnnnnnngtgactggagttccttggcacccgagaattcca

SEQ ID NO: 29 - DNA, 64 bp, synthetic construct
  source 1..64: mol_type = other DNA
tcgtcggcagcgtcggattgctgcgatcgaggacggcagatgtgtataagagacagggattgct

SEQ ID NO: 30 - DNA, 64 bp, synthetic construct
  source 1..64: mol_type = other DNA
tcgtcggcagcgtcgtaatgcagcgatcgaggacggcagatgtgtataagagacaggtaatgca

SEQ ID NO: 31 - DNA, 64 bp, synthetic construct
  source 1..64: mol_type = other DNA
tcgtcggcagcgtcgtcaaggagcgatcgaggacggcagatgtgtataagagacaggtcaagga

SEQ ID NO: 32 - DNA, 64 bp, synthetic construct
  source 1..64: mol_type = other DNA
tcgtcggcagcgtcgtgagcgtgcgatcgaggacggcagatgtgtataagagacaggtgagcgt

SEQ ID NO: 33 - DNA, 64 bp, synthetic construct
  source 1..64: mol_type = other DNA
tcgtcggcagcgtcgtgtgaccgcgatcgaggacggcagatgtgtataagagacaggtgtgacc

SEQ ID NO: 34 - DNA, 64 bp, synthetic construct
  source 1..64: mol_type = other DNA
tcgtcggcagcgtctaaggtgggcgatcgaggacggcagatgtgtataagagacagtaaggtgg

SEQ ID NO: 35 - DNA, 34 bp, synthetic construct; identical to SEQ ID NO: 21
  source 1..34: mol_type = other DNA

SEQ ID NO: 36 - DNA, 34 bp, synthetic construct
  source 1..34: mol_type = other DNA
gcgatcgaggacggcagatgtgtataagagacag

SEQ ID NO: 37 - DNA, 34 bp, synthetic construct
  source 1..34: mol_type = other DNA
ctgtctcttatacacatctgccgtcctcgatcgc